SEQUENCE LISTING (93 sequences)

SEQ ID NO: 1 - protein, 614 aa, Homo sapiens
MSGYSSDRDRGRDRGFGAPRFGGSRAGPLSGKKFGNPGEKLVKKKWNLDELPKFEKNFYQEHPDLARRTAQEVETYRRSKEITVRGHNCPKPVLNFYEANFPANVMDVIARQNFTEPTAIQAQGWPVALSGLDMVGVAQTGSGKTLSYLLPAIVHINHQPFLERGDGPICLVLAPTRELAQQVQQVAAEYCRACRLKSTCIYGGAPKGPQIRDLERGVEICIATPGRLIDFLECGKTNLRRTTYLVLDEADRMLDMGFEPQIRKIVDQIRPDRQTLMWSATWPKEVRQLAEDFLKDYIHINIGALELSANHNILQIVDVCHDVEKDEKLIRLMEEIMSEKENKTIVFVETKRRCDELTRKMRRDGWPAMGIHGDKSQQERDWVLNEFKHGKAPILIATDVASRGLDVEDVKFVINYDYPNSSEDYIHRIGRTARSTKTGTAYTFFTPNNIKQVSDLISVLREANQAINPKLLQLVEDRGSGRSRGRGGMKDDRRDRYSAGKRGGFNTFRDRENYDRGYSSLLKRDFGAKTQNGVYSAANYTNGSFGSNFVSAGIQTSFRTGNPTGTYQNGYDSTQQYGSNVPNMHNGMNQQAYAYPATAAAPMIGYPMPTGYSQ

SEQ ID NO: 2 - DNA, 1845 bp, Homo sapiens
  source 1..1845: mol_type = genomic DNA
atgtcgggttattcgagtgaccgagaccgcggccgggaccgagggtttggtgcacctcgatttggaggaagtagggcagggcccttatctggaaagaagtttggaaaccctggggagaaattagttaaaaagaagtggaatcttgatgagctgcctaaatttgagaagaatttttatcaagagcaccctgatttggctaggcgcacagcacaagaggtggaaacatacagaagaagcaaggaaattacagttagaggtcacaactgcccgaagccagttctaaatttttatgaagccaatttccctgcaaatgtcatggatgttattgcaagacagaatttcactgaacccactgctattcaagctcagggatggccagttgctctaagtggattggatatggttggagtggcacagactggatctgggaaaacattgtcttatttgcttcctgccattgtccacatcaatcatcagccattcctagagagaggcgatgggcctatttgtttggtgctggcaccaactcgggaactggcccaacaggtgcagcaagtagctgctgaatattgtagagcatgtcgcttgaagtctacttgtatctacggtggtgctcctaagggaccacaaatacgtgatttggagagaggtgtggaaatctgtattgcaacacctggaagactgattgactttttagagtgtggaaaaaccaatctgagaagaacaacctaccttgtccttgatgaagcagatagaatgcttgatatgggctttgaaccccaaataaggaagattgtggatcaaataagacctgataggcaaactctaatgtggagtgcgacttggccaaaagaagtaagacagcttgctgaagatttcctgaaagactatattcatataaacattggtgcacttgaactgagtgcaaaccacaacattcttcagattgtggatgtgtgtcatgacgtagaaaaggatgaaaaacttattcgtctaatggaagagatcatgagtgagaaggagaataaaaccattgtttttgtggaaaccaaaagaagatgtgatgagcttaccagaaaaatgaggagagatgggtggcctgccatgggtatccatggtgacaagagtcaacaagagcgtgactgggttctaaatgaattcaaacatggaaaagctcctattctgattgctacagatgtggcctccagagggctagatgtggaagatgtgaaatttgtcatcaattatgactaccctaactcctcagaggattatattcatcgaattggaagaactgctcgcagtaccaaaacaggcacagcatacactttctttacacctaataacataaagcaagtgagcgaccttatctctgtgcttcgtgaagctaatcaagcaattaatcccaagttgcttcagttggtcgaagacagaggttcaggtcgttccaggggtagaggaggcatgaaggatgaccgtcgggacagatactctgcgggcaaaaggggtggatttaatacctttagagacagggaaaattatgacagaggttactctagcctgcttaaaagagattttggggcaaaaactcagaatggtgtttacagtgctgcaaattacaccaatgggagctttggaagtaattttgtgtctgctggtatacagaccagttttaggactggtaatccaacagggacttaccagaatggttatgatagcactcagcaatacggaagtaatgttccaaatatgcacaatggtatgaaccaacaggcatatgcatatcctgctactgcagctgcacctatgattggttatccaatgccaacaggatattcccaataa

SEQ ID NO: 3 - protein, 729 aa, Homo sapiens
MPTGFVAPILCVLLPSPTREAATVASATGDSASERESAAPAAAPTAEAPPPSVVTRPEPQALPSPAIRAPLPDLYPFGTMRGGGFGDRDRDRDRGGFGARGGGGLPPKKFGNPGERLRKKKWDLSELPKFEKNFYVEHPEVARLTPYEVDELRRKKEITVRGGDVCPKPVFAFHHANFPQYVMDVLMDQHFTEPTPIQCQGFPLALSGRDMVGIAQTGSGKTLAYLLPAIVHINHQPYLERGDGPICLVLAPTRELAQQVQQVADDYGKCSRLKSTCIYGGAPKGPQIRDLERGVEICIATPGRLIDFLESGKTNLRRCTYLVLDEADRMLDMGFEPQIRKIVDQIRPDRQTLMWSATWPKEVRQLAEDFLRDYTQINVGNLELSANHNILQIVDVCMESEKDHKLIQLMEEIMAEKENKTIIFVETKRRCDDLTRRMRRDGWPAMCIHGDKSQPERDWVLNEFRSGKAPILIATDVASRGLDVEDVKFVINYDYPNSSEDYVHRIGRTARSTNKGTAYTFFTPGNLKQARELIKVLEEANQAINPKLMQLVDHRGGGGGGGGRSRYRTTSSANNPNLMYQDECDRRLRGVKDGGRRDSASYRDRSETDRAGYANGSGYGSPNSAFGAQAGQYTYGQGTYGAAAYGTSSYTAQEYGAGTYGASSTTSTGRSSQSSSQQFSGIGRSGQQPQPLMSQQFAQPPGATNMIGYMGQTAYQYPPPPPPPPPSRK

SEQ ID NO: 4 - DNA, 2190 bp, Homo sapiens
  source 1..2190: mol_type = genomic DNA
atgcccaccggctttgtagccccgattctctgtgttttgctcccgtctccgacgagagaggcggcgacggtggcgtctgcgacgggagacagcgcgtcggagcgagagagcgctgcgcctgccgccgccccaacagcggaggcgccgccgccatcggtcgtcaccagaccggagccgcaggccctcccgagcccggccatccgtgccccgctcccagatctctatccttttgggaccatgcgcggaggaggctttggggaccgggaccgggatcgtgaccgtggaggatttggagcaagaggtggtggtggccttcccccgaagaaatttggtaatcctggggagcgtttgcgtaaaaaaaagtgggatttgagtgagctccccaagtttgagaaaaatttttatgtggaacatccggaagtagcaaggctgacaccatatgaggttgatgagctacgccgaaagaaggagattacagtgagggggggagatgtttgtcctaaacccgtgtttgccttccatcatgctaacttcccacaatatgtaatggatgtgttgatggatcagcactttacagaaccaactccaattcagtgccagggatttccgttggctcttagtggccgggatatggtgggcattgctcagactggctctgggaagacgttggcgtatctcctgcctgcaattgttcatattaaccaccagccatacttggaaaggggagatggcccaatctgtctagttctggctcctaccagagagcttgcccagcaagtacagcaggtggccgatgactatggcaaatgttctagattgaagagtacttgtatttatggaggtgctcctaaaggtccccagattcgagacttggaaagaggtgttgagatctgcatagccactcctggacgtctgatagatttcctggagtcaggaaagacaaatcttcgccgatgtacttaccttgtattggacgaagctgacagaatgcttgatatggggtttgaaccccagatccgtaaaattgttgaccaaatcaggcctgataggcagacactgatgtggagtgcaacctggccaaaagaagtaagacagcttgcagaggatttccttcgtgattacacccagatcaacgtaggcaatctggagttgagtgccaaccacaacatcctccagatagtggatgtctgcatggaaagtgaaaaagaccacaagttgatccaactaatggaagaaataatggctgaaaaggaaaacaaaacaataatatttgtggagacaaagagacgctgtgatgatctgactcgaaggatgcgcagagatggttggccagctatgtgtatccatggagacaagagtcaaccagaaagagattgggtacttaatgagttccgttctggaaaggcacccatccttattgctacagatgtagcctcccgtgggctagatgtggaagatgtcaagtttgtgatcaactatgactatccaaacagctcagaggattatgtgcaccgtattggccgaacagcccgtagcaccaacaagggtaccgcctataccttcttcaccccagggaacctaaaacaggccagagagcttatcaaagtgctggaagaggccaatcaggctatcaatccaaaactgatgcagcttgtggaccacagaggaggcggcggaggcgggggtggtcgttctcgttaccggaccacttcttcagccaacaatcccaatctgatgtatcaggatgagtgtgaccgaaggcttcgaggagtcaaggatggtggccggagagactctgcaagctatcgggatcgtagtgaaaccgatagagctggttatgctaatggcagtggctatggaagtccaaattctgcctttggagcacaagcaggccaatacacctatggtcaaggcacctatggggcagctgcttatggcaccagtagctatacagctcaagaatatggtgctggcacttatggagctagtagcaccacctcaactgggagaagttcacagagctctagccagcagtttagtgggataggccggtctgggcagcagccacagccactgatgtcacaacagtttgcacagcctccgggagctaccaatatgataggttacatggggcagactgcctaccaataccctcctcctcctccccctcctcctccttcacgtaaatga

SEQ ID NO: 5 - protein, 440 aa, Homo sapiens
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 6 - DNA, 1323 bp, Homo sp.
  source 1..1323: mol_type = genomic DNA
atggcgtcgctcaccgtgaaggcctaccttctgggcaaggaggacgcggcgcgcgagattcgccgcttcagcttctgctgcagccccgagcctgaggcggaagccgaggctgcggcgggtccgggaccctgcgagcggctgctgagccgggtggccgccctgttccccgcgctgcggcctggcggcttccaggcgcactaccgcgatgaggacggggacttggttgccttttccagtgacgaggaattgacaatggccatgtcctacgtgaaggatgacatcttccgaatctacattaaagagaaaaaagagtgccggcgggaccaccgcccaccgtgtgctcaggaggcgccccgcaacatggtgcaccccaatgtgatctgcgatggctgcaatgggcctgtggtaggaacccgctacaagtgcagcgtctgcccagactacgacttgtgtagcgtctgcgagggaaagggcttgcaccgggggcacaccaagctcgcattccccagccccttcgggcacctgtctgagggcttctcgcacagccgctggctccggaaggtgaaacacggacacttcgggtggccaggatgggaaatgggtccaccaggaaactggagcccacgtcctcctcgtgcaggggaggcccgccctggccccacggcagaatcagcttctggtccatcggaggatccgagtgtgaatttcctgaagaacgttggggagagtgtggcagctgcccttagccctctgggcattgaagttgatatcgatgtggagcacggagggaaaagaagccgcctgacccccgtctctccagagagttccagcacagaggagaagagcagctcacagccaagcagctgctgctctgaccccagcaagccgggtgggaatgttgagggcgccacgcagtctctggcggagcagatgaggaagatcgccttggagtccgaggggcgccctgaggaacagatggagtcggataactgttcaggaggagatgatgactggacccatctgtcttcaaaagaagtggacccgtctacaggtgaactccagtccctacagatgccagaatccgaagggccaagctctctggacccctcccaggagggacccacagggctgaaggaagctgccttgtacccacatctcccgccagaggctgacccgcggctgattgagtccctctcccagatgctgtccatgggcttctctgatgaaggcggctggctcaccaggctcctgcagaccaagaactatgacatcggagcggctctggacaccatccagtattcaaagcatcccccgccgttgtag

SEQ ID NO: 7 - DNA, 14 bp, synthetic construct
  source 1..14: mol_type = other DNA
gcctccacctgctc

SEQ ID NO: 8 - DNA, 21 bp, synthetic construct
  source 1..21: mol_type = other DNA
gctagcagatcttagagtcgg

SEQ ID NO: 9 - DNA, 26 bp, synthetic construct
  source 1..26: mol_type = other DNA
agcggtaggcatggcgtcgctcaccg

SEQ ID NO: 10 - DNA, 30 bp, synthetic construct
  source 1..30: mol_type = other DNA
agaagatctgctagtacaacggcgggatgc

SEQ ID NO: 11 - DNA, 22 bp, synthetic construct
  source 1..22: mol_type = other DNA
ggtggctttaccaacagtaccg

SEQ ID NO: 12 - DNA, 21 bp, synthetic construct
  source 1..21: mol_type = other DNA
gctagcagatcttctagtcgg

SEQ ID NO: 13 - DNA, 30 bp, synthetic construct
  source 1..30: mol_type = other DNA
agcggtggcatgtcgggattcgagtgaccg

SEQ ID NO: 14 - DNA, 39 bp, synthetic construct
  source 1..39: mol_type = other DNA
agaagatctgctagcttattgggaatcctgttggcattg

SEQ ID NO: 15 - DNA, 1024 bp, synthetic construct
  source 1..1024: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgac

SEQ ID NO: 16 - DNA, 1024 bp, synthetic construct
  source 1..1024: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtgaccggctaccggctgttcgaggagattctcgggagttccggtggtggcgggagcggaggtggaggcatgtcgggttattcgagtgaccgagaccgcggccgggaccgagggtttggtgcacctcgatttggaggaagtagggcagggcccttatctggaaagaagtttggaaaccctggggagaaattagttaaaaagaagtgg

SEQ ID NO: 17 - DNA, 28 bp, synthetic construct
  source 1..28: mol_type = other DNA
cgcaaatctagacttgtacagctcgtcc

SEQ ID NO: 18 - DNA, 18 bp, synthetic construct
  source 1..18: mol_type = other DNA
ggccgcttcgagcagatg

SEQ ID NO: 19 - DNA, 31 bp, synthetic construct
  source 1..31: mol_type = other DNA
aagtctagatttgcgatgggagtgcaggtgg

SEQ ID NO: 20 - DNA, 32 bp, synthetic construct
  source 1..32: mol_type = other DNA
ctgctcgaagcggcctcattccagttagaagc

SEQ ID NO: 21 - DNA, 1024 bp, synthetic construct
  source 1..1024: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctggtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggc

SEQ ID NO: 22 - DNA, 2220 bp, Homo sapiens
  source 1..2220: mol_type = genomic DNA
gcggccttctccgagatgggtgtaatgcctgagattgcacaagctgtggaagagatggattggctcctcccaactgatatccaggctgaatctatcccattgatcttaggaggaggtgatgtacttatggctgcagaaacaggaagtggcaaaactggtgcttttagtattccagttatccagatagtttatgaaactctgaaagaccaacaggaaggcaaaaaaggaaaaacaacaattaaaactggtgcttcagtgctgaacaaatggcagatgaacccatatgacagaggatctgcttttgcaattgggtcagatggtctttgttgtcaaagcagagaagtaaaggaatggcatgggtgtagagctactaaaggattaatgaaagggaaacactactatgaagtatcctgtcatgaccaagggttatgcagggtcgggtggtctaccatgcaggcctctttggacctaggtactgacaagtttggatttggctttggtggaacaggaaagaaatcccataacaaacaatttgataattatggagaggaattcactatgcatgataccattggatgttacctggatatagataagggacatgtcaagttctccaaaaatggaaaagatcttggtctggcatttgaaataccaccacatatgaaaaaccaagccctctttcctgcctgtgttttgaagaatgctgaactgaaatttaacttcggtgaagaggaatttaagtttccaccaaaagatggctttgttgctctttccaaggcaccggatggttacattgtcaaatcacagcactcaggtaatgcacaggtgacacaaacaaagtttctccccaatgctccgaaagctctcattgttgaaccttcccgggagttagctgaacaaactttgaacaacatcaagcagtttaagaaatacattgataatcctaaattaagggagcttctgataattggaggtgttgcagcccgggatcagctctctgttttggaaaatggagtagatatagttgtaggtactccgggaagactagatgacttggtgtcaactggaaagctgaacttatctcaagttagattcctggtcctggatgaagctgatgggcttctttctcaaggttattctgattttataaataggatgcacaatcagattcctcaggttacctctgatggaaaaagacttcaggtgattgtttgctctgccactttgcattctttcgatgtaaagaaactgtccgagaagataatgcattttcctacatgggttgacttaaaaggagaagactctgttccagatactgtacaccatgttgttgtcccagtaaatcccaaaactgacagactctgggaaaggcttggaaagagccacattagaactgatgatgtacatgcaaaagataacacaagacctggtgctaatagtccagagatgtggtctgaagctattaaaatcctgaaaggggagtatgctgtccgggcaatcaaggaacataagatggatcaagcaattatcttctgtagaaccaaaattgactgtgataacttggagcagtactttatacaacaaggaggaggacctgataaaaaaggacaccagttctcatgtgtttgtcttcatggtgacagaaagcctcatgagagaaagcaaaacttggaaagatttaagaaaggagatgtaagattcttgatttgcacagatgtagctgctagaggaattgatatccacggtgttccttatgttataaatgtcactctgcccgatgaaaagcaaaactacgtacatcgaattggcagagtaggaagagctgaaaggatgggtctggcaatttccctggtggcaacagaaaaagaaaaggtttggtaccatgtatgtagcagccgtggaaaagggtgttataacacaagactcaaggaagatggaggctgtaccatatggtacaacgagatgcagttactatctgagatagaagaacacctgaactgtaccatttctcaggttgagccggatataaaggtaccagtggatgaatttgatgggaaagttacctacggtcagaaaagggctgctggtggtggaagctataaaggccatgtggatattttggcacctactgttcaagagttggctgcccttgaaaaggaggcgcagacatctttcctgcatcttggctaccttcctaaccagctgttcagaaccttctga

SEQ ID NO: 23 - DNA, 1218 bp, Homo sapiens
  source 1..1218: mol_type = genomic DNA
tctgcgagccaggattcccgatccagagacaatggccccgatgggatggagcccgaaggcgtcatcgagagtaactggaatgagattgttgacagctttgatgacatgaacctctcggagtcccttctccgtggcatctacgcgtatggttttgagaagccctctgccatccagcagcgagccattctaccttgtatcaagggttatgatgtgattgctcaagcccaatctgggactgggaaaacggccacatttgccatatcgattctgcagcagattgaattagatctaaaagccacccaggccttggtcctagcacccactcgagaattggctcagcagatacagaaggtggtcatggcactaggagactacatgggcgcctcctgtcacgcctgtatcgggggcaccaacgtgcgtgctgaggtgcagaaactgcagatggaagctccccacatcatcgtgggtacccctggccgtgtgtttgatatgcttaaccggagatacctgtcccccaaatacatcaagatgtttgtactggatgaagctgacgaaatgttaagccgtggattcaaggaccagatctatgacatattccaaaagctcaacagcaacacccaggtagttttgctgtcagccacaatgccttctgatgtgcttgaggtgaccaagaagttcatgagggaccccattcggattcttgtcaagaaggaagagttgaccctggagggtatccgccagttctacatcaacgtggaacgagaggagtggaagctggacacactatgtgacttgtatgaaaccctgaccatcacccaggcagtcatcttcatcaacacccggaggaaggtggactggctcaccgagaagatgcatgctcgagatttcactgtatccgccatgcatggagatatggaccaaaaggaacgagacgtgattatgagggagtttcgttctggctctagcagagttttgattaccactgacctgctggccagaggcattgatgtgcagcaggtttctttagtcatcaactatgaccttcccaccaacagggaaaactatatccacagaatcggtcgaggtggacggtttggccgtaaaggtgtggctattaacatggtgacagaagaagacaagaggactcttcgagacattgagaccttctacaacacctccattgaggaaatgcccctcaatgttgctgacctcatctga

SEQ ID NO: 24 - DNA, 1986 bp, Homo sapiens
  source 1..1986: mol_type = genomic DNA
agtcatgtggcagtggaaaatgcgctcgggctggaccagcagtttgctggcctagacctgaactcttcagataatcagagtggaggaagtacagccagcaaagggcgctatattcctcctcatttaaggaaccgagaagctactaaaggtttctacgataaagacagttcagggtggagttctagcaaagataaggatgcgtatagcagttttggatctcgtagtgattcaagagggaagtctagcttcttcagtgatcgtggaagtggatcaaggggaaggtttgatgatcgtggacggagtgattacgatggcattggcagccgtggtgacagaagtggctttggcaaatttgaacgtggtggaaacagtcgctggtgtgacaaatcagatgaagatgattggtcaaaaccactcccaccaagtgaacgcttggaacaggaactcttttctggaggcaacactgggattaattttgagaaatacgatgacattccagttgaggcaacaggcaacaactgtcctccacatattgaaagtttcagtgatgttgagatgggagaaattatcatgggaaacattgagcttactcgttatactcgcccaactccagtgcaaaagcatgctattcctattatcaaagagaaaagagacttgatggcttgtgcccaaacagggtctggaaaaactgcagcatttctgttgcccatcttgagtcagatttattcagatggtccaggcgaggctttgagggccatgaaggaaaatggaaggtatgggcgccgcaaacaatacccaatctccttggtattagcaccaacgagagagttggcagtacagatctacgaggaagccagaaaattttcataccgatctagagttcgtccttgcgtggtttatggtggtgccgatattggtcagcagattcgagacttggaacgtggatgccatttgttagtagccactccaggacgtctagtggatatgatggaaagaggaaagattggattagacttttgcaaatacttggtgttagatgaagctgatcggatgttggatatggggtttgagcctcagattcgtagaatagtcgaacaagatactatgcctccaaagggtgtccgccacactatgatgtttagtgctacttttcctaaggaaatacagatgctggctcgtgatttcttagatgaatatatcttcttggctgtaggaagagttggctctacctctgaaaacatcacacagaaagtagtttgggtggaagaatcagacaaacggtcatttctgcttgacctcctaaatgcaacaggcaaggattcactgaccttagtgtttgtggagaccaaaaagggtgcagattctctggaggatttcttataccatgaaggatacgcatgtaccagcatccatggagaccgttctcagagggatagagaagaggcccttcaccagttccgctcaggaaaaagcccaattttagtggctacagcagtagcagcaagaggactggacatttcaaatgtgaaacatgttatcaattttgacttgccaagtgatattgaagaatatgtacatcgtattggtcgtacgggacgtgtaggaaaccttggcctggcaacctcattctttaacgagaggaacataaatattactaaggatttgttggatcttcttgttgaagctaaacaagaagtgccgtcttggttagaaaacatggcttatgaacaccactacaagggtagcagtcgtggacgttctaagagtagcagatttagtggagggtttggtgccagagactaccgacaaagtagcggtgccagcagttccagcttcagcagcagccgcgcaagcagcagccgcagtggcggaggtggccacggtagcagcagaggatttggtggaggtggctatggaggcttttacaacagtgatggatatggaggaaattataactcccagggggttgactggtggggtaactga

SEQ ID NO: 25 - DNA, 2172 bp, Homo sapiens
  source 1..2172: mol_type = genomic DNA
ggagatgaagattgggaagcagaaatcaaccctcatatgtcttcctatgttcccatatttgagaaggataggtattctggagaaaatggagacaattttaacaggactccagcttcatcatcagaaatggatgatggaccttctcgaagagatcatttcatgaaaagtggatttgcctctgggcggaattttggaaacagagatgctggtgagtgtaataagcgagataatacatccacaatgggtggttttggagttggaaagagttttggaaacagaggtttttcaaacagcaggtttgaagatggtgatagctctggtttctggagagagtctagtaatgactgcgaagataatccaacacggaacagagggttttccaagagaggcggctatcgagatggaaataattcagaagcttcagggccatacagaagaggtggaagaggtagtttccgaggttgccgtggaggatttggtctaggaagtccaaataatgacttagacccagacgaatgtatgcagcgcactggtggcctttttggttctagaagaccagtattaagtggcacaggtaatggtgatacttctcaaagcagaagtggcagtggaagtgaacgaggtggttacaaaggtttaaatgaagaagtaataacaggctctggaaagaattcttggaagtcagaagcagaaggaggagaaagtagtgatactcaaggaccaaaagtgacctacataccccctcctccacctgaggatgaggactccatctttgcacattatcagacaggcataaacttcgacaaatacgacactattcttgtggaagtgtctggacatgatgcaccaccagcaattctgacttttgaagaagctaatctctgtcagacactgaataacaacattgctaaagctggttatactaagcttactcctgtgcaaaaatacagtattcctatcatacttgcaggacgagatttgatggcttgcgctcaaacagggtctgggaagactgcggcttttctcctaccaattttggctcatatgatgcatgatggaataactgccagtcgttttaaagagttgcaggaaccagagtgtattattgtagcaccaactcgagaattggtcaaccagatttatttggaagccagaaaattttcttttgggacttgtgtaagagctgttgttatatatgggggaacccagctgggacattcaattcgacaaatagtacaaggctgtaatatattatgtgctactcctggaagactgatggatatcataggcaaagaaaagattggtctcaaacagatcaaatacttagttttggatgaagctgatcgcatgttggatatgggttttggtccagaaatgaagaagttaatttcttgcccaggaatgccatcaaaggaacagcgccaaacccttatgttcagtgcaacttttccagaggaaattcaaaggttggctgcagagtttttaaagtcaaattatctgtttgttgctgttggacaagtgggtggagcatgtagagatgttcagcagaccgttctccaagttggccagttctcaaaaagagaaaagctcgttgaaattctgcgaaacataggggatgaaagaactatggtctttgttgaaactaagaaaaaagcagattttattgcaacttttctttgtcaagaaaaaatatcaactacaagtattcatggtgatcgggaacagagagagcgggagcaagctcttggagattttcgctttggaaagtgcccagttcttgttgctacttcagtagctgccagagggctggatattgaaaatgtgcaacatgttatcaattttgatcttccttctaccattgatgaatatgttcatcgaattgggcgtactggtcgttgtgggaatactggcagagcaatttccttttttgatcttgaatcggataaccatttagcacagcctctagtaaaagtattgacagatgctcaacaggatgttcctgcatggttggaagaaattgcctttagtacatacattcctggcttcagtggtagtacaagaggaaacgtgtttgcatcagttgataccagaaagggcaagagcactttgaacacagctgggttttcttcttcacaagctcccaatccagtagatgatgagtcatgggattaa

SEQ ID NO: 26 - DNA, 1842 bp, Homo sapiens
  source 1..1842: mol_type = genomic DNA
tcgggttattcgagtgaccgagaccgcggccgggaccgagggtttggtgcacctcgatttggaggaagtagggcagggcccttatctggaaagaagtttggaaaccctggggagaaattagttaaaaagaagtggaatcttgatgagctgcctaaatttgagaagaatttttatcaagagcaccctgatttggctaggcgcacagcacaagaggtggaaacatacagaagaagcaaggaaattacagttagaggtcacaactgcccgaagccagttctaaatttttatgaagccaatttccctgcaaatgtcatggatgttattgcaagacagaatttcactgaacccactgctattcaagctcagggatggccagttgctctaagtggattggatatggttggagtggcacagactggatctgggaaaacattgtcttatttgcttcctgccattgtccacatcaatcatcagccattcctagagagaggcgatgggcctatttgtttggtgctggcaccaactcgggaactggcccaacaggtgcagcaagtagctgctgaatattgtagagcatgtcgcttgaagtctacttgtatctacggtggtgctcctaagggaccacaaatacgtgatttggagagaggtgtggaaatctgtattgcaacacctggaagactgattgactttttagagtgtggaaaaaccaatctgagaagaacaacctaccttgtccttgatgaagcagatagaatgcttgatatgggctttgaaccccaaataaggaagattgtggatcaaataagacctgataggcaaactctaatgtggagtgcgacttggccaaaagaagtaagacagcttgctgaagatttcctgaaagactatattcatataaacattggtgcacttgaactgagtgcaaaccacaacattcttcagattgtggatgtgtgtcatgacgtagaaaaggatgaaaaacttattcgtctaatggaagagatcatgagtgagaaggagaataaaaccattgtttttgtggaaaccaaaagaagatgtgatgagcttaccagaaaaatgaggagagatgggtggcctgccatgggtatccatggtgacaagagtcaacaagagcgtgactgggttctaaatgaattcaaacatggaaaagctcctattctgattgctacagatgtggcctccagagggctagatgtggaagatgtgaaatttgtcatcaattatgactaccctaactcctcagaggattatattcatcgaattggaagaactgctcgcagtaccaaaacaggcacagcatacactttctttacacctaataacataaagcaagtgagcgaccttatctctgtgcttcgtgaagctaatcaagcaattaatcccaagttgcttcagttggtcgaagacagaggttcaggtcgttccaggggtagaggaggcatgaaggatgaccgtcgggacagatactctgcgggcaaaaggggtggatttaatacctttagagacagggaaaattatgacagaggttactctagcctgcttaaaagagattttggggcaaaaactcagaatggtgtttacagtgctgcaaattacaccaatgggagctttggaagtaattttgtgtctgctggtatacagaccagttttaggactggtaatccaacagggacttaccagaatggttatgatagcactcagcaatacggaagtaatgttccaaatatgcacaatggtatgaaccaacaggcatatgcatatcctgctactgcagctgcacctatgattggttatccaatgccaacaggatattcccaataa

SEQ ID NO: 27 - DNA, 1449 bp, Homo sapiens
  source 1..1449: mol_type = genomic DNA
agcacggccagaacagagaaccctgttataatgggtctgtccagtcaaaatggtcagctgagaggccctgtgaaacccactggtggccctggaggagggggcacacagacacagcaacagatgaaccagctgaaaaacaccaacacaatcaataatggcactcagcagcaagcacagagtatgaccaccactattaaacctggtgatgactggaaaaagactttaaaactccctccaaaggatctaagaatcaaaacttcggatgtgacctccacaaaaggaaatgagtttgaagattactgtttgaaacgggagttactgatgggaatttttgaaatgggctgggaaaagccatctcctattcaggaggagagcattcccattgctttatctggtagggatatcttagctagagcaaaaaatggaacaggcaagagcggtgcctacctcattcccttacttgaacggctagacctgaagaaggacaatatacaagcaatggtgattgttcccactagagaacttgctctacaggtcagtcaaatttgcatccaggtcagcaaacacatgggaggggccaaagtgatggcaaccacaggaggaaccaatttacgagatgacataatgaggcttgatgatacagtgcacgtggtgattgctacccctgggagaatcctggatcttattaagaaaggagtagcaaaggttgatcatgtccagatgatagtattggatgaggcagataagttgctgtcacaggattttgtgcagataatggaggatattattctcacgctacctaaaaacaggcagattttactatattccgctactttccctcttagtgtacagaagttcatgaattcccatttgcagaaaccctatgagattaacctgatggaggaactaactctgaagggagtaacccagtactacgcatatgtaactgagcgccaaaaagtacactgcctcaacacacttttctccaggcttcagataaaccagtcgatcattttctgtaactcctctcagcgagttgaattgctagccaagaagatttctcaactgggttattcttgcttctatattcatgctaaaatgaggcaggaacatcgaaatcgtgtatttcatgatttccgaaatggcttatgccgcaatcttgtttgcactgatctgtttacccgaggtattgatatacaagctgtgaatgtggtaataaactttgatttcccaaagctggcagagacctatctccatcgtattggaagatcaggtcgctttggtcatcttggcttagccatcaacttgatcacatatgatgatcgcttcaacctgaaaagtattgaggagcagctgggaacagaaattaaacctattccgagcaacattgataagagcctgtatgtggcagaataccacagcgagcctgtagaagatgagaaaccttaa

SEQ ID NO: 28 - DNA, 2625 bp, Homo sapiens
  source 1..2625: mol_type = genomic DNA
ggcaaaacggccaactctccgggttcgggagcccgacccgacccggtgcggagcttcaatcgctggaagaaaaaacacagccataggcagaacaaaaagaagcagttgaggaagcaactgaagaaacccgaatggcaggtcgagcgcgagagtatcagccgcctcatgcagaactatgaaaagataaatgtaaatgaaatcacaagattttcagattttcccttgtccaaaaaaacattgaaaggtttgcaagaagctcagtaccgtttggtgactgagatacagaagcagaccattggattggctttgcaaggtaaagatgtacttggagcggccaaaactggatctggcaagactctggcttttcttgttccagtgctggaagccttatatcgtctgcaatggacttcaacagatgggctgggggttctcataatatcacctacgagagaactggcctatcagacctttgaggttctccgaaaagtaggaaagaatcatgacttctcagctggtctcatcattggtggaaaggatctaaaacacgaagctgagaggatcaacaacataaatatactcgtgtgcacaccaggtcggcttcttcaacacatggatgaaacagtatcttttcatgctaccgacctccaaatgttagttcttgatgaagcagatagaatcttggatatgggctttgctgataccatgaatgctgttattgaaaatctccccaagaaacgtcagactttacttttctcagcaacacaaactaaatctgtaaaggaccttgcacgcttgagtttgaaaaaccctgagtatgtctgggttcatgaaaaagcaaaatatagcacccctgccactttggaacagaactacatagtctgtgagctgcagcaaaaaataagtgtgctgtattcctttttgagaagccatctgaagaagaagagcattgtatttttttccagttgcaaagaggtccagtatctgtaccgagtgttttgccggctacgtcctggtgtttctatccttgcactccatggtcgacagcagcaaatgagaagaatggaagtctataatgagtttgtccgtaagagagctgcagtactctttgctactgatattgcagccaggggtctggatttcccggccgtgaattgggttcttcagtttgattgtcctgaggatgccaacacatatattcacagagcaggtagaactgccaggtacaaagaggatggtgaagctttgctaattttgctaccctcagaaaaagctatggtgcagcagcttcttcagaagaaagtacctgtgaaggaaatcaaaatcaatccagaaaaacttatagatgtccagaaaaaattggaatctattttagctcaagatcaagatttaaaagaaagagctcaaaggtgtttcgtctcctatgtacgatctgtatatctgatgaaggataaagaagtatttgatgtgagcaagttacctatacctgaatatgccctgtctcttggtcttgctgtggcaccacgcgtaagatttcttcagaaaatgcagaaacaacccaccaaagaattggtaaggagccaagccgataaagtaattgagccaagggctccctccctcaccaatgacgaagtggaagaatttagagcctacttcaatgagaaaatgtccatccttcaaaaaggtggaaaaagactcgaagggacagagcacagacaggataatgatactggtaatgaagaacaggaagaagaagaagacgatgaagaagaaatggaagagaaactggcaaaagcaaaaggatctcaagccccatctcttcctaacaccagtgaggcacagaagatcaaggaagttcctacacagttcttggacagagatgaggaggaagaagatgctgatttcttgaaggtgaagcggcataatgtgtttggattggaccttaaagacgagaaaacattacagaagaaagaaccttctaaatccagcatcaagaaaaaaatgaccaaagttgcagaagcaaaaaaagtaatgaagagaaattttaaagtgaataagaagataacatttactgatgaaggggagttggttcagcagtggccacaaatgcagaaatctgccatcaaggatgctgaggaagatgatgacacaggtggtatcaacttacataaagcaaaggaaagacttcaggaagaggacaaatttgacaaagaagaatataggaaaaaaattaaggcaaagcatcgggagaaaagactgaaagaaagggaagccagaagagaagccaacaagagacaagcaaaggccaaagatgaagaggaagcctttctggattggagtgatgatgatgatgatgatgatgatggatttgatccaagcacactcccagatccagataaatacagaagctctgaagattcagatagtgaagatatggaaaataaaataagtgataccaagaagaagcaggggatgaagaagaggagcaacagtgaagtggaagacgtgggaccaacaagtcataacagaaagaaggccaggtgggacactttagagcctttggataccggcctgtctttagcagaggatgaagagctggtgttacatctgctaagaagtcaaagctaa

SEQ ID NO: 29 - DNA, 2710 bp, Homo sapiens
  source 1..2710: mol_type = genomic DNA
gctaatgaaacacagaaggttggtgccatccattttccttttcccttcacaccctattccatccaggaagacttcatggcagagctgtaccgggttttggaggctggcaagattgggatatttgagagtccaactggcactgggaagtccttaagtcttatttgtggggccctctcttggctccgtgactttgaacagaagaagcgtgaagaagaggcacgactccttgaaactggaactggccccttacatgatgagaaagatgaatccctgtgtctgtcttcttcctgcgaaggggctgcaggcaccccgaggcctgctggagaaccggcctgggttactcagtttgtgcagaagaaagaagagagggacctggtggaccgactaaaggcggagcaggccaggaggaagcagcgagaagaacgcctgcagcagctgcagcacagggtgcagctcaagtatgcagccaagcgcctgaggcaggaagaagaagaaagagagaatctcctccgcctcagcagggagatgctagagacaggcccggaggctgagcggctggagcagctggagtctggggaggaggagctggtcctcgccgaatacgagagtgatgaggagaaaaaggtggcgagcagagtggatgaggatgaggatgacctggaggaagaacacataactaagatttattactgtagtcggacacactcccagctggcccagtttgtgcatgaggtgaagaagagcccctttggcaaggatgttcggctggtctcccttggctcccggcagaacctttgtgtaaatgaagacgtgaaaagcctaggttctgtgcagcttatcaacgaccgctgtgtggacatgcagagaagcaggcacgagaagaagaaaggagctgaggaggagaagccaaagaggaggaggcaggagaagcaggcagcctgccccttctacaaccacgagcagatgggccttctccgggatgaggccctggcagaggtgaaggacatggagcagctgctggcccttgggaaggaggcccgggcctgtccctattacgggagccgccttgccatccctgcagcccagctggtggtgctgccctatcagatgctgctgcatgcggccactcggcaggccgcgggcatccggctgcaggaccaggtggtgatcatcgacgaggcgcacaacctgatcgacaccatcacgggcatgcacagcgtggaggtcagcggctcccagctctgccaggcccattcccagctgctgcagtacgtggagcgatacgggaagcgtttgaaggccaagaacctgatgtacctgaagcagatcctgtatttgctggagaaattcgtggctgtgctaggggggaacattaagcaaaatcccaatacacagagtctgtcacagacagggacggagctgaagaccatcaacgactttctcttccagagccagatcgacaacatcaacctgttcaaggtgcagcgatactgtgagaagagcatgatcagcagaaagctctttggattcactgaacggtacggagcagtgttctcatcccgggagcagcccaaactggctgggtttcagcaattcctgcagagcctgcagcccaggacgactgaagctcttgcagcccctgcagacgagagtcaggccagcaccctgcgaccagcttctccactgatgcacatccaaggcttcctggcagctctcactacggccaaccaggacggcagggtcatcctgagccgccaaggcagcctcagtcagagcaccctgaagtttttgctcctgaatccagctgtgcactttgcccaagtggtgaaggaatgccgggcagtggtcattgcggggggtaccatgcagccggtgtctgacttccggcagcagctgctggcctgtgccggggtggaagctgagcgcgtggtggagttttcctgtggtcacgtgatccctccagacaacatcctgcccctcgtcatctgcagcgggatctccaaccagccgctggaattcacgttccagaaaagagagctgcctcagatgatggacgaggtgggtcgcattctctgtaacctgtgcggtgtggttcctggaggggtggtctgtttcttcccctcctacgagtacctgcgccaggtccatgcccactgggagaagggtggcctgctgggccgtctggctgccaggaagaagatattccaggaacctaagagcgcacaccaggtggagcaggtgctgctggcatattccaggtgcatccaggcctgtggccaggagagaggccaggtgacaggggccctgctcctctctgtggttggaggaaagatgagtgaagggatcaacttctctgacaacctaggccggtgtgtggtgatggtgggcatgcccttccccaacatcaggtctgcagagctgcaggagaagatggcctacttggatcaaaccctcagcccccggccaggcacccccagggaaggctctggtggagaacctgtgcatgaaggccgtcaaccagtccataggcagggccatcaggcaccagaaggattttgccagcgtagtgctcctggaccagcgatatgcccggccccctgtcctggccaagctgccggcctggatccgagcccgtgtggaggtcaaagctacctttggccccgccattgctgctgtgcagaagtttcaccgggagaagtcggcctcttcc

SEQ ID NO: 30 - DNA, 2187 bp, Homo sapiens
  source 1..2187: mol_type = genomic DNA
cccaccggctttgtagccccgattctctgtgttttgctcccgtctccgacgagagaggcggcgacggtggcgtctgcgacgggagacagcgcgtcggagcgagagagcgctgcgcctgccgccgccccaacagcggaggcgccgccgccatcggtcgtcaccagaccggagccgcaggccctcccgagcccggccatccgtgccccgctcccagatctctatccttttgggaccatgcgcggaggaggctttggggaccgggaccgggatcgtgaccgtggaggatttggagcaagaggtggtggtggccttcccccgaagaaatttggtaatcctggggagcgtttgcgtaaaaaaaagtgggatttgagtgagctccccaagtttgagaaaaatttttatgtggaacatccggaagtagcaaggctgacaccatatgaggttgatgagctacgccgaaagaaggagattacagtgagggggggagatgtttgtcctaaacccgtgtttgccttccatcatgctaacttcccacaatatgtaatggatgtgttgatggatcagcactttacagaaccaactccaattcagtgccagggatttccgttggctcttagtggccgggatatggtgggcattgctcagactggctctgggaagacgttggcgtatctcctgcctgcaattgttcatattaaccaccagccatacttggaaaggggagatggcccaatctgtctagttctggctcctaccagagagcttgcccagcaagtacagcaggtggccgatgactatggcaaatgttctagattgaagagtacttgtatttatggaggtgctcctaaaggtccccagattcgagacttggaaagaggtgttgagatctgcatagccactcctggacgtctgatagatttcctggagtcaggaaagacaaatcttcgccgatgtacttaccttgtattggacgaagctgacagaatgcttgatatggggtttgaaccccagatccgtaaaattgttgaccaaatcaggcctgataggcagacactgatgtggagtgcaacctggccaaaagaagtaagacagcttgcagaggatttccttcgtgattacacccagatcaacgtaggcaatctggagttgagtgccaaccacaacatcctccagatagtggatgtctgcatggaaagtgaaaaagaccacaagttgatccaactaatggaagaaataatggctgaaaaggaaaacaaaacaataatatttgtggagacaaagagacgctgtgatgatctgactcgaaggatgcgcagagatggttggccagctatgtgtatccatggagacaagagtcaaccagaaagagattgggtacttaatgagttccgttctggaaaggcacccatccttattgctacagatgtagcctcccgtgggctagatgtggaagatgtcaagtttgtgatcaactatgactatccaaacagctcagaggattatgtgcaccgtattggccgaacagcccgtagcaccaacaagggtaccgcctataccttcttcaccccagggaacctaaaacaggccagagagcttatcaaagtgctggaagaggccaatcaggctatcaatccaaaactgatgcagcttgtggaccacagaggaggcggcggaggcgggggtggtcgttctcgttaccggaccacttcttcagccaacaatcccaatctgatgtatcaggatgagtgtgaccgaaggcttcgaggagtcaaggatggtggccggagagactctgcaagctatcgggatcgtagtgaaaccgatagagctggttatgctaatggcagtggctatggaagtccaaattctgcctttggagcacaagcaggccaatacacctatggtcaaggcacctatggggcagctgcttatggcaccagtagctatacagctcaagaatatggtgctggcacttatggagctagtagcaccacctcaactgggagaagttcacagagctctagccagcagtttagtgggataggccggtctgggcagcagccacagccactgatgtcacaacagtttgcacagcctccgggagctaccaatatgataggttacatggggcagactgcctaccaataccctcctcctcctccccctcctcctccttcacgtaaatga

SEQ ID NO: 31 - DNA, 2010 bp, Homo sapiens
  source 1..2010: mol_type = genomic DNA
tcacacctgccgatgaaactcctgcgtaagaagatcgagaagcggaacctcaaattgcggcagcggaacctaaagtttcagggggcctcaaatctgaccctatcggaaactcaaaatggagatgtatctgaagaaacaatgggaagtagaaaggttaaaaaatcaaaacaaaagcccatgaatgtgggcttatcagaaactcaaaatggaggcatgtctcaagaagcagtgggaaatataaaagttacaaagtctccccagaaatccactgtattaaccaatggagaagcagcaatgcagtcttccaattcagaatcaaaaaagaaaaagaagaaaaagagaaaaatggtgaatgatgctgagcctgatacgaaaaaagcaaaaactgaaaacaaagggaaatctgaagaagaaagtgccgagactactaaagaaacagaaaataatgtggagaagccagataatgatgaagatgagagtgaggtgcccagtctgcccctgggactgacaggagcttttgaggatacttcgtttgcttctctatgtaatcttgtcaatgaaaacactctgaaggcaataaaagaaatgggttttacaaacatgactgaaattcagcataaaagtatcagaccacttctggaaggcagggatcttctagcagctgcaaaaacaggcagtggtaaaaccctggcttttctcatccctgcagttgaactcattgttaagttaaggttcatgcccaggaatggaacaggagtccttattctctcacctactagagaactagccatgcaaacctttggtgttcttaaggagctgatgactcaccacgtgcatacctatggcttgataatgggtggcagtaacagatctgctgaagcacagaaacttggtaatgggatcaacatcattgtggccacaccaggccgtctgctggaccatatgcagaataccccaggatttatgtataaaaacctgcagtgtctggttattgatgaagctgatcgtatcttggatgtggggtttgaagaggaattaaagcaaattattaaacttttgccaacacgtagacagactatgctcttttctgccacccaaactcgaaaagttgaagacctggcaaggatttctctgaaaaaggagccattgtatgttggcgttgatgatgataaagcgaatgcaacagtggatggtcttgaacagggatatgttgtttgtccttctgaaaagagattccttctgctctttacattccttaagaagaaccgaaagaagaagcttatggtcttcttttcatcttgtatgtctgtgaaataccactatgagttgctgaactacattgatttgcccgtcttggccattcatggaaagcaaaagcaaaataagcgtacaaccacattcttccagttctgcaatgcagattcgggaacactattgtgtacggatgtggcagcgagaggactagacattcctgaagtcgactggattgttcagtatgaccctccggatgaccctaaggaatatattcatcgtgtgggtagaacagccagaggcctaaatgggagagggcatgccttgctcattttgcgcccagaagaattgggttttcttcgctacttgaaacaatccaaggttccattaagtgaatttgacttttcctggtctaaaatttctgacattcagtctcagcttgagaaattgattgaaaagaattactttcttcataagtcagcccaggaagcatataagtcatacatacgagcctatgattcccattctctgaaacagatctttaatgttaataacctaaatttgcctcaggttgctctgtcatttggtttcaaggtgcctcccttcgttgatctgaacgtcaacagtaatgaaggcaagcagaaaaagcgaggaggtggtggtggatttggctaccagaaaaccaagaaagttgagaaatccaaaatctttaaacacattagcaagaaatcatctgacagcaggcagttctctcactga

SEQ ID NO: 32 - DNA, 1437 bp, Homo sapiens
  source 1..1437: mol_type = genomic DNA
gccactgactcatgggccctggcggtggacgagcaggaagctgcggctgagtcgttgagcaacttgcatcttaaggaagagaaaatcaaaccagataccaatggtgctgttgtcaagaccaatgccaatgcagagaagacagatgaagaagagaaagaggacagagctgcccagtccttactcaacaagctgatcagaagcaaccttgttgataacacaaaccaagtggaagtcctgcaacgggatccaaactcccctctgtactcggtgaagtcgtttgaagagcttcggctgaaaccacagcttctccagggagtctatgccatgggcttcaatcgaccctccaagatacaagagaacgcattacccatgatgcttgctgaacccccacagaatctgattgcccagtctcagtctggcactggtaaaacagctgcctttgtcttagccatgctcagccgagtggagccatcagacagatacccccagtgtctgtgcctctccccaacatatgagctggcgcttcaaacaggaaaagtgattgagcagatgggcaaattttacccagaactgaagcttgcctatgccgttcgaggcaataaattggaaagaggccagaagatcagtgagcagattgtcattggcacccctgggaccgtgctggactggtgctccaagctcaagttcattgatcccaagaaaatcaaggtgtttgttctggatgaggctgatgtcatgatagccactcagggccaccaagatcagagcatccgcatccagaggatgctgcccaggaactgccagatgctgcttttctccgccacctttgaagactctgtgtggaagtttgcccagaaagtggtcccagacccaaatgttatcaaactgaagcgtgaggaagagaccctggataccatcaagcagtactatgtcctgtgcagcagcagagacgagaagttccaggccttgtgtaacctctacggggccatcaccattgctcaagccatgatcttctgccatactcgcaaaacagctagttggctggcagcagagctctcaaaagaaggccaccaggtggctctgctgagtggggagatgatggtggagcagagggctgcggtgattgagcgcttccgagagggcaaagagaaggttttggtgaccaccaacgtgtgtgcccgcggcattgatgttgaacaagtgtctgtcgtcatcaactttgatcttcccgtggacaaggacgggaatcctgacaatgagacctacctgcaccggatcgggcgcacgggccgctttggcaagaggggcctggcagtgaacatggtggacagcaagcacagcatgaacatcctgaacagaatccaggagcattttaataagaagatagaaagattggacacagatgatttggacgagattgagaaaatagccaactga

SEQ ID NO: 33 - DNA, 2472 bp, Homo sapiens
  source 1..2472: mol_type = genomic DNA
gcggcggcatttgaagcctcgggagccttagcagcagtggcgactgctatgccggctgagcatgtggccgtgcaggtcccggccccagagccaacacccgggcctgtgaggatcctgcggaccgctcaggatctcagcagcccgcggacccgcacgggggatgtgctgttggcggagccggccgacttcgagtcactgctgctttcgcggccggtgctggaggggctgcgggcggccggcttcgagaggccctcgccggtgcagctcaaggccatcccgttggggcgctgcgggctcgatttaattgttcaagctaaatctggcaccgggaaaacctgtgtgttctccaccatagctttggactctcttgttcttgaaaacttaagtacccagattttgatcttggctcctacaagagaaattgctgtacagatacattctgttattacagccattggaataaaaatggaaggcttagagtgtcatgtctttattggagggaccccattatcacaagacaaaaccagacttaaaaagtgtcatattgctgttggatctcctggcagaattaagcaactcatagaacttgactacttgaacccaggcagtatacgcctctttattcttgatgaagcagataagcttttagaagaaggcagcttccaggagcaaataaattggatttattcttccttgcctgccagtaaacagatgctggcagtatcagctacttatcccgaatttttggctaatgctttgacaaagtacatgagagatcccacttttgtaagactgaattccagtgatccaagtctcataggtttgaagcagtattacaaagttgtcaattcataccctttggcacataaggtttttgaggaaaagactcagcatttacaggaactgttcagcagaattccatttaatcaagctttagtcttttctaatttgcacagcagagcacaacatttggctgatatcctttcttctaaaggctttcctgctgagtgcatttcaggcaatatgaatcagaatcagcgtcttgatgctatggctaaactgaagcactttcattgcagagtcctcatttccacagatttgacttctcgtgggattgatgctgagaaggtgaatctggttgtaaatctggatgtaccattggattgggagacatacatgcatcggattgggagagctggccgttttggtacattggggctgacagtgacctactgttgccggggagaggaagaaaatatgatgatgagaattgcccagaaatgtaatatcaaccttctccctttaccagatcccattccttctggtctgatggaagaatgtgtggattgggatgtggaagttaaagctgctgtgcatacatatggtatagcaagtgtacctaaccaacccttaaaaaagcaaattcagaaaatagagagaacccttcaaattcagaaagctcatggtgaccacatggcttcctctagaaataattctgtatctggactatcagtcaaatcaaaaaataataccaaacaaaagcttcctgtgaaaagccactcagaatgtggaatcatagaaaaagcaacgtcaccaaaagaactgggctgtgacaggcaatccgaagagcaaatgaagaattctgttcagactcccgttgaaaactccaccaacagtcagcaccaggtcaaagaagctttacctgtgtcactcccccagattccttgtctgtcttcctttaaaatccatcagccatacacgttgacttttgctgaattggtagaggattatgaacattatattaaagaggggttagagaaacctgtggaaatcatcaggcactacacaggccctggggatcagactgtgaatcctcaaaatggttttgtgagaaataaagttattgaacagagagtccctgtgttggcaagtagtagccaatctggagactctgagagtgacagtgattcttacagctcaagaacctcttcccagagcaaaggaaataagtcatacttggaaggctcttctgataatcagctgaaagactctgaatctacgcctgtggatgatcgtatttctttggaacaaccaccaaatggaagtgacacccccaatccagagaaatatcaagaatcacctggaatccagatgaagacaagacttaaagagggggctagccagagagctaagcagagccggagaaacctacccaggcggtcttccttcagattgcagactgaagcccaggaagatgattggtatgactgtcatagggaaatacgtctgagtttttctgatacctatcaggattatgaggagtactggagagcttactacagggcatggcaagaatattatgctgccgcttctcattcatattattggaatgctcagagacatccaagttggatggcagcttatcacatgaataccatttatctacaagaaatgatgcatagtaaccagtga

SEQ ID NO: 34 - DNA, 2349 bp, Homo sapiens
  source 1..2349: mol_type = genomic DNA
ccgggaaaactccgtagtgacgctggtttggaatcagacaccgcaatgaaaaaaggggagacactgcgaaagcaaaccgaggagaaagagaaaaaagagaagccaaaatctgataagactgaagagatagcagaagaggaagaaactgttttccccaaagctaaacaagttaaaaagaaagcagagccttctgaagttgacatgaattctcctaaatccaaaaaggcaaaaaagaaagaggagccatctcaaaatgacatttctcctaaaaccaaaagtttgagaaagaaaaaggagcccattgaaaagaaagtggtttcttctaaaaccaaaaaagtgacaaaaaatgaggagccttctgaggaagaaatagatgctcctaagcccaagaagatgaagaaagaaaaggaaatgaatggagaaactagagagaaaagccccaaactgaagaatggatttcctcatcctgaaccggactgtaaccccagtgaagctgccagtgaagaaagtaacagtgagatagagcaggaaatacctgtggaacaaaaagaaggcgctttctctaattttcccatatctgaagaaactattaaacttctcaaaggccgaggagtgaccttcctatttcctatacaagcaaagacattccatcatgtttacagcgggaaggacttaattgcacaggcacggacaggaactgggaagacattctcctttgccatccctttgattgagaaacttcatggggaactgcaagacaggaagagaggccgtgcccctcaggtactggttcttgcacctacaagagagttggcaaatcaagtaagcaaagacttcagtgacatcacaaaaaagctgtcagtggcttgtttttatggtggaactccctatggaggtcaatttgaacgcatgaggaatgggattgatatcctggttggaacaccaggtcgtatcaaagaccacatacagaatggcaaactagatctcaccaaacttaagcatgttgtcctggatgaagtggaccagatgttggatatgggatttgctgatcaagtggaagagattttaagtgtggcatacaagaaagattctgaagacaatccccaaacattgcttttttctgcaacttgccctcattgggtatttaatgttgccaagaaatacatgaaatctacatatgaacaggtggacctgattggtaaaaagactcagaaaacggcaataactgtggagcatctggctattaagtgccactggactcagagggcagcagttattggggatgtcatccgagtatatagtggtcatcaaggacgcactatcatcttttgtgaaaccaagaaagaagcccaggagctgtcccagaattcagctataaagcaggatgctcagtccttgcatggagacattccacagaagcaaagggaaatcaccctgaaaggttttagaaatggtagttttggagttttggtggcaaccaatgttgctgcacgtgggttagacatccctgaggttgatttggttatacaaagctctccaccaaaggatgtagagtcctacattcatcgatccgggcggacaggcagagctggaaggacgggggtgtgcatctgcttttatcagcacaaggaagaatatcagttagtacaagtggagcaaaaagcgggaattaagttcaaacgaataggtgttccttctgcaacagaaataataaaagcttccagcaaagatgccatcaggcttttggattccgtgcctcccactgccattagtcacttcaaacaatcagctgagaagctgatagaggagaagggagctgtggaagctctggcagcagcactggcccatatttcaggtgccacgtccgtagaccagcgctccttgatcaactcaaatgtgggttttgtgaccatgatcttgcagtgctcaattgaaatgccaaatattagttatgcttggaaagaacttaaagagcagctgggcgaggagattgattccaaagtgaagggaatggtttttctcaaaggaaagctgggtgtttgctttgatgtacctaccgcatcagtaacagaaatacaggagaaatggcatgattcacgacgctggcagctctctgtggccacagagcaaccagaactggaaggaccacgggaaggatatggaggcttcaggggacagcgggaaggcagtcgaggcttcaggggacagcgggacggaaacagaagattcagaggacagcgggaaggcagtagaggcccgagaggacagcgatcaggaggtggcaacaaaagtaacagatcccaaaacaaaggccagaagcggagtttcagtaaagcatttggtcaataa

SEQ ID NO: 35 - DNA, 2460 bp, Homo sapiens
  source 1..2460: mol_type = genomic DNA
gcaggagagctggctgacaaaaaggaccgtgatgcatcaccttccaaggaggaaaggaagcgatcacggactcctgacagagagcgggatagagaccgggaccggaagtcttccccatctaaagatagaaagcggcatcgttcaagggatagacgtcgaggaggcagccgttctcgctctcgttcccgttccaaatctgcagaaagagaacgacggcacaaagaacgagaacgagataaggagcgggatcggaataagaaggaccgagatcgagacaaggatgggcacagacgggacaaggaccgtaaacgatccagcttatctcctggtcgaggaaaagactttaaatctcggaaggacagagactctaagaaggatgaagaggatgaacatggtgataagaagcctaaggcccagccattatccctggaggagcttctggccaagaaaaaggctgaggaagaagctgaggctaagcccaagttcctctctaaagcagaacgagaggctgaagctctaaagcgacggcagcaggaggtggaagagcggcagaggatgcttgaagaagagaggaagaaaaggaaacagttccaagacttgggcaggaagatgttggaagatcctcaggaacgggaacgtcgggaacgcagggagaggatggaacgggagaccaatggaaatgaggatgaggaagggcggcagaagatccgggaagagaaggataagagcaaggaactgcatgccattaaggagcgttacctgggtggcatcaaaaagcggcgccgaacgagacatctcaatgaccggaaatttgtttttgagtgggatgcatctgaggacacatccattgactacaaccccctgtacaaagaacggcaccaggtgcagttgttagggcgaggcttcattgcaggcattgacctcaagcagcagaagcgagagcagtcacgtttctatggagacctaatggagaagaggcgaaccctggaagaaaaggagcaggaggaggcaagactccgcaaacttcgtaagaaggaagccaagcagcgctgggatgatcgtcattggtctcagaaaaagttagatgagatgacggacagggactggcggatcttccgtgaggactacagcatcaccaccaaaggtggcaagatccccaatcccatccgatcctggaaagactcttctctgcccccacacatcttggaggtcattgataagtgtggctacaaggaaccaacacctatacagcgtcaggcaattcccattgggctacagaatcgtgacatcattggtgtggctgagactggcagtggcaagacagcagccttcctcatccctctgctggtctggatcaccacacttcccaaaattgacaggatcgaagagtcagaccaaggcccttatgccatcatcctggctcccacccgtgagttggctcaacagattgaggaagagaccatcaagtttgggaaaccgctaggtatccgcactgtggctgtcattggtggcatctccagagaagaccagggcttcaggctgcgcatgggttgtgagattgtgattgctacccctgggcgtttgattgatgtgctggagaaccgctacctggtgctgagccgctgtacctatgtggttctggatgaggcagataggatgattgacatgggctttgagccagatgtccagaagatcctggagcacatgcctgtcagcaaccagaagccagacacggatgaggctgaggaccctgagaagatgctggccaactttgagtcgggaaaacataagtaccgccaaacagtcatgttcacggccaccatgcccccagcggtggagcgtctggccaggagctatcttcggcgacctgctgtggtgtacattggctccgcaggcaagccccatgagcgtgtggaacagaaggtcttcctcatgtcagagtcagaaaagaggaaaaagctgctggcaatcttggagcaaggctttgacccacccatcattatttttgtcaaccagaagaagggctgcgacgtgttggccaaatccctggagaagatggggtacaatgcttgcacactgcacggtggaaaaggccaggagcagcgagagtttgcgttgtccaacctcaaggctggggccaaggatattttggtggctacagatgtggctggtcgtggtattgacatccaagatgtgtctatggttgtcaactatgatatggccaaaaatattgaagattacatccaccgcattggccgcacgggacgagcaggcaagagtggggtggccatcaccttcctcacaaaagaggactctgctgtgttctacgagctgaagcaagctatcctggaaagcccagtgtcttcctgtccccccgaactagccaaccacccagatgcccagcataagccaggcaccatcctcaccaagaagcgccgggaagagaccatctttgcctga

SEQ ID NO: 36 - DNA, 2577 bp, Homo sapiens
  source 1..2577: mol_type = genomic DNA
aagttgaaggacacaaaatcaaggccaaagcagtcaagctgtggcaaatttcagacaaagggaatcaaagttgtgggaaaatggaaggaagtgaagattgacccaaatatgtttgcagatggacagatggatgacttggtgtgctttgaggaattgacagattaccagttggtctcccctgccaagaatccctccagtctcttctcaaaggaagcacccaagagaaaggcacaagctgtttcagaagaagaggaggaggaggagggaaagtctagctcaccaaagaaaaagatcaagttgaagaaaagtaaaaatgtagcaactgaaggaaccagtacccagaaagaatttgaagtgaaagatcctgagctggaggcccagggagatgacatggtttgtgatgatccggaggctggggagatgacatcagaaaacctggtccaaactgctccaaaaaagaagaaaaataaagggaaaaaagggttggagccttctcagagcactgctgccaaggtgcccaaaaaagcgaagacatggattcctgaagttcatgatcagaaagcagatgtgtcagcttggaaggacctgtttgttcccaggccggttctccgagcactcagctttctaggcttctctgcacccacaccaatccaagccctgaccttggcacctgccatccgtgacaaactggacatccttggggctgctgagacaggaagtgggaaaactcttgcctttgccatcccaatgattcatgcggtgttgcagtggcagaagaggaatgctgcccctcctccaagtaacaccgaagcaccacctggagagaccagaactgaggccggagctgagactagatcaccaggcaaggctgaagctgagtctgatgcattgcctgacgatactgtaattgagagtgaagcactgcccagtgatattgcagccgaggccagagccaagactggaggcactgtctcagaccaggcgttgctctttggtgacgatgatgctggtgaagggccttcttccctgatcagggagaaacctgttcccaaacagaatgagaatgaggaggaaaatcttgataaagagcagactggaaatctaaaacaggagttggatgacaaaagcgccacctgtaaggcatatccaaagcgtcctctgcttggactggttctgactcccactcgagagctggccgtccaggtcaaacagcacattgatgctgtggccaggtttacaggaattaaaactgctattttggttggtggaatgtccacgcagaaacagcagaggatgctgaaccgtcgtcctgagattgtggttgctactccaggccggctgtgggaattaattaaagaaaagcattatcatttgaggaaccttcggcagctcaggtgcctggtagtggatgaggctgaccggatggttgagaaaggccattttgctgagctctcacagctgctagagatgctcaatgactcccaatacaacccaaagagacaaacgcttgttttttctgccacactcaccctggtgcatcaggctcctgctcgaatccttcataagaagcacaccaagaaaatggataaaacagccaaacttgacctccttatgcagaaaattggcatgaggggcaagcccaaggtcattgacctcacaaggaatgaggccacggtggagacgctaacagagaccaagatccattgtgagactgatgagaaagacttctacttgtactacttcctgatgcagtatccaggccgcagcttagtgtttgccaacagtatctcctgcatcaaacgcctctctgggctcctcaaagtccttgatatcatgcccttgaccctgcatgcctgtatgcaccagaagcagaggctcagaaacctggagcagtttgcccgtctggaagactgtgttctcttggcaacagatgtggcagctcggggtctggatattcctaaagtccagcatgtcatccattaccaggtcccacgtacctcggagatttatgtccaccgaagtggtcgaactgctcgagctaccaatgaaggcctcagtctgatgctcattgggcctgaggatgtgatcaactttaagaagatttacaaaacgctcaagaaagatgaggatatcccactgttccccgtgcagacaaaatacatggatgtggtcaaggagcgaatccgtttagctcgacagattgagaaatctgagtatcggaacttccaggcttgcctgcacaactcttggattgagcaggcagcagctgccctggagattgagctggaagaagacatgtataagggaggaaaagctgaccagcaagaagaacgtcggagacaaaagcagatgaaggttctgaagaaggagctgcgccacctgctgtcccagccactgtttacggagagccagaaaaccaagtatcccactcagtctggcaagccgcccctgcttgtgtctgccccaagtaagagcgagtctgctttgagctgtctctccaagcagaagaagaagaagacaaagaagccgaaggagccacagccggaacagccacagccaagtacaagtgcaaattaa

SEQ ID NO: 37 - DNA, 1449 bp, Homo sapiens
  source 1..1449: mol_type = genomic DNA
gcgtcgttactgtggggaggcgacgcaggggcggcggagagcgagcggctgaacagccacttttcaaacctcagccaaccccggaagaacctttggggtattaagagtactgcagtccgaaacatagatggttctattaataacatcaatgaagatgatgaagaagatgtagtggatttggcagctaattcactcttaaacaagttaatccatcaatccttagtagaatccagtcaccgtgtggaagttttacagaaggatcccagctctccactttactcagtaaagacatttgaagagctgcggctaaaggaagagttactaaaaggaatctatgcaatgggatttaataggccatctaaaatccaagagatggctctccctatgatgctggcacatccaccccagaacctcatagcacagagccagtctggaacaggaaagacagcggcatttgtgttggcaatgttaagcagagttaatgccttggaattgttcccacagtgcctctgcctagctcctacttatgaattggctctgcaaactggccgtgtggttgagcagatgggaaaattctgtgtggatgttcaagtgatgtatgccattcgagggaatcgaattcccagaggcaccgacatcactaaacagattataattggcactcctgggactgtcctagattggtgttttaaactaaaattgattgatttgactaagattcgtgtgtttgtcctggatgaagcagatgtgatgattgacactcaaggattctcagatcatagtattcgtattcaaagagctctaccctccgaatgccaaatgctcctcttttcagcaacctttgaggactctgtgtggcactttgctgagcgaatcatccctgaccctaatgttatcaagttacgcaaagaggagctcacactgaacaacatccggcaatattacgtgctgtgtgagcacaggaaagacaaataccaagctctgtgcaacatttatggcagcatcaccattggtcaggccatcatcttctgccagactcgtcgaaacgctaagtggttgaccgtggagatgatacaggatggccaccaggtgtctttgttaagcggggagctgaccgtggagcagcgagcttccatcattcagaggtttcgggatgggaaagagaaggttctcataacaactaatgtttgtgcccgagggattgatgtgaagcaggtcacaattgttgtgaactttgatctccctgtaaaacaaggagaggagccggactatgagacctacctccaccgcatagggcggacggggcgctttgggaaaaaaggccttgccttcaacatgattgaagtagatgagctgccctcgctcatgaaaatccaggaccactttaacagcagtattaagcaactcaacgctgaagacatggatgaaattgaaaagattgactattga

SEQ ID NO: 38 - DNA, 2295 bp, Homo sapiens
  source 1..2295: mol_type = genomic DNA
cttgcggacctcggcttaatcggaaccataggcgaggatgacgaggtgccggtggagcccgagtctgactccggggacgaggaagaggaggggcccattgtgctgggcagacgacaaaaagctttggggaagaaccgcagtgctgatttcaaccctgatttcgttttcactgagaaggaggggacgtacgatggcagctgggccctggctgatgtcatgagccaactcaagaagaagagggcagccactacattagatgagaagattgagaaagttcgaaagaaaaggaaaacagaggataaagaagccaagtctgggaagttggaaaaggagaaagaagcaaaggaaggctctgaaccaaaggagcaggaagaccttcaagagaatgatgaggaaggctcagaagatgaagcctcggagactgactactcatcagctgatgagaacatcctcaccaaagcagatacactcaaagtaaaggatcggaagaagaagaagaagaaaggacaggaagcaggaggattttttgaagatgcatctcagtacgatgaaaacctctcgttccaggacatgaacctttcccgccctcttctgaaggccattacagccatgggcttcaagcagcccaccccgatccagaaggcgtgcatacctgtgggtctattggggaaggacatctgtgcctgtgcagccactgggacaggtaaaactgccgcctttgccctgcctgttttggagcgtctgatttataaaccccgccaggctccagtcacccgcgtgctggtgctagtgcccacccgagagctgggcatccaggtgcactctgtcaccagacagctggcccagttctgcaacatcaccacctgcctggctgtgggcggcttggatgtgaagtctcaggaagcagctcttcgggcagcgcctgacatcctcatcgccaccccaggccggctcatcgatcacctccacaactgcccttccttccacctgagcagcatcgaggtgctcatcctggacgaggctgacaggatgctggatgagtactttgaggagcagatgaaggagatcatccgaatgtgttcccaccaccgccagaccatgctcttctcggccaccatgacagacgaggtgaaagatctggcttctgtctccttgaagaatcctgtccggatatttgtgaacagcaacacagatgtggctcccttcctgcggcaggagttcatccggatccggcctaatcgtgaaggagaccgggaagccatcgtggcagctttgttgacgaggaccttcactgaccatgtgatgctgttcacgcaaaccaagaagcaggcccaccgcatgcacatcctcctggggctcatggggctgcaggtgggtgagctccatggcaacttgtcacagacgcagcggctggaggccctccggcgttttaaggatgaacagattgacatcctcgtggccactgatgtggcagcccgtggacttgacattgagggggtcaaaacggtaatcaacttcacaatgcctaataccatcaaacattatgtccaccgggtggggcgaacagcacgtgctggcagggctgggcgctcagtctctctggtgggagaagatgagcggaagatgctgaaggagattgtaaaagctgccaaggcccctgtgaaggccaggatacttccccaagatgtcatcctcaaattccgggacaagattgagaaaatggagaaagatgtgtatgcagttctgcagctagaggcggaggaaaaagagatgcagcagtcagaagcccagatcaatacagcaaagcggctcctggagaaggggaaggaggcagtggtccaagagcccgagaggagctggttccagaccaaagaagagaggaagaaggagaaaattgccaaagctctgcaggaatttgacttggccttaagaggaaagaagaaaaggaagaagtttatgaaggatgccaaaaaaaagggggagatgacagcagaggaaaggtctcagtttgaaatcctcaaggcgcagatgtttgctgaacggctagcgaagaggaatcgcagagccaagcgggcccgagcaatgcccgaggaggagccagtgagaggtcctgccaagaagcaaaagcaggggaagaaatctgtatttgatgaagaactcaccaacacaagcaagaaggccctgaaacagtatcgagctggcccttcctttgaagaaaggaaacagttgggcttgccccaccagagacgaggaggaaactttaaatctaaatccagatacaagaggaggaagtag

SEQ ID NO: 39 - DNA, 1620 bp, Homo sapiens
  source 1..1620: mol_type = genomic DNA
gctctaacgcggccggtgcggctcttttccctcgtgactcggttgctcctggcgccgcgacggggcctcacggtccgcagtcccgacgaacccctgccggtggtgcgcatcccagtggctctacagcggcagttggaacagcggcagagcaggcggcggaacctcccgaggccggtgctggttcgacccggaccgctgctggtttcggcgcggcggccggagttgaaccagccggcgcgcctcacactgggccgttgggagcgcgcgccgctagcctctcaaggctggaagagtcgacgcgcgcgtcgggaccacttctccatcgagcgcgcgcaacaggaggcgccagcggtgcgaaagctctcgtctaagggcagctttgctgacctgggcctggagccccgtgtgctgcacgcactacaggaggctgcgcctgaagtcgttcagcccacaaccgtgcagtctagcaccatcccctcactacttcgcggccgccatgtcgtttgcgccgcagaaaccggcagtggcaagactctcagctacctcctgccgctgcttcaacggctcttgggccagccaagcctggactcccttcctatccccgcgccccgaggcctggtccttgttccttcccgagaattggcccaacaggtgcgggctgtggcccaacccttgggccgctccttgggcctgctggtgcgggacctggagggaggccacggcatgcgtaggatcaggctgcagctgtccagacagccttcagcagatgtgcttgtggccactccaggggctctgtggaaggccctgaaaagtcgactgatcagtctggagcaactctccttcttggtgttggatgaggcagacacactgctggatgaaagcttcctggaactggtggactacatcttagagaagagccacatagcagaaggcccagctgacttggaagaccccttcaatcccaaagctcagttagtgctggtaggagccacatttcccgaaggtgtaggccagttgctgaataaagtcgccagcccagatgctgtcaccaccatcaccagctccaagctccactgtatcatgcctcatgtgaaacagacatttctgagactgaagggagcagataaggtggccgagctggtgcacatcctcaagcatcgtgacagagcagaaaggactggtccctcaggaactgttctggtgttctgtaatagctccagcactgtgaactggctgggatatattctggatgaccacaaaatccaacacctaaggttgcaggggcaaatgccagccttgatgagggtaggaatcttccagtccttccagaagagctcccgagacatacttctctgcacagacatagcctctcggggcctggacagcactggtgtggagctggttgtcaattatgatttccccccaacgctgcaagattacatccacagagcagggagagtgggccgtgtggggagcgaggtgccaggcaccgtcatcagttttgtgacccatccctgggatgtgagcctggttcagaagattgagctggcggctcgccgaaggagaagtcttccaggactagcatcctcggtgaaagagcctttgccccaagcaacctga

SEQ ID NO: 40 - DNA, 2238 bp, Homo sapiens
  source 1..2238: mol_type = genomic DNA
gcagccgccgacggttcgctcttcgacaaccccaggacgttctccagacgtcccccagcccaggcgagtcggcaagcaaaggctacgaaaagaaaataccaagcgtccagtgaggctcccccagcgaaacggaggaacgaaacttcatttctcccagccaagaaaactagtgttaaagaaactcagaggacttttaaggggaacgcacaaaaaatgttttctccaaagaagcattcggttagcacaagtgatagaaaccaggaggagagacagtgcattaagacttcatcactgtttaaaaacaaccctgacattccagaactccacagacctgtggtaaagcaggtgcaagaaaaagtgtttacttcagctgcttttcatgagctgggcctccacccacatttaatttccacaataaatacggtcttaaaaatgtctagtatgaccagtgttcagaagcaaagtattcctgtgttgctggaaggcagagatgctctcgtgagatcccagacgggctcaggtaaaactcttgcctattgcatccctgtggtccagtcccttcaagcaatggagtcaaaaatacagcgcagtgatggcccctatgccctggtgctcgtgccaacgagagagctagctctacaaagctttgacactgtccagaaactgcttaagcctttcacctggattgtgcctggagtgttaatgggaggagagaagagaaaatcagaaaaggccagactccgcaaaggaataaatatccttatctcaactcctggacgcctggtggatcatataaaatccacaaagaacattcattttagtcggctgcggtggttggtgtttgatgaagcagacagaatcttggatttgggttttgaaaaggacatcacagtgatacttaatgctgtaaatgctgaatgccaaaaacgacagaatgtcttgctatcagcgacactcacagaaggtgtaacgcggctagctgatatcagtttgcatgatccagtcagtatttctgtcctggacaagagccatgaccagttgaacccaaaggacaaagcggtccaggaggtctgtcctccaccagctggcgacaagctggacagctttgcaataccagagagtctcaagcagcatgtgactgtggttcccagcaaactgaggcttgtctgcctagcggccttcatccttcagaaatgcaagtttgaggaagaccagaagatggttgtctttttctcaagttgcgagctggtggagttccactacagcctcttcctacagaccctgctgagcagctcaggggcgccggcatcagggcagttgccatctgcctccatgcgattaaaattcctacggctgcatggcggcatggagcaggaggaaagaacagcagtgtttcaggaattttcacattccagaagaggcgtccttctttgcacggatgttgcagctcggggcttagatctccctcaagtcacgtggattgttcagtacaacgctccatcttcacctgcagaatacatccaccggattggaagaaccgcccggattggctgccatgggagcagcctgctcattttggctccttcggaggcagaatatgtcaactcgttggcttctcacaaaatcaacgtttctgagattaagatggaagatattttgtgtgttctgacaagagatgattgttttaaagggaaacgatggggagcccagaaatcccatgctgttggcccccaggaaatccgagagcgagccacagtcttgcagacggtatttgaagattacgtgcactccagtgagaggagggtctcctgggcaaagaaagctctgcagtccttcatccaagcctacgccacctaccccagggagctgaagcacatcttccacgtccgatccctccaccttgggcatgtggcgaagagcttcggactaagagatgcccccaggaatcttagtgccttgactagaaagaagaggaaagcacacgtgaaaaggcctgaccttcataagaagacccagagtaaacacagcctcgctgaaatcctacgttcggaatactcaagcggcatggaggccgacatcgccaaggtcaaaaagcaaaacgcacctggagagcctggtggccggcccctgcagcacagtctgcagccgacaccctgctttggccgtgggaaaacattaaaatggagaaaaacccaaaaaggtgtacagcgggacagcaagacttcccagaaagtttaa

SEQ ID NO: 41 - DNA, 1284 bp, Homo sapiens
  source 1..1284: mol_type = genomic DNA
gcagagaacgatgtggacaatgagctcttggactatgaagatgatgaggtggagacagcagctgggggagatggggctgaggcccctgccaagaaggatgtcaagggctcctatgtctccatccacagctctggctttcgtgacttcctgctcaagccagagttgctccgggccattgtcgactgtggctttgagcatccgtcagaagtccagcatgagtgcatccctcaggccattctgggaatggatgtcctgtgccaggccaagtcgggcatgggaaagacagcagtgtttgtcttggccacactgcaacagctggagccagttactgggcaggtgtctgtactggtgatgtgtcacactcgggagttggcttttcagatcagcaaggaatatgagcgcttctctaaatacatgcccaatgtcaaggttgctgttttttttggtggtctgtctatcaagaaggatgaagaggtgctgaagaagaactgcccgcatatcgtcgtggggactccaggccgtatcctagccctggctcgaaataagagcctcaacctcaaacacattaaacactttattttggatgaatgtgataagatgcttgaacagctcgacatgcgtcgggatgtccaggaaatttttcgcatgaccccccacgagaagcaggtcatgatgttcagtgctaccttgagcaaagagatccgtccagtctgccgcaagttcatgcaagatccaatggagatcttcgtggatgatgagacgaagttgacgctgcatgggttgcagcagtactacgtgaaactgaaggacaacgagaagaaccggaagctctttgaccttctggatgtccttgagttcaaccaggtggtgatctttgtgaagtctgtgcagcggtgcattgccttggcccagctactagtggagcagaacttcccagccattgccatccaccgtgggatgccccaggaggagaggctttctcggtatcagcagtttaaagattttcaacgacgaattcttgtggctaccaacctatttggccgaggcatggacatcgagcgggtgaacattgcttttaattatgacatgcctgaggattctgacacctacctgcatcgggtggccagagcaggccggtttggcaccaagggcttggctatcacatttgtgtccgatgagaatgatgccaagatcctcaatgatgtgcaggatcgctttgaggtcaatattagtgagctgcctgatgagatagacatctcctcctacattgaacagacacggtag

SEQ ID NO: 42 - DNA, 1866 bp, Homo sapiens
  source 1..1866: mol_type = genomic DNA
gaggagtcggaacccgaacggaagcgggctcgcaccgacgaggtgcctgccggaggaagccgctccgaggcggaagatgaggacgacgaggactacgtgccctatgtgccgttacggcagcgccggcagctactgctccagaagctgctgcagcgaagacgcaagggagctgcggaggaagagcagcaggacagcggtagtgaaccccggggagatgaggacgacatcccgctaggccctcagtccaacgtcagcctcctggatcagcaccagcaccttaaagagaaggctgaagcgcgcaaagagtctgccaaggagaagcagctgaaggaagaagagaagatcctggagagtgttgccgagggccgagcattgatgtcagtgaaggagatggctaagggcattacgtatgatgaccccatcaaaaccagctggactccaccccgttatgttctgagcatgtctgaagagcgacatgagcgcgtgcggaagaaataccacatcctggtggagggagacggtatcccaccacccatcaagagcttcaaggaaatgaagtttcctgcagccatcctgagaggcctgaagaagaaaggcattcaccacccaacacccattcagatccagggcatccccaccattctatctggccgtgacatgataggcatcgctttcacgggttcaggcaagacactggtgttcacgttgcccgtcatcatgttctgcctggaacaagagaagaggttacccttctcaaagcgcgaggggccctatggactcatcatctgcccctcgcgggagctggcccggcagacccatggcatcctggagtactactgccgcctgctgcaggaggacagctcaccactcctgcgctgcgccctctgcattgggggcatgtccgtgaaagagcagatggagaccatccgacacggtgtacacatgatggtggccaccccggggcgcctcatggatttgctgcagaagaagatggtcagcctagacatctgtcgctacctggccctggacgaggctgaccgcatgatcgacatgggcttcgagggtgacatccgtaccatcttctcctacttcaagggccagcgacagaccctgctcttcagtgccaccatgccgaagaagattcagaactttgctaagagtgcccttgtaaagcctgtgaccatcaatgtggggcgcgctggggctgccagcctggatgtcatccaggaggtagaatatgtgaaggaggaggccaagatggtgtacctgctcgagtgcctgcagaagacacccccgcctgtactcatctttgcagagaagaaggcagacgtggacgccatccacgagtacctgctgctcaagggggttgaggccgtagccatccatgggggcaaagaccaggaggaacggactaaggccatcgaggcattccgggagggcaagaaggatgtcctagtagccacagacgttgcctccaagggcctggacttccctgccatccagcacgtcatcaattatgacatgccagaggagattgagaactatgtacaccggattggccgcaccgggcgctcgggaaacacaggcatcgccactaccttcatcaacaaagcgtgtgatgagtcagtgctgatggacctcaaagcgctgctgctagaagccaagcagaaggtgccgcccgtgctgcaggtgctgcattgcggggatgagtccatgctggacattggaggagagcgcggctgtgccttctgcgggggcctgggtcatcggatcactgactgccccaaactcgaggctatgcagaccaagcaggtcagcaacatcggtcgcaaggactacctggcccacagctccatggacttctga

SEQ ID NO: 43 - DNA, 2814 bp, Homo sapiens
  source 1..2814: mol_type = genomic DNA
aactggaataaaggtggtcctggcactaagcgaggatttggctttggaggttttgccatcagtgctgggaaaaaggaggaacccaaactcccacagcagtcccacagtgcctttggggcaaccagctcttcttctggatttggaaagtcagctccaccacagcttccttctttctacaaaattggatctaagcgggccaactttgatgaagaaaatgcctattttgaagatgaggaagaagattctagcaacgttgatttaccttacattcctgctgaaaactcaccaactcgccagcaattccattccaagccagtagattctgacagcgatgatgatcccttggaggcattcatggctgaagtggaggatcaggcagctagagacatgaagaggcttgaagaaaaggacaaggaaagaaaaaacgtaaagggtattcgagatgacattgaagaggaagatgaccaagaagcttattttcgatacatggcagaaaacccaactgctggtgtggttcaggaggaagaggaagacaatctagaatatgatagtgacggaaatccaattgcacctaccaaaaaaatcattgatcctcttccccccattgatcattcagagattgactatccaccatttgaaaaaaacttttacaatgagcatgaagagataaccaacctcactccacagcagttaatagatctccggcataagctcaatcttcgggtctctggtgctgcacctcctagaccaggaagtagctttgctcattttgggtttgacgaacaacttatgcaccagattcggaaatctgaatacacacagcccactccaatacagtgccagggtgtgcctgtggcattaagtggtagagacatgattggtattgccaaaacaggtagtgggaaaactgcagccttcatttggcccatgttgattcatataatggaccagaaggagttggaaccaggtgatggaccaattgcagtgattgtgtgtcctaccagggagctttgccagcagatccatgcagaatgtaagcggtttggaaaagcatataatcttcgatcagtggccgtatatggaggagggagtatgtgggagcaggccaaggcccttcaggagggggcagagattgttgtgtgtaccccaggtcgactgatagatcatgtgaaaaagaaagctaccaatcttcaaagagtctcttaccttgtgtttgatgaagcagatcgaatgtttgacatgggatttgagtaccaagttcgatccatagcaagtcatgttcgtcctgacaggcagactctcttatttagtgcaacttttcggaagaagattgaaaagttggccagagacatcctgatcgaccctattcgagtggtgcagggagatattggagaggcaaatgaagatgtgacacagattgtggagattctccattctggacctagtaaatggaactggcttacccggcgtctggtagaatttacctcttcagggagtgtcctcctctttgttactaaaaaagccaatgctgaagagctagcgaataaccttaaacaggagggtcataatcttgggctgctccatggggatatggatcagagtgagagaaacaaggtcatttcagactttaagaaaaaggacatcccagtcctggtggccacagatgttgcagcccgtggtctggacattccttcaattaagactgtcattaactatgatgtggcacgagacattgatacccacacgcataggattggccgcacaggaagagcgggtgagaaaggtgtggcctataccctactcactcccaaggacagcaattttgctggtgacctggtccggaacttggaaggagccaatcaacacgtttctaaggaactcctagatctggcaatgcagaatgcctggtttcggaaatctcgattcaaaggagggaaaggaaaaaagctgaacattggtggaggaggcctaggctacagggagcggcctggcctgggctctgagaacatggatcgaggaaataacaatgtaatgagcaattatgaggcctacaagccttccacaggagctatgggagatcgactaacggcaatgaaagcagctttccagtcacagtacaagagtcactttgttgcagccagtttaagtaatcagaaggctggaagttctgctgctggggcaagtgggtggactagtgcagggagcttgaattctgttccaactaactcagcacaacagggccataacagtcctgacagccccgtcaccagtgccgccaagggcatcccaggctttggcaatactggcaacatcagtggtgcccctgtgacctacccgtctgccggagcccaaggagtcaacaacacagcttcagggaataacagccgagaagggactgggggcagcaacgggaaaagagagagatatactgagaaccggggcagcagccgtcacagtcacggagagactggcaatcggcatagcgatagtccacgtcacggagatggtggtcgccatggagatggataccgccatccagaaagcagcagccgtcatactgatggccatcggcacggggagaacagacatggaggaagcgcaggccggcatggggagaaccggggtgcaaatgatggtcggaatggggaaagcaggaaagaagcttttaatcgtgagagcaagatggagcccaagatggaacccaaagtggacagcagcaagatggacaaggtggacagcaagacagataagacagctgacggctttgctgtcccagagccgcctaaacgcaagaaaagtcgatgggacagttag

SEQ ID NO: 44 - DNA, 1944 bp, Homo sapiens
  source 1..1944: mol_type = genomic DNA
tcccaccacggaggagctcccaaggcctctacgtgggtcgttgctagtcggcgaagctcgacagtgtcccgagcgccagagaggaggccggcggaggagttgaatcgaacaggtcctgagggatatagtgtcggcagaggtggtcgctggagaggcacctctaggcccccggaggccgtggccgctggtcacgaggaactgccgctgtgttttgctttgaagagccactttgttggcgcggtaatcggtcgtggtgggtcaaaaataaagaatatacaaagtacaacaaacaccacaatccaaataatacaagaacaaccagaatcattagtcaaaatttttggcagcaaggcaatgcaaacgaaagcaaaagcagtgatagacaattttgttaaaaagctagaagaaaattacaattcagaatgcggaattgatactgcattccaaccttctgttggaaaagatggaagcacagataacaatgttgttgcaggagatcggccattgatagattgggatcaaattagagaggaaggtttgaaatggcaaaaaacaaagtgggcagatttaccaccaattaagaaaaacttttataaagagtccactgccacaagtgccatgtcaaaagtagaagcagatagttggaggaaagaaaattttaatataacgtgggatgacttgaaggatggggagaaacgacctatccccaatcctacctgcacatttgatgacgcctttcaatgttatcctgaggttatggaaaacattaaaaaggcaggttttcaaaagccaacacctattcagtcacaggcatggcccattgtgttgcaaggaatagatcttataggagtagcccagactggaacaggaaagacattgtgttatttaatgcctggatttattcatctggtccttcaacccagccttaaaggtcaaaggaatagacccggcatgttagttctaactcccactcgggaattagcacttcaagtagaaggagaatgttgcaaatattcatataaagggcttcggagtgtttgtgtatatggtggtggaaatagagatgaacaaatagaagagcttaaaaaaggtgtagatatcataattgcaactcccggaagattgaatgatctgcaaatgagtaacttcgtcaatctgaagaatataacctacttggttttagatgaagcagacaagatgttggacatgggatttgaaccccagataatgaagattttgttagatgtgcgcccagataggcagacagttatgaccagtgctacatggcctcattcagttcatcgcctcgcacaatcttatttgaaagaaccaatgattgtctatgttggtacattggatctagttgctgtaagttcagtgaagcaaaatataattgtaaccaccgaggaagagaaatggagtcacatgcaaacttttctacagagtatgtcatccacagacaaagtcattgtcttcgtttctcgaaaagctgttgcggatcacttatcaagtgacctaatacttggaaatatatcagtagagtctctgcatggagatagagaacagagagatcgggagaaagcattagagaactttaaaacaggcaaagtgagaatactaattgcaactgatctagcctctagaggacttgatgtccatgacgttacacatgtctataattttgactttccacggaatattgaagaatacgtacaccgaatagggcgcacgggaagagcagggaggactggtgtttccattacaactttgactagaaatgattggagggttgcctctgaattgattaatattctggaaagagcaaatcagagtattccagaggagcttgtatcaatggctgagaggtttaaggcacatcaacagaaaagggaaatggaaagaaaaatggaaagacctcaaggaaggcccaagaagtttcattaa

SEQ ID NO: 45 - DNA, 3096 bp, Homo sapiens
  source 1..3096: mol_type = genomic DNA
ggtcgggagtcacgccactatcgaaaacgatcggcatcccggggtcgctctggaagtcggtctagaagtcgctcaccctcagacaaaagaagtaaacgtggagatgacagacggtctagaagtagagatagagataggaggagagagaggtctcgtagcagggataaaagaagatctcggtcaagggacaggaagcgtctgagacgttccagaagtagagagagagacagaagccgagagcgaagaagatctcgaagtagagacaggagacgctcaaggagtagaagccggggccggcgatcccgatcctccagtcctggaaataaaagcaagaaaactgagaatagatctaggtccaaagagaaaactgatggtggggaaagttctaaagagaagaaaaaagacaaagatgacaaggaggatgaaaaagaaaaagatgctggcaactttgaccagaataagctggaagaagaaatgagaaagcgaaaagaaagagtagaaaaatggcgagaagagcaacgtaaaaaggctatggaaaacataggagaactgaaaaaggaaatcgaagagatgaaacaagggaaaaagtggagtttagaggacgatgatgatgacgaagatgatcctgcagaagctgaaaaggagggaaatgaaatggagggtgaggagttagatccattagatgcttacatggaagaagtgaaagaggaagtaaaaaaatttaacatgagaagtgtaaaaggtggtgggggaaatgaaaagaagtctgggccaacggtcacaaaagttgtcactgttgtgacaaccaaaaaagcagttgtggattctgataagaagaaaggtgagctgatggagaatgaccaggatgccatggagtattcttcagaggaggaagaagttgatcttcagacagcccttacagggtatcaaacaaaacagcgaaagcttctagaaccagttgatcatggaaaaattgagtatgagccatttaggaaaaacttctatgttgaagttccagaactagcaaaaatgtctcaagaagaggtaaatgtgtttcgattggaaatggagggcattacagttaaaggaaaaggttgccccaaaccaattaaatcctgggtccagtgtggaatttccatgaagatcttaaattccctcaagaagcatggctatgaaaagcccacgcccatccaaacccaagctattcctgctataatgtctggacgagatttgattggcattgccaaaacaggaagtggaaagaccattgcttttctgttgcccatgtttagacacatcatggatcagaggtcattagaggaaggagaggggccaatagctgtcatcatgactccaactcgagaactggctttacagattactaaagagtgtaagaagttttccaagactttgggacttagagtggtctgtgtttacggaggaacaggaatcagtgagcagattgctgagctgaaaagaggtgctgaaattattgtttgcacacctggtcgaatgattgacatgttagccgctaacagtggtcgggtcacaaatcttcgaagagtgacatatgttgttttagatgaagcagacagaatgtttgacatgggttttgaaccccaggtcatgcgcatcgtggataatgttcgtcctgatcgacagacggttatgttttcagctactttccccagagctatggaggctttggctcgcaggatcctcagtaaacctattgaagtacaagttggaggcaggagtgtggtttgctcagatgtggagcaacaagtgattgtgattgaagaagaaaagaaattcttgaagttacttgagcttctaggccattatcaagagtcaggatctgtcattatatttgtggataagcaggaacatgctgatggtcttcttaaggatttaatgagagcatcttatccttgcatgtctcttcatggaggcattgatcaatatgacagagatagcatcataaatgactttaagaatgggacctgcaaacttcttgtggctacctctgttgctgcccgaggtctagatgtgaaacatctgattcttgtagtaaattatagctgccccaaccattatgaggattatgtacacagagcagggcggactggaagagcaggaaacaagggttatgcttatacttttatcacagaggatcaagctcgctatgctggtgacataattaaagctcttgaattgtcagggactgcagtacctcctgatttagagaaactgtggagtgatttcaaagatcagcagaaagctgaggggaaaataattaaaaagagtagtgggttctctggtaagggattcaagtttgatgaaacagaacaagctttggctaatgagaggaagaagttacaaaaagcagctcttggtctacaagattcagatgatgaggatgctgcagttgatattgatgagcaaattgaaagcatgtttaattcaaagaagagagtaaaggatatggctgctcctggaacatcaagtgttcctgctccaactgcaggaaatgctgagaaattagaaattgctaagagattggctcttagaatcaatgcccagaagaatttgggcatcgagtctcaggtagatgtgatgcagcaggccaccaatgcaattcttaggggtggcaccattctggctcccactgtttctgcaaaaaccattgcagaacaacttgctgaaaagatcaatgccaagctcaattatgtgccgttagagaaacaagaagaagagagacaggatggtggacagaatgaatcttttaagagatatgaagaagaattagagatcaatgacttcccacagactgctaggtggaaagttacctctaaggaagctctgcagagaatcagtgaatactctgaagccgcaattacaatcagaggaacctacttccctcctggcaaagaacccaaggaaggcgagcggaagatttacttggcaattgaaagtgccaatgaactggctgtgcagaaagcaaaggcagaaatcaccaggctcataaaagaagagctgatccggctgcaaaattcataccaaccaacaaataaaggaagatacaaagtcttatag

SEQ ID NO: 46 - DNA, 1365 bp, Homo sapiens
  source 1..1365: mol_type = genomic DNA
gcggcacccgaggaacacgattctccgaccgaagcgtcccagccgattgtggaagaggaggaaactaaaacatttaaagacctgggtgtgacagatgtgttgtgtgaagcttgtgaccagttgggatggacaaaacccaccaagatccagattgaagctattcctttggccttacaaggtcgtgatatcattgggcttgcagaaactggctctggaaagacaggcgcctttgctttgcccattctaaacgcactgctggagaccccgcagcgtttgtttgccctagttcttaccccgactcgggagctggcctttcagatctcagagcagtttgaagccctggggtcctctattggagtgcagagtgctgtgattgtaggtggaattgattcaatgtctcaatctttggcccttgcaaaaaaaccacatataataatagcaactcctggtcgactgattgaccacttggaaaatacgaaaggtttcaacttgagagctctcaaatacttggtcatggatgaagccgaccgaatactgaatatggattttgagacagaggttgacaagatcctcaaagtgattcctcgagatcggaaaacattcctcttctctgccaccatgaccaagaaggttcaaaaacttcagcgagcagctctgaagaatcctgtgaaatgtgccgtttcctctaaataccagacagttgaaaaattacagcaatattatatttttattccctctaaattcaaggatacctacctggtttatattctaaatgaattggctggaaactcctttatgatattctgcagcacctgtaataatacccagagaacagctttgctactgcgaaatcttggcttcactgccatccccctccatggacaaatgagtcagagtaagcgcctaggatcccttaataagtttaaggccaaggcccgttccattcttctagcaactgacgttgccagccgaggtttggacatacctcatgtagatgtggttgtcaactttgacattcctacccattccaaggattacatccatcgagtaggtcgaacagctagagctgggcgctccggaaaggctattacttttgtcacacagtatgatgtggaactcttccagcgcatagaacacttaattgggaagaaactaccaggttttccaacacaggatgatgaggttatgatgctgacagaacgcgtcgctgaagcccaaaggtttgcccgaatggagttaagggagcatggagaaaagaagaaacgctcgcgagaggatgctggagataatgatgacacagagggtgctattggtgtcaggaacaaggtggctggaggaaaaatgaagaagcggaaaggccgttaa

SEQ ID NO: 47 - DNA, 1233 bp, Homo sapiens
  source 1..1233: mol_type = genomic DNA
gcgaccacggccacgatggcgacctcgggctcggcgcgaaagcggctgctcaaagaggaagacatgactaaagtggaattcgagaccagcgaggaggtggatgtgacccccacgttcgacaccatgggcctgcgggaggacctgctgcggggcatctacgcttacggttttgaaaaaccatcagcaatccagcaacgagcaatcaagcagatcatcaaagggagagatgtcatcgcacagtctcagtccggcacaggaaaaacagccaccttcagtatctcagtcctccagtgtttggatattcaggttcgtgaaactcaagctttgatcttggctcccacaagagagttggctgtgcagatccagaaggggctgcttgctctcggtgactacatgaatgtccagtgccatgcctgcattggaggcaccaatgttggcgaggacatcaggaagctggattacggacagcatgttgtcgcgggcactccagggcgtgtttttgatatgattcgtcgcagaagcctaaggacacgtgctatcaaaatgttggttttggatgaagctgatgaaatgttgaataaaggtttcaaagagcagatttacgatgtatacaggtacctgcctccagccacacaggtggttctcatcagtgccacgctgccacacgagattctggagatgaccaacaagttcatgaccgacccaatccgcatcttggtgaaacgtgatgaattgactctggaaggcatcaagcaatttttcgtggcagtggagagggaagagtggaaatttgacactctgtgtgacctctacgacacactgaccatcactcaggcggtcatcttctgcaacaccaaaagaaaggtggactggctgacggagaaaatgagggaagccaacttcactgtatcctcaatgcatggagacatgccccagaaagagcgggagtccatcatgaaggagttccggtcgggcgccagccgagtgcttatttctacagatgtctgggccagggggttggatgtccctcaggtgtccctcatcattaactatgatctccctaataacagagaattgtacatacacagaattgggagatcaggtcgatacggccggaagggtgtggccattaactttgtaaagaatgacgacatccgcatcctcagagatatcgagcagtactattccactcagattgatgagatgccgatgaacgttgctgatcttatctga

SEQ ID NO: 48 - DNA, 1449 bp, Homo sapiens
  source 1..1449: mol_type = genomic DNA
gcaggcttcgcggagctcgggctgtcatcgtggctcgtggaacaatgtcggcagctgggtttgaagcagcccacgcccgtgcagctcggctgcatccccgccatcctggagggtcgagactgcttgggctgtgctaagacaggcagtgggaagacagcagcgtttgtccttcccatcttgcagaagctgtctgaggatccctatggcatcttctgcctcgtcctgacacccaccagggagctggcctaccagatcgcagagcagttccgggtcctggggaagcctctagggctgaaagactgcatcatcgtcggtggcatggacatggtggcccaggcgctggagctctctcggaaaccacacgtggtcatcgccacgccggggcgcctggcagatcacctgcgcagctccaacacttttagtataaagaagatccgcttcctggtgatggatgaggcagaccggctgctggaacagggctgcactgacttcaccgtggacctggaggccatcctggcggctgtgccggcccgcaggcagacactgctgttcagcgccacgctgaccgacacactccgggagctgcagggtctggccaccaaccagcccttcttctgggaagcacaggccccggtgagcaccgtggagcagctggaccagcgctacctgctggtgcctgagaaggtcaaggacgcctacctggtccacctgatccagcgcttccaggatgagcacgaggactggtccattatcatcttcaccaacacgtgcaagacctgccagattctgtgcatgatgctgcgcaaattcagcttccccaccgtggctctgcactccatgatgaagcagaaagaacgctttgccgccctagccaagttcaagtccagcatctaccggatcctgatcgcaacagacgtggcctcccggggcctggacatccctacggtacaggtggtcatcaaccacaacacccccgggctccccaagatctacatccaccgagtcggccggacggcccgtgcagggcggcagggtcaggccatcacgctggtgacacagtacgacatccacctggtgcacgccatcgaggagcagatcaagaagaagctggaggagttctccgtggaagaggccgaggtgctacagatcctcacacaggtcaacgtggtgcgaagagagtgtgagatcaaactggaggcggcccactttgacgaaaagaaggagatcaacaaacggaagcagctgatcctggaggggaaggaccctgacctggaggccaagcgcaaggctgagctggccaagatcaagcagaagaaccggcgcttcaaggagaaggtggaggagacgctgaagcgacagaaggctggcagggctggccacaaggggcgtccacccaggacaccgtctgggtcccactcaggcccagtcccctcccagggcctggtctga

SEQ ID NO: 49 - DNA, 2211 bp, Homo sapiens
  source 1..2211: mol_type = genomic DNA
cctgggaaactcctctggggggacattatggagctggaagcacccttggaggagtccgagagccagaagaaggagaggcaaaagagtgacagaaggaagtcaaggcaccattatgactcggatgagaaatcagaaacaagagaaaatggtgttacagatgacctggatgctcccaaggccaaaaaatctaaaatgaaagagaagctaaatggagacactgaagaaggatttaatagactttcagatgaattctccaaatctcataagtcaagaagaaaagatctaccaaatggagatatagatgaatatgaaaaaaaatcaaagcgagtatcatctttagatacttctactcataaatcaagtgataataaactagaggagaccttaacacgtgaacagaaagaaggagccttctccaattttcctatttctgaagagactataaagcttctgaaaggtcgaggggtaacatatctctttcctattcaagttaagacctttggtcctgtatatgaaggaaaagatttaatagctcaagcacggacaggaacaggaaagacattctcttttgccatccccttaattgaaagactccaaagaaatcaagaaacaattaaaaaaagccgctcaccaaaggtacttgttttggctccaacaagggaactggcaaaccaagtagccaaagacttcaaagatataactaggaaactcagcgtggcgtgtttttatggtggaacatcatatcaaagccaaattaatcatattcgaaatggtattgacatcttggttggaacacctggtcgtatcaaagaccatctgcagagtggccgattggatctttctaaactgcgacatgttgtgcttgatgaagtggatcagatgttagatttaggtttcgctgaacaagttgaagatattattcatgaatcctacaaaactgattctgaagacaatcctcagactttacttttttctgcaacttgcccacagtgggtatacaaagttgcaaaaaaatacatgaaatccagatatgaacaggttgaccttgttggaaaaatgactcaaaaggctgcaactactgtggaacatttggccatccagtgtcattggtctcagaggccagcagttattggagatgtccttcaagtctacagtgggtctgaagggagggctattattttctgtgagaccaagaagaatgtaactgaaatggccatgaatccacacataaaacagaatgcccagtgtttacatggggacattgcacagtcacaaagagaaattacactaaaaggcttcagagaaggtagttttaaagttttggtggcaaccaatgtggctgcccgtggtttggacattcctgaagttgacctggtgattcaaagttctcctcctcaggatgttgagtcctatatccatcgctctggacgcacaggtagagctggacggacagggatttgtatatgtttttatcaaccaagagaaagaggtcaactaagatatgtggaacaaaaagcaggaattacttttaaacgtgtaggtgttccttctacaatggatttagttaaatctaaaagcatggatgccatcaggtctctggcttccgtttcttacgctgctgttgattttttccgaccatcagctcagagactgatagaagagaaaggtgcagtggatgcattggctgcagctttagcccacatttctggtgcatcaagctttgaaccacgatctttgatcacctctgataaggggtttgtgaccatgactctggaaagcctagaggaaatacaggatgtcagctgtgcttggaaagaacttaacagaaagctgagtagtaatgcagtgtctcagattaccagaatgtgcctcctgaaaggaaatatgggtgtttgctttgatgttcctacaactgagtcagaaaggttacaggcagagtggcatgattccgactggatactctcagtgccagccaaattacctgaaattgaagaatattatgatggaaacacatcttctaattccagacagaggagtggctggtcaagtggtcgatcaggccggtcaggccggtcaggtggtcgatctggcggccggtcaggtagacagagtcgacaaggaagtcgctcaggaagtcgacaagatggtagaagacgaagtgggaatagaaatcgatcaagaagtgggggccacaaacggagttttgactga

SEQ ID NO: 50 - DNA, 1998 bp, Homo sapiens
  source 1..1998: mol_type = genomic DNA
gcgctgttctacgtcgcgcggtacccgggccccgatgcggcagctgcggcggggccggagggcgcggaggccggggcgcacggcagggcccgcgcgctgctcgagcggctgcagagccgggcccgcgaacggcagcagcagcgggagcccgcgcagaccgaggcggctgcatcgaccgagccggcgaccaggaggcgacggcggccccggcggcggcggcgggtgaacgacgcggagccggggagcccggaggcgccgcagggaaagcgacggaaggcggacggcgaggacgcgggcgcagaaagcaacgaggaggcgccaggggagcccagcgcagggagcagcgaggaggcgccaggggagcccagcgcagggagcagcgaggaggcgccgggggagcgcagcaccagcgccagcgccgaggcggccccagatggaccggccctggaggaggcggccggacccctggtccccggcctggtgctgggggggttcgggaagaggaaggcgccgaaggtccagcctttcctgccaaggtggctggctgagcctaactgtgtcagaaggaatgtcaccgaagacctggttcctatcgaggacatccctgacgtccatcctgacctgcagaagcagctgcgggcacacggcatctcgtcctactttccagtccaggcagctgtgattcctgccctcctggagagcgcagcctgtgggtttctggtgggcagaggtggctaccggcctagcgacctctgtgtttctgccccaacaggcagtgggaagacactggccttcgtcatccctgtggtgcaggccctgctttcgagagtggtctgccacatccgtgccctggttgtgctgcccaccaaggagctggcccagcaggtgagcaaagttttcaacatctacacagatgccacacctctgagagtctccctggttacgggacagaagtctctggccaaggagcaggagagcctcgtccagaaaacagctgatgggtaccgctgcttggctgacatcgtggtagccacccccggccgcctggtggaccacatcgaccagaccccaggattcagcctccagcagctccgcttcctgattatcgacgaggctgaccggatgattgacagcatgcatcagtcctggctgccgcgggtggtggcggccgccttccagagcgaggaccccgcggacccctgtgccctgctccagcgaaggcaggcccaggctgtgacagccgccagcacctgctgtccccagatgcccctgcagaagctgctcttctcagctactctgacccagaaccctgaaaagctgcagcagctgggcctccaccagccccggcttttctccacagggctagcacacaggggcctggaagatacagatggggacggggattcggggaagtatgcctttcctgttgggctcacgcaccactacgtgccctgcagcctcagctctaagccgctggtcgtcctgcacctggtcctggagatgggcttctcgagggttctctgcttcactaactcccgagagaactcccacaggctcttcctgctggtgcaagcttttgggggtgtggacgtggctgagttctcctcgcgctacgggcctggccagaggaggatgatcctgaagcagtttgaacaggggaagatccagctgctcatcagcacggacgccaccgcgcgaggcatcgacgtgcagggtgtggagctggtggtgaactacgacgccccccagtacctgagaacctacgtgcaccgggttgggaggacagctcgcgctgggaaaactggacaggccttcacactgctcctgaaagtgcaggagaggagattcctccgaatgctaactgaagctggggcacctgagttgcagcggcacgagctctccagcaagctgctgcagccgctggttcctcggtacgaggaggccctgtcccagctggaggagtctgtcaaggaagagcgcaagcagagggcggcctag

SEQ ID NO: 51 - DNA, 1797 bp, Homo sapiens
  source 1..1797: mol_type = genomic DNA
gacgtccacgatctctttcgccggctcggcgcgggggccaaattcgacacgagacgcttctcggcagacgcagctcgattccagataggaaaaaggaaatatgactttgattcttcggaggtgcttcagggactggacttttttggaaacaagaagtctgtcccaggtgtgtgtggagcatcacaaacacatcagaagccccaaaatggagagaaaaaagaagagagcctaactgaaaggaagagggagcagagcaagaaaaaaaggaagacgatgacttcagaaattgcttcccaagaagaaggtgctactatacagtggatgtcatctgtagaagcaaagattgaagacaaaaaagttcagagagaaagtaaactaacttccggaaagttggagaatctcagaaaagaaaagataaacttcttgcggaataaacacaaaattcacgtccaaggaaccgatcttcctgacccaattgctacatttcagcaacttgaccaggaatataaaatcaattctcgactacttcagaacattctagatgcaggtttccaaatgcctacgccaatccaaatgcaagccatcccagttatgctgcatggtcgggaacttctggcttctgctccaactggatctggaaaaacattagcttttagcattcctattttaatgcagctgaaacaacccgcaaataaaggcttcagagccctgattatatcaccaacacgagaacttgccagccagattcacagagagttaataaaaatttctgagggaacaggattcaggatacacatgatccacaaagcagcagtggcagccaagaaatttggacctaaatcatctaaaaagtttgatattcttgtgactactccaaatcgactaatctatttattaaagcaagatccccccggaatcgacctagcaagtgttgagtggcttgtagtagacgaatcagataaactgtttgaagatggcaaaactgggttcagagaccagctggcttccattttcctggcctgcacatcccacaaggtccgaagagctatgttcagtgcaacttttgcatatgatgttgaacagtggtgcaaactcaacctggacaatgtcatcagtgtgtccattggagcaaggaattctgcagtagaaactgtagaacaagagcttctctttgttggatctgagaccggaaaacttctggccatgagagaacttgttaaaaagggtttcaatccacctgttcttgtttttgttcagtccattgaaagggctaaagaactttttcatgagctcatatatgaaggtattaatgtggatgttattcatgcagagagaacacaacaacagagagataacacagtccacagtttcagagcaggaaaaatctgggttctgatttgtacagccttgctagcaagagggattgattttaaaggtgtgaacttggtgatcaactatgactttccaactagctcagtggaatatatccacaggataggtcgaactggaagagcagggaataagggaaaagcaattacatttttcactgaggatgataagccattattaagaagcgttgctaatgttatacagcaggctgggtgtcctgtaccagaatacataaaaggttttcagaaactactaagcaaacaaaagaaaaagatgattaagaaaccattggaaagggagagcattagtacaactccaaaatgtttcttagaaaaagctaaggataaacagaaaaaggtcactggtcagaacagcaagaagaaagtagctcttgaagacaaaagttaa

SEQ ID NO: 52 - DNA, 1893 bp, Homo sapiens
  source 1..1893: mol_type = genomic DNA
tcccactgggccccagagtggaagagggcggaggctaatccaagagaccttggggccagctgggatgtcaggggcagcagaggcagtggctggagtggccccttcggccatcagggaccgagagcagcaggctcccgtgaaccaccactctgctttaaaataaagaacaatatggttggtgtggtcattggttacagtggatcaaaaataaaagatctacaacattcgacaaacactaaaatacagatcataaacggggaatctgaagcaaaagtcagaatttttggcaatagggaaatgaaagcaaaggccaaagcggctatagaaacacttattagaaaacaagaaagctacaactcagaatccagtgtggataatgctgcatcccaaacccctattggaagaaatctaggcagaaatgacattgttggagaagctgagccattgtcaaattgggatcgcattagggcagcagtcgtggagtgcgaaaagagaaaatgggcagatctaccaccagttaagaaaaacttttacatagaatccaaagcaacaagctgcatgtctgaaatgcaggtgattaactggagaaaggaaaatttcaacataacgtgtgatgacttgaaaagtggtgaaaagcgtctcattccaaaaccaacttgcaggtttaaagacgcttttcagcaataccctgatcttctgaaaagcataataagggtagggattgtaaagccaacgccaattcagtcacaggcatggccaattattctacaaggaatagatcttatagtagttgcacaaaccggaacagggaaaacattgtcctatctaatgcctgggtttattcatcttgattctcaaccaatatctagagagcaaaggaatgggcctgggatgctagtccttacacccactagagagttggctcttcacgtggaagctgaatgttcaaagtattcatataaaggtctcaaaagcatttgcatatatggtggtagaaacagaaatggacaaatagaagacattagcaaaggtgtagatatcattattgcaactcctgggaggctgaatgacctacaaatgaataactctgtcaacctaagaagcataacctacttggttatagatgaggcagataaaatgctggatatggaatttgaaccccagataaggaagattttattagatgtgcgcccagaccgacagactgttatgacaagtgcaacttggccagatactgtacgtcaactagcactttcttatttgaaagatcctatgattgtttatgttggtaatctgaatctagtggctgtaaatacagtgaagcaaaatataattgttaccacagaaaaagaaaaacgagctctcacccaagaattcgtagagaacatgtcacccaacgacaaagtcatcatgtttgtcagccaaaaacatattgctgatgacttgtcaagcgacttcaatatccaaggcatatctgcagaatcattacatggcaacagtgaacagagtgatcaagagcgagcagtagaggactttaaaagcggaaacataaagatactgattacaactgatatagtatcccgaggtcttgatcttaatgatgtcacacatgtatataattatgatttcccaaggaatattgacgtatatgtacacagagtagggtacattggacggacaggaaagactggcacatcagttaccctcatcactcagagagattcgaaaatggccggtgaattgattaaaattctggacagagcaaatcagagtgttccggaagatcttgtagtaatggctgagcaatacaagttaaatcaacaaaagaggcacagagaaacacgatcaagaaaacctggacaaagacgcaaggagttttattttttaagttga

SEQ ID NO: 53 - DNA, 2643 bp, Homo sapiens
  source 1..2643: mol_type = genomic DNA
gcggccgacaagggcccggcggctggacctcggtcgcgagctgccatggcccagtggaggaagaagaaagggctccggaagcgccgaggcgcggcctcccaggcccgcggcagcgactcggaggacggcgagtttgagatccaggcggaagatgacgcccgggcccggaagctgggacctggaagacccctgcccaccttccccacctcggaatgcacctcggatgtggagccggacacccgggagatggtgcgtgcccagaacaagaagaagaagaagtctggaggcttccagtccatgggcctgagctacccggtgttcaaaggcatcatgaagaaggggtacaaggtgccaacacccatccagaggaagaccatcccggtgatcttggatggcaaggacgtggtggccatggcccggacgggcagtggcaagacagcctgcttcctcctcccaatgttcgagcggctcaagacccacagtgcccagaccggggcccgcgccctcatcctctcgccgacccgagagctggccctgcagaccctgaagttcactaaggagctaggcaagttcactggcctcaagactgccctgatcctgggtggagacaggatggaagaccagtttgcagccctgcacgaaaatcccgacataattattgccacgcccggacggttggtgcatgtggctgtggaaatgagcctgaagctgcagagtgtggaatacgtggtgttcgatgaagctgaccggctttttgaaatgggtttcgcagagcagctgcaggagatcatcgcccgcctccccgggggccaccagacggtgctgttctccgccacgctgcccaaactgctggtggaatttgcccgggctggcctcacggagcccgtgctcatccggcttgacgtggataccaagctcaacgagcagctgaagacctccttcttcctcgtgcgggaggacaccaaggctgccgtgctgctccacctgctgcacaacgtggtgcggccccaggaccagaccgtggtgtttgtggccacgaagcaccacgccgagtacctcactgagctgctgacgacccagcgggtgagctgcgcccacatctacagtgccctagacccgacagcccgcaagatcaatctcgccaaattcacgcttggcaagtgctccactctcattgtgactgacctggccgcccgaggcctggacatcccgctgctggacaatgtcatcaactacagcttccccgccaagggcaaactcttcctgcaccgcgtgggccgtgtggctcgggctggccgaagtggcacagcctactccttggtggcccctgatgaaatcccctacctgctggatctgcacctgttcctgggccgctccctcaccctcgcccgacccctcaaggagccctcaggtgtggccggtgtggatggcatgctgggtcgggtgccacagagtgtggtggacgaggaggacagtggtctgcagagcaccctggaggcatcgctggagctacggggcctggcccgcgttgctgataacgcccagcagcagtatgtgcgctcacgcccggcgccctcgcctgagtccatcaagagggccaaggagatggaccttgtggggctgggcctgcaccccctcttcagctcgcgttttgaggaggaggagctgcagcggctgaggctggtggacagcataaagaactaccgctcccgggcgactatctttgagatcaacgcctccagccgagacctgtgcagccaggtgatgcgcgccaagcggcagaaggaccgcaaggccatcgcccgcttccagcagggacagcaggggcggcaggagcagcaggagggcccagtgggcccagccccgagccgcccagcactgcaggagaagcagcctgagaaggaggaggaggaggaggcgggagagagtgtggaggacattttctcagaggtcgtgggccggaagcggcagcggtcaggacccaacaggggagccaagaggcggagggaggaggcccggcagcgggaccaggaattctacatcccctaccggcccaaggactttgacagcgagcggggcctgagcatcagcggggaagggggagcctttgagcagcaggcagctggcgctgtcctggacttgatgggggatgaagcccagaacctgacgaggggccggcagcagctcaagtgggaccgtaagaagaagcggtttgtgggacagtcaggacaggaagacaagaagaagattaagacagagagcggccgctacatcagcagctcctacaagcgagacctctatcagaagtggaaacagaaacagaaaattgatgatcgtgactcggacgaagaaggggcatctgaccggcgaggcccagagcgaagaggtgggaagcgagaccgtggccaaggtgcatcccggccccacgccccaggcacccctgcaggccgagtccgcccggaactcaagaccaagcagcagatcctgaagcagcggcgccgggcccagaagctgcacttcctgcagcgtggtggcctcaagcagctctctgcccgcaaccgccgccgcgtccaggagctgcagcagggcgccttcggccggggtgcccgctccaagaagggcaagatgcggaagaggatgtga

SEQ ID NO: 54 - DNA, 1800 bp, Homo sapiens
  source 1..1800: mol_type = genomic DNA
gagcatgtgacagagggctcctgggagtcgctgcctgtgccgctgcacccgcaggtgctgggcgcgctgcgggagctgggcttcccgtacatgacgccggtgcagtccgcaaccatccctctgttcatgcgaaacaaagatgtcgctgcagaagcggtcacaggtagtggcaaaacactcgcttttgtcatccccatcctggaaattcttctgagaagagaagagaagttaaaaaagagtcaggttggagccataatcatcacccccactcgagagctggccattcaaatagacgaggtcctgtcgcatttcacgaagcacttccccgagttcagccagattctttggatcggaggcaggaatcctggagaagatgttgagaggtttaagcaacaaggtgggaacatcattgtggccactccaggccgcttggaggacatgttccggaggaaggccgaaggcttggatctggccagctgtgtgcgatccctggatgtcctggtgttggatgaggcagacagacttctggacatggggtttgaggcaagcatcaacaccattctggagtttttgccaaagcagaggagaacaggccttttctctgccactcagacgcaggaagtggagaacctggtgagagcgggcctccggaaccctgtccgggtctcagtgaaggagaagggcgtggcagccagcagtgcccagaagaccccctcccgcctggaaaactactacatggtatgcaaggcagatgagaaatttaatcagctggtccattttcttcgcaatcataagcaggagaaacacctggtcttcttcagcacctgtgcctgtgtggaatactatgggaaggctctggaagtgctggtgaagggcgtgaagattatgtgcattcacggaaagatgaaatataaacgcaataagatcttcatggagttccgcaaattgcaaagtgggattttagtgtgcactgatgtgatggcccggggaattgatattcctgaagtcaactgggttttgcagtatgaccctcccagcaatgcaagtgccttcgtgcatcgctgcggtcgcacagctcgcattggccacgggggcagcgctctggtgttcctcctgcccatggaagagtcatacatcaatttccttgcaattaaccaaaaatgccccctgcaggagatgaagccccagagaaacacagcggaccttctgccaaaactcaagtccatggccctggctgacagagctgtgtttgaaaagggcatgaaagcttttgtgtcatatgtccaagcttatgcaaagcatgaatgcaacctgattttcagattaaaggatcttgattttgccagccttgctcgaggttttgccctgctgaggatgcccaagatgccagaattgagaggaaagcagtttccagattttgtgcccgtggacgttaataccgacacgattccatttaaagataaaatcagagaaaagcagaggcagaaactcctggagcaacaaagaagagagaaaacagaaaatgaagggagaagaaaattcataaaaaataaagcttggtcaaagcagaaggccaaaaaagaaaagaagaaaaaaatgaatgagaaaaggaaaagggaagagggttctgatattgaagatgaggacatggaagaacttcttaatgacacaagactcttgaaaaaacttaagaaaggcaaaataactgaagaagaatttgagaagggcttgttgacaactggcaaaagaacaatcaagacagtggatttagggatctcagatttggaagatgactgctga

SEQ ID NO: 55 - DNA, 1641 bp, Homo sapiens
  source 1..1641: mol_type = genomic DNA
gaggactctgaagcactgggcttcgaacacatgggcctcgatccccggctccttcaggctgtcaccgatctgggctggtcgcgacctacgctgatccaggagaaggccatcccactggccctagaagggaaggacctcctggctcgggcccgcacgggctccgggaagacggccgcttatgctattccgatgctgcagctgttgctccataggaaggcgacaggtccggtggtagaacaggcagtgagaggccttgttcttgttcctaccaaggagctggcacggcaagcacagtccatgattcagcagctggctacctactgtgctcgggatgtccgagtggccaatgtctcagctgctgaagactcagtctctcagagagctgtgctgatggagaagccagatgtggtagtagggaccccatctcgcatattaagccacttgcagcaagacagcctgaaacttcgtgactccctggagcttttggtggtggacgaagctgaccttcttttttcctttggctttgaagaagagctcaagagtctcctctgtcacttgccccggatttaccaggcttttctcatgtcagctacttttaacgaggacgtacaagcactcaaggagctgatattacataacccggttacccttaagttacaggagtcccagctgcctgggccagaccagttacagcagtttcaggtggtctgtgagactgaggaagacaaattcctcctgctgtatgccctgctcaagctgtcattgattcggggcaagtctctgctctttgtcaacactctagaacggagttaccggctacgcctgttcttggaacagttcagcatccccacctgtgtgctcaatggagagcttccactgcgctccaggtgccacatcatctcacagttcaaccaaggcttctacgactgtgtcatagcaactgatgctgaagtcctgggggccccagtcaagggcaagcgtcggggccgagggcccaaaggggacaaggcctctgatccggaagcaggtgtggcccggggcatagacttccaccatgtgtctgctgtgctcaactttgatcttcccccaacccctgaggcctacatccatcgagctggcaggacagcacgcgctaacaacccaggcatagtcttaacctttgtgcttcccacggagcagttccacttaggcaagattgaggagcttctcagtggagagaacaggggccccattctgctcccctaccagttccggatggaggagatcgagggcttccgctatcgctgcagggatgccatgcgctcagtgactaagcaggccattcgggaggcaagattgaaggagatcaaggaagagcttctgcattctgagaagcttaagacatactttgaagacaaccctagggacctccagctgctgcggcatgacctacctttgcaccccgcagtggtgaagccccacctgggccatgttcctgactacctggttcctcctgctctccgtggcctggtgcgccctcacaagaagcggaagaagctgtcttcctcttgtaggaaggccaagagagcaaagtcccagaacccactgcgcagcttcaagcacaaaggaaagaaattcagacccacagccaagccctcctga

SEQ ID NO: 56 - DNA, 2640 bp, Homo sapiens
  source 1..2640: mol_type = genomic DNA
accaccgagcagcgacgcagcctgcaagccttccaggattatatccggaagaccctggaccctacctacatcctgagctacatggccccctggtttagggagggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacagatttgctctactaaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatga

SEQ ID NO: 57 - DNA, 1857 bp, Homo sapiens
  source 1..1857: mol_type = genomic DNA
tttgttccaagatctctaaaaatcaagaggaatgctaatgatgatggcaaaagttgtgtggctaagataattaaaccagacccagaagaccttcagttggacaaaagcagagatgttcccgttgatgctgtagctacagaagcagccacaatagacaggcacatcagcgaatcatgccctttccccagcccaggtggccagttggcagaggttcattcagtaagtcccgagcagggtgcgaaggacagccatccttctgaagagcccgttaagtcattttccaaaacacagcgctgggcagaaccaggggaacccatctgtgttgtctgtggtcgttatggagagtatatctgtgataagacagatgaagatgtgtgtagtttggagtgtaaagcgaaacatcttctacaagttaaggaaaaggaagagaaatcaaaactcagcaatccacagaaggctgattctgagccagagtctccactgaatgcttcctatgtctacaaagagcacccctttattttgaaccttcaggaagaccagattgaaaatcttaaacagcagctgggaattttagttcaagggcaagaagtcaccaggcccattattgactttgaacattgtagtctccctgaggtcttaaatcacaacttgaagaaatcaggctatgaggtgccaactcccattcaaatgcagatgattcctgtgggacttctgggaagagacattctggccagtgcagatactggctcaggaaaaacagctgcttttcttcttcctgttatcatgcgagctttattcgagagcaaaactccatctgcgctcattcttacaccaaccagagagttagccattcagatagagagacaagctaaagaattgatgagtggcctgccacgcatgaaaactgtgcttcttgtagggggcttacccttacccccacagctttatcgtctgcaacaacatgttaaggttatcatagcaacccctgggcgacttctggatataataaagcagagctctgtagaactctgtggtgtaaagattgtggtagtagatgaagctgataccatgttaaagatgggttttcaacaacaagtgcttgacattttggaaaacattcctaatgattgtcagaccattttggtttcagccacaattccaactagcatagaacagctagcaagccagcttctgcataatcctgtgagaattatcactggagaaaagaacctaccttgtgccaatgtacgtcagattattttgtgggtagaagacccagccaaaaagaaaaaattatttgaaattttaaatgataagaaactctttaagcctccagtgttagtatttgtggactgcaaactaggagcagatcttttgagtgaagccgttcagaaaatcacagggctgaaaagcatatctatacattcagagaagtcgcaaatagaaaggaaaaacatattgaagggattacttgaaggagactatgaagttgtagtgagcacaggagtcttgggacgaggcctagacttgatcagtgtcaggctggttgtcaattttgatatgccttcaagtatggatgagtatgtccatcagattggaagagtaggaagattaggtcaaaatggaacagcgattactttcatcaataataattcaaaaagactcttctgggatattgcaaaacgagtaaagcccacaggatccattcttccccctcagttattaaattccccataccttcatgaccagaagagaaaggaacaacagaaagataaacagacacagaatgatctggttacaggagctaatcttatggatattatcagaaaacatgataaaagtaattctcagaaatga

SEQ ID NO: 58 - DNA, 2209 bp, synthetic construct
  source 1..2209: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtgaccggctaccggctgttcgaggagattctcgggagttccggtggtggcgggagcggaggtggaggcatggcgtcgctcaccgtgaaggcctaccttctgggcaaggaggacgcggcgcgcgagattcgccgcttcagcttctgctgcagccccgagcctgaggcggaagccgaggctgcggcgggtccgggaccctgcgagcggctgctgagccgggtggccgccctgttccccgcgctgcggcctggcggcttccaggcgcactaccgcgatgaggacggggacttggttgccttttccagtgacgaggaattgacaatggccatgtcctacgtgaaggatgacatcttccgaatctacattaaagagaaaaaagagtgccggcgggaccaccgcccaccgtgtgctcaggaggcgccccgcaacatggtgcaccccaatgtgatctgcgatggctgcaatgggcctgtggtaggaacccgctacaagtgcagcgtctgcccagactacgacttgtgtagcgtctgcgagggaaagggcttgcaccgggggcacaccaagctcgcattccccagccccttcgggcacctgtctgagggcttctcgcacagccgctggctccggaaggtgaaacacggacacttcgggtggccaggatgggaaatgggtccaccaggaaactggagcccacgtcctcctcgtgcaggggaggcccgccctggccccacggcagaatcagcttctggtccatcggaggatccgagtgtgaatttcctgaagaacgttggggagagtgtggcagctgcccttagccctctgggcattgaagttgatatcgatgtggagcacggagggaaaagaagccgcctgacccccgtctctccagagagttccagcacagaggagaagagcagctcacagccaagcagctgctgctctgaccccagcaagccgggtgggaatgttgagggcgccacgcagtctctggcggagcagatgaggaagatcgccttggagtccgaggggcgccctgaggaacagatggagtcggataactgttcaggaggagatgatgactggacccatctgtcttcaaaagaagtggacccgtctacaggtgaactccagtccctacagatgccagaatccgaagggccaagctctctggacccctcccaggagggacccacagggctgaaggaagctgccttgtacccacatctcccgccagaggctgacccgcggctgattgagtccctctcccagatgctgtccatgggcttctctgatgaaggcggctggctcaccaggctcctgcagaccaagaactatgacatcggagcggctctggacaccatccagtattcaaagcatcccccgccgttgtga

SEQ ID NO: 59 - DNA, 3550 bp, synthetic construct
  source 1..3550: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggcggccttctccgagatgggtgtaatgcctgagattgcacaagctgtggaagagatggattggctcctcccaactgatatccaggctgaatctatcccattgatcttaggaggaggtgatgtacttatggctgcagaaacaggaagtggcaaaactggtgcttttagtattccagttatccagatagtttatgaaactctgaaagaccaacaggaaggcaaaaaaggaaaaacaacaattaaaactggtgcttcagtgctgaacaaatggcagatgaacccatatgacagaggatctgcttttgcaattgggtcagatggtctttgttgtcaaagcagagaagtaaaggaatggcatgggtgtagagctactaaaggattaatgaaagggaaacactactatgaagtatcctgtcatgaccaagggttatgcagggtcgggtggtctaccatgcaggcctctttggacctaggtactgacaagtttggatttggctttggtggaacaggaaagaaatcccataacaaacaatttgataattatggagaggaattcactatgcatgataccattggatgttacctggatatagataagggacatgtcaagttctccaaaaatggaaaagatcttggtctggcatttgaaataccaccacatatgaaaaaccaagccctctttcctgcctgtgttttgaagaatgctgaactgaaatttaacttcggtgaagaggaatttaagtttccaccaaaagatggctttgttgctctttccaaggcaccggatggttacattgtcaaatcacagcactcaggtaatgcacaggtgacacaaacaaagtttctccccaatgctccgaaagctctcattgttgaaccttcccgggagttagctgaacaaactttgaacaacatcaagcagtttaagaaatacattgataatcctaaattaagggagcttctgataattggaggtgttgcagcccgggatcagctctctgttttggaaaatggagtagatatagttgtaggtactccgggaagactagatgacttggtgtcaactggaaagctgaacttatctcaagttagattcctggtcctggatgaagctgatgggcttctttctcaaggttattctgattttataaataggatgcacaatcagattcctcaggttacctctgatggaaaaagacttcaggtgattgtttgctctgccactttgcattctttcgatgtaaagaaactgtccgagaagataatgcattttcctacatgggttgacttaaaaggagaagactctgttccagatactgtacaccatgttgttgtcccagtaaatcccaaaactgacagactctgggaaaggcttggaaagagccacattagaactgatgatgtacatgcaaaagataacacaagacctggtgctaatagtccagagatgtggtctgaagctattaaaatcctgaaaggggagtatgctgtccgggcaatcaaggaacataagatggatcaagcaattatcttctgtagaaccaaaattgactgtgataacttggagcagtactttatacaacaaggaggaggacctgataaaaaaggacaccagttctcatgtgtttgtcttcatggtgacagaaagcctcatgagagaaagcaaaacttggaaagatttaagaaaggagatgtaagattcttgatttgcacagatgtagctgctagaggaattgatatccacggtgttccttatgttataaatgtcactctgcccgatgaaaagcaaaactacgtacatcgaattggcagagtaggaagagctgaaaggatgggtctggcaatttccctggtggcaacagaaaaagaaaaggtttggtaccatgtatgtagcagccgtggaaaagggtgttataacacaagactcaaggaagatggaggctgtaccatatggtacaacgagatgcagttactatctgagatagaagaacacctgaactgtaccatttctcaggttgagccggatataaaggtaccagtggatgaatttgatgggaaagttacctacggtcagaaaagggctgctggtggtggaagctataaaggccatgtggatattttggcacctactgttcaagagttggctgcccttgaaaaggaggcgcagacatctttcctgcatcttggctaccttcctaaccagctgttcagaaccttctga

SEQ ID NO: 60 - DNA, 2548 bp, synthetic construct
  source 1..2548: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgtctgcgagccaggattcccgatccagagacaatggccccgatgggatggagcccgaaggcgtcatcgagagtaactggaatgagattgttgacagctttgatgacatgaacctctcggagtcccttctccgtggcatctacgcgtatggttttgagaagccctctgccatccagcagcgagccattctaccttgtatcaagggttatgatgtgattgctcaagcccaatctgggactgggaaaacggccacatttgccatatcgattctgcagcagattgaattagatctaaaagccacccaggccttggtcctagcacccactcgagaattggctcagcagatacagaaggtggtcatggcactaggagactacatgggcgcctcctgtcacgcctgtatcgggggcaccaacgtgcgtgctgaggtgcagaaactgcagatggaagctccccacatcatcgtgggtacccctggccgtgtgtttgatatgcttaaccggagatacctgtcccccaaatacatcaagatgtttgtactggatgaagctgacgaaatgttaagccgtggattcaaggaccagatctatgacatattccaaaagctcaacagcaacacccaggtagttttgctgtcagccacaatgccttctgatgtgcttgaggtgaccaagaagttcatgagggaccccattcggattcttgtcaagaaggaagagttgaccctggagggtatccgccagttctacatcaacgtggaacgagaggagtggaagctggacacactatgtgacttgtatgaaaccctgaccatcacccaggcagtcatcttcatcaacacccggaggaaggtggactggctcaccgagaagatgcatgctcgagatttcactgtatccgccatgcatggagatatggaccaaaaggaacgagacgtgattatgagggagtttcgttctggctctagcagagttttgattaccactgacctgctggccagaggcattgatgtgcagcaggtttctttagtcatcaactatgaccttcccaccaacagggaaaactatatccacagaatcggtcgaggtggacggtttggccgtaaaggtgtggctattaacatggtgacagaagaagacaagaggactcttcgagacattgagaccttctacaacacctccattgaggaaatgcccctcaatgttgctgacctcatctga

SEQ ID NO: 61 - DNA, 3502 bp, synthetic construct
  source 1..3502: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgggagatgaagattgggaagcagaaatcaaccctcatatgtcttcctatgttcccatatttgagaaggataggtattctggagaaaatggagacaattttaacaggactccagcttcatcatcagaaatggatgatggaccttctcgaagagatcatttcatgaaaagtggatttgcctctgggcggaattttggaaacagagatgctggtgagtgtaataagcgagataatacatccacaatgggtggttttggagttggaaagagttttggaaacagaggtttttcaaacagcaggtttgaagatggtgatagctctggtttctggagagagtctagtaatgactgcgaagataatccaacacggaacagagggttttccaagagaggcggctatcgagatggaaataattcagaagcttcagggccatacagaagaggtggaagaggtagtttccgaggttgccgtggaggatttggtctaggaagtccaaataatgacttagacccagacgaatgtatgcagcgcactggtggcctttttggttctagaagaccagtattaagtggcacaggtaatggtgatacttctcaaagcagaagtggcagtggaagtgaacgaggtggttacaaaggtttaaatgaagaagtaataacaggctctggaaagaattcttggaagtcagaagcagaaggaggagaaagtagtgatactcaaggaccaaaagtgacctacataccccctcctccacctgaggatgaggactccatctttgcacattatcagacaggcataaacttcgacaaatacgacactattcttgtggaagtgtctggacatgatgcaccaccagcaattctgacttttgaagaagctaatctctgtcagacactgaataacaacattgctaaagctggttatactaagcttactcctgtgcaaaaatacagtattcctatcatacttgcaggacgagatttgatggcttgcgctcaaacagggtctgggaagactgcggcttttctcctaccaattttggctcatatgatgcatgatggaataactgccagtcgttttaaagagttgcaggaaccagagtgtattattgtagcaccaactcgagaattggtcaaccagatttatttggaagccagaaaattttcttttgggacttgtgtaagagctgttgttatatatgggggaacccagctgggacattcaattcgacaaatagtacaaggctgtaatatattatgtgctactcctggaagactgatggatatcataggcaaagaaaagattggtctcaaacagatcaaatacttagttttggatgaagctgatcgcatgttggatatgggttttggtccagaaatgaagaagttaatttcttgcccaggaatgccatcaaaggaacagcgccaaacccttatgttcagtgcaacttttccagaggaaattcaaaggttggctgcagagtttttaaagtcaaattatctgtttgttgctgttggacaagtgggtggagcatgtagagatgttcagcagaccgttctccaagttggccagttctcaaaaagagaaaagctcgttgaaattctgcgaaacataggggatgaaagaactatggtctttgttgaaactaagaaaaaagcagattttattgcaacttttctttgtcaagaaaaaatatcaactacaagtattcatggtgatcgggaacagagagagcgggagcaagctcttggagattttcgctttggaaagtgcccagttcttgttgctacttcagtagctgccagagggctggatattgaaaatgtgcaacatgttatcaattttgatcttccttctaccattgatgaatatgttcatcgaattgggcgtactggtcgttgtgggaatactggcagagcaatttccttttttgatcttgaatcggataaccatttagcacagcctctagtaaaagtattgacagatgctcaacaggatgttcctgcatggttggaagaaattgcctttagtacatacattcctggcttcagtggtagtacaagaggaaacgtgtttgcatcagttgataccagaaagggcaagagcactttgaacacagctgggttttcttcttcacaagctcccaatccagtagatgatgagtcatgggattaa

SEQ ID NO: 62 - DNA, 3172 bp, synthetic construct
  source 1..3172: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgtcgggttattcgagtgaccgagaccgcggccgggaccgagggtttggtgcacctcgatttggaggaagtagggcagggcccttatctggaaagaagtttggaaaccctggggagaaattagttaaaaagaagtggaatcttgatgagctgcctaaatttgagaagaatttttatcaagagcaccctgatttggctaggcgcacagcacaagaggtggaaacatacagaagaagcaaggaaattacagttagaggtcacaactgcccgaagccagttctaaatttttatgaagccaatttccctgcaaatgtcatggatgttattgcaagacagaatttcactgaacccactgctattcaagctcagggatggccagttgctctaagtggattggatatggttggagtggcacagactggatctgggaaaacattgtcttatttgcttcctgccattgtccacatcaatcatcagccattcctagagagaggcgatgggcctatttgtttggtgctggcaccaactcgggaactggcccaacaggtgcagcaagtagctgctgaatattgtagagcatgtcgcttgaagtctacttgtatctacggtggtgctcctaagggaccacaaatacgtgatttggagagaggtgtggaaatctgtattgcaacacctggaagactgattgactttttagagtgtggaaaaaccaatctgagaagaacaacctaccttgtccttgatgaagcagatagaatgcttgatatgggctttgaaccccaaataaggaagattgtggatcaaataagacctgataggcaaactctaatgtggagtgcgacttggccaaaagaagtaagacagcttgctgaagatttcctgaaagactatattcatataaacattggtgcacttgaactgagtgcaaaccacaacattcttcagattgtggatgtgtgtcatgacgtagaaaaggatgaaaaacttattcgtctaatggaagagatcatgagtgagaaggagaataaaaccattgtttttgtggaaaccaaaagaagatgtgatgagcttaccagaaaaatgaggagagatgggtggcctgccatgggtatccatggtgacaagagtcaacaagagcgtgactgggttctaaatgaattcaaacatggaaaagctcctattctgattgctacagatgtggcctccagagggctagatgtggaagatgtgaaatttgtcatcaattatgactaccctaactcctcagaggattatattcatcgaattggaagaactgctcgcagtaccaaaacaggcacagcatacactttctttacacctaataacataaagcaagtgagcgaccttatctctgtgcttcgtgaagctaatcaagcaattaatcccaagttgcttcagttggtcgaagacagaggttcaggtcgttccaggggtagaggaggcatgaaggatgaccgtcgggacagatactctgcgggcaaaaggggtggatttaatacctttagagacagggaaaattatgacagaggttactctagcctgcttaaaagagattttggggcaaaaactcagaatggtgtttacagtgctgcaaattacaccaatgggagctttggaagtaattttgtgtctgctggtatacagaccagttttaggactggtaatccaacagggacttaccagaatggttatgatagcactcagcaatacggaagtaatgttccaaatatgcacaatggtatgaaccaacaggcatatgcatatcctgctactgcagctgcacctatgattggttatccaatgccaacaggatattcccaataa

SEQ ID NO: 63 - DNA, 2779 bp, synthetic construct
  source 1..2779: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgagcacggccagaacagagaaccctgttataatgggtctgtccagtcaaaatggtcagctgagaggccctgtgaaacccactggtggccctggaggagggggcacacagacacagcaacagatgaaccagctgaaaaacaccaacacaatcaataatggcactcagcagcaagcacagagtatgaccaccactattaaacctggtgatgactggaaaaagactttaaaactccctccaaaggatctaagaatcaaaacttcggatgtgacctccacaaaaggaaatgagtttgaagattactgtttgaaacgggagttactgatgggaatttttgaaatgggctgggaaaagccatctcctattcaggaggagagcattcccattgctttatctggtagggatatcttagctagagcaaaaaatggaacaggcaagagcggtgcctacctcattcccttacttgaacggctagacctgaagaaggacaatatacaagcaatggtgattgttcccactagagaacttgctctacaggtcagtcaaatttgcatccaggtcagcaaacacatgggaggggccaaagtgatggcaaccacaggaggaaccaatttacgagatgacataatgaggcttgatgatacagtgcacgtggtgattgctacccctgggagaatcctggatcttattaagaaaggagtagcaaaggttgatcatgtccagatgatagtattggatgaggcagataagttgctgtcacaggattttgtgcagataatggaggatattattctcacgctacctaaaaacaggcagattttactatattccgctactttccctcttagtgtacagaagttcatgaattcccatttgcagaaaccctatgagattaacctgatggaggaactaactctgaagggagtaacccagtactacgcatatgtaactgagcgccaaaaagtacactgcctcaacacacttttctccaggcttcagataaaccagtcgatcattttctgtaactcctctcagcgagttgaattgctagccaagaagatttctcaactgggttattcttgcttctatattcatgctaaaatgaggcaggaacatcgaaatcgtgtatttcatgatttccgaaatggcttatgccgcaatcttgtttgcactgatctgtttacccgaggtattgatatacaagctgtgaatgtggtaataaactttgatttcccaaagctggcagagacctatctccatcgtattggaagatcaggtcgctttggtcatcttggcttagccatcaacttgatcacatatgatgatcgcttcaacctgaaaagtattgaggagcagctgggaacagaaattaaacctattccgagcaacattgataagagcctgtatgtggcagaataccacagcgagcctgtagaagatgagaaaccttaa

SEQ ID NO: 64 - DNA, 3955 bp, synthetic construct
  source 1..3955: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgggcaaaacggccaactctccgggttcgggagcccgacccgacccggtgcggagcttcaatcgctggaagaaaaaacacagccataggcagaacaaaaagaagcagttgaggaagcaactgaagaaacccgaatggcaggtcgagcgcgagagtatcagccgcctcatgcagaactatgaaaagataaatgtaaatgaaatcacaagattttcagattttcccttgtccaaaaaaacattgaaaggtttgcaagaagctcagtaccgtttggtgactgagatacagaagcagaccattggattggctttgcaaggtaaagatgtacttggagcggccaaaactggatctggcaagactctggcttttcttgttccagtgctggaagccttatatcgtctgcaatggacttcaacagatgggctgggggttctcataatatcacctacgagagaactggcctatcagacctttgaggttctccgaaaagtaggaaagaatcatgacttctcagctggtctcatcattggtggaaaggatctaaaacacgaagctgagaggatcaacaacataaatatactcgtgtgcacaccaggtcggcttcttcaacacatggatgaaacagtatcttttcatgctaccgacctccaaatgttagttcttgatgaagcagatagaatcttggatatgggctttgctgataccatgaatgctgttattgaaaatctccccaagaaacgtcagactttacttttctcagcaacacaaactaaatctgtaaaggaccttgcacgcttgagtttgaaaaaccctgagtatgtctgggttcatgaaaaagcaaaatatagcacccctgccactttggaacagaactacatagtctgtgagctgcagcaaaaaataagtgtgctgtattcctttttgagaagccatctgaagaagaagagcattgtatttttttccagttgcaaagaggtccagtatctgtaccgagtgttttgccggctacgtcctggtgtttctatccttgcactccatggtcgacagcagcaaatgagaagaatggaagtctataatgagtttgtccgtaagagagctgcagtactctttgctactgatattgcagccaggggtctggatttcccggccgtgaattgggttcttcagtttgattgtcctgaggatgccaacacatatattcacagagcaggtagaactgccaggtacaaagaggatggtgaagctttgctaattttgctaccctcagaaaaagctatggtgcagcagcttcttcagaagaaagtacctgtgaaggaaatcaaaatcaatccagaaaaacttatagatgtccagaaaaaattggaatctattttagctcaagatcaagatttaaaagaaagagctcaaaggtgtttcgtctcctatgtacgatctgtatatctgatgaaggataaagaagtatttgatgtgagcaagttacctatacctgaatatgccctgtctcttggtcttgctgtggcaccacgcgtaagatttcttcagaaaatgcagaaacaacccaccaaagaattggtaaggagccaagccgataaagtaattgagccaagggctccctccctcaccaatgacgaagtggaagaatttagagcctacttcaatgagaaaatgtccatccttcaaaaaggtggaaaaagactcgaagggacagagcacagacaggataatgatactggtaatgaagaacaggaagaagaagaagacgatgaagaagaaatggaagagaaactggcaaaagcaaaaggatctcaagccccatctcttcctaacaccagtgaggcacagaagatcaaggaagttcctacacagttcttggacagagatgaggaggaagaagatgctgatttcttgaaggtgaagcggcataatgtgtttggattggaccttaaagacgagaaaacattacagaagaaagaaccttctaaatccagcatcaagaaaaaaatgaccaaagttgcagaagcaaaaaaagtaatgaagagaaattttaaagtgaataagaagataacatttactgatgaaggggagttggttcagcagtggccacaaatgcagaaatctgccatcaaggatgctgaggaagatgatgacacaggtggtatcaacttacataaagcaaaggaaagacttcaggaagaggacaaatttgacaaagaagaatataggaaaaaaattaaggcaaagcatcgggagaaaagactgaaagaaagggaagccagaagagaagccaacaagagacaagcaaaggccaaagatgaagaggaagcctttctggattggagtgatgatgatgatgatgatgatgatggatttgatccaagcacactcccagatccagataaatacagaagctctgaagattcagatagtgaagatatggaaaataaaataagtgataccaagaagaagcaggggatgaagaagaggagcaacagtgaagtggaagacgtgggaccaacaagtcataacagaaagaaggccaggtgggacactttagagcctttggataccggcctgtctttagcagaggatgaagagctggtgttacatctgctaagaagtcaaagctaa

SEQ ID NO: 65 - DNA, 4040 bp, synthetic construct
  source 1..4040: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggctaatgaaacacagaaggttggtgccatccattttccttttcccttcacaccctattccatccaggaagacttcatggcagagctgtaccgggttttggaggctggcaagattgggatatttgagagtccaactggcactgggaagtccttaagtcttatttgtggggccctctcttggctccgtgactttgaacagaagaagcgtgaagaagaggcacgactccttgaaactggaactggccccttacatgatgagaaagatgaatccctgtgtctgtcttcttcctgcgaaggggctgcaggcaccccgaggcctgctggagaaccggcctgggttactcagtttgtgcagaagaaagaagagagggacctggtggaccgactaaaggcggagcaggccaggaggaagcagcgagaagaacgcctgcagcagctgcagcacagggtgcagctcaagtatgcagccaagcgcctgaggcaggaagaagaagaaagagagaatctcctccgcctcagcagggagatgctagagacaggcccggaggctgagcggctggagcagctggagtctggggaggaggagctggtcctcgccgaatacgagagtgatgaggagaaaaaggtggcgagcagagtggatgaggatgaggatgacctggaggaagaacacataactaagatttattactgtagtcggacacactcccagctggcccagtttgtgcatgaggtgaagaagagcccctttggcaaggatgttcggctggtctcccttggctcccggcagaacctttgtgtaaatgaagacgtgaaaagcctaggttctgtgcagcttatcaacgaccgctgtgtggacatgcagagaagcaggcacgagaagaagaaaggagctgaggaggagaagccaaagaggaggaggcaggagaagcaggcagcctgccccttctacaaccacgagcagatgggccttctccgggatgaggccctggcagaggtgaaggacatggagcagctgctggcccttgggaaggaggcccgggcctgtccctattacgggagccgccttgccatccctgcagcccagctggtggtgctgccctatcagatgctgctgcatgcggccactcggcaggccgcgggcatccggctgcaggaccaggtggtgatcatcgacgaggcgcacaacctgatcgacaccatcacgggcatgcacagcgtggaggtcagcggctcccagctctgccaggcccattcccagctgctgcagtacgtggagcgatacgggaagcgtttgaaggccaagaacctgatgtacctgaagcagatcctgtatttgctggagaaattcgtggctgtgctaggggggaacattaagcaaaatcccaatacacagagtctgtcacagacagggacggagctgaagaccatcaacgactttctcttccagagccagatcgacaacatcaacctgttcaaggtgcagcgatactgtgagaagagcatgatcagcagaaagctctttggattcactgaacggtacggagcagtgttctcatcccgggagcagcccaaactggctgggtttcagcaattcctgcagagcctgcagcccaggacgactgaagctcttgcagcccctgcagacgagagtcaggccagcaccctgcgaccagcttctccactgatgcacatccaaggcttcctggcagctctcactacggccaaccaggacggcagggtcatcctgagccgccaaggcagcctcagtcagagcaccctgaagtttttgctcctgaatccagctgtgcactttgcccaagtggtgaaggaatgccgggcagtggtcattgcggggggtaccatgcagccggtgtctgacttccggcagcagctgctggcctgtgccggggtggaagctgagcgcgtggtggagttttcctgtggtcacgtgatccctccagacaacatcctgcccctcgtcatctgcagcgggatctccaaccagccgctggaattcacgttccagaaaagagagctgcctcagatgatggacgaggtgggtcgcattctctgtaacctgtgcggtgtggttcctggaggggtggtctgtttcttcccctcctacgagtacctgcgccaggtccatgcccactgggagaagggtggcctgctgggccgtctggctgccaggaagaagatattccaggaacctaagagcgcacaccaggtggagcaggtgctgctggcatattccaggtgcatccaggcctgtggccaggagagaggccaggtgacaggggccctgctcctctctgtggttggaggaaagatgagtgaagggatcaacttctctgacaacctaggccggtgtgtggtgatggtgggcatgcccttccccaacatcaggtctgcagagctgcaggagaagatggcctacttggatcaaaccctcagcccccggccaggcacccccagggaaggctctggtggagaacctgtgcatgaaggccgtcaaccagtccataggcagggccatcaggcaccagaaggattttgccagcgtagtgctcctggaccagcgatatgcccggccccctgtcctggccaagctgccggcctggatccgagcccgtgtggaggtcaaagctacctttggccccgccattgctgctgtgcagaagtttcaccgggagaagtcggcctcttcc

SEQ ID NO: 66 - DNA, 3517 bp, synthetic construct
  source 1..3517: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgcccaccggctttgtagccccgattctctgtgttttgctcccgtctccgacgagagaggcggcgacggtggcgtctgcgacgggagacagcgcgtcggagcgagagagcgctgcgcctgccgccgccccaacagcggaggcgccgccgccatcggtcgtcaccagaccggagccgcaggccctcccgagcccggccatccgtgccccgctcccagatctctatccttttgggaccatgcgcggaggaggctttggggaccgggaccgggatcgtgaccgtggaggatttggagcaagaggtggtggtggccttcccccgaagaaatttggtaatcctggggagcgtttgcgtaaaaaaaagtgggatttgagtgagctccccaagtttgagaaaaatttttatgtggaacatccggaagtagcaaggctgacaccatatgaggttgatgagctacgccgaaagaaggagattacagtgagggggggagatgtttgtcctaaacccgtgtttgccttccatcatgctaacttcccacaatatgtaatggatgtgttgatggatcagcactttacagaaccaactccaattcagtgccagggatttccgttggctcttagtggccgggatatggtgggcattgctcagactggctctgggaagacgttggcgtatctcctgcctgcaattgttcatattaaccaccagccatacttggaaaggggagatggcccaatctgtctagttctggctcctaccagagagcttgcccagcaagtacagcaggtggccgatgactatggcaaatgttctagattgaagagtacttgtatttatggaggtgctcctaaaggtccccagattcgagacttggaaagaggtgttgagatctgcatagccactcctggacgtctgatagatttcctggagtcaggaaagacaaatcttcgccgatgtacttaccttgtattggacgaagctgacagaatgcttgatatggggtttgaaccccagatccgtaaaattgttgaccaaatcaggcctgataggcagacactgatgtggagtgcaacctggccaaaagaagtaagacagcttgcagaggatttccttcgtgattacacccagatcaacgtaggcaatctggagttgagtgccaaccacaacatcctccagatagtggatgtctgcatggaaagtgaaaaagaccacaagttgatccaactaatggaagaaataatggctgaaaaggaaaacaaaacaataatatttgtggagacaaagagacgctgtgatgatctgactcgaaggatgcgcagagatggttggccagctatgtgtatccatggagacaagagtcaaccagaaagagattgggtacttaatgagttccgttctggaaaggcacccatccttattgctacagatgtagcctcccgtgggctagatgtggaagatgtcaagtttgtgatcaactatgactatccaaacagctcagaggattatgtgcaccgtattggccgaacagcccgtagcaccaacaagggtaccgcctataccttcttcaccccagggaacctaaaacaggccagagagcttatcaaagtgctggaagaggccaatcaggctatcaatccaaaactgatgcagcttgtggaccacagaggaggcggcggaggcgggggtggtcgttctcgttaccggaccacttcttcagccaacaatcccaatctgatgtatcaggatgagtgtgaccgaaggcttcgaggagtcaaggatggtggccggagagactctgcaagctatcgggatcgtagtgaaaccgatagagctggttatgctaatggcagtggctatggaagtccaaattctgcctttggagcacaagcaggccaatacacctatggtcaaggcacctatggggcagctgcttatggcaccagtagctatacagctcaagaatatggtgctggcacttatggagctagtagcaccacctcaactgggagaagttcacagagctctagccagcagtttagtgggataggccggtctgggcagcagccacagccactgatgtcacaacagtttgcacagcctccgggagctaccaatatgataggttacatggggcagactgcctaccaataccctcctcctcctccccctcctcctccttcacgtaaatga

SEQ ID NO: 67 - DNA, 3340 bp, synthetic construct
  source 1..3340: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgtcacacctgccgatgaaactcctgcgtaagaagatcgagaagcggaacctcaaattgcggcagcggaacctaaagtttcagggggcctcaaatctgaccctatcggaaactcaaaatggagatgtatctgaagaaacaatgggaagtagaaaggttaaaaaatcaaaacaaaagcccatgaatgtgggcttatcagaaactcaaaatggaggcatgtctcaagaagcagtgggaaatataaaagttacaaagtctccccagaaatccactgtattaaccaatggagaagcagcaatgcagtcttccaattcagaatcaaaaaagaaaaagaagaaaaagagaaaaatggtgaatgatgctgagcctgatacgaaaaaagcaaaaactgaaaacaaagggaaatctgaagaagaaagtgccgagactactaaagaaacagaaaataatgtggagaagccagataatgatgaagatgagagtgaggtgcccagtctgcccctgggactgacaggagcttttgaggatacttcgtttgcttctctatgtaatcttgtcaatgaaaacactctgaaggcaataaaagaaatgggttttacaaacatgactgaaattcagcataaaagtatcagaccacttctggaaggcagggatcttctagcagctgcaaaaacaggcagtggtaaaaccctggcttttctcatccctgcagttgaactcattgttaagttaaggttcatgcccaggaatggaacaggagtccttattctctcacctactagagaactagccatgcaaacctttggtgttcttaaggagctgatgactcaccacgtgcatacctatggcttgataatgggtggcagtaacagatctgctgaagcacagaaacttggtaatgggatcaacatcattgtggccacaccaggccgtctgctggaccatatgcagaataccccaggatttatgtataaaaacctgcagtgtctggttattgatgaagctgatcgtatcttggatgtggggtttgaagaggaattaaagcaaattattaaacttttgccaacacgtagacagactatgctcttttctgccacccaaactcgaaaagttgaagacctggcaaggatttctctgaaaaaggagccattgtatgttggcgttgatgatgataaagcgaatgcaacagtggatggtcttgaacagggatatgttgtttgtccttctgaaaagagattccttctgctctttacattccttaagaagaaccgaaagaagaagcttatggtcttcttttcatcttgtatgtctgtgaaataccactatgagttgctgaactacattgatttgcccgtcttggccattcatggaaagcaaaagcaaaataagcgtacaaccacattcttccagttctgcaatgcagattcgggaacactattgtgtacggatgtggcagcgagaggactagacattcctgaagtcgactggattgttcagtatgaccctccggatgaccctaaggaatatattcatcgtgtgggtagaacagccagaggcctaaatgggagagggcatgccttgctcattttgcgcccagaagaattgggttttcttcgctacttgaaacaatccaaggttccattaagtgaatttgacttttcctggtctaaaatttctgacattcagtctcagcttgagaaattgattgaaaagaattactttcttcataagtcagcccaggaagcatataagtcatacatacgagcctatgattcccattctctgaaacagatctttaatgttaataacctaaatttgcctcaggttgctctgtcatttggtttcaaggtgcctcccttcgttgatctgaacgtcaacagtaatgaaggcaagcagaaaaagcgaggaggtggtggtggatttggctaccagaaaaccaagaaagttgagaaatccaaaatctttaaacacattagcaagaaatcatctgacagcaggcagttctctcactga

SEQ ID NO: 68 - DNA, 2767 bp, Homo sapiens
  source 1..2767: mol_type = genomic DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggccactgactcatgggccctggcggtggacgagcaggaagctgcggctgagtcgttgagcaacttgcatcttaaggaagagaaaatcaaaccagataccaatggtgctgttgtcaagaccaatgccaatgcagagaagacagatgaagaagagaaagaggacagagctgcccagtccttactcaacaagctgatcagaagcaaccttgttgataacacaaaccaagtggaagtcctgcaacgggatccaaactcccctctgtactcggtgaagtcgtttgaagagcttcggctgaaaccacagcttctccagggagtctatgccatgggcttcaatcgaccctccaagatacaagagaacgcattacccatgatgcttgctgaacccccacagaatctgattgcccagtctcagtctggcactggtaaaacagctgcctttgtcttagccatgctcagccgagtggagccatcagacagatacccccagtgtctgtgcctctccccaacatatgagctggcgcttcaaacaggaaaagtgattgagcagatgggcaaattttacccagaactgaagcttgcctatgccgttcgaggcaataaattggaaagaggccagaagatcagtgagcagattgtcattggcacccctgggaccgtgctggactggtgctccaagctcaagttcattgatcccaagaaaatcaaggtgtttgttctggatgaggctgatgtcatgatagccactcagggccaccaagatcagagcatccgcatccagaggatgctgcccaggaactgccagatgctgcttttctccgccacctttgaagactctgtgtggaagtttgcccagaaagtggtcccagacccaaatgttatcaaactgaagcgtgaggaagagaccctggataccatcaagcagtactatgtcctgtgcagcagcagagacgagaagttccaggccttgtgtaacctctacggggccatcaccattgctcaagccatgatcttctgccatactcgcaaaacagctagttggctggcagcagagctctcaaaagaaggccaccaggtggctctgctgagtggggagatgatggtggagcagagggctgcggtgattgagcgcttccgagagggcaaagagaaggttttggtgaccaccaacgtgtgtgcccgcggcattgatgttgaacaagtgtctgtcgtcatcaactttgatcttcccgtggacaaggacgggaatcctgacaatgagacctacctgcaccggatcgggcgcacgggccgctttggcaagaggggcctggcagtgaacatggtggacagcaagcacagcatgaacatcctgaacagaatccaggagcattttaataagaagatagaaagattggacacagatgatttggacgagattgagaaaatagccaactga

SEQ ID NO: 69 - DNA, 3802 bp, synthetic construct
  source 1..3802: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggcggcggcatttgaagcctcgggagccttagcagcagtggcgactgctatgccggctgagcatgtggccgtgcaggtcccggccccagagccaacacccgggcctgtgaggatcctgcggaccgctcaggatctcagcagcccgcggacccgcacgggggatgtgctgttggcggagccggccgacttcgagtcactgctgctttcgcggccggtgctggaggggctgcgggcggccggcttcgagaggccctcgccggtgcagctcaaggccatcccgttggggcgctgcgggctcgatttaattgttcaagctaaatctggcaccgggaaaacctgtgtgttctccaccatagctttggactctcttgttcttgaaaacttaagtacccagattttgatcttggctcctacaagagaaattgctgtacagatacattctgttattacagccattggaataaaaatggaaggcttagagtgtcatgtctttattggagggaccccattatcacaagacaaaaccagacttaaaaagtgtcatattgctgttggatctcctggcagaattaagcaactcatagaacttgactacttgaacccaggcagtatacgcctctttattcttgatgaagcagataagcttttagaagaaggcagcttccaggagcaaataaattggatttattcttccttgcctgccagtaaacagatgctggcagtatcagctacttatcccgaatttttggctaatgctttgacaaagtacatgagagatcccacttttgtaagactgaattccagtgatccaagtctcataggtttgaagcagtattacaaagttgtcaattcataccctttggcacataaggtttttgaggaaaagactcagcatttacaggaactgttcagcagaattccatttaatcaagctttagtcttttctaatttgcacagcagagcacaacatttggctgatatcctttcttctaaaggctttcctgctgagtgcatttcaggcaatatgaatcagaatcagcgtcttgatgctatggctaaactgaagcactttcattgcagagtcctcatttccacagatttgacttctcgtgggattgatgctgagaaggtgaatctggttgtaaatctggatgtaccattggattgggagacatacatgcatcggattgggagagctggccgttttggtacattggggctgacagtgacctactgttgccggggagaggaagaaaatatgatgatgagaattgcccagaaatgtaatatcaaccttctccctttaccagatcccattccttctggtctgatggaagaatgtgtggattgggatgtggaagttaaagctgctgtgcatacatatggtatagcaagtgtacctaaccaacccttaaaaaagcaaattcagaaaatagagagaacccttcaaattcagaaagctcatggtgaccacatggcttcctctagaaataattctgtatctggactatcagtcaaatcaaaaaataataccaaacaaaagcttcctgtgaaaagccactcagaatgtggaatcatagaaaaagcaacgtcaccaaaagaactgggctgtgacaggcaatccgaagagcaaatgaagaattctgttcagactcccgttgaaaactccaccaacagtcagcaccaggtcaaagaagctttacctgtgtcactcccccagattccttgtctgtcttcctttaaaatccatcagccatacacgttgacttttgctgaattggtagaggattatgaacattatattaaagaggggttagagaaacctgtggaaatcatcaggcactacacaggccctggggatcagactgtgaatcctcaaaatggttttgtgagaaataaagttattgaacagagagtccctgtgttggcaagtagtagccaatctggagactctgagagtgacagtgattcttacagctcaagaacctcttcccagagcaaaggaaataagtcatacttggaaggctcttctgataatcagctgaaagactctgaatctacgcctgtggatgatcgtatttctttggaacaaccaccaaatggaagtgacacccccaatccagagaaatatcaagaatcacctggaatccagatgaagacaagacttaaagagggggctagccagagagctaagcagagccggagaaacctacccaggcggtcttccttcagattgcagactgaagcccaggaagatgattggtatgactgtcatagggaaatacgtctgagtttttctgatacctatcaggattatgaggagtactggagagcttactacagggcatggcaagaatattatgctgccgcttctcattcatattattggaatgctcagagacatccaagttggatggcagcttatcacatgaataccatttatctacaagaaatgatgcatagtaaccagtga

SEQ ID NO: 70 - DNA, 3679 bp, synthetic construct
  source 1..3679: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgccgggaaaactccgtagtgacgctggtttggaatcagacaccgcaatgaaaaaaggggagacactgcgaaagcaaaccgaggagaaagagaaaaaagagaagccaaaatctgataagactgaagagatagcagaagaggaagaaactgttttccccaaagctaaacaagttaaaaagaaagcagagccttctgaagttgacatgaattctcctaaatccaaaaaggcaaaaaagaaagaggagccatctcaaaatgacatttctcctaaaaccaaaagtttgagaaagaaaaaggagcccattgaaaagaaagtggtttcttctaaaaccaaaaaagtgacaaaaaatgaggagccttctgaggaagaaatagatgctcctaagcccaagaagatgaagaaagaaaaggaaatgaatggagaaactagagagaaaagccccaaactgaagaatggatttcctcatcctgaaccggactgtaaccccagtgaagctgccagtgaagaaagtaacagtgagatagagcaggaaatacctgtggaacaaaaagaaggcgctttctctaattttcccatatctgaagaaactattaaacttctcaaaggccgaggagtgaccttcctatttcctatacaagcaaagacattccatcatgtttacagcgggaaggacttaattgcacaggcacggacaggaactgggaagacattctcctttgccatccctttgattgagaaacttcatggggaactgcaagacaggaagagaggccgtgcccctcaggtactggttcttgcacctacaagagagttggcaaatcaagtaagcaaagacttcagtgacatcacaaaaaagctgtcagtggcttgtttttatggtggaactccctatggaggtcaatttgaacgcatgaggaatgggattgatatcctggttggaacaccaggtcgtatcaaagaccacatacagaatggcaaactagatctcaccaaacttaagcatgttgtcctggatgaagtggaccagatgttggatatgggatttgctgatcaagtggaagagattttaagtgtggcatacaagaaagattctgaagacaatccccaaacattgcttttttctgcaacttgccctcattgggtatttaatgttgccaagaaatacatgaaatctacatatgaacaggtggacctgattggtaaaaagactcagaaaacggcaataactgtggagcatctggctattaagtgccactggactcagagggcagcagttattggggatgtcatccgagtatatagtggtcatcaaggacgcactatcatcttttgtgaaaccaagaaagaagcccaggagctgtcccagaattcagctataaagcaggatgctcagtccttgcatggagacattccacagaagcaaagggaaatcaccctgaaaggttttagaaatggtagttttggagttttggtggcaaccaatgttgctgcacgtgggttagacatccctgaggttgatttggttatacaaagctctccaccaaaggatgtagagtcctacattcatcgatccgggcggacaggcagagctggaaggacgggggtgtgcatctgcttttatcagcacaaggaagaatatcagttagtacaagtggagcaaaaagcgggaattaagttcaaacgaataggtgttccttctgcaacagaaataataaaagcttccagcaaagatgccatcaggcttttggattccgtgcctcccactgccattagtcacttcaaacaatcagctgagaagctgatagaggagaagggagctgtggaagctctggcagcagcactggcccatatttcaggtgccacgtccgtagaccagcgctccttgatcaactcaaatgtgggttttgtgaccatgatcttgcagtgctcaattgaaatgccaaatattagttatgcttggaaagaacttaaagagcagctgggcgaggagattgattccaaagtgaagggaatggtttttctcaaaggaaagctgggtgtttgctttgatgtacctaccgcatcagtaacagaaatacaggagaaatggcatgattcacgacgctggcagctctctgtggccacagagcaaccagaactggaaggaccacgggaaggatatggaggcttcaggggacagcgggaaggcagtcgaggcttcaggggacagcgggacggaaacagaagattcagaggacagcgggaaggcagtagaggcccgagaggacagcgatcaggaggtggcaacaaaagtaacagatcccaaaacaaaggccagaagcggagtttcagtaaagcatttggtcaataa

SEQ ID NO: 71 - DNA, 3790 bp, synthetic construct
  source 1..3790: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggcaggagagctggctgacaaaaaggaccgtgatgcatcaccttccaaggaggaaaggaagcgatcacggactcctgacagagagcgggatagagaccgggaccggaagtcttccccatctaaagatagaaagcggcatcgttcaagggatagacgtcgaggaggcagccgttctcgctctcgttcccgttccaaatctgcagaaagagaacgacggcacaaagaacgagaacgagataaggagcgggatcggaataagaaggaccgagatcgagacaaggatgggcacagacgggacaaggaccgtaaacgatccagcttatctcctggtcgaggaaaagactttaaatctcggaaggacagagactctaagaaggatgaagaggatgaacatggtgataagaagcctaaggcccagccattatccctggaggagcttctggccaagaaaaaggctgaggaagaagctgaggctaagcccaagttcctctctaaagcagaacgagaggctgaagctctaaagcgacggcagcaggaggtggaagagcggcagaggatgcttgaagaagagaggaagaaaaggaaacagttccaagacttgggcaggaagatgttggaagatcctcaggaacgggaacgtcgggaacgcagggagaggatggaacgggagaccaatggaaatgaggatgaggaagggcggcagaagatccgggaagagaaggataagagcaaggaactgcatgccattaaggagcgttacctgggtggcatcaaaaagcggcgccgaacgagacatctcaatgaccggaaatttgtttttgagtgggatgcatctgaggacacatccattgactacaaccccctgtacaaagaacggcaccaggtgcagttgttagggcgaggcttcattgcaggcattgacctcaagcagcagaagcgagagcagtcacgtttctatggagacctaatggagaagaggcgaaccctggaagaaaaggagcaggaggaggcaagactccgcaaacttcgtaagaaggaagccaagcagcgctgggatgatcgtcattggtctcagaaaaagttagatgagatgacggacagggactggcggatcttccgtgaggactacagcatcaccaccaaaggtggcaagatccccaatcccatccgatcctggaaagactcttctctgcccccacacatcttggaggtcattgataagtgtggctacaaggaaccaacacctatacagcgtcaggcaattcccattgggctacagaatcgtgacatcattggtgtggctgagactggcagtggcaagacagcagccttcctcatccctctgctggtctggatcaccacacttcccaaaattgacaggatcgaagagtcagaccaaggcccttatgccatcatcctggctcccacccgtgagttggctcaacagattgaggaagagaccatcaagtttgggaaaccgctaggtatccgcactgtggctgtcattggtggcatctccagagaagaccagggcttcaggctgcgcatgggttgtgagattgtgattgctacccctgggcgtttgattgatgtgctggagaaccgctacctggtgctgagccgctgtacctatgtggttctggatgaggcagataggatgattgacatgggctttgagccagatgtccagaagatcctggagcacatgcctgtcagcaaccagaagccagacacggatgaggctgaggaccctgagaagatgctggccaactttgagtcgggaaaacataagtaccgccaaacagtcatgttcacggccaccatgcccccagcggtggagcgtctggccaggagctatcttcggcgacctgctgtggtgtacattggctccgcaggcaagccccatgagcgtgtggaacagaaggtcttcctcatgtcagagtcagaaaagaggaaaaagctgctggcaatcttggagcaaggctttgacccacccatcattatttttgtcaaccagaagaagggctgcgacgtgttggccaaatccctggagaagatggggtacaatgcttgcacactgcacggtggaaaaggccaggagcagcgagagtttgcgttgtccaacctcaaggctggggccaaggatattttggtggctacagatgtggctggtcgtggtattgacatccaagatgtgtctatggttgtcaactatgatatggccaaaaatattgaagattacatccaccgcattggccgcacgggacgagcaggcaagagtggggtggccatcaccttcctcacaaaagaggactctgctgtgttctacgagctgaagcaagctatcctggaaagcccagtgtcttcctgtccccccgaactagccaaccacccagatgcccagcataagccaggcaccatcctcaccaagaagcgccgggaagagaccatctttgcctga

SEQ ID NO: 72 - DNA, 3907 bp, synthetic construct
  source 1..3907: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgaagttgaaggacacaaaatcaaggccaaagcagtcaagctgtggcaaatttcagacaaagggaatcaaagttgtgggaaaatggaaggaagtgaagattgacccaaatatgtttgcagatggacagatggatgacttggtgtgctttgaggaattgacagattaccagttggtctcccctgccaagaatccctccagtctcttctcaaaggaagcacccaagagaaaggcacaagctgtttcagaagaagaggaggaggaggagggaaagtctagctcaccaaagaaaaagatcaagttgaagaaaagtaaaaatgtagcaactgaaggaaccagtacccagaaagaatttgaagtgaaagatcctgagctggaggcccagggagatgacatggtttgtgatgatccggaggctggggagatgacatcagaaaacctggtccaaactgctccaaaaaagaagaaaaataaagggaaaaaagggttggagccttctcagagcactgctgccaaggtgcccaaaaaagcgaagacatggattcctgaagttcatgatcagaaagcagatgtgtcagcttggaaggacctgtttgttcccaggccggttctccgagcactcagctttctaggcttctctgcacccacaccaatccaagccctgaccttggcacctgccatccgtgacaaactggacatccttggggctgctgagacaggaagtgggaaaactcttgcctttgccatcccaatgattcatgcggtgttgcagtggcagaagaggaatgctgcccctcctccaagtaacaccgaagcaccacctggagagaccagaactgaggccggagctgagactagatcaccaggcaaggctgaagctgagtctgatgcattgcctgacgatactgtaattgagagtgaagcactgcccagtgatattgcagccgaggccagagccaagactggaggcactgtctcagaccaggcgttgctctttggtgacgatgatgctggtgaagggccttcttccctgatcagggagaaacctgttcccaaacagaatgagaatgaggaggaaaatcttgataaagagcagactggaaatctaaaacaggagttggatgacaaaagcgccacctgtaaggcatatccaaagcgtcctctgcttggactggttctgactcccactcgagagctggccgtccaggtcaaacagcacattgatgctgtggccaggtttacaggaattaaaactgctattttggttggtggaatgtccacgcagaaacagcagaggatgctgaaccgtcgtcctgagattgtggttgctactccaggccggctgtgggaattaattaaagaaaagcattatcatttgaggaaccttcggcagctcaggtgcctggtagtggatgaggctgaccggatggttgagaaaggccattttgctgagctctcacagctgctagagatgctcaatgactcccaatacaacccaaagagacaaacgcttgttttttctgccacactcaccctggtgcatcaggctcctgctcgaatccttcataagaagcacaccaagaaaatggataaaacagccaaacttgacctccttatgcagaaaattggcatgaggggcaagcccaaggtcattgacctcacaaggaatgaggccacggtggagacgctaacagagaccaagatccattgtgagactgatgagaaagacttctacttgtactacttcctgatgcagtatccaggccgcagcttagtgtttgccaacagtatctcctgcatcaaacgcctctctgggctcctcaaagtccttgatatcatgcccttgaccctgcatgcctgtatgcaccagaagcagaggctcagaaacctggagcagtttgcccgtctggaagactgtgttctcttggcaacagatgtggcagctcggggtctggatattcctaaagtccagcatgtcatccattaccaggtcccacgtacctcggagatttatgtccaccgaagtggtcgaactgctcgagctaccaatgaaggcctcagtctgatgctcattgggcctgaggatgtgatcaactttaagaagatttacaaaacgctcaagaaagatgaggatatcccactgttccccgtgcagacaaaatacatggatgtggtcaaggagcgaatccgtttagctcgacagattgagaaatctgagtatcggaacttccaggcttgcctgcacaactcttggattgagcaggcagcagctgccctggagattgagctggaagaagacatgtataagggaggaaaagctgaccagcaagaagaacgtcggagacaaaagcagatgaaggttctgaagaaggagctgcgccacctgctgtcccagccactgtttacggagagccagaaaaccaagtatcccactcagtctggcaagccgcccctgcttgtgtctgccccaagtaagagcgagtctgctttgagctgtctctccaagcagaagaagaagaagacaaagaagccgaaggagccacagccggaacagccacagccaagtacaagtgcaaattaa

SEQ ID NO: 73 - DNA, 2779 bp, synthetic construct
  source 1..2779: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggcgtcgttactgtggggaggcgacgcaggggcggcggagagcgagcggctgaacagccacttttcaaacctcagccaaccccggaagaacctttggggtattaagagtactgcagtccgaaacatagatggttctattaataacatcaatgaagatgatgaagaagatgtagtggatttggcagctaattcactcttaaacaagttaatccatcaatccttagtagaatccagtcaccgtgtggaagttttacagaaggatcccagctctccactttactcagtaaagacatttgaagagctgcggctaaaggaagagttactaaaaggaatctatgcaatgggatttaataggccatctaaaatccaagagatggctctccctatgatgctggcacatccaccccagaacctcatagcacagagccagtctggaacaggaaagacagcggcatttgtgttggcaatgttaagcagagttaatgccttggaattgttcccacagtgcctctgcctagctcctacttatgaattggctctgcaaactggccgtgtggttgagcagatgggaaaattctgtgtggatgttcaagtgatgtatgccattcgagggaatcgaattcccagaggcaccgacatcactaaacagattataattggcactcctgggactgtcctagattggtgttttaaactaaaattgattgatttgactaagattcgtgtgtttgtcctggatgaagcagatgtgatgattgacactcaaggattctcagatcatagtattcgtattcaaagagctctaccctccgaatgccaaatgctcctcttttcagcaacctttgaggactctgtgtggcactttgctgagcgaatcatccctgaccctaatgttatcaagttacgcaaagaggagctcacactgaacaacatccggcaatattacgtgctgtgtgagcacaggaaagacaaataccaagctctgtgcaacatttatggcagcatcaccattggtcaggccatcatcttctgccagactcgtcgaaacgctaagtggttgaccgtggagatgatacaggatggccaccaggtgtctttgttaagcggggagctgaccgtggagcagcgagcttccatcattcagaggtttcgggatgggaaagagaaggttctcataacaactaatgtttgtgcccgagggattgatgtgaagcaggtcacaattgttgtgaactttgatctccctgtaaaacaaggagaggagccggactatgagacctacctccaccgcatagggcggacggggcgctttgggaaaaaaggccttgccttcaacatgattgaagtagatgagctgccctcgctcatgaaaatccaggaccactttaacagcagtattaagcaactcaacgctgaagacatggatgaaattgaaaagattgactattga

SEQ ID NO: 74 - DNA, 3625 bp, synthetic construct
  source 1..3625: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgcttgcggacctcggcttaatcggaaccataggcgaggatgacgaggtgccggtggagcccgagtctgactccggggacgaggaagaggaggggcccattgtgctgggcagacgacaaaaagctttggggaagaaccgcagtgctgatttcaaccctgatttcgttttcactgagaaggaggggacgtacgatggcagctgggccctggctgatgtcatgagccaactcaagaagaagagggcagccactacattagatgagaagattgagaaagttcgaaagaaaaggaaaacagaggataaagaagccaagtctgggaagttggaaaaggagaaagaagcaaaggaaggctctgaaccaaaggagcaggaagaccttcaagagaatgatgaggaaggctcagaagatgaagcctcggagactgactactcatcagctgatgagaacatcctcaccaaagcagatacactcaaagtaaaggatcggaagaagaagaagaagaaaggacaggaagcaggaggattttttgaagatgcatctcagtacgatgaaaacctctcgttccaggacatgaacctttcccgccctcttctgaaggccattacagccatgggcttcaagcagcccaccccgatccagaaggcgtgcatacctgtgggtctattggggaaggacatctgtgcctgtgcagccactgggacaggtaaaactgccgcctttgccctgcctgttttggagcgtctgatttataaaccccgccaggctccagtcacccgcgtgctggtgctagtgcccacccgagagctgggcatccaggtgcactctgtcaccagacagctggcccagttctgcaacatcaccacctgcctggctgtgggcggcttggatgtgaagtctcaggaagcagctcttcgggcagcgcctgacatcctcatcgccaccccaggccggctcatcgatcacctccacaactgcccttccttccacctgagcagcatcgaggtgctcatcctggacgaggctgacaggatgctggatgagtactttgaggagcagatgaaggagatcatccgaatgtgttcccaccaccgccagaccatgctcttctcggccaccatgacagacgaggtgaaagatctggcttctgtctccttgaagaatcctgtccggatatttgtgaacagcaacacagatgtggctcccttcctgcggcaggagttcatccggatccggcctaatcgtgaaggagaccgggaagccatcgtggcagctttgttgacgaggaccttcactgaccatgtgatgctgttcacgcaaaccaagaagcaggcccaccgcatgcacatcctcctggggctcatggggctgcaggtgggtgagctccatggcaacttgtcacagacgcagcggctggaggccctccggcgttttaaggatgaacagattgacatcctcgtggccactgatgtggcagcccgtggacttgacattgagggggtcaaaacggtaatcaacttcacaatgcctaataccatcaaacattatgtccaccgggtggggcgaacagcacgtgctggcagggctgggcgctcagtctctctggtgggagaagatgagcggaagatgctgaaggagattgtaaaagctgccaaggcccctgtgaaggccaggatacttccccaagatgtcatcctcaaattccgggacaagattgagaaaatggagaaagatgtgtatgcagttctgcagctagaggcggaggaaaaagagatgcagcagtcagaagcccagatcaatacagcaaagcggctcctggagaaggggaaggaggcagtggtccaagagcccgagaggagctggttccagaccaaagaagagaggaagaaggagaaaattgccaaagctctgcaggaatttgacttggccttaagaggaaagaagaaaaggaagaagtttatgaaggatgccaaaaaaaagggggagatgacagcagaggaaaggtctcagtttgaaatcctcaaggcgcagatgtttgctgaacggctagcgaagaggaatcgcagagccaagcgggcccgagcaatgcccgaggaggagccagtgagaggtcctgccaagaagcaaaagcaggggaagaaatctgtatttgatgaagaactcaccaacacaagcaagaaggccctgaaacagtatcgagctggcccttcctttgaagaaaggaaacagttgggcttgccccaccagagacgaggaggaaactttaaatctaaatccagatacaagaggaggaagtag

SEQ ID NO: 75 - DNA, 2950 bp, synthetic construct
  source 1..2950: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggctctaacgcggccggtgcggctcttttccctcgtgactcggttgctcctggcgccgcgacggggcctcacggtccgcagtcccgacgaacccctgccggtggtgcgcatcccagtggctctacagcggcagttggaacagcggcagagcaggcggcggaacctcccgaggccggtgctggttcgacccggaccgctgctggtttcggcgcggcggccggagttgaaccagccggcgcgcctcacactgggccgttgggagcgcgcgccgctagcctctcaaggctggaagagtcgacgcgcgcgtcgggaccacttctccatcgagcgcgcgcaacaggaggcgccagcggtgcgaaagctctcgtctaagggcagctttgctgacctgggcctggagccccgtgtgctgcacgcactacaggaggctgcgcctgaagtcgttcagcccacaaccgtgcagtctagcaccatcccctcactacttcgcggccgccatgtcgtttgcgccgcagaaaccggcagtggcaagactctcagctacctcctgccgctgcttcaacggctcttgggccagccaagcctggactcccttcctatccccgcgccccgaggcctggtccttgttccttcccgagaattggcccaacaggtgcgggctgtggcccaacccttgggccgctccttgggcctgctggtgcgggacctggagggaggccacggcatgcgtaggatcaggctgcagctgtccagacagccttcagcagatgtgcttgtggccactccaggggctctgtggaaggccctgaaaagtcgactgatcagtctggagcaactctccttcttggtgttggatgaggcagacacactgctggatgaaagcttcctggaactggtggactacatcttagagaagagccacatagcagaaggcccagctgacttggaagaccccttcaatcccaaagctcagttagtgctggtaggagccacatttcccgaaggtgtaggccagttgctgaataaagtcgccagcccagatgctgtcaccaccatcaccagctccaagctccactgtatcatgcctcatgtgaaacagacatttctgagactgaagggagcagataaggtggccgagctggtgcacatcctcaagcatcgtgacagagcagaaaggactggtccctcaggaactgttctggtgttctgtaatagctccagcactgtgaactggctgggatatattctggatgaccacaaaatccaacacctaaggttgcaggggcaaatgccagccttgatgagggtaggaatcttccagtccttccagaagagctcccgagacatacttctctgcacagacatagcctctcggggcctggacagcactggtgtggagctggttgtcaattatgatttccccccaacgctgcaagattacatccacagagcagggagagtgggccgtgtggggagcgaggtgccaggcaccgtcatcagttttgtgacccatccctgggatgtgagcctggttcagaagattgagctggcggctcgccgaaggagaagtcttccaggactagcatcctcggtgaaagagcctttgccccaagcaacctga

SEQ ID NO: 76 - DNA, 3568 bp, synthetic construct
  source 1..3568: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggcagccgccgacggttcgctcttcgacaaccccaggacgttctccagacgtcccccagcccaggcgagtcggcaagcaaaggctacgaaaagaaaataccaagcgtccagtgaggctcccccagcgaaacggaggaacgaaacttcatttctcccagccaagaaaactagtgttaaagaaactcagaggacttttaaggggaacgcacaaaaaatgttttctccaaagaagcattcggttagcacaagtgatagaaaccaggaggagagacagtgcattaagacttcatcactgtttaaaaacaaccctgacattccagaactccacagacctgtggtaaagcaggtgcaagaaaaagtgtttacttcagctgcttttcatgagctgggcctccacccacatttaatttccacaataaatacggtcttaaaaatgtctagtatgaccagtgttcagaagcaaagtattcctgtgttgctggaaggcagagatgctctcgtgagatcccagacgggctcaggtaaaactcttgcctattgcatccctgtggtccagtcccttcaagcaatggagtcaaaaatacagcgcagtgatggcccctatgccctggtgctcgtgccaacgagagagctagctctacaaagctttgacactgtccagaaactgcttaagcctttcacctggattgtgcctggagtgttaatgggaggagagaagagaaaatcagaaaaggccagactccgcaaaggaataaatatccttatctcaactcctggacgcctggtggatcatataaaatccacaaagaacattcattttagtcggctgcggtggttggtgtttgatgaagcagacagaatcttggatttgggttttgaaaaggacatcacagtgatacttaatgctgtaaatgctgaatgccaaaaacgacagaatgtcttgctatcagcgacactcacagaaggtgtaacgcggctagctgatatcagtttgcatgatccagtcagtatttctgtcctggacaagagccatgaccagttgaacccaaaggacaaagcggtccaggaggtctgtcctccaccagctggcgacaagctggacagctttgcaataccagagagtctcaagcagcatgtgactgtggttcccagcaaactgaggcttgtctgcctagcggccttcatccttcagaaatgcaagtttgaggaagaccagaagatggttgtctttttctcaagttgcgagctggtggagttccactacagcctcttcctacagaccctgctgagcagctcaggggcgccggcatcagggcagttgccatctgcctccatgcgattaaaattcctacggctgcatggcggcatggagcaggaggaaagaacagcagtgtttcaggaattttcacattccagaagaggcgtccttctttgcacggatgttgcagctcggggcttagatctccctcaagtcacgtggattgttcagtacaacgctccatcttcacctgcagaatacatccaccggattggaagaaccgcccggattggctgccatgggagcagcctgctcattttggctccttcggaggcagaatatgtcaactcgttggcttctcacaaaatcaacgtttctgagattaagatggaagatattttgtgtgttctgacaagagatgattgttttaaagggaaacgatggggagcccagaaatcccatgctgttggcccccaggaaatccgagagcgagccacagtcttgcagacggtatttgaagattacgtgcactccagtgagaggagggtctcctgggcaaagaaagctctgcagtccttcatccaagcctacgccacctaccccagggagctgaagcacatcttccacgtccgatccctccaccttgggcatgtggcgaagagcttcggactaagagatgcccccaggaatcttagtgccttgactagaaagaagaggaaagcacacgtgaaaaggcctgaccttcataagaagacccagagtaaacacagcctcgctgaaatcctacgttcggaatactcaagcggcatggaggccgacatcgccaaggtcaaaaagcaaaacgcacctggagagcctggtggccggcccctgcagcacagtctgcagccgacaccctgctttggccgtgggaaaacattaaaatggagaaaaacccaaaaaggtgtacagcgggacagcaagacttcccagaaagtttaa

SEQ ID NO: 77 - DNA, 2614 bp, synthetic construct
  source 1..2614: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggcagagaacgatgtggacaatgagctcttggactatgaagatgatgaggtggagacagcagctgggggagatggggctgaggcccctgccaagaaggatgtcaagggctcctatgtctccatccacagctctggctttcgtgacttcctgctcaagccagagttgctccgggccattgtcgactgtggctttgagcatccgtcagaagtccagcatgagtgcatccctcaggccattctgggaatggatgtcctgtgccaggccaagtcgggcatgggaaagacagcagtgtttgtcttggccacactgcaacagctggagccagttactgggcaggtgtctgtactggtgatgtgtcacactcgggagttggcttttcagatcagcaaggaatatgagcgcttctctaaatacatgcccaatgtcaaggttgctgttttttttggtggtctgtctatcaagaaggatgaagaggtgctgaagaagaactgcccgcatatcgtcgtggggactccaggccgtatcctagccctggctcgaaataagagcctcaacctcaaacacattaaacactttattttggatgaatgtgataagatgcttgaacagctcgacatgcgtcgggatgtccaggaaatttttcgcatgaccccccacgagaagcaggtcatgatgttcagtgctaccttgagcaaagagatccgtccagtctgccgcaagttcatgcaagatccaatggagatcttcgtggatgatgagacgaagttgacgctgcatgggttgcagcagtactacgtgaaactgaaggacaacgagaagaaccggaagctctttgaccttctggatgtccttgagttcaaccaggtggtgatctttgtgaagtctgtgcagcggtgcattgccttggcccagctactagtggagcagaacttcccagccattgccatccaccgtgggatgccccaggaggagaggctttctcggtatcagcagtttaaagattttcaacgacgaattcttgtggctaccaacctatttggccgaggcatggacatcgagcgggtgaacattgcttttaattatgacatgcctgaggattctgacacctacctgcatcgggtggccagagcaggccggtttggcaccaagggcttggctatcacatttgtgtccgatgagaatgatgccaagatcctcaatgatgtgcaggatcgctttgaggtcaatattagtgagctgcctgatgagatagacatctcctcctacattgaacagacacggtag

SEQ ID NO: 78 - DNA, 3196 bp, synthetic construct
  source 1..3196: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggaggagtcggaacccgaacggaagcgggctcgcaccgacgaggtgcctgccggaggaagccgctccgaggcggaagatgaggacgacgaggactacgtgccctatgtgccgttacggcagcgccggcagctactgctccagaagctgctgcagcgaagacgcaagggagctgcggaggaagagcagcaggacagcggtagtgaaccccggggagatgaggacgacatcccgctaggccctcagtccaacgtcagcctcctggatcagcaccagcaccttaaagagaaggctgaagcgcgcaaagagtctgccaaggagaagcagctgaaggaagaagagaagatcctggagagtgttgccgagggccgagcattgatgtcagtgaaggagatggctaagggcattacgtatgatgaccccatcaaaaccagctggactccaccccgttatgttctgagcatgtctgaagagcgacatgagcgcgtgcggaagaaataccacatcctggtggagggagacggtatcccaccacccatcaagagcttcaaggaaatgaagtttcctgcagccatcctgagaggcctgaagaagaaaggcattcaccacccaacacccattcagatccagggcatccccaccattctatctggccgtgacatgataggcatcgctttcacgggttcaggcaagacactggtgttcacgttgcccgtcatcatgttctgcctggaacaagagaagaggttacccttctcaaagcgcgaggggccctatggactcatcatctgcccctcgcgggagctggcccggcagacccatggcatcctggagtactactgccgcctgctgcaggaggacagctcaccactcctgcgctgcgccctctgcattgggggcatgtccgtgaaagagcagatggagaccatccgacacggtgtacacatgatggtggccaccccggggcgcctcatggatttgctgcagaagaagatggtcagcctagacatctgtcgctacctggccctggacgaggctgaccgcatgatcgacatgggcttcgagggtgacatccgtaccatcttctcctacttcaagggccagcgacagaccctgctcttcagtgccaccatgccgaagaagattcagaactttgctaagagtgcccttgtaaagcctgtgaccatcaatgtggggcgcgctggggctgccagcctggatgtcatccaggaggtagaatatgtgaaggaggaggccaagatggtgtacctgctcgagtgcctgcagaagacacccccgcctgtactcatctttgcagagaagaaggcagacgtggacgccatccacgagtacctgctgctcaagggggttgaggccgtagccatccatgggggcaaagaccaggaggaacggactaaggccatcgaggcattccgggagggcaagaaggatgtcctagtagccacagacgttgcctccaagggcctggacttccctgccatccagcacgtcatcaattatgacatgccagaggagattgagaactatgtacaccggattggccgcaccgggcgctcgggaaacacaggcatcgccactaccttcatcaacaaagcgtgtgatgagtcagtgctgatggacctcaaagcgctgctgctagaagccaagcagaaggtgccgcccgtgctgcaggtgctgcattgcggggatgagtccatgctggacattggaggagagcgcggctgtgccttctgcgggggcctgggtcatcggatcactgactgccccaaactcgaggctatgcagaccaagcaggtcagcaacatcggtcgcaaggactacctggcccacagctccatggacttctga

SEQ ID NO: 79 - DNA, 4144 bp, synthetic construct
  source 1..4144: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgaactggaataaaggtggtcctggcactaagcgaggatttggctttggaggttttgccatcagtgctgggaaaaaggaggaacccaaactcccacagcagtcccacagtgcctttggggcaaccagctcttcttctggatttggaaagtcagctccaccacagcttccttctttctacaaaattggatctaagcgggccaactttgatgaagaaaatgcctattttgaagatgaggaagaagattctagcaacgttgatttaccttacattcctgctgaaaactcaccaactcgccagcaattccattccaagccagtagattctgacagcgatgatgatcccttggaggcattcatggctgaagtggaggatcaggcagctagagacatgaagaggcttgaagaaaaggacaaggaaagaaaaaacgtaaagggtattcgagatgacattgaagaggaagatgaccaagaagcttattttcgatacatggcagaaaacccaactgctggtgtggttcaggaggaagaggaagacaatctagaatatgatagtgacggaaatccaattgcacctaccaaaaaaatcattgatcctcttccccccattgatcattcagagattgactatccaccatttgaaaaaaacttttacaatgagcatgaagagataaccaacctcactccacagcagttaatagatctccggcataagctcaatcttcgggtctctggtgctgcacctcctagaccaggaagtagctttgctcattttgggtttgacgaacaacttatgcaccagattcggaaatctgaatacacacagcccactccaatacagtgccagggtgtgcctgtggcattaagtggtagagacatgattggtattgccaaaacaggtagtgggaaaactgcagccttcatttggcccatgttgattcatataatggaccagaaggagttggaaccaggtgatggaccaattgcagtgattgtgtgtcctaccagggagctttgccagcagatccatgcagaatgtaagcggtttggaaaagcatataatcttcgatcagtggccgtatatggaggagggagtatgtgggagcaggccaaggcccttcaggagggggcagagattgttgtgtgtaccccaggtcgactgatagatcatgtgaaaaagaaagctaccaatcttcaaagagtctcttaccttgtgtttgatgaagcagatcgaatgtttgacatgggatttgagtaccaagttcgatccatagcaagtcatgttcgtcctgacaggcagactctcttatttagtgcaacttttcggaagaagattgaaaagttggccagagacatcctgatcgaccctattcgagtggtgcagggagatattggagaggcaaatgaagatgtgacacagattgtggagattctccattctggacctagtaaatggaactggcttacccggcgtctggtagaatttacctcttcagggagtgtcctcctctttgttactaaaaaagccaatgctgaagagctagcgaataaccttaaacaggagggtcataatcttgggctgctccatggggatatggatcagagtgagagaaacaaggtcatttcagactttaagaaaaaggacatcccagtcctggtggccacagatgttgcagcccgtggtctggacattccttcaattaagactgtcattaactatgatgtggcacgagacattgatacccacacgcataggattggccgcacaggaagagcgggtgagaaaggtgtggcctataccctactcactcccaaggacagcaattttgctggtgacctggtccggaacttggaaggagccaatcaacacgtttctaaggaactcctagatctggcaatgcagaatgcctggtttcggaaatctcgattcaaaggagggaaaggaaaaaagctgaacattggtggaggaggcctaggctacagggagcggcctggcctgggctctgagaacatggatcgaggaaataacaatgtaatgagcaattatgaggcctacaagccttccacaggagctatgggagatcgactaacggcaatgaaagcagctttccagtcacagtacaagagtcactttgttgcagccagtttaagtaatcagaaggctggaagttctgctgctggggcaagtgggtggactagtgcagggagcttgaattctgttccaactaactcagcacaacagggccataacagtcctgacagccccgtcaccagtgccgccaagggcatcccaggctttggcaatactggcaacatcagtggtgcccctgtgacctacccgtctgccggagcccaaggagtcaacaacacagcttcagggaataacagccgagaagggactgggggcagcaacgggaaaagagagagatatactgagaaccggggcagcagccgtcacagtcacggagagactggcaatcggcatagcgatagtccacgtcacggagatggtggtcgccatggagatggataccgccatccagaaagcagcagccgtcatactgatggccatcggcacggggagaacagacatggaggaagcgcaggccggcatggggagaaccggggtgcaaatgatggtcggaatggggaaagcaggaaagaagcttttaatcgtgagagcaagatggagcccaagatggaacccaaagtggacagcagcaagatggacaaggtggacagcaagacagataagacagctgacggctttgctgtcccagagccgcctaaacgcaagaaaagtcgatgggacagttag

SEQ ID NO: 80 - DNA, 3274 bp, synthetic construct
  source 1..3274: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgtcccaccacggaggagctcccaaggcctctacgtgggtcgttgctagtcggcgaagctcgacagtgtcccgagcgccagagaggaggccggcggaggagttgaatcgaacaggtcctgagggatatagtgtcggcagaggtggtcgctggagaggcacctctaggcccccggaggccgtggccgctggtcacgaggaactgccgctgtgttttgctttgaagagccactttgttggcgcggtaatcggtcgtggtgggtcaaaaataaagaatatacaaagtacaacaaacaccacaatccaaataatacaagaacaaccagaatcattagtcaaaatttttggcagcaaggcaatgcaaacgaaagcaaaagcagtgatagacaattttgttaaaaagctagaagaaaattacaattcagaatgcggaattgatactgcattccaaccttctgttggaaaagatggaagcacagataacaatgttgttgcaggagatcggccattgatagattgggatcaaattagagaggaaggtttgaaatggcaaaaaacaaagtgggcagatttaccaccaattaagaaaaacttttataaagagtccactgccacaagtgccatgtcaaaagtagaagcagatagttggaggaaagaaaattttaatataacgtgggatgacttgaaggatggggagaaacgacctatccccaatcctacctgcacatttgatgacgcctttcaatgttatcctgaggttatggaaaacattaaaaaggcaggttttcaaaagccaacacctattcagtcacaggcatggcccattgtgttgcaaggaatagatcttataggagtagcccagactggaacaggaaagacattgtgttatttaatgcctggatttattcatctggtccttcaacccagccttaaaggtcaaaggaatagacccggcatgttagttctaactcccactcgggaattagcacttcaagtagaaggagaatgttgcaaatattcatataaagggcttcggagtgtttgtgtatatggtggtggaaatagagatgaacaaatagaagagcttaaaaaaggtgtagatatcataattgcaactcccggaagattgaatgatctgcaaatgagtaacttcgtcaatctgaagaatataacctacttggttttagatgaagcagacaagatgttggacatgggatttgaaccccagataatgaagattttgttagatgtgcgcccagataggcagacagttatgaccagtgctacatggcctcattcagttcatcgcctcgcacaatcttatttgaaagaaccaatgattgtctatgttggtacattggatctagttgctgtaagttcagtgaagcaaaatataattgtaaccaccgaggaagagaaatggagtcacatgcaaacttttctacagagtatgtcatccacagacaaagtcattgtcttcgtttctcgaaaagctgttgcggatcacttatcaagtgacctaatacttggaaatatatcagtagagtctctgcatggagatagagaacagagagatcgggagaaagcattagagaactttaaaacaggcaaagtgagaatactaattgcaactgatctagcctctagaggacttgatgtccatgacgttacacatgtctataattttgactttccacggaatattgaagaatacgtacaccgaatagggcgcacgggaagagcagggaggactggtgtttccattacaactttgactagaaatgattggagggttgcctctgaattgattaatattctggaaagagcaaatcagagtattccagaggagcttgtatcaatggctgagaggtttaaggcacatcaacagaaaagggaaatggaaagaaaaatggaaagacctcaaggaaggcccaagaagtttcattaa

SEQ ID NO: 81 - DNA, 4426 bp, synthetic construct
  source 1..4426: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgggtcgggagtcacgccactatcgaaaacgatcggcatcccggggtcgctctggaagtcggtctagaagtcgctcaccctcagacaaaagaagtaaacgtggagatgacagacggtctagaagtagagatagagataggaggagagagaggtctcgtagcagggataaaagaagatctcggtcaagggacaggaagcgtctgagacgttccagaagtagagagagagacagaagccgagagcgaagaagatctcgaagtagagacaggagacgctcaaggagtagaagccggggccggcgatcccgatcctccagtcctggaaataaaagcaagaaaactgagaatagatctaggtccaaagagaaaactgatggtggggaaagttctaaagagaagaaaaaagacaaagatgacaaggaggatgaaaaagaaaaagatgctggcaactttgaccagaataagctggaagaagaaatgagaaagcgaaaagaaagagtagaaaaatggcgagaagagcaacgtaaaaaggctatggaaaacataggagaactgaaaaaggaaatcgaagagatgaaacaagggaaaaagtggagtttagaggacgatgatgatgacgaagatgatcctgcagaagctgaaaaggagggaaatgaaatggagggtgaggagttagatccattagatgcttacatggaagaagtgaaagaggaagtaaaaaaatttaacatgagaagtgtaaaaggtggtgggggaaatgaaaagaagtctgggccaacggtcacaaaagttgtcactgttgtgacaaccaaaaaagcagttgtggattctgataagaagaaaggtgagctgatggagaatgaccaggatgccatggagtattcttcagaggaggaagaagttgatcttcagacagcccttacagggtatcaaacaaaacagcgaaagcttctagaaccagttgatcatggaaaaattgagtatgagccatttaggaaaaacttctatgttgaagttccagaactagcaaaaatgtctcaagaagaggtaaatgtgtttcgattggaaatggagggcattacagttaaaggaaaaggttgccccaaaccaattaaatcctgggtccagtgtggaatttccatgaagatcttaaattccctcaagaagcatggctatgaaaagcccacgcccatccaaacccaagctattcctgctataatgtctggacgagatttgattggcattgccaaaacaggaagtggaaagaccattgcttttctgttgcccatgtttagacacatcatggatcagaggtcattagaggaaggagaggggccaatagctgtcatcatgactccaactcgagaactggctttacagattactaaagagtgtaagaagttttccaagactttgggacttagagtggtctgtgtttacggaggaacaggaatcagtgagcagattgctgagctgaaaagaggtgctgaaattattgtttgcacacctggtcgaatgattgacatgttagccgctaacagtggtcgggtcacaaatcttcgaagagtgacatatgttgttttagatgaagcagacagaatgtttgacatgggttttgaaccccaggtcatgcgcatcgtggataatgttcgtcctgatcgacagacggttatgttttcagctactttccccagagctatggaggctttggctcgcaggatcctcagtaaacctattgaagtacaagttggaggcaggagtgtggtttgctcagatgtggagcaacaagtgattgtgattgaagaagaaaagaaattcttgaagttacttgagcttctaggccattatcaagagtcaggatctgtcattatatttgtggataagcaggaacatgctgatggtcttcttaaggatttaatgagagcatcttatccttgcatgtctcttcatggaggcattgatcaatatgacagagatagcatcataaatgactttaagaatgggacctgcaaacttcttgtggctacctctgttgctgcccgaggtctagatgtgaaacatctgattcttgtagtaaattatagctgccccaaccattatgaggattatgtacacagagcagggcggactggaagagcaggaaacaagggttatgcttatacttttatcacagaggatcaagctcgctatgctggtgacataattaaagctcttgaattgtcagggactgcagtacctcctgatttagagaaactgtggagtgatttcaaagatcagcagaaagctgaggggaaaataattaaaaagagtagtgggttctctggtaagggattcaagtttgatgaaacagaacaagctttggctaatgagaggaagaagttacaaaaagcagctcttggtctacaagattcagatgatgaggatgctgcagttgatattgatgagcaaattgaaagcatgtttaattcaaagaagagagtaaaggatatggctgctcctggaacatcaagtgttcctgctccaactgcaggaaatgctgagaaattagaaattgctaagagattggctcttagaatcaatgcccagaagaatttgggcatcgagtctcaggtagatgtgatgcagcaggccaccaatgcaattcttaggggtggcaccattctggctcccactgtttctgcaaaaaccattgcagaacaacttgctgaaaagatcaatgccaagctcaattatgtgccgttagagaaacaagaagaagagagacaggatggtggacagaatgaatcttttaagagatatgaagaagaattagagatcaatgacttcccacagactgctaggtggaaagttacctctaaggaagctctgcagagaatcagtgaatactctgaagccgcaattacaatcagaggaacctacttccctcctggcaaagaacccaaggaaggcgagcggaagatttacttggcaattgaaagtgccaatgaactggctgtgcagaaagcaaaggcagaaatcaccaggctcataaaagaagagctgatccggctgcaaaattcataccaaccaacaaataaaggaagatacaaagtcttatag

SEQ ID NO: 82 - DNA, 2695 bp, synthetic construct
  source 1..2695: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggcggcacccgaggaacacgattctccgaccgaagcgtcccagccgattgtggaagaggaggaaactaaaacatttaaagacctgggtgtgacagatgtgttgtgtgaagcttgtgaccagttgggatggacaaaacccaccaagatccagattgaagctattcctttggccttacaaggtcgtgatatcattgggcttgcagaaactggctctggaaagacaggcgcctttgctttgcccattctaaacgcactgctggagaccccgcagcgtttgtttgccctagttcttaccccgactcgggagctggcctttcagatctcagagcagtttgaagccctggggtcctctattggagtgcagagtgctgtgattgtaggtggaattgattcaatgtctcaatctttggcccttgcaaaaaaaccacatataataatagcaactcctggtcgactgattgaccacttggaaaatacgaaaggtttcaacttgagagctctcaaatacttggtcatggatgaagccgaccgaatactgaatatggattttgagacagaggttgacaagatcctcaaagtgattcctcgagatcggaaaacattcctcttctctgccaccatgaccaagaaggttcaaaaacttcagcgagcagctctgaagaatcctgtgaaatgtgccgtttcctctaaataccagacagttgaaaaattacagcaatattatatttttattccctctaaattcaaggatacctacctggtttatattctaaatgaattggctggaaactcctttatgatattctgcagcacctgtaataatacccagagaacagctttgctactgcgaaatcttggcttcactgccatccccctccatggacaaatgagtcagagtaagcgcctaggatcccttaataagtttaaggccaaggcccgttccattcttctagcaactgacgttgccagccgaggtttggacatacctcatgtagatgtggttgtcaactttgacattcctacccattccaaggattacatccatcgagtaggtcgaacagctagagctgggcgctccggaaaggctattacttttgtcacacagtatgatgtggaactcttccagcgcatagaacacttaattgggaagaaactaccaggttttccaacacaggatgatgaggttatgatgctgacagaacgcgtcgctgaagcccaaaggtttgcccgaatggagttaagggagcatggagaaaagaagaaacgctcgcgagaggatgctggagataatgatgacacagagggtgctattggtgtcaggaacaaggtggctggaggaaaaatgaagaagcggaaaggccgttaa

SEQ ID NO: 83 - DNA, 2563 bp, synthetic construct
  source 1..2563: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggcgaccacggccacgatggcgacctcgggctcggcgcgaaagcggctgctcaaagaggaagacatgactaaagtggaattcgagaccagcgaggaggtggatgtgacccccacgttcgacaccatgggcctgcgggaggacctgctgcggggcatctacgcttacggttttgaaaaaccatcagcaatccagcaacgagcaatcaagcagatcatcaaagggagagatgtcatcgcacagtctcagtccggcacaggaaaaacagccaccttcagtatctcagtcctccagtgtttggatattcaggttcgtgaaactcaagctttgatcttggctcccacaagagagttggctgtgcagatccagaaggggctgcttgctctcggtgactacatgaatgtccagtgccatgcctgcattggaggcaccaatgttggcgaggacatcaggaagctggattacggacagcatgttgtcgcgggcactccagggcgtgtttttgatatgattcgtcgcagaagcctaaggacacgtgctatcaaaatgttggttttggatgaagctgatgaaatgttgaataaaggtttcaaagagcagatttacgatgtatacaggtacctgcctccagccacacaggtggttctcatcagtgccacgctgccacacgagattctggagatgaccaacaagttcatgaccgacccaatccgcatcttggtgaaacgtgatgaattgactctggaaggcatcaagcaatttttcgtggcagtggagagggaagagtggaaatttgacactctgtgtgacctctacgacacactgaccatcactcaggcggtcatcttctgcaacaccaaaagaaaggtggactggctgacggagaaaatgagggaagccaacttcactgtatcctcaatgcatggagacatgccccagaaagagcgggagtccatcatgaaggagttccggtcgggcgccagccgagtgcttatttctacagatgtctgggccagggggttggatgtccctcaggtgtccctcatcattaactatgatctccctaataacagagaattgtacatacacagaattgggagatcaggtcgatacggccggaagggtgtggccattaactttgtaaagaatgacgacatccgcatcctcagagatatcgagcagtactattccactcagattgatgagatgccgatgaacgttgctgatcttatctga

SEQ ID NO: 84 - DNA, 2779 bp, synthetic construct
  source 1..2779: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggcaggcttcgcggagctcgggctgtcatcgtggctcgtggaacaatgtcggcagctgggtttgaagcagcccacgcccgtgcagctcggctgcatccccgccatcctggagggtcgagactgcttgggctgtgctaagacaggcagtgggaagacagcagcgtttgtccttcccatcttgcagaagctgtctgaggatccctatggcatcttctgcctcgtcctgacacccaccagggagctggcctaccagatcgcagagcagttccgggtcctggggaagcctctagggctgaaagactgcatcatcgtcggtggcatggacatggtggcccaggcgctggagctctctcggaaaccacacgtggtcatcgccacgccggggcgcctggcagatcacctgcgcagctccaacacttttagtataaagaagatccgcttcctggtgatggatgaggcagaccggctgctggaacagggctgcactgacttcaccgtggacctggaggccatcctggcggctgtgccggcccgcaggcagacactgctgttcagcgccacgctgaccgacacactccgggagctgcagggtctggccaccaaccagcccttcttctgggaagcacaggccccggtgagcaccgtggagcagctggaccagcgctacctgctggtgcctgagaaggtcaaggacgcctacctggtccacctgatccagcgcttccaggatgagcacgaggactggtccattatcatcttcaccaacacgtgcaagacctgccagattctgtgcatgatgctgcgcaaattcagcttccccaccgtggctctgcactccatgatgaagcagaaagaacgctttgccgccctagccaagttcaagtccagcatctaccggatcctgatcgcaacagacgtggcctcccggggcctggacatccctacggtacaggtggtcatcaaccacaacacccccgggctccccaagatctacatccaccgagtcggccggacggcccgtgcagggcggcagggtcaggccatcacgctggtgacacagtacgacatccacctggtgcacgccatcgaggagcagatcaagaagaagctggaggagttctccgtggaagaggccgaggtgctacagatcctcacacaggtcaacgtggtgcgaagagagtgtgagatcaaactggaggcggcccactttgacgaaaagaaggagatcaacaaacggaagcagctgatcctggaggggaaggaccctgacctggaggccaagcgcaaggctgagctggccaagatcaagcagaagaaccggcgcttcaaggagaaggtggaggagacgctgaagcgacagaaggctggcagggctggccacaaggggcgtccacccaggacaccgtctgggtcccactcaggcccagtcccctcccagggcctggtctga

SEQ ID NO: 85 - DNA, 3541 bp, synthetic construct
  source 1..3541: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgcctgggaaactcctctggggggacattatggagctggaagcacccttggaggagtccgagagccagaagaaggagaggcaaaagagtgacagaaggaagtcaaggcaccattatgactcggatgagaaatcagaaacaagagaaaatggtgttacagatgacctggatgctcccaaggccaaaaaatctaaaatgaaagagaagctaaatggagacactgaagaaggatttaatagactttcagatgaattctccaaatctcataagtcaagaagaaaagatctaccaaatggagatatagatgaatatgaaaaaaaatcaaagcgagtatcatctttagatacttctactcataaatcaagtgataataaactagaggagaccttaacacgtgaacagaaagaaggagccttctccaattttcctatttctgaagagactataaagcttctgaaaggtcgaggggtaacatatctctttcctattcaagttaagacctttggtcctgtatatgaaggaaaagatttaatagctcaagcacggacaggaacaggaaagacattctcttttgccatccccttaattgaaagactccaaagaaatcaagaaacaattaaaaaaagccgctcaccaaaggtacttgttttggctccaacaagggaactggcaaaccaagtagccaaagacttcaaagatataactaggaaactcagcgtggcgtgtttttatggtggaacatcatatcaaagccaaattaatcatattcgaaatggtattgacatcttggttggaacacctggtcgtatcaaagaccatctgcagagtggccgattggatctttctaaactgcgacatgttgtgcttgatgaagtggatcagatgttagatttaggtttcgctgaacaagttgaagatattattcatgaatcctacaaaactgattctgaagacaatcctcagactttacttttttctgcaacttgcccacagtgggtatacaaagttgcaaaaaaatacatgaaatccagatatgaacaggttgaccttgttggaaaaatgactcaaaaggctgcaactactgtggaacatttggccatccagtgtcattggtctcagaggccagcagttattggagatgtccttcaagtctacagtgggtctgaagggagggctattattttctgtgagaccaagaagaatgtaactgaaatggccatgaatccacacataaaacagaatgcccagtgtttacatggggacattgcacagtcacaaagagaaattacactaaaaggcttcagagaaggtagttttaaagttttggtggcaaccaatgtggctgcccgtggtttggacattcctgaagttgacctggtgattcaaagttctcctcctcaggatgttgagtcctatatccatcgctctggacgcacaggtagagctggacggacagggatttgtatatgtttttatcaaccaagagaaagaggtcaactaagatatgtggaacaaaaagcaggaattacttttaaacgtgtaggtgttccttctacaatggatttagttaaatctaaaagcatggatgccatcaggtctctggcttccgtttcttacgctgctgttgattttttccgaccatcagctcagagactgatagaagagaaaggtgcagtggatgcattggctgcagctttagcccacatttctggtgcatcaagctttgaaccacgatctttgatcacctctgataaggggtttgtgaccatgactctggaaagcctagaggaaatacaggatgtcagctgtgcttggaaagaacttaacagaaagctgagtagtaatgcagtgtctcagattaccagaatgtgcctcctgaaaggaaatatgggtgtttgctttgatgttcctacaactgagtcagaaaggttacaggcagagtggcatgattccgactggatactctcagtgccagccaaattacctgaaattgaagaatattatgatggaaacacatcttctaattccagacagaggagtggctggtcaagtggtcgatcaggccggtcaggccggtcaggtggtcgatctggcggccggtcaggtagacagagtcgacaaggaagtcgctcaggaagtcgacaagatggtagaagacgaagtgggaatagaaatcgatcaagaagtgggggccacaaacggagttttgactga

SEQ ID NO: 86 - DNA, 3328 bp, synthetic construct
  source 1..3328: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggcgctgttctacgtcgcgcggtacccgggccccgatgcggcagctgcggcggggccggagggcgcggaggccggggcgcacggcagggcccgcgcgctgctcgagcggctgcagagccgggcccgcgaacggcagcagcagcgggagcccgcgcagaccgaggcggctgcatcgaccgagccggcgaccaggaggcgacggcggccccggcggcggcggcgggtgaacgacgcggagccggggagcccggaggcgccgcagggaaagcgacggaaggcggacggcgaggacgcgggcgcagaaagcaacgaggaggcgccaggggagcccagcgcagggagcagcgaggaggcgccaggggagcccagcgcagggagcagcgaggaggcgccgggggagcgcagcaccagcgccagcgccgaggcggccccagatggaccggccctggaggaggcggccggacccctggtccccggcctggtgctgggggggttcgggaagaggaaggcgccgaaggtccagcctttcctgccaaggtggctggctgagcctaactgtgtcagaaggaatgtcaccgaagacctggttcctatcgaggacatccctgacgtccatcctgacctgcagaagcagctgcgggcacacggcatctcgtcctactttccagtccaggcagctgtgattcctgccctcctggagagcgcagcctgtgggtttctggtgggcagaggtggctaccggcctagcgacctctgtgtttctgccccaacaggcagtgggaagacactggccttcgtcatccctgtggtgcaggccctgctttcgagagtggtctgccacatccgtgccctggttgtgctgcccaccaaggagctggcccagcaggtgagcaaagttttcaacatctacacagatgccacacctctgagagtctccctggttacgggacagaagtctctggccaaggagcaggagagcctcgtccagaaaacagctgatgggtaccgctgcttggctgacatcgtggtagccacccccggccgcctggtggaccacatcgaccagaccccaggattcagcctccagcagctccgcttcctgattatcgacgaggctgaccggatgattgacagcatgcatcagtcctggctgccgcgggtggtggcggccgccttccagagcgaggaccccgcggacccctgtgccctgctccagcgaaggcaggcccaggctgtgacagccgccagcacctgctgtccccagatgcccctgcagaagctgctcttctcagctactctgacccagaaccctgaaaagctgcagcagctgggcctccaccagccccggcttttctccacagggctagcacacaggggcctggaagatacagatggggacggggattcggggaagtatgcctttcctgttgggctcacgcaccactacgtgccctgcagcctcagctctaagccgctggtcgtcctgcacctggtcctggagatgggcttctcgagggttctctgcttcactaactcccgagagaactcccacaggctcttcctgctggtgcaagcttttgggggtgtggacgtggctgagttctcctcgcgctacgggcctggccagaggaggatgatcctgaagcagtttgaacaggggaagatccagctgctcatcagcacggacgccaccgcgcgaggcatcgacgtgcagggtgtggagctggtggtgaactacgacgccccccagtacctgagaacctacgtgcaccgggttgggaggacagctcgcgctgggaaaactggacaggccttcacactgctcctgaaagtgcaggagaggagattcctccgaatgctaactgaagctggggcacctgagttgcagcggcacgagctctccagcaagctgctgcagccgctggttcctcggtacgaggaggccctgtcccagctggaggagtctgtcaaggaagagcgcaagcagagggcggcctag

SEQ ID NO: 87 - DNA, 3127 bp, synthetic construct
  source 1..3127: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggacgtccacgatctctttcgccggctcggcgcgggggccaaattcgacacgagacgcttctcggcagacgcagctcgattccagataggaaaaaggaaatatgactttgattcttcggaggtgcttcagggactggacttttttggaaacaagaagtctgtcccaggtgtgtgtggagcatcacaaacacatcagaagccccaaaatggagagaaaaaagaagagagcctaactgaaaggaagagggagcagagcaagaaaaaaaggaagacgatgacttcagaaattgcttcccaagaagaaggtgctactatacagtggatgtcatctgtagaagcaaagattgaagacaaaaaagttcagagagaaagtaaactaacttccggaaagttggagaatctcagaaaagaaaagataaacttcttgcggaataaacacaaaattcacgtccaaggaaccgatcttcctgacccaattgctacatttcagcaacttgaccaggaatataaaatcaattctcgactacttcagaacattctagatgcaggtttccaaatgcctacgccaatccaaatgcaagccatcccagttatgctgcatggtcgggaacttctggcttctgctccaactggatctggaaaaacattagcttttagcattcctattttaatgcagctgaaacaacccgcaaataaaggcttcagagccctgattatatcaccaacacgagaacttgccagccagattcacagagagttaataaaaatttctgagggaacaggattcaggatacacatgatccacaaagcagcagtggcagccaagaaatttggacctaaatcatctaaaaagtttgatattcttgtgactactccaaatcgactaatctatttattaaagcaagatccccccggaatcgacctagcaagtgttgagtggcttgtagtagacgaatcagataaactgtttgaagatggcaaaactgggttcagagaccagctggcttccattttcctggcctgcacatcccacaaggtccgaagagctatgttcagtgcaacttttgcatatgatgttgaacagtggtgcaaactcaacctggacaatgtcatcagtgtgtccattggagcaaggaattctgcagtagaaactgtagaacaagagcttctctttgttggatctgagaccggaaaacttctggccatgagagaacttgttaaaaagggtttcaatccacctgttcttgtttttgttcagtccattgaaagggctaaagaactttttcatgagctcatatatgaaggtattaatgtggatgttattcatgcagagagaacacaacaacagagagataacacagtccacagtttcagagcaggaaaaatctgggttctgatttgtacagccttgctagcaagagggattgattttaaaggtgtgaacttggtgatcaactatgactttccaactagctcagtggaatatatccacaggataggtcgaactggaagagcagggaataagggaaaagcaattacatttttcactgaggatgataagccattattaagaagcgttgctaatgttatacagcaggctgggtgtcctgtaccagaatacataaaaggttttcagaaactactaagcaaacaaaagaaaaagatgattaagaaaccattggaaagggagagcattagtacaactccaaaatgtttcttagaaaaagctaaggataaacagaaaaaggtcactggtcagaacagcaagaagaaagtagctcttgaagacaaaagttaa

SEQ ID NO: 88 - DNA, 3223 bp, synthetic construct
  source 1..3223: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgtcccactgggccccagagtggaagagggcggaggctaatccaagagaccttggggccagctgggatgtcaggggcagcagaggcagtggctggagtggccccttcggccatcagggaccgagagcagcaggctcccgtgaaccaccactctgctttaaaataaagaacaatatggttggtgtggtcattggttacagtggatcaaaaataaaagatctacaacattcgacaaacactaaaatacagatcataaacggggaatctgaagcaaaagtcagaatttttggcaatagggaaatgaaagcaaaggccaaagcggctatagaaacacttattagaaaacaagaaagctacaactcagaatccagtgtggataatgctgcatcccaaacccctattggaagaaatctaggcagaaatgacattgttggagaagctgagccattgtcaaattgggatcgcattagggcagcagtcgtggagtgcgaaaagagaaaatgggcagatctaccaccagttaagaaaaacttttacatagaatccaaagcaacaagctgcatgtctgaaatgcaggtgattaactggagaaaggaaaatttcaacataacgtgtgatgacttgaaaagtggtgaaaagcgtctcattccaaaaccaacttgcaggtttaaagacgcttttcagcaataccctgatcttctgaaaagcataataagggtagggattgtaaagccaacgccaattcagtcacaggcatggccaattattctacaaggaatagatcttatagtagttgcacaaaccggaacagggaaaacattgtcctatctaatgcctgggtttattcatcttgattctcaaccaatatctagagagcaaaggaatgggcctgggatgctagtccttacacccactagagagttggctcttcacgtggaagctgaatgttcaaagtattcatataaaggtctcaaaagcatttgcatatatggtggtagaaacagaaatggacaaatagaagacattagcaaaggtgtagatatcattattgcaactcctgggaggctgaatgacctacaaatgaataactctgtcaacctaagaagcataacctacttggttatagatgaggcagataaaatgctggatatggaatttgaaccccagataaggaagattttattagatgtgcgcccagaccgacagactgttatgacaagtgcaacttggccagatactgtacgtcaactagcactttcttatttgaaagatcctatgattgtttatgttggtaatctgaatctagtggctgtaaatacagtgaagcaaaatataattgttaccacagaaaaagaaaaacgagctctcacccaagaattcgtagagaacatgtcacccaacgacaaagtcatcatgtttgtcagccaaaaacatattgctgatgacttgtcaagcgacttcaatatccaaggcatatctgcagaatcattacatggcaacagtgaacagagtgatcaagagcgagcagtagaggactttaaaagcggaaacataaagatactgattacaactgatatagtatcccgaggtcttgatcttaatgatgtcacacatgtatataattatgatttcccaaggaatattgacgtatatgtacacagagtagggtacattggacggacaggaaagactggcacatcagttaccctcatcactcagagagattcgaaaatggccggtgaattgattaaaattctggacagagcaaatcagagtgttccggaagatcttgtagtaatggctgagcaatacaagttaaatcaacaaaagaggcacagagaaacacgatcaagaaaacctggacaaagacgcaaggagttttattttttaagttga

SEQ ID NO: 89 - DNA, 3973 bp, synthetic construct
  source 1..3973: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggcggccgacaagggcccggcggctggacctcggtcgcgagctgccatggcccagtggaggaagaagaaagggctccggaagcgccgaggcgcggcctcccaggcccgcggcagcgactcggaggacggcgagtttgagatccaggcggaagatgacgcccgggcccggaagctgggacctggaagacccctgcccaccttccccacctcggaatgcacctcggatgtggagccggacacccgggagatggtgcgtgcccagaacaagaagaagaagaagtctggaggcttccagtccatgggcctgagctacccggtgttcaaaggcatcatgaagaaggggtacaaggtgccaacacccatccagaggaagaccatcccggtgatcttggatggcaaggacgtggtggccatggcccggacgggcagtggcaagacagcctgcttcctcctcccaatgttcgagcggctcaagacccacagtgcccagaccggggcccgcgccctcatcctctcgccgacccgagagctggccctgcagaccctgaagttcactaaggagctaggcaagttcactggcctcaagactgccctgatcctgggtggagacaggatggaagaccagtttgcagccctgcacgaaaatcccgacataattattgccacgcccggacggttggtgcatgtggctgtggaaatgagcctgaagctgcagagtgtggaatacgtggtgttcgatgaagctgaccggctttttgaaatgggtttcgcagagcagctgcaggagatcatcgcccgcctccccgggggccaccagacggtgctgttctccgccacgctgcccaaactgctggtggaatttgcccgggctggcctcacggagcccgtgctcatccggcttgacgtggataccaagctcaacgagcagctgaagacctccttcttcctcgtgcgggaggacaccaaggctgccgtgctgctccacctgctgcacaacgtggtgcggccccaggaccagaccgtggtgtttgtggccacgaagcaccacgccgagtacctcactgagctgctgacgacccagcgggtgagctgcgcccacatctacagtgccctagacccgacagcccgcaagatcaatctcgccaaattcacgcttggcaagtgctccactctcattgtgactgacctggccgcccgaggcctggacatcccgctgctggacaatgtcatcaactacagcttccccgccaagggcaaactcttcctgcaccgcgtgggccgtgtggctcgggctggccgaagtggcacagcctactccttggtggcccctgatgaaatcccctacctgctggatctgcacctgttcctgggccgctccctcaccctcgcccgacccctcaaggagccctcaggtgtggccggtgtggatggcatgctgggtcgggtgccacagagtgtggtggacgaggaggacagtggtctgcagagcaccctggaggcatcgctggagctacggggcctggcccgcgttgctgataacgcccagcagcagtatgtgcgctcacgcccggcgccctcgcctgagtccatcaagagggccaaggagatggaccttgtggggctgggcctgcaccccctcttcagctcgcgttttgaggaggaggagctgcagcggctgaggctggtggacagcataaagaactaccgctcccgggcgactatctttgagatcaacgcctccagccgagacctgtgcagccaggtgatgcgcgccaagcggcagaaggaccgcaaggccatcgcccgcttccagcagggacagcaggggcggcaggagcagcaggagggcccagtgggcccagccccgagccgcccagcactgcaggagaagcagcctgagaaggaggaggaggaggaggcgggagagagtgtggaggacattttctcagaggtcgtgggccggaagcggcagcggtcaggacccaacaggggagccaagaggcggagggaggaggcccggcagcgggaccaggaattctacatcccctaccggcccaaggactttgacagcgagcggggcctgagcatcagcggggaagggggagcctttgagcagcaggcagctggcgctgtcctggacttgatgggggatgaagcccagaacctgacgaggggccggcagcagctcaagtgggaccgtaagaagaagcggtttgtgggacagtcaggacaggaagacaagaagaagattaagacagagagcggccgctacatcagcagctcctacaagcgagacctctatcagaagtggaaacagaaacagaaaattgatgatcgtgactcggacgaagaaggggcatctgaccggcgaggcccagagcgaagaggtgggaagcgagaccgtggccaaggtgcatcccggccccacgccccaggcacccctgcaggccgagtccgcccggaactcaagaccaagcagcagatcctgaagcagcggcgccgggcccagaagctgcacttcctgcagcgtggtggcctcaagcagctctctgcccgcaaccgccgccgcgtccaggagctgcagcagggcgccttcggccggggtgcccgctccaagaagggcaagatgcggaagaggatgtga

SEQ ID NO: 90 - DNA, 3130 bp, synthetic construct
  source 1..3130: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggagcatgtgacagagggctcctgggagtcgctgcctgtgccgctgcacccgcaggtgctgggcgcgctgcgggagctgggcttcccgtacatgacgccggtgcagtccgcaaccatccctctgttcatgcgaaacaaagatgtcgctgcagaagcggtcacaggtagtggcaaaacactcgcttttgtcatccccatcctggaaattcttctgagaagagaagagaagttaaaaaagagtcaggttggagccataatcatcacccccactcgagagctggccattcaaatagacgaggtcctgtcgcatttcacgaagcacttccccgagttcagccagattctttggatcggaggcaggaatcctggagaagatgttgagaggtttaagcaacaaggtgggaacatcattgtggccactccaggccgcttggaggacatgttccggaggaaggccgaaggcttggatctggccagctgtgtgcgatccctggatgtcctggtgttggatgaggcagacagacttctggacatggggtttgaggcaagcatcaacaccattctggagtttttgccaaagcagaggagaacaggccttttctctgccactcagacgcaggaagtggagaacctggtgagagcgggcctccggaaccctgtccgggtctcagtgaaggagaagggcgtggcagccagcagtgcccagaagaccccctcccgcctggaaaactactacatggtatgcaaggcagatgagaaatttaatcagctggtccattttcttcgcaatcataagcaggagaaacacctggtcttcttcagcacctgtgcctgtgtggaatactatgggaaggctctggaagtgctggtgaagggcgtgaagattatgtgcattcacggaaagatgaaatataaacgcaataagatcttcatggagttccgcaaattgcaaagtgggattttagtgtgcactgatgtgatggcccggggaattgatattcctgaagtcaactgggttttgcagtatgaccctcccagcaatgcaagtgccttcgtgcatcgctgcggtcgcacagctcgcattggccacgggggcagcgctctggtgttcctcctgcccatggaagagtcatacatcaatttccttgcaattaaccaaaaatgccccctgcaggagatgaagccccagagaaacacagcggaccttctgccaaaactcaagtccatggccctggctgacagagctgtgtttgaaaagggcatgaaagcttttgtgtcatatgtccaagcttatgcaaagcatgaatgcaacctgattttcagattaaaggatcttgattttgccagccttgctcgaggttttgccctgctgaggatgcccaagatgccagaattgagaggaaagcagtttccagattttgtgcccgtggacgttaataccgacacgattccatttaaagataaaatcagagaaaagcagaggcagaaactcctggagcaacaaagaagagagaaaacagaaaatgaagggagaagaaaattcataaaaaataaagcttggtcaaagcagaaggccaaaaaagaaaagaagaaaaaaatgaatgagaaaaggaaaagggaagagggttctgatattgaagatgaggacatggaagaacttcttaatgacacaagactcttgaaaaaacttaagaaaggcaaaataactgaagaagaatttgagaagggcttgttgacaactggcaaaagaacaatcaagacagtggatttagggatctcagatttggaagatgactgctga

SEQ ID NO: 91 - DNA, 2971 bp, synthetic construct
  source 1..2971: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatggaggactctgaagcactgggcttcgaacacatgggcctcgatccccggctccttcaggctgtcaccgatctgggctggtcgcgacctacgctgatccaggagaaggccatcccactggccctagaagggaaggacctcctggctcgggcccgcacgggctccgggaagacggccgcttatgctattccgatgctgcagctgttgctccataggaaggcgacaggtccggtggtagaacaggcagtgagaggccttgttcttgttcctaccaaggagctggcacggcaagcacagtccatgattcagcagctggctacctactgtgctcgggatgtccgagtggccaatgtctcagctgctgaagactcagtctctcagagagctgtgctgatggagaagccagatgtggtagtagggaccccatctcgcatattaagccacttgcagcaagacagcctgaaacttcgtgactccctggagcttttggtggtggacgaagctgaccttcttttttcctttggctttgaagaagagctcaagagtctcctctgtcacttgccccggatttaccaggcttttctcatgtcagctacttttaacgaggacgtacaagcactcaaggagctgatattacataacccggttacccttaagttacaggagtcccagctgcctgggccagaccagttacagcagtttcaggtggtctgtgagactgaggaagacaaattcctcctgctgtatgccctgctcaagctgtcattgattcggggcaagtctctgctctttgtcaacactctagaacggagttaccggctacgcctgttcttggaacagttcagcatccccacctgtgtgctcaatggagagcttccactgcgctccaggtgccacatcatctcacagttcaaccaaggcttctacgactgtgtcatagcaactgatgctgaagtcctgggggccccagtcaagggcaagcgtcggggccgagggcccaaaggggacaaggcctctgatccggaagcaggtgtggcccggggcatagacttccaccatgtgtctgctgtgctcaactttgatcttcccccaacccctgaggcctacatccatcgagctggcaggacagcacgcgctaacaacccaggcatagtcttaacctttgtgcttcccacggagcagttccacttaggcaagattgaggagcttctcagtggagagaacaggggccccattctgctcccctaccagttccggatggaggagatcgagggcttccgctatcgctgcagggatgccatgcgctcagtgactaagcaggccattcgggaggcaagattgaaggagatcaaggaagagcttctgcattctgagaagcttaagacatactttgaagacaaccctagggacctccagctgctgcggcatgacctacctttgcaccccgcagtggtgaagccccacctgggccatgttcctgactacctggttcctcctgctctccgtggcctggtgcgccctcacaagaagcggaagaagctgtcttcctcttgtaggaaggccaagagagcaaagtcccagaacccactgcgcagcttcaagcacaaaggaaagaaattcagacccacagccaagccctcctga

SEQ ID NO: 92 - DNA, 3970 bp, synthetic construct
  source 1..3970: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgaccaccgagcagcgacgcagcctgcaagccttccaggattatatccggaagaccctggaccctacctacatcctgagctacatggccccctggtttagggagggttattctggactttatgaagccattgaaagttgggatttcaaaaaaattgaaaagttggaggagtatagattacttttaaaacgtttacaaccagaatttaaaaccagaattatcccaaccgatatcatttctgatctgtctgaatgtttaattaatcaggaatgtgaagaaattctacagatttgctctactaaggggatgatggcaggtgcagagaaattggtggaatgccttctcagatcagacaaggaaaactggcccaaaactttgaaacttgctttggagaaagaaaggaacaagttcagtgaactgtggattgtagagaaaggtataaaagatgttgaaacagaagatcttgaggataagatggaaacttctgacatacagattttctaccaagaagatccagaatgccagaatcttagtgagaattcatgtccaccttcagaagtgtctgatacaaacttgtacagcccatttaaaccaagaaattaccaattagagcttgctttgcctgctatgaaaggaaaaaacacaataatatgtgctcctacaggttgtggaaaaacctttgtttcactgcttatatgtgaacatcatcttaaaaaattcccacaaggacaaaaggggaaagttgtcttttttgcgaatcagatcccagtgtatgaacagcagaaatctgtattctcaaaatactttgaaagacatgggtatagagttacaggcatttctggagcaacagctgagaatgtcccagtggaacagattgttgagaacaatgacatcatcattttaactccacagattcttgtgaacaaccttaaaaagggaacgattccatcactatccatctttactttgatgatatttgatgaatgccacaacactagtaaacaacacccgtacaatatgatcatgtttaattatctagatcagaaacttggaggatcttcaggcccactgccccaggtcattgggctgactgcctcggttggtgttggggatgccaaaaacacagatgaagccttggattatatctgcaagctgtgtgcttctcttgatgcgtcagtgatagcaacagtcaaacacaatctggaggaactggagcaagttgtttataagccccagaagtttttcaggaaagtggaatcacggattagcgacaaatttaaatacatcatagctcagctgatgagggacacagagagtctggcaaagagaatctgcaaagacctcgaaaacttatctcaaattcaaaatagggaatttggaacacagaaatatgaacaatggattgttacagttcagaaagcatgcatggtgttccagatgccagacaaagatgaagagagcaggatttgtaaagccctgtttttatacacttcacatttgcggaaatataatgatgccctcattatcagtgagcatgcacgaatgaaagatgctctggattacttgaaagacttcttcagcaatgtccgagcagcaggattcgatgagattgagcaagatcttactcagagatttgaagaaaagctgcaggaactagaaagtgtttccagggatcccagcaatgagaatcctaaacttgaagacctctgcttcatcttacaagaagagtaccacttaaacccagagacaataacaattctctttgtgaaaaccagagcacttgtggacgctttaaaaaattggattgaaggaaatcctaaactcagttttctaaaacctggcatattgactggacgtggcaaaacaaatcagaacacaggaatgaccctcccggcacagaagtgtatattggatgcattcaaagccagtggagatcacaatattctgattgccacctcagttgctgatgaaggcattgacattgcacagtgcaatcttgtcatcctttatgagtatgtgggcaatgtcatcaaaatgatccaaaccagaggcagaggaagagcaagaggtagcaagtgcttccttctgactagtaatgctggtgtaattgaaaaagaacaaataaacatgtacaaagaaaaaatgatgaatgactctattttacgccttcagacatgggacgaagcagtatttagggaaaagattctgcatatacagactcatgaaaaattcatcagagatagtcaagaaaaaccaaaacctgtacctgataaggaaaataaaaaactgctctgcagaaagtgcaaagccttggcatgttacacagctgacgtaagagtgatagaggaatgccattacactgtgcttggagatgcttttaaggaatgctttgtgagtagaccacatcccaagccaaagcagttttcaagttttgaaaaaagagcaaagatattctgtgcccgacagaactgcagccatgactggggaatccatgtgaagtacaagacatttgagattccagttataaaaattgaaagttttgtggtggaggatattgcaactggagttcagacactgtactcgaagtggaaggactttcattttgagaagataccatttgatccagcagaaatgtccaaatga

SEQ ID NO: 93 - DNA, 3187 bp, synthetic construct
  source 1..3187: mol_type = other DNA
ggcctaactggccggtacctgagtctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaagtcgaacacgcagatgcagtcggggcggcgcggtcccaggtccacttcgcatattaaggtgacgcgtgtggcctcgaacaccgagcgaccctgcagcgacccgcttaaaagcttggcaatccggtactgttggtaaagccaccatggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtgggagttccggtggtggcgggagcggaggtggaggcatgtttgttccaagatctctaaaaatcaagaggaatgctaatgatgatggcaaaagttgtgtggctaagataattaaaccagacccagaagaccttcagttggacaaaagcagagatgttcccgttgatgctgtagctacagaagcagccacaatagacaggcacatcagcgaatcatgccctttccccagcccaggtggccagttggcagaggttcattcagtaagtcccgagcagggtgcgaaggacagccatccttctgaagagcccgttaagtcattttccaaaacacagcgctgggcagaaccaggggaacccatctgtgttgtctgtggtcgttatggagagtatatctgtgataagacagatgaagatgtgtgtagtttggagtgtaaagcgaaacatcttctacaagttaaggaaaaggaagagaaatcaaaactcagcaatccacagaaggctgattctgagccagagtctccactgaatgcttcctatgtctacaaagagcacccctttattttgaaccttcaggaagaccagattgaaaatcttaaacagcagctgggaattttagttcaagggcaagaagtcaccaggcccattattgactttgaacattgtagtctccctgaggtcttaaatcacaacttgaagaaatcaggctatgaggtgccaactcccattcaaatgcagatgattcctgtgggacttctgggaagagacattctggccagtgcagatactggctcaggaaaaacagctgcttttcttcttcctgttatcatgcgagctttattcgagagcaaaactccatctgcgctcattcttacaccaaccagagagttagccattcagatagagagacaagctaaagaattgatgagtggcctgccacgcatgaaaactgtgcttcttgtagggggcttacccttacccccacagctttatcgtctgcaacaacatgttaaggttatcatagcaacccctgggcgacttctggatataataaagcagagctctgtagaactctgtggtgtaaagattgtggtagtagatgaagctgataccatgttaaagatgggttttcaacaacaagtgcttgacattttggaaaacattcctaatgattgtcagaccattttggtttcagccacaattccaactagcatagaacagctagcaagccagcttctgcataatcctgtgagaattatcactggagaaaagaacctaccttgtgccaatgtacgtcagattattttgtgggtagaagacccagccaaaaagaaaaaattatttgaaattttaaatgataagaaactctttaagcctccagtgttagtatttgtggactgcaaactaggagcagatcttttgagtgaagccgttcagaaaatcacagggctgaaaagcatatctatacattcagagaagtcgcaaatagaaaggaaaaacatattgaagggattacttgaaggagactatgaagttgtagtgagcacaggagtcttgggacgaggcctagacttgatcagtgtcaggctggttgtcaattttgatatgccttcaagtatggatgagtatgtccatcagattggaagagtaggaagattaggtcaaaatggaacagcgattactttcatcaataataattcaaaaagactcttctgggatattgcaaaacgagtaaagcccacaggatccattcttccccctcagttattaaattccccataccttcatgaccagaagagaaaggaacaacagaaagataaacagacacagaatgatctggttacaggagctaatcttatggatattatcagaaaacatgataaaagtaattctcagaaatga

(19) United States
(12) Patent Application Publication
(10) Pub. No.: US 2026/0276625 A1
(43) Pub. Date: Sep. 17, 2026

(54) SCREENING METHOD FOR COMPOUND INDUCING AUTOPHAGY

(71) Applicant: TOHOKU UNIVERSITY, Sendai-shi, Miyagi (JP)

(72) Inventors: Hirokazu ARIMOTO, Sendai (JP); Daiki TAKAHASHI, Sendai (JP)

(73) Assignee: TOHOKU UNIVERSITY, Sendai-shi, Miyagi (JP)

(21) Appl. No.: 19/469,266

(22) PCT Filed: Mar. 28, 2024
(86) PCT No.: PCT/JP2024/012899

§ 371 Date: Sep. 25, 2025

(30) Foreign Application Priority Data
Mar. 28, 2023 (WO) PCT/JP2023/012419

(51) Int. Cl. G01N 33/50; C12Q 1/25

(52) U.S. Cl. CPC G01N 33/502; C12Q 1/25; G01N 2500/02; G01N 2500/04; G01N 2500/10

(57) ABSTRACT

Provided is a screening method for searching for a new autophagy-inducing compound. In the present invention, a new autophagy-inducing compound is searched for using at least one of the following characteristics: (1) having high binding affinity for DEAD-box RNA helicase; (2) approximating DEAD-box RNA helicase and p62 to promote interaction; and (3) promoting droplet formation of DEAD-box RNA helicase and p62.

### TECHNICAL FIELD

The present invention relates to a method for screening a compound that induces autophagy and a method for producing a compound that induces degradation of a target intracellular molecule or an organelle by autophagy.

### BACKGROUND ART

Autophagy is one of the mechanisms of degradation of intracellular molecules in cells and is observed in eukaryotes ranging from yeast to humans. When autophagy is induced, a membrane vesicle called an autophagosome is formed, and subsequently, the autophagosome fuses with a lysosome, resulting in the degradation of molecules taken up into the autophagosome.

It is known that autophagy is induced by starvation of cells, and it has also become clear that autophagy is involved in physiological functions such as development and differentiation, and infection defense functions such as elimination of viruses that have invaded cells.

Various reports have also revealed the mechanism by which autophagy is induced. Mohammad Omar Faruk et al. reported in detail in a review article the mechanism of selective autophagy which degrades specific soluble proteins, supramolecular complexes, liquid-phase separated droplets, abnormal or excess organelles, pathogenic invasive bacteria and the like to contribute to cellular homeostasis (NON PATENT LITERATURE 1). Specifically, they reported that p62 bound to a ubiquitinated protein and liquid phase separation occurred, forming a droplet in which p62 and the ubiquitinated protein were condensed, and this droplet recruited other proteins to form an isolation membrane/phagocyte membrane, and p62 and other proteins in the droplet are degraded by a lysosome.

Lin Ma et al. suggested that in esophageal squamous cell carcinoma (ESCC), knockdown of DDX5 inhibited endoplasmic reticulum stress and promoted the recovery of autophagy flux (NON PATENT LITERATURE 2).

Hao Zhang et al. reported that DDX5 interacted with the autophagy receptor p62 to stimulate autophagy and suppressed the development of liver cancer (NON PATENT LITERATURE 3). They also reported that DDX5 promoted degradation of p62 and significantly reduced its half-life.

Yongsang Jo et al. disclosed in a report on interplay between intrinsically disordered proteins inside membrane-less protein liquid droplets that the addition of Streptavidin formed droplets of DDX4 (NON PATENT LITERATURE 4).

In addition, attempts to actively induce autophagy to provide new therapeutic methods (sometimes referred to as degraders) have also made significant progress (for example, NON PATENT LITERATURES 5 to 8).

Representative examples of degraders are Proteolysis-targeting chimeras (PROTAC)/specific non-genetic IAP-based protein erasers (SNIPERs), which are heterologous bifunctional molecules consisting of a ligand that binds to a target molecule and a degradation tag that induces degradation by the ubiquitin-proteasome system (UPS) (NON PATENT LITERATURES 5, 9 and 10). These heterologous bifunctional molecules have the advantage of being able to target a wide range of substrates by optimizing the degradation tag and replacing the target ligands. However, the degradation system using the UPS are limited to protein targets.

On the other hand, recently, a lysosome-based degradation system, lysosome-targeting chimeras (LYTACs) that degrade extracellular matrix, and autophagy-targeting chimeras (AUTACs) have been developed (NON PATENT LITERATURES 11 and 12), and creation of further novel chimeric molecules is expected.

### CITATION LIST

### Non Patent Literatures

NON PATENT LITERATURE 1: Cancer Sci, 2021, 112 (10): 3972-3978
NON PATENT LITERATURE 2: Biochem Biophys Res Commun, 2020, 533 (4): 1449-1456
NON PATENT LITERATURE 3: Hepatology, 2019, 69 (3): 1046-1063
NON PATENT LITERATURE 4: Chem. Sci., 2020, 11, 1269
NON PATENT LITERATURE 5: Samarasinghe, K. T. G.; Crews, C. M. Targeted protein degradation: a promise for undruggable proteins. Cell Chem. Biol. 2021, 28, 934-951.
NON PATENT LITERATURE 6: Li, K.; M. Crews, C. PROTACs: Past, Present and Future. Chem. Soc. Rev. 2022. doi: 10.1039/d2cs00193d.
NON PATENT LITERATURE 7: Burslem, G. M., Crews, C. M. Proteolysis-targeting chimeras as therapeutics and tools for biological discovery. Cell 2020, 181, 102-114.
NON PATENT LITERATURE 8: Paiva, S.-L.; Crews, C. M. Targeted protein degradation: elements of PROTAC design. Curr. Opin. Chem. Biol. 2019, 50, 111-119.
NON PATENT LITERATURE 9: Shusuke Tomoshige, Minoru Ishikawa, PROTACs and other chemical protein degradation technologies for the treatment of neurodegenerative disorders. Angew. Chem. Int. Ed. 2021, 60, 3346-3354.
NON PATENT LITERATURE 10: Mikihiko Naito, Nobumichi Ohoka, Norihito Shibata, SNIPERs-hijacking IAP activity to induce protein degradation. Drug Discov. Today Technol. 2019, 31, 35-42.
NON PATENT LITERATURE 11: Banik, S. M.; Pedram, K., Wisnovsky, S.; Ahn, G.; Riley, N. M.; Bertozzi, C. R.; Lysosome-targeting Chimaeras for degradation of extracellular proteins. Nature 2020, 584, 291-297.
NON PATENT LITERATURE 12: Daiki Takahashi and Hirokazu Arimoto, Selective autophagy as the basis of autophagy-base. Cell Chem. Biol. 2021, 28, 1061-1071.
NON PATENT LITERATURE 13: Yoshinobu Ichimura, Masaaki Komatsu, Roles of p62/SQSTM1 in autophagy, Journal of Japanese Biochemical Society 91 (3): 380-387 (2019) doi: 10.14952/SEIKAGAKU.2019.910380

### SUMMARY OF INVENTION

### Technical Problem

However, the design of these degradation tags in the chimeric molecules that induce selective autophagy has been made empirically by trial and error, and in order to efficiently create a novel chimeric molecule, there has been a need for the development of a tool for designing a degradation tag.

The present invention addresses this need and provides a tool for obtaining a degradation tag of a chimeric molecule that induces selective autophagy.

### Solution to Problem

The present inventors investigated the autophagy-inducing compounds and tag moieties developed by themselves for a mechanism of inducing autophagy and found that they exhibit the following properties:
(1) High binding affinity for DEAD-box RNA helicase;
(2) Bring DEAD-box RNA helicase and p62 close to each other to promote interaction; and
(3) Promoting droplet formation of DEAD-box RNA helicase and p62.

The present inventors focused on these properties and have constructed a screening method for searching for new autophagy-inducing compounds and used it to successfully find novel autophagy-inducing compounds.

The present invention is based on these findings and provides the following methods.
[1] A method for screening autophagy-inducing compounds, comprising:
contacting a test compound with a DEAD-box RNA helicase to measure a binding affinity of the test compound for the DEAD-box RNA helicase; and
selecting a compound having a binding affinity greater than or equal to a predetermined value.
[2] A method for screening autophagy-inducing compounds, comprising:
contacting a test compound with a DEAD-box RNA helicase in the presence of p62;
measuring an interaction between the DEAD-box RNA helicase and the p62; and
selecting a compound that induced the interaction.
[3] The method according to [2], wherein the DEAD-box RNA helicase and the p62 are expressed in a cell, and the cell is contacted with the test compound.
[4] A method for screening autophagy-inducing compounds, comprising:
contacting a test compound with a DEAD-box RNA helicase in the presence of p62;
measuring droplet formation of DEAD-box RNA helicase and p62; and
selecting a compound that induced the droplet formation.
[5] The method according to any one of [1] to [4], wherein the DEAD-box RNA helicase is DDX5 or DDX17.
[6] A method for producing a compound that induces degradation of a target intracellular molecule or organelle by autophagy, comprising:
performing the method of any one of [1] to [5]; and
linking a ligand that specifically binds to the target intracellular molecule or organelle to the resulting compound via a linker.
[7] The method according to [6], further comprising the step of contacting the compound to which the ligand is linked with a cell having the target intracellular molecule or organelle in vivo, ex vivo, or in vitro to confirm the degradation of the target intracellular molecule or organelle by autophagy.
[8] A method for inducing degradation of a target intracellular molecule or organelle by autophagy by contacting a compound obtained or obtainable by performing the method according to [6] or [7] with a DEAD-box RNA helicase in the presence of p62 in a cell.
[9] The method according to [8], wherein the compound obtained or obtainable by performing the method according to [6] or [7] is contacted with a DEAD-box RNA helicase in the presence of p62 in a cell to form a droplet containing the compound, the p62 and the DEAD-box RNA helicase.
[10] The method according to [8] or [9], wherein the method prevents or treats cancer.

### Advantageous Effects of Invention

According to a method according to an embodiment of the present invention, an autophagy-inducing compound can be efficiently obtained, and a compound that induces degradation of a target molecule or organelle by autophagy can be synthesized by linking the autophagy-inducing compound to a ligand that specifically binds to the target intracellular molecule via a linker as a degradation tag moiety.

### BRIEF DESCRIPTION OF DRAWINGS

FIG. 1 shows the emission amount 10 minutes after the start of measurement of luminescence in Examples 1 to 4.

FIG. 2 shows the emission amount 10 minutes after the start of measurement of luminescence in Examples 5 to 33.

FIG. 3 shows EGFP fluorescence levels in cases that cells transfected with pEGFP-FKBP12 were cultured in fresh DMEM containing 0.01 mM, 0.1 mM, 1 mass mM, and 10 mM AUTAC2-2G, respectively, and in fresh DMEM containing DMSO as a comparative control.

FIG. 4 shows fluorescence microscopic images showing the results of a mutant FKBP-tagged synuclein degradation test by SLF′-AUTAC.

FIG. 5 shows images of HeLa cells transiently expressing GFP-FKBP^{F36V} treated with SLF′-AUTAC, fixed with paraformaldehyde, then immunostained with an anti-DDX5 antibody or an anti-p62 antibody, and observed with a fluorescence microscope. The square box to the upper right is an enlarged image of a cytoplasm portion set forth in the small square box slightly to the upper left.

FIG. 6A shows images of HeLa cells transiently expressing GFP-FKBP12 and RFP-DDX5 treated with AUTAC2-2G and observed with a fluorescence microscope, and FIG. 6B shows images of HeLa cells transiently expressing GFP-FKBP12 and RFP-p62 treated with AUTAC2-2G and observed with a fluorescence microscope.

### DESCRIPTION OF EMBODIMENTS

Embodiments of the present invention will be described in detail. However, the present invention should not be understood as limited to the following embodiments.

### 1. Method for Screening Autophagy-Inducing Compounds

The screening method according to the present invention screens autophagy-inducing compounds utilizing at least one of the following properties:
(1) High binding affinity for DEAD-box RNA helicase;
(2) Bring DEAD-box RNA helicase and p62 close to each other to promote interaction; and
(3) Promoting droplet formation of DEAD-box RNA helicase and p62.

As demonstrated in the Examples described below, since compounds having an autophagy-inducing activity have these properties, autophagy-inducing compounds can be obtained by selecting compounds having at least one of these properties.

### 1-1. DEAD-Box RNA Helicase

The DEAD-box RNA helicase is a superfamily of RNA helicases having a motif DEAD (Asp-Glu-Ala-Asp). It is known that the DEAD-box RNA helicase is involved in the disassembly and formation of double-stranded RNA and RNA-protein complex using the energy of ATP hydrolysis, and plays an important role in transcription, splicing, RNA nuclear export and degradation, translation, ribosome biosynthesis, and the like.

Members of the DEAD-box RNA helicase include DDX1 (DEAD box protein 1), DDX2A (DEAD box protein 2A), DDX3X (DEAD box protein 3X), DDX4 (DEAD box protein 4), DDX5 (DEAD box protein 5), DDX6 (DEAD box protein 6), DDX10 (DEAD box protein 10), DDX11 (DEAD box protein 11), DDX17 (DEAD box protein 17), DDX18 (DEAD box protein 18), DDX19B (DEAD box protein 19B), DDX20 (DEAD box protein 20), DDX21 (DEAD box protein 21), DDX23 (DEAD box protein 23), DDX24 (DEAD box protein 24), DDX25 (DEAD box protein 25), DDX27 (DEAD box protein 27), DDX28 (DEAD box protein 28), DDX31 (DEAD box protein 31), DDX39B (DEAD box protein 39B), DDX41 (DEAD box protein 41), DDX42 (DEAD box protein 42), DDX43 (DEAD box protein 43), DDX46 (DEAD box protein 46), DDX47 (DEAD box protein 47), DDX48 (DEAD box protein 48), DDX49 (DEAD box protein 49), DDX50 (DEAD box protein 50), DDX51 (DEAD box protein 51), DDX52 (DEAD box protein 52), DDX53 (DEAD box protein 53), DDX54 (DEAD box protein 54), DDX55 (DEAD box protein 55), DDX56 (DEAD box protein 56), DDX58 (DEAD box protein 58), and DDX59 (DEAD box protein 59); and DEAD-Box Helicase 5 (DDX5) is also known as P68.

The amino acid sequences of these DEAD-box RNA helicase members and the nucleic acid sequences encoding the same are as follows.

[Chemical Formula 1]
DDX5 Amino acid sequence
(SEQ ID NO: 1)
MSGYSSDRDRGRDRGFGAPRFGGSRAGPLSGKKFGNPGEKLVKKKWNLDELPKFEKNFYQEHPDL
ARRTAQEVETYRRSKEITVRGHNCPKPVLNFYEANFPANVMDVIARQNFTEPTAIQAQGWPVALS
GLDMVGVAQTGSGKTLSYLLPAIVHINHQPFLERGDGPICLVLAPTRELAQQVQQVAAEYCRACR
LKSTCIYGGAPKGPQIRDLERGVEICIATPGRLIDFLECGKTNLRRTTYLVLDEADRMLDMGFEP
QIRKIVDQIRPDRQTLMWSATWPKEVRQLAEDFLKDYIHINIGALELSANHNILQIVDYCHDVEK
DEKLIRLMEEIMSEKENKTIVFVETKRRCDELTRKMRRDGWPAMGIHGDKSQQERDWVLNBFKHG
KAPILIATDVASRGLDVEDVKFVINYDYPNSSEDYIHRIGRTARSTKTGTAYTFFTPNNIKQVSD
LISVLREANQAINPKLLQLVEDRGSGRSRGRGGMKDDRRDRYSAGKRGGFNTFRDRENYDRGYSS
LLKRDFGAKTQNGVYSAANYINGSFGSNFVSAGIQTSFRTGNPTGTYQNGYDSTQQYGSNVPNMH
NGMNQQAYAYPATAAAPMIGYPMPTGYSQ
[Chemical Formula 2]
DDX5 DNA sequence
(SEQ ID NO: 2)
atgtcgggttattcgagtgaccgagaccgcggccgggaccgagggtttggtgcacctcgatttgg
aggaagtagggcagggcccttatctggaaagaagtttggaaaccctggggagaaattagttaaaa
agaagtggaatcttgatgagctgcctaaatttgagaagaatttttatcaagagcaccctgatttg
gctaggcgcacagcacaagaggtggaaacatacagaagaagcaaggaaattacagttagaggtca
caactgcccgaagccagttctaaatttttatgaagccaatttccctgcaaatgtcatggatgtta
ttgcaagacagaatttcactgaacccactgctattcaagctcagggatggccagttgctctaagt
ggattggatatggttggagtggcacagactggatctgggaaaacattgtcttatttgcttcctgc
cattgtccacatcaatcatcagccattcctagagagaggcgatgggcctatttgtttggtgctgg
caccaactcgggaactggcccaacaggtgcagcaagtagctgctgaatattgtagagcatgtcgc
ttgaagtctacttgtatctacggtggtgctcctaagggaccacaaatacgtgatttggagagagg
tgtggaaatctgtattgcaacacctggaagactgattgactttttagagtgtggaaaaaccaatc
tgagaagaacaacctaccttgtccttgatgaagcagatagaatgcttgatatgggctttgaaccc
caaataaggaagattgtggatcaaataagacctgataggcaaactctaatgtggagtgcgacttg
gccaaaagaagtaagacagcttgctgaagatttcctgaaagactatattcatataaacattggtg
cacttgaactgagtgcaaaccacaacattcttcagattgtggatgtgtgtcatgacgtagaaaag
gatgaaaaacttattcgtctaatggaagagatcatgagtgagaaggagaataaaaccattgtttt
tgtggaaaccaaaagaagatgtgatgagcttaccagaaaaatgaggagagatgggtggcctgcca
tgggtatccatggtgacaagagtcaacaagagcgtgactgggttctaaatgaattcaaacatgga
aaagctcctattctgattgctacagatgtggcctccagagggctagatgtggaagatgtgaaatt
tgtcatcaattatgactaccctaactcctcagaggattatattcatcgaattggaagaactgctc
gcagtaccaaaacaggcacagcatacactttctttacacctaataacataaagcaagtgagcgac
cttatctctgtgcttcgtgaagctaatcaagcaattaatcccaagttgcttcagttggtcgaaga
cagaggttcaggtcgttccaggggtagaggaggcatgaaggatgaccgtcgggacagatactctg
cgggcaaaaggggtggatttaatacctttagagacagggaaaattatgacagaggttactctagc
ctgcttaaaagagattttggggcaaaaactcagaatggtgtttacagtgctgcaaattacaccaa
tgggagctttggaagtaattttgtgtctgctggtatacagaccagttttaggactggtaatccaa
cagggacttaccagaatggttatgatagcactcagcaatacggaagtaatgttccaaatatgcac
aatggtatgaaccaacaggcatatgcatatcctgctactgcagctgcacctatgattggttatcc
aatgccaacaggatattcccaataa
[Chemical Formula 3]
DDX17 Amino acid sequence
(SEQ ID NO: 3)
MPTGFVAPILCVLLPSPTREAATVASATGDSASERESAAPAAAPTAEAPPPSVVTRPEPQALPSP
AIRAPLPDLYPFGTMRGGGFGDRDRDRDRGGFGARGGGGLPPKKFGNPGERLRKKKWDLSELPKF
EKNFYVEHPEVARLTPYEVDELRRKKEITVRGGDVCPKPVFAFHHANFPQYVMDVLMDQHFTEPT
PIQCQGFPLALSGRDMVGIAQTGSGKTLAYLLPAIVHINHQPYLERGDGPICLVLAPTRELAQQV
QQVADDYGKCSRLKSTCIYGGAPKGPQIRDLERGVEICIATPGRLIDFLESGKTNLRRCTYLVLD
EADRMLDMGFEPQIRKIVDQIRPDRQTLMWSATWPKEVRQLAEDFLRDYTQINVGNLELSANHNI
LQIVDVCMESEKDHKLIQLMEEIMAEKENKTIIFVETKRRCDDLTRRMRRDGWPAMCIHGDKSQP
ERDWVLNEFRSGKAPILIATDVASRGLDVEDVKFVINYDYPNSSEDYVHRIGRTARSTNKGTAYT
FFTPGNLKQARELIKVLEEANQAINPKLMQLVDHRGGGGGGGGRSRYRTTSSANNPNLMYQDECD
RRLRGVKDGGRRDSASYRDRSETDRAGYANGSGYGSPNSAFGAQAGQYTYGQGTYGAAAYGTSSY
TAQEYGAGTYGASSTTSTGRSSQSSSQQPSGIGRSGQQPQPLMSQQFAQPPGATNMIGYMGQTAY
QYPPPPPPPPPSRK
[Chemical Formula 4]
DDX17 DNA sequence
(SEQ ID NO: 4)
atgcccaccggctttgtagccccgattctctgtgttttgctcccgtctccgacgagagaggcggc
gacggtggcgtctgcgacgggagacagcgcgtcggagcgagagagcgctgcgcctgccgccgccc
caacagcggaggcgccgccgccatcggtcgtcaccagaccggagccgcaggccctcccgagcccg
gccatccgtgccccgctcccagatctctatccttttgggaccatgcgcggaggaggctttgggga
ccgggaccgggatcgtgaccgtggaggatttggagcaagaggtggtggtggccttcccccgaaga
aatttggtaatcctggggagcgtttgcgtaaaaaaaagtgggatttgagtgagctccccaagttt
gagaaaaatttttatgtggaacatccggaagtagcaaggctgacaccatatgaggttgatgagct
acgccgaaagaaggagattacagtgagggggggagatgtttgtcctaaacccgtgtttgccttcc
atcatgctaacttcccacaatatgtaatggatgtgttgatggatcagcactttacagaaccaact
ccaattcagtgccagggatttccgttggctcttagtggccgggatatggtgggcattgctcagac
tggctctgggaagacgttggcgtatctcctgcctgcaattgttcatattaaccaccagccatact
tggaaaggggagatggcccaatctgtctagttctggctcctaccagagagcttgcccagcaagta
cagcaggtggccgatgactatggcaaatgttctagattgaagagtacttgtatttatggaggtgc
tcctaaaggtccccagattcgagacttggaaagaggtgttgagatctgcatagccactcctggac
gtctgatagatttcctggagtcaggaaagacaaatcttcgccgatgtacttaccttgtattggac
gaagctgacagaatgcttgatatggggtttgaaccccagatccgtaaaattgttgaccaaatcag
gcctgataggcagacactgatgtggagtgcaacctggcccaaagaagtaagacagcttgcagagg
atttccttcgtgattacacccagatcaacgtaggcaatctggagttgagtgccaaccacaacatc
ctccagatagtggatgtctgcatggaaagtgaaaaagaccacaagttgatccaactaatggaaga
aataatggctgaaaaggaaaacaaaacaataatatttgtggagacaaagagacgctgtgatgatc
tgactcgaaggatgcgcagagatggttggccagctatgtgtatccatggagacaagagtcaacca
gaaagagattgggtacttaatgagttccgttctggaaaggcacccatccttattgctacagatgt
agcctcccgtgggctagatgtggaagatgtcaagtttgtgatcaactatgactatccaaacagct
cagaggattatgtgcaccgtattggccgaacagcccgtagcaccaacaagggtaccgcctatacc
ttcttcaccccagggaacctaaaacaggccagagagcttatcaaagtgctggaagaggccaatca
ggctatcaatccaaaactgatgcagcttgtggaccacagaggaggcggcggaggcgggggtggtc
gttctcgttaccggaccacttcttcagccaacaatcccaatctgatgtatcaggatgagtgtgac
cgaaggcttcgaggagtcaaggatggtggccggagagactctgcaagctatcgggatcgtagtga
aaccgatagagctggttatgctaatggcagtggctatggaagtccaaattctgcctttggagcac
aagcaggccaatacacctatggtcaaggcacctatggggcagctgcttatggcaccagtagctat
acagctcaagaatatggtgctggcacttatggagctagtagcaccacctcaactgggagaagttc
acagagctctagccagcagtttagtgggataggccggtctgggcagcagccacagccactgatgt
cacaacagtttgcacagcctccgggagctaccaatatgataggttacatggggcagactgcctac
caataccctcctcctcctccccctcctcctccttcacgtaaatga
[Chemical Formula 5]
DDX1 DNA sequence
(SEQ ID NO: 22)
gcggccttctccgagatgggtgtaatgcctgagattgcacaagctgtggaagagatggattggct
cctcccaactgatatccaggctgaatctatcccattgatcttaggaggaggtgatgtacttatgg
ctgcagaaacaggaagtggcaaaactggtgcttttagtattccagttatccagatagtttatgaa
actctgaaagaccaacaggaaggcaaaaaaggaaaaacaacaattaaaactggtgcttcagtgct
gaacaaatggcagatgaacccatatgacagaggatctgcttttgcaattgggtcagatggtcttt
gttgtcaaagcagagaagtaaaggaatggcatgggtgtagagctactaaaggattaatgaaaggg
aaacactactatgaagtatcctgtcatgaccaagggttatgcagggtcgggggtctaccatgcag
gcctctttggacctaggtactgacaagtttggatttggctttggtggaacaggaaagaaatccca
taacaaacaatttgataattatggagaggaattcactatgcatgataccattggatgttacctgg
atatagataagggacatgtcaagttctccaaaaatggaaaagatcttggtctggcatttgaaata
ccaccacatatgaaaaaccaagccctctttcctgèctgtgttttgaagaatgctgaactgaaatt
taacttcggtgaagaggaatttaagtttccaccaaaagatggctttgttgctctttccaaggcac
cggatggttacattgtcaaatcacagcactcaggtaatgcacaggtgacacaaacaaagtttctc
cccaatgctccgaaagctctcattgttgaaccttcccgggagttagctgaacaaactttgaacaa
catcaagcagtttaagaaatacattgataatcctaaattaagggagcttctgataattggaggtg
ttgcagcccgggatcagctctctgttttggaaaarggagtagatatagttgtaggtactccggga
agactagatgacttggtgtcaactggaaagctgaacttatctcaagttagattcctggtcctgga
tgaagctgatgggcttctttctcaaggttattctgattttataaataggatgcacaatcagattc
ctcaggttacctctgatggaaaaagacttcaggtgattgtttgctctgccactttgcattctttc
gatgtaaagaaactgtccgagaagataatgcattttcctacatgggttgacttaaaaggagaaga
ctctgttccagatactgtacaccatgttgttgtcccagtaaatcccaaaactgacagactctggg
aaaggcttggaaagagccacattagaactgatgatgtacatgcaaaagataacacaagacctggt
gctaatagtccagagatgtggtctgaagctattaaaatcctgaaaggggagtatgctgtccgggc
aatcaaggaacataagatggatcaagcaattatcttctgtagaaccaaaattgactgtgataact
tggagcagtactttatacaacaaggaggaggacctgataaaaaaggacaccagttctcatgtgtt
tgtcttcatggtgacagaaagcctcatgagagaaagcaaaacttggaaagatttaagaaaggaga
tgtaagattcttgatttgcacagatgtagctgctagaggaattgatatccacggtgttccttatg
ttataaatgtcactctgcccgatgaaaagcaaaactacgtacatcgaattggcagagtaggaaga
gctgaaaggatgggtctggcaatttccctggtggcaacagaaaaagaaaaggtttggtaccatgt
atgtagcagccgtggaaaagggtgttataacacaagactcaaggaagatggaggctgtaccatat
ggtacaacgagatgcagttactatctgagatagaagaacacctgaactgtaccatttctcaggtt
gagccggatataaaggtaccagtggatgaatttgatgggaaagttacctacggtcagaaaagggc
tgctggtggtggaagctataaaggccatgtggatattttggcacctactgttcaagagttggctg
cccttgaaaaggaggcgcagacatctttcctgcatcttggctaccucctaaccagctgttcagaa
ccttctga
[Chemical Formula 6]
DDX2A DNA sequence
(SEQ ID NO: 23)
tctgcgagccaggattcccgatccagagacaatggccccgatgggatggagcccgaaggcgtcat
cgagagtaactggaatgagattgttgacagctttgatgacatgaacctctcggagtcccttctcc
gtggcatctacgcgtatggttttgagaagccctctgccatccagcagcgagccattctaccttgt
atcaagggttatgatgtgattgctcaagcccaatctgggactgggaaaacggccacatttgccat
atcgattctgcagcagattgaattagatctaaaagccacccaggccttggtcctagcacccactc
gagaattggctcagcagatacagaaggggtcatggcactaggagactacatgggcgcctcctgtc
acgcctgtatcgggggcaccaacgtgcgtgctgaggtgcagaaactgcagatggaagctccccac
atcatcgtgggtacccctggccgtgtgtttgatatgcttaaccggagatacctgtcccccaaata
catcaagatgtttgtactggatgaagctgacgaaatgttaagccgtggattcaaggaccagatct
atgacatattccaaaagctcaacagcaacacccaggtagttttgctgtcagccacaatgccttct
gatgtgcttgaggtgaccaagaagttcatgagggaccccattcggattcttgtcaagaaggaaga
gttgaccctggagggtatccgccagttctacatcaacgtggaacgagaggagtggaagctggaca
cactatgtgacttgtatgaaaccctgaccatcacccaggcagtcatcttcatcaacacccggagg
aaggtggactggctcaccgagaagatgcatgctcgagatttcactgtatccgccatgcatggaga
tatggaccaaaaggaacgagacgtgattatgagggagtttcgttctggctctagcagagttttga
ttaccactgacctgctggccagaggcattgatgtgcagcaggtttctttagtcatcaactatgac
cttcccaccaacagggaaaactatatccacagaatcggtcgaggtggacggtttggccgtaaagg
tgtggctattaacatggtgacagaagaagacaagaggactcttcgagacattgagaccttctaca
acacctccattgaggaaatgcccctcaatgttgctgacctcatctga
[Chemical Formula 7]
DDX3X DNA sequence
(SEQ ID NO: 24)
agtcatgtggcagtggaaaatgcgctcgggctggaccagcagtttgctggcctagacctgaactc
ttcagataatcagagtggaggaagtacagccagcaaagggcgctatattcctcctcatttaagga
accgagaagctactaaaggtttctacgataaagacagttcagggtggagttctagcaaagataag
gatgcgtatagcagttttggatctcgtagtgattcaagagggaagtctagcttcttcagtgatcg
tggaagtggatcaaggggaaggtttgatgatcgtggacggagtgattacgatggcattggcagcc
gtggtgacagaagtggctttggcaaatttgaacgtggtggaaacagtcgctggtgtgacaaatca
gatgaagatgattggtcaaaaccactcccaccaagtgaacgcttggaacaggaactcttttctgg
aggcaacactgggattaattttgagaaatacgatgacattccagttgaggcaacaggcaacaact
gtcctccacatattgaaagtttcagtgatgttgagatgggagaaattatcatgggaaacattgag
cttactcgttatactcgcccaactccagtgcaaaagcatgctattcctattatcaaagagaaaag
agacttgatggcttgtgcccaaacagggtctggaaaaactgcagcatttctgttgcccatcttga
gtcagatttattcagatggtccaggcgaggctttgagggccatgaaggaaaatggaaggtatggg
cgccgcaaacaatacccaatctccttggtattagcaccaacgagagagttggcagtacagatcta
cgaggaagccagaaaattttcataccgatctagagttcgtccttgcgtggtttatggtggtgccg
atattggtcagcagattcgagacttggaacgtggatgccatttgttagtagccactccaggacgt
ctagtggatatgatggaaagaggaaagattggattagacttttgcaaatacttggtgttagatga
agctgatcggatgttggatatggggtttgagcctcagattcgtagaatagtcgaacaagatacta
tgcctccaaagggtgtccgccacactatgatgtttagtgctacttttcctaaggaaatacagatg
ctggctcgtgatttcttagatgaatatatcttcttggctgtaggaagagttggctctacctctga
aaacatcacacagaaagtagtttgggtggaagaatcagacaaacggtcatttctgcttgacctcc
taaatgcaacaggcaaggattcactgaccttagtgtttgtggagaccaaaaagggtgcagattct
ctggaggatttcttataccatgaaggatacgcatgtaccagcatccarggagaccgttctcagag
ggatagagaagaggcccttcaccagttccgctcaggaaaaagcccaattttagtggctacagcag
tagcagcaagaggactggacatttcaaatgtgaaacatgttatcaattttgacttgccaagtga
tattgaagaatatgtacatcgtattggtcgtacgggacgtgtaggaaaccttggccEggcaacct
cattctttaacgagaggaacataaatattactaaggatttgttggatcttcttgttgaagctaaa
caagaagtgccgtcttggttagaaaacatggcttatgaacaccactacaagggtagcagtcgtgg
acgttctaagagtagcagatttagtggagggtttggtgccagagactaccgacaaagtagcggtg
ccagcagttccagcttcagcagcagccgcgcaagcagcagccgcagtggcggaggtggccacggt
agcagcagaggatttggtggaggtggctatggaggcttttacaacagtgatggatatggaggaaa
ttataactcccagggggttgactggtggggtaactga
[Chemical Formula 8]
DDX4 DNA sequence
(SEQ ID NO: 25)
ggagatgaagattgggaagcagaaatcaaccctcatatgtcttcctatgttcccatatttgagaa
ggataggtattctggagaaaatggagacaattttaacaggactccagcttcatcatcagaaatgg
atgatggaccttctcgaagagatcatttcatgaaaagtggatttgcctctgggcggaattttgga
aacagagatgctggtgagtgtaataagcgagataatacatccacaatgggggttttggagttgga
aagagttttggaaacagaggtttttcaaacagcaggtttgaagatggtgatagctctggtttctg
gagagagtctagtaatgactgcgaagataatccaacacggaacagagggttttccaagagaggcg
gctatcgagatggaaataattcagaagcttcagggccatacagaagaggtggaagaggtagtttc
cgaggttgccgtggaggatttggtctaggaagtccaaataatgacttagacccagacgaatgtat
gcagcgcactggtggcctttttggttctagaagaccagtattaagtggcacaggtaatggtgata
cttctcaaagcagaagtggcagtggaagtgaacgaggtggttacaaaggtttaaatgaagaagta
ataacaggctctggaaagaattcttggaagtcagaagcagaaggaggagaaagtagtgatactca
aggaccaaaagtgacctacataccccctcctccacctgaggatgaggactccatctttgcacatt
atcagacaggcataaacttcgacaaatacgacactattcttgtggaagtgtctggacatgatgca
ccaccagcaattctgacttttgaagaagctaatctctgtcagacactgaataacaacattgctaa
agctggttatactaagcttactcctgtgcaaaaatacagtattcctatcatacttgcaggacgag
atttgatggcttgcgctcaaacagggtctgggaagactgcggcttttctcctaccaattttggct
catatgatgcatgatggaataactgccagtcgttttaaagagttgcaggaaccagagtgtattat
tgtagcaccaactcgagaattggtcaaccagatttatttggaagccagaaaattttcttttggga
cttgtgtaagagctgttgttatatatgggggaacccagctgggacattcaattcgacaaatagta
caaggctgtaatatattatgtgctactcctggaagactgatggatatcataggcaaagaaaagat
tggtctcaaacagatcaaatacttagttttggatgaagctgatcgcatgttggatatgggttugg
tccagaaatgaagaagttaatttcttgcccaggaatgccatcaaaggaacagcgccaaaccctta
tgttcagtgcaacttttccagaggaaattcaaaggttggctgcagagtttttaaagtcaaattat
ctgtttgttgctgttggacaagtgggtggagcatgtagagatgttcagcagaccgttctccaagt
tggccagttctcaaaaagagaaaagctcgttgaaattctgcgaaacataggggatgaaagaacta
tggtctttgttgaaactaagaaaaaagcagattttattgcaacttttctttgtcaagaaaaaata
tcaactacaagtattcatggtgatcgggaacagagagagcgggagcaagctcttggagattttcg
ctttggaaagtgcccagttcttgttgctacttcagtagctgccagagggctggatattgaaaatg
tgcaacatgttatcaattttgatcttccttctaccattgatgaatatgttcatcgaattgggcgt
actggtcgttgtgggaatactggcagagcaatttccttttttgatcttgaatcggataaccattt
agcacagcctctagtaaaagtattgacagatgctcaacaggatgttcctgcatggttggaagaaa
ttgcctttagtacatacattcctggcttcagtggtagtacaagaggaaacgtgtttgcatcagtt
gataccagaaagggcaagagcactttgaacacagctgggttttcttcttcacaagctcccaatcc
agtagatgatgagtcatgggattaa
[Chemical Formula 9]
DDX5 DNA sequence
(SEQ ID NO: 26)
tcgggttattcgagtgaccgagaccgcggccgggaccgagggtttggtgcacctcgatttggagg
aagtagggcagggcccttatctggaaagaagtttggaaaccctggggagaaattagttaaaaaga
agtggaatcttgatgagctgcctaaatttgagaagaatttttatcaagagcaccctgatttggct
aggcgcacagcacaagaggtggaaacatacagaagaagcaaggaaattacagttagaggtcacaa
ctgcccgaagccagttctaaatttttatgaagccaatttccctgcaaatgtcatggatgttattg
caagacagaatttcactgaacccactgctattcaagctcagggatggccagttgctctaagtgga
ttggatatggttggagtggcacagactggatctgggaaaacattgtcttatttgcttcctgccat
tgtccacatcaatcatcagccattcctagagagaggcgatgggcctatttgtttggtgctggcac
caactcgggaactggcccaacaggtgcagcaagtagctgctgaatattgtagagcatgtcgcttg
aagtctacttgtatctacggtggtgctcctaagggaccacaaatacgtgatttggagagaggtgt
ggaaatctgtattgcaacacctggaagactgattgactttttagagtgtggaaaaaccaatctga
gaagaacaacctaccttgtccttgatgaagcagatagaatgcttgatatgggctttgaaccccaa
ataaggaagattgtggatcaaataagacctgataggcaaactctaatgtggagtgcgacttggcc
aaaagaagtaagacagcttgctgaagatttcctgaaagactatattcatataaacattggtgcac
ttgaactgagtgcaaaccacaacattcttcagattgtggatgtgtgtcatgacgtagaaaaggat
gaaaaacttattcgtctaatggaagagatcatgagtgagaaggagaataaaaccattgtttttgt
ggaaaccaaaagaagatgtgatgagcttaccagaaaaatgaggagagatgggtggcctgccatgg
gtatccatggtgacaagagtcaacaagagcgtgactgggttctaaatgaattcaaacatggaaaa
gctcctattctgattgctacagatgtggcctccagagggctagatgtggaagatgtgaaatttgt
catcaattatgactaccctaactcctcagaggattatattcatcgaattggaagaactgctcgca
gtaccaaaacaggcacagcatacactttctttacacctaataacataaagcaagtgagcgacctt
atctctgtgcttcgtgaagctaatcaagcaattaatcccaagttgcttcagttggtcgaagacag
aggttcaggtcgttccaggggtagaggaggcatgaaggatgaccgtcgggacagatactctgcgg
gcaaaaggggggatttaatacctttagagacagggaaaattatgacagaggttactctagcctgc
ttaaaagagattttggggcaaaaactcagaatggtgtttacagtgctgcaaattacaccaatggg
agctttggaagtaattttgtgtctgctggtatacagaccagttttaggactggtaatccaacagg
gacttaccagaatggttatgatagcactcagcaatacggaagtaatgttccaaatatgcacaatg
gtatgaaccaacaggcatatgcatatcctgctactgcagctgcacctatgattggttatccaatg
ccaacaggatattcccaataa
[Chemical Formula 10]
DDX6 DNA sequence
(SEQ ID NO: 27)
agcacggccagaacagagaaccctgttataatgggtctgtccagtcaaaatggtcagctgagagg
ccctgtgaaacccactggtggccctggaggagggggcacacagacacagcaacagatgaaccagc
tgaaaaacaccaacacaatcaataatggcactcagcagcaagcacagagtatgaccaccactatt
aaacctggtgatgactggaaaaagactttaaaactccctccaaaggatctaagaatcaaaacttc
ggatgtgacctccacaaaaggaaatgagtttgaagattactgtttgaaacgggagttactgatgg
gaatttttgaaatgggctgggaaaagccatctcctattcaggaggagagcattcccattgcttta
tctggtagggatatcttagctagagcaaaaaatggaacaggcaagagcggtgcctacctcattcc
cttacttgaacggctagacctgaagaaggacaatatacaagcaatggtgattgttcccactagag
aacttgctctacaggtcagtcaaatttgcatccaggtcagcaaacacatgggaggggccaaagtg
atggcaaccacaggaggaaccaatttacgagatgacataatgaggcttgatgatacagtgcacgt
ggtgattgctacccctgggagaatcctggatcttattaagaaaggagtagcaaaggttgatcatg
tccagatgatagtattggatgaggcagataagttgctgtcacaggattttgtgcagataatggag
gatattattctcacgctacctaaaaacaggcagattttactatattccgctactttccctcttag
tgtacagaagttcatgaattcccatttgcagaaaccctatgagattaacctgatggaggaactaa
ctctgaagggagtaacccagtactacgcatatgtaactgagcgccaaaaagtacactgcctcaac
acacttttctccaggcttcagataaaccagtcgatcattttctgtaactcctctcagcgagttga
attgctagccaagaagatttctcaactgggttattcttgcttctatattcatgctaaaatgaggc
aggaacatcgaaatcgtgtatttcatgatttccgaaatggcttatgccgcaatcttgtttgcact
gatctgtttacccgaggtattgatatacaagctgtgaatgtggtaataaactttgatttcccaaa
gctggcagagacctatctccatcgtattggaagatcaggtcgctttggtcatcttggcttagcca
tcaacttgatcacatatgatgatcgcttcaacctgaaaagtattgaggagcagctgggaacagaa
attaaacctattccgagcaacattgataagagcctgtatgtggcagaataccacagcgagcctgt
agaagatgagaaaccttaa
[Chemical Formula 11]
DDX10 DNA sequence
(SEQ ID NO: 28)
ggcaaaacggccaactctccgggttcgggagcccgacccgacccggtgcggagcttcaatcgctg
gaagaaaaaacacagccataggcagaacaaaaagaagcagttgaggaagcaactgaagaaacccg
aatggcaggtcgagcgcgagagtatcagccgcctcatgcagaactatgaaaagataaatgtaaat
gaaatcacaagattttcagattttcccttgtccaaaaaaacattgaaaggtttgcaagaagctca
gtaccgtttggtgactgagatacagaagcagaccattggattggctttgcaaggtaaagatgtac
ttggagcggccaaaactggatctggcaagactctggcttttcttgttccagtgctggaagcctta
tatcgtctgcaatggacttcaacagatgggctgggggttctcataatatcacctacgagagaact
ggcctatcagacctttgaggttctccgaaaagtaggaaagaatcatgacttctcagctggtctca
tcattggtggaaaggatctaaaacacgaagctgagaggatcaacaacataaatatactcgtgtgc
acaccaggtcggcttcttcaacacatggatgaaacagtatcttttcatgctaccgacctccaaat
gttagttcttgatgaagcagatagaatcttggatatgggctttgctgataccatgaatgctgtta
ttgaaaatctccccaagaaacgtcagactttacttttctcagcaacacaaactaaatctgtaaag
gaccttgcacgcttgagtttgaaaaaccctgagtatgtctgggttcatgaaaaagcaaaatatag
cacccctgccactttggaacagaactacatagtctgtgagctgcagcaaaaaataagtgtgctgt
attcctttttgagaagccatctgaagaagaagagcattgtatttttttccagttgcaaagaggtc
cagtatctgtaccgagtgttttgccggctacgtcctggtgtttctatccttgcactccatggtcg
acagcagcaaatgagaagaatggaagtctataatgagtttgtccgtaagagagctgcagtactct
ttgctactgatattgcagccaggggtctggatttcccggccgtgaattgggttcttcagtttgat
tgtcctgaggatgccaacacatatattcacagagcaggtagaactgccaggtacaaagaggatgg
tgaagctttgctaattttgctaccctcagaaaaagctaggtgcagcagcttcttcagaagaaagt
acctgtgaaggaaatcaaaatcaatccagaaaaacttatagatgtccagaaaaaattggaatcta
ttttagctcaagatcaagatttaaaagaaagagctcaaaggtgtttcgtctcctatgtacgatct
gtatatctgatgaaggataaagaagtatttgatgtgagcaagttacctatacctgaatatgccct
gtctcttggtcttgctgtggcaccacgcgtaagatttcttcagaaaatgcagaaacaacccacca
aagaattggtaaggagccaagccgataaagtaattgagccaagggctccctccctcaccaaIgac
gaagtggaagaatttagagcctacttcaatgagaaaatgtccatccttcaaaaaggtggaaaaag
actcgaagggacagagcacagac-aggataatgatactggtaatgaagaacaggaagaagaagaa
gacgatgaagaagaaatggaagagaaactggcaaaagcaaaaggatctcaagccccatctcttcc
taacaccagtgaggcacagaagatcaaggaagttcctacacagttcttggacagagatgaggagg
aagaagatgctgatttcttgaaggtgaagcggcataatgtgtttggattggaccttaaagacgag
aaaacattacagaagaaagaaccttctaaatccagcatcaagaaaaaaatgaccaaagttgcaga
agcaaaaaaagtaatgaagagaaattttaaagtgaataagaagataacatttactgatgaagggg
agttggttcagcagtggccacaaatgcagaaatctgccatcaaggatgctgaggaagatgatgac
acaggtggtatcaacttacataaagcaaaggaaagacttcaggaagaggacaaatttgacaaaga
agaatataggaaaaaaattaaggcaaagcatcgggagaaaagactgaaagaaagggaagccagaa
gagaagccaacaagagacaagcaaaggccaaagatgaagaggaagcctttctggattggagtgat
gatgatgatgatgatgatgatggatttgatccaagcacactcccagatccagataaatacagaag
ctctgaagattcagatagtgaagatatggaaaataaaataagtgataccaagaagaagcagggga
tgaagaagaggagcaacagtgaagtggaagacgtgggaccaacaagtcataacagaaagaaggcc
aggtgggacactttagagcctttggataccggcctgtctttagcagaggatgaagagctggtgtt
acatctgctaagaagtcaaagctaa
[Chemical Formula 12]
DDX11 DNA sequence
(SEQ ID NO: 29)
gctaatgaaacacagaaggttggtgccatccattttccttttcccttcacaccctattccatcca
ggaagacttcatggcagagctgtaccgggttttggaggctggcaagattgggatatttgagagtc
caactggcactgggaagtccttaagtcttatttgtggggccctctcttggctccgtgactttgaa
cagaagaagcgtgaagaagaggcacgactccttgaaactggaactggccccttacatgatgagaa
agatgaatccctgtgtctgtcttcttcctgcgaaggggctgcaggcaccccgaggcctgctggag
aaccggcctgggttactcagtttgtgcagaagaaagaagagagggacctggtggaccgactaaag
gcggagcaggccaggaggaagcagcgagaagaacgcctgcagcagctgcagcacagggtgcagct
caagtatgcagccaagcgcctgaggcaggaagaagaagaaagagagaatctcctccgcctcagca
gggagatgctagagacaggcccggaggctgagcggctggagcagctggagtctggggaggaggag
ctggtcctcgccgaatacgagagtgatgaggagaaaaaggtggcgagcagagtggatgaggatga
ggatgacctggaggaagaacacataactaagatttattactgtagtcggacacactcccagctgg
cccagtttgtgcatgaggtgaagaagagcccctttggcaaggatgttcggctggtctcccttggc
tcccggcagaacctttgtgtaaatgaagacgtgaaaagcctaggttctgtgcagcttatcaacga
ccgctgtgtggacatgcagagaagcaggcacgagaagaagaaaggagctgaggaggagaagccaa
agaggaggaggcaggagaagcaggcagcctgccccttctacaaccacgagcagatgggccttctc
cgggatgaggccctggcagaggtgaaggacatggagcagctgctggcccttgggaaggaggcccg
ggcctgtccctattacgggagccgccttgccatccctgcagcccagctggtggtgctgccctatc
agatgctgctgcatgcggccactcggcaggccgcgggcatccggctgcaggaccaggtggtgatc
atcgacgaggcgcacaacctgatcgacaccatcacgggcatgcacagcgtggaggtcagcggctc
ccagctctgccaggcccattcccagctgctgcagtacgtggagcgatacgggaagcgtttgaagg
ccaagaacctgatgtacctgaagcagatcctgtatttgctggagaaattcgtggctgtgctaggg
gggaacattaagcaaaatcccaatacacagagtctgtcacagacagggacggagctgaagaccaE
caacgactttctcttccagagccagatcgacaacatcaacctgttcaagggcagcgatactgtga
gaagagcatgatcagcagaaagctctttggattcactgaacggtacggagcagtgttctcatccc
gggagcagcccaaactggctgggtttcagcaattcctgcagagcctgcagcccaggacgactgaa
gctcttgcagcccctgcagacgagagtcaggccagcaccctgcgaccagcttctccactgatgca
catccaaggcttcctggcagctctcactacggccaaccaggacggcagggtcatcctgagccgcc
aaggcagcctcagtcagagcaccctgaagtttttgctcctgaatccagctgtgcactttgcccaa
gtggtgaaggaatgccgggcagtggtcattgcggggggtaccatgcagccggtgtctgacttccg
gcagcagctgctggcctgtgccggggtggaagctgagcgcgtggtggagttttcctgtggtcacg
tgatccctccagacaacatcctgcccctcgtcatctgcagcgggatctccaaccagccgctggaa
ttcacgttccagaaaagagagctgcctcagatgatggacgaggtgggtcgcattctctgtaacct
gtgcggtgtggttcctggaggggtggtctgtttcttcccctcctacgagtacctgcgccaggtcc
atgcccactgggagaagggtggcctgctgggccgtctggctgccaggaagaagatattccaggaa
cctaagagcgcacaccaggtggagcaggtgctgctggcatattccaggtgcatccaggcctgtgg
ccaggagagaggccaggtgacaggggccctgctcctctctgtggttggaggaaagatgagtgaag
ggatcaacttctctgacaacctaggccggtgtgtggtgatggtgggcatgcccttccccaacatc
aggtctgcagagctgcaggagaagatggcctacttggatcaaaccctcagcccccggccaggcac
ccccagggaaggctctggtggagaacctgtgcatgaaggccgtcaaccagtccataggcagggc
catcaggcaccagaaggattttgccagcgtagtgctcctggaccagcgatatgcccggccccct
gtcctggccaagctgccggcctggatccgagcccgtgtggaggtcaaagctacctttggccccgc
cattgctgctgtgcagaagtttcaccgggagaagtcggcctcttcc
[Chemical Formula 13]
DDX17 DNA sequence
(SEQ ID NO: 30)
cccaccggctttgtagccccgattctctgtgttttgctcccgtctccgacgagagaggcggcgac
ggtggcgtctgcgacgggagacagcgcgtcggagcgagagagcgctgcgcctgccgccgccccaa
cagcggaggcgccgccgccatcggtcgtcaccagaccggagccgcaggccctcccgagcccggcc
atccgtgccccgctcccagatctctatccttttgggaccatgcgcggaggaggctttggggaccg
ggaccgggatcgtgaccgtggaggatttggagcaagaggtggtggtggccttcccccgaagaaat
ttggtaatcctggggagcgtttgcgtaaaaaaaagtgggattrgagtgagctccccaagtttgag
aaaaatttttatgtggaacatccggaagtagcaaggctgacaccatatgaggttgatgagctacg
ccgaaagaaggagattacagtgagggggggagatgtugtcctaaacccgtgtttgccttccatca
tgctaacttcccacaatatgtaatggatgtgttgatggatcagcactttacagaaccaactccaa
ttcagtgccagggatttccgttggctcttagtggccgggatatggtgggcattgctcagactggc
tctgggaagacgttggcgtatctcctgcctgcaattgttcatattaaccaccagccatacttgga
aaggggagatggcccaatctgtctagttctggctcctaccagagagcttgcccagcaagtacagc
aggtggccgatgactatggcaaatgttctagattgaagagtacttgtatttatggaggtgctcct
aaaggtccccagattcgagacttggaaagaggtgttgagatctgcatagccactcctggacgtct
gatagatttcctggagtcaggaaagacaaatcttcgccgatgtacttaccttgtattggacgaag
ctgacagaatgcttgatatggggtttgaaccccagatccgtaaaattgttgaccaaatcaggcct
gataggcagacactgatgtggagtgcaacctggccaaaagaagtaagacagcttgcagaggattt
ccttcgtgattacacccagatcaacgtaggcaatctggagttgagtgccaaccacaacatcctcc
agatagtggatgtctgcatggaaagtgaaaaagaccacaagttgatccaactaatggaagaaata
atggctgaaaaggaaaaaaaacaataatatttgtggagacaaagagacgctgtgatgatctgact
cgaaggatgcgcagagatggttggccagctatgtgtatccatggagacaagagtcaaccagaaag
agattgggtacttaatgagttccgttctggaaaggcacccatccttattgctacagatgtagcct
cccgtgggctagatgtggaagatgtcaagtttgtgatcaactatgactatccaaacagctcagag
gattatgtgcaccgtattggccgaacagcccgtagcaccaacaagggtaccgcctataccttctt
caccccagggaacctaaaacaggccagagagcttatcaaagtgctggaagaggccaatcaggcta
tcaatccaaaactgatgcagcttgtggaccacagaggaggggcggaggcgggggtggtcgttctc
gttaccggaccacttcttcagccaacaatcccaatctgatgtatcaggatgagtgtgaccgaagg
cttcgaggagtcaaggatggtggccggagagactctgcaagctatcgggatcgtagtgaaaccga
tagagctggttatgctaatggcagtggctatggaagtccaaattctgcctttggagcacaagcag
gccaatacacctatggtcaaggcacctatggggcagctgcttatggcaccagtagctatacagct
caagaatatggtgctggcacttatggagctagtagcaccacctcaactgggagaagttcacagag
ctctagccagcagtttagtgggataggccggtctgggcagcagccacagccactgatgtcacaac
agtttgcacagcctccgggagctaccaatatgataggttacatggggcagactgcctaccaatac
cctcctcctcctccccctcctcctccttcacgtaaatga
[Chemical Formula 14]
DDX18 DNA sequence
(SEQ ID NO: 31)
tcacacctgccgatgaaactcctgcgtaagaagatcgagaagcggaacctcaaattgcggcagcg
gaacctaaagtttcagggggcctcaaatctgaccctatcggaaactcaaaatggagatgtatctg
aagaaacaatgggaagtagaaaggttaaaaaatcaaaacaaaagcccatgaatgtgggcttatca
gaaactcaaaatggaggcatgtctcaagaagcagtgggaaatataaaagttacaaagtctcccca
gaaatccactgtattaaccaatggagaagcagcaatgcagtcttccaattcagaatcaaaaaaga
aaaagaagaaaaagagaaaaatggtgaatgatgctgagcctgatacgaaaaaagcaaaaactgaa
aacaaagggaaatctgaagaagaaagtgccgagactactaaagaaacagaaaataatgtggagaa
gccagataatgatgaagatgagagtgaggtgcccagtctgcccctgggactgacaggagcttttg
aggatacttcgtttgcttctctatgtaatcttgtcaatgaaaacactctgaaggcaataaaagaa
atgggttttacaaacatgactgaaattcagcataaaagtatcagaccacttctggaaggcaggga
tcttctagcagctgcaaaaacaggcagtggtaaaaccctggcttttctcatccctgcagttgaac
tcattcttaagttaaggttcatgcccaggaatggaacaggagtccttattctctcacctactaga
gaactagccatgcaaacctttggtgttcttaaggagctgatgactcaccacgtgcatacctatgg
cttgataatgggtggcagtaacagatctgctgaagcacagaaacttggtaatgggatcaacatca
ttgtggccacaccaggccgtctgctggaccatatgcagaataccccaggatttatgtataaaaac
ctgcagtgtctggttattgatgaagctgatgtatcttggatgtggggtttgaagaggaattaaag
caaattattaaacttttgccaacacgtagacagactatgctcttttctgccacccaaactcgaaa
agttgaagacctggcaaggatttctctgaaaaaggagccattgtatgttggcgttgatgatgata
aagcgaatgcaacagtggatggtcttgaacagggatatgttgtttgtccttctgaaaagagattc
cttctgctctttacattccttaagaagaaccgaaagaagaagcttatggtcttcttttcatcttg
tatgtctgtgaaataccactatgagttgctgaactacattgatttgcccgtcttggccattcatg
gaaagcaaaagcaaaataagcgtacaaccacattcttccagttctgcaatgcagattcgggaaca
ctattgtgtacggatgtggcagcgagaggactagacattcctgaagtcgactggattgttcagta
tgaccctccggatgaccctaaggaatatattcatcgtgtgggtagaacagccagaggcctaaatg
ggagagggcatgccttgctcattttgcgcccagaagaattgggttttcttcgctacttgaaacaa
tccaaggttccattaagtgaatttgacttttcctggtctaaaatttctgacattcagtctcagct
tgagaaattgattgaaaagaattactttcttcataagtcagcccaggaagcatataagtcataca
tacgagcctatgattcccattctctgaaacagatctttaatgttaataacctaaatttgcctcag
gttgctctgtcatttggtttcaaggtgcctcccttcgttgatctgaacgtcaacagtaatgaagg
caagcagaaaaagcgaggaggtggtggtggatttggctaccagaaaaccaagaaagttgagaaat
ccaaaatctttaaacacattagcaagaaatcatctgacagcaggcagttctctcactga
[Chemical Formula 15]
DDX19B DNA sequence
(SEQ ID NO: 32)
gccactgactcatgggccctggcggtggacgagcaggaagctgcggctgagtcgttgagcaactt
gcatcttaaggaagagaaaatcaaaccagataccaatggtgctgttgtcaagaccaatgccaatg
cagagaagacagatgaagaagagaaagaggacagagctgcccagtccttactcaacaagctgatc
agaagcaaccttgttgataacacaaaccaagtggaagtcctgcaacgggatccaaactcccctct
gtactcggtgaagtcgtttgaagagcttcggctgaaaccacagcttctccagggagtctatgcca
tgggcttcaatcgaccctccaagatacaagagaacgcattacccatgatgcttgctgaaccccca
cagaatctgattgcccagtctcagtctggcactggtaaaacagctgcctttgtcttagccatgct
cagccgagtggagccatcagacagatacccccagtgtctgtgcctctccccaacatatgagctgg
cgcttcaaacaggaaaagtgattgagcagatgggcaaattttacccagaactgaagcttgcctat
gccgttcgaggcaataaattggaaagaggccagaagatcagtgagcagattgtcattggcacccc
tgggaccgtgctggactggtgctccaagctcaagttcattgatcccaagaaaatcaaggtgtttg
ttctggatgaggctgatgtcargatagccactcagggccaccaagatcagagcatccgcatccag
aggatgctgcccaggaactgccagatgctgcttttctccgccacctttgaagactctgtgtggaa
gtttgcccagaaagtggtcccagacccaaatgttatcaaactgaagcgtgaggaagagaccctgg
ataccatcaagcagtactatgtcctgtgcagcagcagagacgagaagttccaggccttgtgtaac
ctctacggggccatcaccattgctcaagccatgatcttctgccatactcgcaaaacagctagttg
gctggcaacagagctctcaaaagaaggccaccaggtggctctgctgagtggggagatgatggtgg
agcagagggctgcggtgattgagcgcttccgagagggcaaagagaaggttttggtgaccaccaac
gtgtgtgcccgcggcattgatgttgaacaagtgtctgtcgtcatcaactttgatcttcccgtgga
caaggacgggaatcctgacaatgagacctacctgcaccggatcgggcgcacgggccgctttggca
agaggggcctggcagtgaacatggtggacagcaagcacagcatgaacatcctgaacagaatccag
gagcattttaataagaagatagaaagattggacacagatgatttggacgagattgagaaaatagc
caactga
[Chemical Formula 16]
DDX20 DNA sequence
(SEQ ID NO: 33)
gcggcggcatttgaagcctcgggagccttagcagcagtggcgactgctatgccggctgagcatgt
ggccgtgcaggtcccggccccagagccaacacccgggcctgtgaggatcctgcggaccgctcagg
atctcagcagcccgcggacccgcacgggggatgtgctgttggcggagccggccgacttcgagtca
ctgctgctttcgcggccggtgctggaggggctgcgggcggccggcttcgagaggccctcgccggt
gcagctcaaggccatcccgttggggcgctgcgggctcgatttaattgttcaagctaaatctggca
ccgggaaaacctgtgtgttctccaccatagctttggactctcttgttcttgaaaacttaagtacc
cagattttgatcttggctcctacaagagaaattgctgtacagatacattctgttattacagccat
tggaataaaaatggaaggcttagagtgtcatgtctttattggagggaccccattatcacaagaca
aaaccagacttaaaaagtgtcatattgctgttggatctcctggcagaattaagcaactcatagaa
cttgactacttgaacccaggcagtatacgcctctttattcttgatgaagcagataagcttttaga
agaaggcagcttccaggagcaaataaattggatttattcttccttgcctgccagtaaacagatgc
tggcagtatcagctacttatcccgaatttttggctaatgctttgacaaagtacatgagagatccc
acttttgtaagactgaattccagtgatccaagtctcataggtttgaagcagtattacaaagttgt
caattcataccctttggcacataaggtttttgaggaaaagactcagcatttacaggaactgttca
gcagaattccatttaatcaagctttagtcttttctaatttgcacagcagagcacaacatttggct
gatatcctttcttctaaaggctttcctgctgagtgcatttcaggcaatatgaatcagaatcagcg
tcttgatgctatggctaaactgaagcactttcattgcagagtcctcatttccacagatttgactt
ctcgtgggattgatgctgagaaggtgaatctggttgtaaatctggatgtaccattggattgggag
acatacatgcatcggattgggagagctggccgttttggtacattggggctgacagtgacctactg
ttgccggggagaggaagaaaatatgatgatgagaattgcccagaaatgtaatatcaaccttctcc
ctttaccagatcccattccttctggtctgatggaagaatgtgtggattgggatgtggaagttaaa
gctgctgtgcatacatatggtatagcaagtgtacctaaccaacccttaaaaaagcaaattcagaa
aatagagagaacccttcaaattcagaaagctcatggtgaccacatggcttcctctagaaataatt
ctgtatctggactatcagtcaaatcaaaaaataataccaaacaaaagcttcctgtgaaaagccac
tcagaatgtggaatcatagaaaaagcaacgtcaccaaaagaactgggctgtgacaggcaatccga
agagcaaatgaagaattctgttcagactcccgttgaaaactccaccaacagtcagcaccaggtca
aagaagctttacctgtgtcactcccccagattccttgtctgtcttcctttaaaatccatcagcca
tacacgttgacttttgctgaattggtagaggattatgaacattatattaaagaggggttagagaa
acctgtggaaatcatcaggcactacacaggccctggggatcagactgtgaatcctcaaaatggtt
ttgtgagaaataaagttattgaacagagagtccctgtgttggcaagtagtagccaatctggagac
tctgagagtgacagtgattcttacagctcaagaacctcttcccagagcaaaggaaataagtcata
cttggaaggctcttctgataatcagctgaaagactctgaatctacgcctgtggatgatcgtattt
ctttggaacaaccaccaaatggaagtgacacccccaatccagagaaatatcaagaatcacctgga
atccagatgaagacaagacttaaagagggggctagccagagagctaagcagagccggagaaacct
acccaggcggtcttccttcagattgcagactgaagcccaggaagatgattggtatgactgtcata
gggaaatacgtctgagtttttctgatacctatcaggattatgaggagtactggagagcttactac
agggcatggcaagaatattatgctgccgcttctcattcatattattggaatgctcagagacatcc
aagttggatggcagcttatcacatgaataccatttatctacaagaaatgatgcatagtaaccagt
ga
[Chemical Formula 17]
DDX21 DNA sequence
(SEQ ID NO: 34)
ccgggaaaactccgtagtgacgctggtttggaatcagacaccgcaatgaaaaaaggggagacact
gcgaaagcaaaccgaggagaaagagaaaaaagagaagccaaaatctgataagactgaagagatag
cagaagaggaagaaactgttttccccaaagctaaacaagttaaaaagaaagcagagccttctgaa
gttgacatgaattctcctaaatccaaaaaggcaaaaaagaaagaggagccatctcaaaatgacat
ttctcctaaaaccaaaagtttgagaaagaaaaaggagcccattgaaaagaaagtggtttcttcta
aaaccaaaaaagtgacaaaaaatgaggagccttctgaggaagaaatagatgctcctaagcccaag
aagatgaagaaagaaaaggaaatgaatggagaaactagagagaaaagccccaaactgaagaatgg
atttcctcatcctgaaccggactgtaaccccagtgaagctgccagtgaagaaagtaacagtgaga
tagagcaggaaatacctgtggaacaaaaagaaggcgctttctctaattttcccatatctgaagaa
actattaaacttctcaaaggccgaggagtgaccttcctatttcctatacaagcaaagacattcca
tcatgtttacagcgggaaggacttaattgcacaggcacggacaggaactgggaagacattctcct
ttgccatccctttgattgagaaacttcatggggaactgcaagacaggaagagaggccgtgcccct
caggtactggttcttgcacctacaagagagttggcaaatcaagtaagcaaagacttcagtgacat
cacaaaaaagctgtcagtggcttgtttttatggtggaactccctatggaggtcaatttgaacgca
tgaggaatgggattgatatcctggttggaacaccaggtcgtatcaaagaccacatacagaaLggc
aaactagatctcaccaaacttaagcatgttgtcctggatgaagtggaccagatgttggatatggg
atttgctgatcaagtggaagagattttaagtgtggcatacaagaaagattctgaagacaatcccc
aaacattgcttttttctgcaacttgccctcattgggtatttaatgttgccaagaaatacatgaaa
tctacatatgaacaggtggacctgattggtaaaaagactcagaaaacggcaataactgtggagca
tctggctattaagtgccactggactcagagggcagcagttattggggatgtcatccgagtatata
gtggtcatcaaggacgcactatcatcttttgtgaaaccaagaaagaagcccaggagctgtcccag
aattcagctataaagcaggatgctcagtccttgcatggagacattccacagaagcaaagggaaat
caccctgåaaggttttagaaatggtagttttggagttttggtggcaaccaatgttgctgcacgtg
ggttagacatccctgaggttgatttggttatacaaagctctccaccaaaggatgtagagtcctac
attcatcgatccgggggacaggcagagctggaaggacgggggtgtgcatctgcttttatcagcac
aaggaagaatatcagttagtacaagtggagcaaaaagcgggaattaagttcaaacgaataggtgt
tccttctgcaacagaaataataaaagcttccagcaaagatgccatcaggcttttggattccgtgc
ctcccactgccattagtcacttcaaacaatcagctgagaagctgatagaggagaagggagctgtg
gaagctctggcagcagcactggcccatatttcaggtgccacgtccgtagaccagcgctccttgat
caactcaaatgtgggttttgtgaccatgatcttgcagtgctcaattgaaatgccaaatattagtt
atgcttggaaagaacttaaagagcagctgggcgaggagattgattccaaagtgaagggaatggtt
tttctcaaaggaaagctgggtgtttgctttgatgtacctaccgcatcagtaacagaaatacagga
gaaatggcatgattcacgacgctggcagctctctgtggccacagagcaaccagaactggaaggac
cacgggaaggatatggaggcttcaggggacagcgggaaggcagtcgaggcttcaggggacagcgg
gacggaaacagaagattcagaggacagcgggaaggcagtagaggcccgagaggacagcgatcagg
aggtggcaacaaaagtaacagatcccaaaacaaaggccagaagcggagtttcagtaaagcatttg
gtcaataa
[Chemical Formula 18]
DDX23 DNA sequence
(SEQ ID NO: 35)
gcaggagagctggctgacaaaaaggaccgtgatgcatcaccttccaaggaggaaaggaagcgatc
acggactcctgacagagagcgggatagagaccgggaccggaagtcttccccatctaaagatagaa
agcggcatcgttcaagggatagacgtcgaggaggcagccgttctcgctctcgttcccgttccaaa
tctgcagaaagagaacgacggcacaaagaacgagaacgagataaggagcgggatcggaataagaa
ggaccgagatcgagacaaggatgggcacagacgggacaaggaccgtaaacgatccagcttatctc
ctggtcgaggaaaagactttaaatctcggaaggacagagactctaagaaggatgaagaggatgaa
catggtgataagaagcctaaggcccagccattatccctggaggagcttctggccaagaaaaaggc
tgaggaagaagctgaggctaagcccaagttcctctctaaagcagaacgagaggctgaagctctaa
agcgacggcagcaggaggtggaagagcggcagaggatgcttgaagaagagaggaagaaaaggaaa
cagttccaagacttgggcaggaagatgttggaagatcctcaggaacgggaacgtcgggaacgcag
ggagaggatggaacgggagaccaatggaaatgaggatgaggaagggcggcagaagatccgggaag
agaaggataagagcaaggaactgcatgccattaaggagcgttacctgggtggcatcaaaaagcgg
cgccgaacgagacatctcaatgaccggaaatttgtttttgagtgggatgcatctgaggacacatc
cattgactacaaccccctgtacaaagaacggcaccaggtgcagttgttagggcgaggcttcattg
caggcattgacctcaagcagcagaagcgagagcagtcacgtttctatggagacctaatggagaag
aggcgaaccctggaagaaaaggagcaggaggaggcaagactccgcaaacttcgtaugaaggaagc
caagcagcgctgggatgatcgtcattggtctcagaaaaagttagatgagatgacggacagggact
ggcggatcttccgtgaggactacagcatcaccaccaaaggtggcaagatccccaatcccatccga
tcctggaaagactcttctctgcccccacacatcttggaggtcattgataagtgtggctacaagga
accaacacctatacagcgtcaggcaattcccattgggctacagaatcgtgacatcattggtgtgg
ctgagactggcagtggcaagacagcagccttcctcatccctctgctggtctggatcaccacactt
cccaaaattgacaggatcgaagagtcagaccaaggcccttatgccatcatcctggctcccacccg
tgagttggctcaacagattgaggaagagaccatcaagtttgggaaaccgctaggtatccgcactg
tggctgtcattggtggcatctccagagaagaccagggcttcaggctgcgcatgggttgtgagatt
gtgattgctacccctgggcgtttgattgatgtgctggagaaccgctacctggtgctgagccgctg
tacctatgtggttctggatgaggcagataggatgattgacatgggctttgagccagatgtccaga
agatcctggagcacatgcctgtcagcaaccagaagccagacacggatgaggctgaggaccctgag
aagatgctggccaactttgagtcgggaaaacataagtaccgccaaacagtcatgttcacggccac
catgcccccagcggtggagcgtctggccaggagcrätcttcggcgacctgctgtggtgtacattg
gctccgcaggcaagccccatgagcgtgtggaacagaaggtcttcctcatgtcagagtcagaaaag
aggaaaaagctgctggcaatcttggagcaaggctttgacccacccatcattatttttgtcaacca
gaagaagggctgcgacgtgttggccaaatccctggagaagatggggtacaatgcttgcacactgc
acggtggaaaaggccaggagcagcgagagtttgcgttgtccaacctcaaggctggggccaaggat
attttggtggctacagatgtggctggtcgtggtattgacatccaagatgtgtctatggttgtcaa
ctatgatatggccaaaaatattgaagattacatccaccgcattggccgcacgggacgagcaggca
agagtggggtggccatcaccttcctcacaaaagaggactctgctgtgttctacgagctgaagcaa
gctatcctggaaagcccagtgtcttcctgtccccccgaactagccaaccacccagatgcccagca
taagccaggcaccatcctcaccaagaagcgcgggaagagaccatctttgcctga
[Chemical Formula 19]
DDX24 DNA sequence
(SEQ ID NO: 36)
aagttgaaggacacaaaatcaaggccaaagcagtcaagctgtggcaaatttcagacaaagggaat
caaagttgtgggaaaatggaaggaagtgaagattgacccaaatatgtutgcagatggacagatgg
atgacttggtgtgctttgaggaattgacagattaccagttggtctcccctgccaagaatccctcc
agtctcttctcaaaggaagcacccaagagaaaggcacaagctgtttcagaagaagaggaggagga
ggagggaaagtctagctcaccaaagaaaaagatcaagttgaagaaaagtaaaaatgtagcaactg
aaggaaccagtacccagaaagaatttgaagtgaaagatcctgagctggaggcccagggagatgac
atggtttgtgatgatccggaggctggggagatgacatcagaaaacctggtccaaactgctccaaa
aaagaagaaaaataaagggaaaaaagggttggagccttctcagagcactgctgccaaggtgccca
aaaaagcgaagacatggattcctgaagttcatgatcagaaagcagatgtgtcagcttggaaggac
ctgtttgttcccaggccggttctccgagcactcagctttctaggcttctctgcacccacaccaat
ccaagccctgaccttggcacctgccatccgtgacaaactggacatccttggggctgctgagacag
gaagtgggaaaactcttgcctttgccatcccaatgattcatgcggtgttgcagtggcagaagagg
aatgctgcccctcctccaagtaacaccgaagcaccacctggagagaccagaactgaggccggagc
tgagactagatcaccaggcaaggctgaagctgagtctgatgcattgcctgacgatactgtaattg
agagtgaagcactgcccagtgatattgcagccgaggccagagccaagactggaggcactgtctca
gaccaggcgttgctctttggtgacgatgatgctggtgaagggccttcttccctgatcagggagaa
acctgttcccaaacagaatgagaatgaggaggaaaatcttgataaagagcagactggaxatctaa
aacaggagttggatgacaaaagcgccacctgtaaggcatatccaaagcgtcctctgcttggactg
gttctgactcccactcgagagctggccgtccaggtcaaacagcacattgatgctgtggccaggtt
tacaggaattaaaactgctattttggttggtggaatgtccacgcagaaacagcagaggatgctga
accgtcgtcctgagattgtggttgctactccaggccggctgtgggaattaattaaagaaaagcat
tatcatttgaggaaccttcggcagctcaggtgcctggtagtggatgaggctgaccggatggttga
gaaaggccattttgctgagctctcacagctgctagagatgctcaatgactcccaatacaacccaa
agagacaaacgcttgttttttctgccacactcaccctggtgcatcaggctcctgctcgaatcctt
cataagaagcacaccaagaaaatggataaaacagccaaacttgacctccttatgcagaaaattgg
catgaggggcaagcccaaggtcattgacctcacaaggaatgaggccacggtggagacgctaacag
agaccaagatccattgtgagactgatgagaaagacttctacttgtactacttcctgatgcagtat
ccaggccgcagcttagtgtttgccaacagtatctcctgcatcaaacgcctctctgggctcctcaa
agtccttgatatcatgcccttgaccctgcatgcctgtatgcaccagaagcagaggctcagaaacc
tggagcagtttgcccgtctggaagactgtgttctcttggcaacagatgtggcagctcggggtctg
gatattcctaaagtccagcatgtcatccattaccaggtcccacgtacctcggagatttatgtcca
ccgaagtggtcgaactgctcgagctaccaatgaaggcctcagtctgatgctcattgggcctgagg
atgtgatcaactttaagaagatttacaaaacgctcaagaaagatgaggatatcccactgttcccc
gtgcagacaaaatacatggatgtggtcaaggagcgaatccgtttagctcgacagartgagaaatc
tgagtatcggaacttccaggcttgcctgcacaactcttggattgagcaggcagcagctgccctgg
agattgagctggaagaagacatgtataagggaggaaaagctgaccagcaagaagaacgtcggag
acaaaagcagatgaaggttctgaagaaggagctgcgccacctgctgtcccagccactgtttacgg
agagccagaaaaccaagtatcccactcagtctggcaagccgcccctgcttgtgtctgccccaagt
aagagcgagtctgctttgagctgtctctccangcagaagaagaagaagacaaagaagccgaagga
gccacagccggaacagccacagccaagtacaagtgcaaattaa
[Chemical Formula 20]
DDX25 DNA sequence
(SEQ ID NO: 37)
gcgtcgttactgtggggaggcgacgcaggggcggcggagagcgagcggctgaacagccacttttc
aaacctcagccaaccccggaagaacctttggggtattaagagtactgcagtccgaaacatagatg
gttctattaataacatcaatgaagatgatgaagaagatgtagtggatttggcagctaattcactc
ttaaacaagttaatccatcaatccttagtagaatccagtcaccgtgtggaagttttacagaagga
tcccagctctccactttactcagtaaagacatttgaagagctgcggctaaaggaagagttactaa
aaggaatctatgcaatgggatttaataggccatctaaaatccaagagatggctctccctatgatg
ctggcacatccaccccagaacctcatagcacagagccagtctggaacaggaaagacagcggcatt
tgtgttggcaatgttaagcagagttaatgccttggaattgttcccacagtgcctctgcctagctc
ctacttatgaattggctctgcaaactggccgtgtggttgagcagatgggaaaattctgtgtggat
gttcaagtgatgtatgccattcgagggaatcgaattcccagaggcaccgacatcactaaacagat
tataattggcactcctgggactgtcctagattggtgttttaaactaaaattgattgatttgacta
agattcgtgtgtttgtcctggatgaagcagatgtgatgattgacactcaaggattctcagatcat
agtattcgtattcaaagagctctaccctccgaatgccaaatgctcctcttttcagcaacctttga
ggactctgtgtggcactttgctgagcgaatcatccctgaccctaatgttatcaagttacgcaaag
aggagctcacactgaacaacatccggcaatattacgtgtgtgtgagcacaggaaagacaaatacc
aagctctgtgcaacatttatggcagcatcaccattggtcaggccatcatcttctgccagactcgt
cgaaacgctaagtggttgaccgtggagatgatacaggatggccaccaggtgtctttgttaagcgg
ggagctgaccgtggagcagcgagcttccatcattcagaggtttcgggatgggaaagagaaggttc
tcataacaactaatgtttgtgcccgagggattgatgtgaagcaggtcacaattgttgtgaacttt
gatctccctgtaaaacaaggagaggagccggactatgagacctacctccaccgcatagggcggac
ggggcgctttgggaaaaaaggccttgccttcaacatgattgaagtagatgagctgccctcgctca
tgaaaatccaggaccactttaacagcagtattaagcaactcaacgctgaagacatggatgaaatt
gaaaagattgactattga
[Chemical Formula 21]
DDX27 DNA sequence
(SEQ ID NO: 38)
cttgcggacctcggcttaatcggaaccataggcgaggatgacgaggtgccggtggagcccgagtc
tgactccggggacgaggaagaggaggggcccattgtgctgggcagacgacaaaaagctttgggga
agaaccgcagtgctgatttcaaccctgatttcgttttcactgagaaggaggggacgtacgatggc
agctgggccctggctgatgtcatgagccaactcaagaagaagagggcagccactacattagatga
gaagattgagaaagttcgaaagaaaaggaaaacagaggataaagaagccaagtctgggaagttgg
aaaaggagaaagaagcaaaggaaggctctgaaccaaaggagcaggaagaccttcaagagaatgat
gaggaaggctcagaagatgaagcctcggagactgactactcatcagctgatgagaacatcctcac
caaagcagatacactcaaagtaaaggatcggaagaagaagaagaagaaaggacaggaagcaggag
gattttttgaagatgcatctcagtacgatgaaaacctctcgttccaggacatgaacctttcccgc
cctcttctgaaggccattacagccatgggcttcaagcagcccaccccgatccagaaggcgtgcat
acctgtgggtctattggggaaggacatctgtgcctgtgcagccactgggacaggtaaaactgccg
cctttgccctgcctgttttggagcgtctgatttataaaccccgccaggctccagtcacccgcgtg
ctggtgctagtgcccacccgagagctgggcatccaggtgcactctgtcaccagacagctggccca
gttctgcaacatcaccacctgcctggctgtgggcggcttggatgtgaagtctcaggaagcagctc
ttcgggcagcgcctgacatcctcatcgccaccccaggccggctcatcgatcacctccacaactgc
ccttccttccacctgagcagcatcgaggtgctcatcctggacgaggctgacaggatgctggatga
gtactttgaggagcagatgaaggagatcatccgaatgtgttcccaccaccgccagaccatgctct
tctcggccaccatgacagacgaggtgaaagatctggcttctgtctccttgaagaatcctgtccgg
atatttgtgaacagcaacacagatgtggctcccttcctgcggcaggagttcatccggatccggcc
taatcgtgaaggagaccgggaagccatcgtggcagctttgttgacgaggaccttcactgaccatg
tgatgctgttcacgcaaaccaagaagcaggcccaccgcatgcacatcctcctggggctcatgggg
ctgcaggtgggtgagctccatggcaacttgtcacagacgcagcggctggaggccctccggcgttt
taaggatgaacagattgacatcctcgtggccactgatgtggcagcccgtggacttgacattgagg
gggtcaaaacggtaatcaacttcacaatgcctaataccatcaaacattatgtccaccgggtgggg
cgaacagcacgtgctggcagggctgggcgctcagtctctctggtgggagaagatgagcggaagat
gctgaaggagattgtaaaagctgccaaggcccctgtgaaggccaggatacttccccaagatgtca
tcctcaaattccgggacaagattgagaaaatggagaaagatgtgtatgcagttctgcagctagag
gcggaggaaaaagagatgcagcagtcagaagcccagatcaatacagcaaagcggctcctggagaa
ggggaaggaggcagtggtccaagagcccgagaggagctggttccagaccaaagaagagaggaaga
aggagaaaattgccaaagctctgcaggaatttgacttggccttaagaggaaagaagaaaaggaag
aagtttatgaaggatgccaaaaaaaagggggagatgacagcagaggaaaggtctcagtttgaaat
cctcaaggcgcagatgtttgctgaacggctagcgaagaggaatcgcagagccaagcgggcccgag
caatgcccgaggaggagccagtgagaggtcctgccaagaagcaaaagcaggggaagaaatctgta
tttgatgaagaactcaccaacacaagcaagaaggccctgaaacagtatcgagctggcccttcctt
tgaagaaaggaaacagttgggcttgccccaccagagacgaggaggaaactttaaatctaaatcca
gatacaagaggaggaagtag
[Chemical Formula 22]
DDX28 DNA sequence
(SEQ ID NO: 39)
gctctaacgcggccggtgcggctcttttccctcgtgactcggttgctcctggcgccgcgacgggg
cctcacggtccgcagtcccgacgaacccctgccggtggtgcgcatcccagtggctctacagcggc
agttggaacagcggcagagcaggcggcggaacctcccgaggccggtgctggttcgacccggaccg
ctgctggtttcggcgcggcggccggagttgaaccagccggcgcgcctcacactgggccgttggga
gcgcgcgccgctagcctctcaaggctggaagagtcgcgcgcgcgtcgggaccacttctccatcga
gcgcgcgcaacaggaggcgccagcggtgcgaaagctctcgtctaagggcagctttgctgacctgg
gcctggagccccgtgtgctgcacgcactacaggaggctgcgcctgaagtcgttcagcccacaacc
gtgcagtctagcaccatcccctcactacttcgcggccgccatgtcgtttgcgccgcagaaaccgg
cagtggcaagactctcagctacctcctgccgctgcttcaacggctcttgggccagccaagcctgg
actcccttcctatccccgcgccccgaggcctggtccttgttccttcccgagaattggcccaacag
gtgcgggctgtggcccaacccttgggccgctccttgggcctgctggtgcgggacctggagggagg
ccacggcatgcgtaggatcaggctgcagctgtccagacagccttcagcagatgtgcttgtggcca
ctccaggggctctgtggaaggccctgaaaagtcgactgatcagtctggagcaactctccttcttg
gtgttggatgaggcagacacactgctggatgaaagcttcctggaactggtggactacatcttaga
gaagagccacatagcagaaggcccagctgacttggaagaccccttcaatcccaaagctcagttag
tgctggtaggagccacatttcccgaaggtgtaggccagttgctgaataaagtcgccagcccagat
gctgtcaccaccatcaccagctccaagctccactgtatcatgcctcatgtgaaacagacatttct
gagactgaagggagcagataaggtggccgagctggtgcacatcctcaagcatcgtgacagagcag
aaaggactggtccctcaggaactgttctggtgttctgtaatagctccagcactgtgaactggctg
ggatatattctggatgaccacaaaatccaacacctaaggttgcaggggcaaatgccagccttgat
gagggtaggaatcttccagtccttccagaagagctcccgagacatacttctctgcacagacatag
cctctcggggcctggacagcactggtgtggagctggttgtcaattatgatttccccccaacgctg
caagattacatccacagagcagggagagtgggccgtgtggggagcgaggtgccaggcaccgtcat
cagttttgtgacccatccctgggatgtgagcctggttcagaagattgagctggcggctcgccgaa
ggagaagtcttccaggactagcatcctcggtgaaagagcctttgccccaagcaacctga
[Chemical Formula 23]
DDX31 DNA sequence
(SEQ ID NO: 40)
gcagccgccgacggttcgctcttcgacaaccccaggacgttctccagacgtcccccagcccaggc
gagtcggcaagcaaaggctacgaaaagaaaataccaagcgtccagtgaggctcccccagcgaaac
ggaggaacgaaacttcatttctcccagccaagaaaactagtgttaaagaaactcagaggactttt
aaggggaacgcacaaaaaatgttttctccaaagaagcattcggttagcacaagtgatagaaacca
ggaggagagacagtgcattaagacttcatcactgtttaaaaacaaccctgacattccagaactcc
acagacctgtggtaaagcaggtgcaagaaaaagtgtttacttcagctgcttttcatgagctgggc
ctccacccacatttaatttccacaataaatacggtcttaaaaatgtctagtatgaccagtgttca
gaagcaaagtattcctgtgttgctggaaggcagagatgctctcgtgagatcccagacgggctcag
gtaaaactcttgcctattgcatccctgtggtccagtcccttcaagcaatggagtcaaaaatacag
cgcagtgatggcccctatgccctggtgctcgtgccaacgagagagctagctctacaaagctttga
cactgtccagaaactgcttaagcctttcacctggattgtgcctggagtgttaatgggaggagaga
agagaaaatcagaaaaggccagactccgcaaaggaataaatatccttatctcaactcctggacgc
ctggtggatcatataaaatccacaaagaacattcattttagtcggctgcggtggttggtgtttga
tgaagcagacagaatcttggatttgggttttgaaaaggacatcacagtgatacttaatgctgtaa
atgctgaatgccaaaaacgacagaatgtcttgctatcagcgacactcacagaaggtgtaacgcgg
ctagctgatatcagtttgcatgatccagtcagtatttctgtcctggacaagagccatgaccagtt
gaacccaaaggacaaagcggtccaggaggtctgtcctccaccagctggcgacaagctggacagct
ttgcaataccagagagtctcaagcagcatgtgactgtggttcccagcaaactgaggcttgtctgc
ctagcggccttcatccttcagaaatgcaagtttgaggaagaccagaagatggttgtctttttct
caagttgcgagctggtggagttccactacagcctcttcctacagaccctgctgagcagctcaggg
gcgccggcatcagggcagttgccatctgcctccatgcgattaaaattcctacggctgcatggcgg
catggagcaggaggaaagaacagcagtgtttcaggaattttcacattccagaagaggcgtccttc
tttgcacggatgttgcagctcggggcttagatctccctcaagtcacgtggattgttcagtacaac
gctccatcttcacctgcagaatacatccaccggattggaagaaccgcccggattggctgccatgg
gagcagcctgctcattttggctccttcggaggcagaatatgtcaactcgttggcttctcacaaaa
tcaacgtttctgagattaagatggaagatattttgtgtgttctgacaagagatgattgttttaaa
gggaaacgatggggagcccagaaatcccatgctgttggcccccaggaaatccgagagcgagccac
agtcttgcagacggtatttgaagattacgtgcactccagtgagaggagggtctcctgggcaaaga
aagctctgcagtccttcatccaagcctacgccacctaccccagggagctgaagcacatcttccac
gtccgatccctccaccttgggcatgtggcgaagagcttcggactaagagatgcccccaggaatct
tagtgccttgactagaaagaagaggaaagcacacgtgaaaaggcctgaccttcataagaagaccc
agagtaaacacagcctcgctgaaatcctacgttcggaatactcaagcggcatggaggccgacatc
gccaaggtcaaaaagcaaaacgcacctggagagcctggtggccggcccctgcagcacagtctgca
gccgacaccctgctttggccgtgggaaaacattaaaatggagaaaaacccaaaaaggtgtacagc
gggacagcaagacttcccagaaagtttaa
[Chemical Formula 24]
DDX39B DNA sequence
(SEQ ID NO: 41)
gcagagaacgatgtggacaatgagctcttggactatgaagatgatgaggtggagacagcagctgg
gggagatggggctgaggcccctgccaagaaggatgtcaagggctcctatgtctccatccacagct
ctggctttcgtgacttcctgctcaagccagagttgctccgggccattgtcgactgtggctttgag
catccgtcagaagtccagcatgagtgcatccctcaggccattctgggaatggatgtcctgtgcca
ggccaagtgggcatgggaaagacagcagtgtttgtcttggccacactgcaacagctggagccagt
tactgggcaggtgtctgtactggtgatgtgtcacactcgggagttggcttutcagatcagcaagg
aatatgagcgcttctctaaatacatgcccaatgtcaaggttgctgttttttttggtggtctgtct
atcaagaaggatgaagaggtgctgaagaagaactgcccgcatatcgtcgtggggactccaggccg
tatcctagccctggctcgaaataagagcctcaacctcaaacacattaaacactttattttggatg
aatgtgataagatgcttgaacagctcgacatgcgtcgggatgtccaggaaatttttcgcatgacc
ccccacgagaagcaggtcatgatgttcagtgctaccttgagcaaagagatccgtccagtctgccg
caagttcatgcaagatccaatggagatcttcgtggatgatgagacgaagttgacgctgcatgggt
tgcagcagtactacgtgaaactgaaggacaacgagaagaaccggaagctctttgaccttctggat
gtccttgagttcaaccaggtggtgatctttgtgaagtctgtgcagcggtgcattgccttggccca
gctactagtggagcagaacttcccagccattgccatccaccgtgggatgccccaggaggagaggc
tttctcggtatcagcagtttaaagattttcaacgacgaattcttgtggctaccaacctatttggc
cgaggcatggacatcgagcgggtgaacattgcttttaattatgacatgcctgaggattctgacac
ctacctgcatcgggggccagagcaggccggtttggcaccaagggcttggctatcacatttgtgtc
cgatgagaatgatgccaagatcctcaatgatgtgcaggatcgctttgaggtcaatattagtgagc
tgcctgatgagatagacatctcctcctacattgaacagacacggtag
[Chemical Formula 25]
DDX41 DNA sequence
(SEQ ID NO: 42)
gaggagtcggaacccgaacggaagcgggctcgcaccgacgaggtgcctgccggaggaagccgctc
cgaggcggaagatgaggacgacgaggactacgtgccctatgtgccgttacggcagcgccggcagc
tactgctccagaagctgctgcagcgaagacgcaagggagctgcggaggaagagcagcaggacagc
ggtagtgaaccccggggagatgaggacgacatcccgctaggccctcagtccaacgtcagcctcct
ggatcagcaccagcaccttaaagagaaggctgaagcgcgcaaagagtctgccaaggagaagcagc
tgaaggaagaagagaagatcctggagagtgttgccgagggccgagcattgatgtcagtgaaggag
atggctaagggcattacgtatgatgaccccatcaaaaccagctggactccaccccgttatgttct
gagcatgtctgaagagcgacatgagcgcgtgcggaagaaataccacatcctggtggagggagacg
gtatcccaccacccatcaagagcttcaaggaaatgaagtttcctgcagccatcctgagaggcctg
aagaagaaaggcattcaccacccaacacccattcagatccagggcatccccaccattctatctgg
ccgtgacatgataggcatcgctttcacgggttcaggcaagacactggtgttcacgttgcccgtca
tcatgttctgcctggaacaagagaagaggttacccttctcaaagcgcgaggggccctatggactc
atcatctgcccctcgcgggagctggcccggcagacccatggcatcctggagtactactgccgcct
gctgcaggaggacagctcaccactcctgcgctgcgccctctgcattgggggcatgtccgtgaaag
agcagatggagaccatccgacacggtgtacacatgatggtggccaccccggggcgcctcatggat
ttgctgcagaagaagatggtcagcctagacatctgtcgctacctggccctggacgaggctgaccg
catgatcgacatgggcttcgagggtgacatccgtaccatcttctcctacttcaagggccagcgac
agaccctgctcttcagtgccaccatgccgaagaagattcagaactttgctaagagtgcccttgta
aagcctgtgaccatcaatgtggggcgcgctggggctgccagcctggatgtcatccaggaggtaga
atatgtgaaggaggaggccaagatggtgtacctgctcgagtgcctgcagaagacacccccgcctg
tactcatctttgcagagaagaaggcagacgtggacgccatccacgagtacctgctgctcaagggg
gttgaggccgtagccatccatgggggcaaagaccaggaggaacggactaaggccatcgaggcatt
ccgggagggcaagaaggatgtcctagtagccacagacgttgcctccaagggcctggacttccctg
ccatccagcacgtcatcaattatgacatgccagaggagattgagaactatgtacaccggattggc
cgcaccgggcgctcgggaaacacaggcatcgccactaccttcatcaacaaagcgtgtgatgagtc
agtgctgatggacctcaaagcgctgctgctagaagccaagcagaaggtgccgcccgtgctgcagg
tgctgcattgcggggatgagtccatgctggacattggaggagagcgcggctgtgccttctgcggg
ggcctgggtcatcggatcactgactgccccaaactcgaggctatgcagaccaagcaggtcagcaa
catcggtcgcaaggactacctggcccacagctccatggacttctga
[Chemical Formula 26]
DDX42 DNA sequence
(SEQ ID NO: 43)
aactggaataaaggtggtcctggcactaagcgaggatttggctttggaggttttgccatcagtgc
tgggaaaaaggaggaacccaaactcccacagcagtcccacagtgcctttggggcaaccagctctt
cttctggatttggaaagtcagctccaccacagcttccttctttctacaaaattggatctaagcgg
gccaactttgatgaagaaaatgcctattttgaagatgaggaagaagattctagcaacgttgattt
accttacattcctgctgaaaactcaccaactcgccagcaattccattccaagccagtagattctg
acagcgatgatgatcccttggaggcattcatggctgaagtggaggatcaggcagctagagacatg
aagaggcttgaagaaaaggacaaggaaagaaaaaacgtaaagggtattcgagatgacattgaaga
ggaagatgaccaagaagcttattttcgatacatggcagaaaacccaactgctggtgtggttcagg
aggaagaggaagacaatctagaatatgatagtgacggaaatccaattgcacctaccaaaaaaatc
attgatcctcttccccccattgatcattcagagattgactatccaccatttgaaaaaaactttta
caatgagcatgaagagataaccaacctcactccacagcagttaatagatctccggcataagctca
atcttcgggtctctggtgctgcacctcctagaccaggaagtagctttgctcattttgggtttgac
gaacaacttatgcaccagattcggaaatctgaatacacacagcccactccaatacagtgccaggg
tgtgcctgtggcattaagtggtagagacatgattggtattgccaaaacaggtagtgggaaaactg
cagccttcatttggcccatgttgattcatataatggaccagaaggagttggaaccaggtgatgga
ccaattgcagtgattgtgtgtcctaccagggagctttgccagcagatccatgcagaatgtaagcg
gtttggaaaagcatataatcttcgatcagtggccgtatatggaggagggagtatgtgggagcagg
ccaaggcccttcaggagggggcagagattgttgtgtgtaccccaggtcgactgatagatcatgtg
aaaaagaaagctaccaatcttcaaagagtctcttaccttgtgtttgatgaagcagatcgaatgtt
tgacatgggatttgagtaccaagttcgatccatagcaagtcatgttcgtcctgacaggcagactc
tcttatttagtgcaacttttcggaagaagattgaaaagttggccagagacatcctgatcgaccct
attcgagtggtgcagggagatattggagaggcaaatgaagatgtgacacagattgtggagattct
ccattctggacctagtaaatggaactggcttacccggcgtctggtagaatttacctcttcaggga
gtgtcctcctctttgttactaaaaaagccaatgctgaagagctagcgaataaccttaaacaggag
ggtcataatcttgggctgctccatggggatatggatcagagtgagagaaacaaggtcatttcaga
ctttaagaaaaaggacatcccagtcctggtggccacagatgttgcagcccgtggtctggacattc
cttcaattaagactgtcattaactatgatgtggcacgagacattgatacccacacgcataggatt
ggccgcacaggaagagcgggtgagaaaggtgtggcctataccctactcactcccaaggacagcaa
ttttgctggtgacctggtccggaacttggaaggagccaatcaacacgtttctaaggaactcctag
atctggcaatgcagaatgcctggtttcggaaatctcgattcaaaggagggaaaggaaaaaagctg
aacattggtggaggaggcctaggctacagggagcggcctggcctgggctctgagaacatggatcg
aggaaataacaatgtaatgagcaattatgaggcctacaagccttccacaggagctatgggagatc
gactaacggcaatgaaagcagctttccagtcacagtacaagagtcactttgttgcagccagttta
agtaatcagaaggctggaagttctgctgctggggcaagtgggggactagtgcagggagcttgaat
tctgttccaactaactcagcacaacagggccataacagtcctgacagccccgtcaccagtgccgc
caagggcatcccaggctttggcaatactggcaacatcagtggtgcccctgtgacctacccgtctg
ccggagcccaaggagtcaacaacacagcttcagggaataacagccgagaagggactgggggcagc
aacgggaaaagagagagatatactgagaaccggggcagcagccgtcacagtcacggagagactgg
caatcggcatagcgatagtccacgtcacggagatggtggtcgccatggagatggataccgccatc
cagaaagcagcagccgtcatactgatggccatcggcacggggagaacagacatggaggaagcgca
ggccggcatggggagaaccggggtgcaaatgatggtcggaatggggaaagcaggaaagaagcttt
taatcgtgagagcaagatggagcccaagatggaacccaaagtggacagcagcaagatggacaagg
tggacagcaagacagataagacagctgacggctttgctgtcccagagccgcctaaacgcaagaaa
agtcgatgggacagttag
[Chemical Formula 27]
DDX43 DNA sequence
(SEQ ID NO: 44)
tcccaccacggaggagctcccaaggcctctacgtgggtcgttgctagtcggcgaagctcgacagt
gtcccgagcgccagagaggaggccggcggaggagttgaatcgaacaggtcctgagggatatagtg
tcggcagaggtggtcgctggagaggcacctctaggcccccggaggccgtggccgctggtcacgag
gaactgccgctgtgttttgctttgaagagccactttgttggcgcggtaatcggtcgtggtgggtc
aaaaataaagaatatacaaagtacaacaaacaccacaatccaaataatacaagaacaaccagaat
cattagtcaaaatttttggcagcaaggcaatgcaaacgaaagcaaaagcagtgatagacaatttt
gttaaaaagctagaagaaaattacaattcagaatgcggaattgatactgcattccaaccttctgt
tggaaaagatggaagcacagataacaatgttgttgcaggagatcggccattgatagattgggatc
aaattagagaggaaggtttgaaatggcaaaaaacaaagtgggcagatttaccaccaattaagaaa
aacttttataaagagtccactgccacaagtgccatgtcaaaagtagaagcagatagttggaggaa
agaaaattttaatataacgtgggatgacttgaaggatggggagaaacgacctatccccaatccta
cctgcacatttgatgacgcctttcaatgttatcctgaggttatggaaaacattaaaaaggcaggt
tttcaaaagccaacacctattcagtcacaggcatggcccattgtgttgcaaggaatagatcttat
aggagtagcccagactggaacaggaaagacattgtgttatttaatgcctggatttattcatctgg
tccttcaacccagccttaaaggtcaaaggaatagacccggcatgttagttctaactcccactcgg
gaattagcacttcaagtagaaggagaatgttgcaaatattcatataaagggcttcggagtgtttg
tgtatatggtggtggaaatagagatgaacaaatagaagagcttaaaaaaggtgtagatatcataa
ttgcaactcccggaagattgaatgatctgcaaatgagtaacttcgtcaatctgaagaatataacc
tacttggttttagatgaagcagacaagatgttggacatgggatttgaaccccagataatgaagat
tttgttagatgtgcgcccagataggcagacagttatgaccagtgctacatggcctcattcagttc
atcgcctcgcacaatcttatttgaaagaaccaatgattgtctatgttggtacattggatctagtt
gctgtaagttcagtgaagcaaaatataattgtaaccaccgaggaagagaaatggagtcacatgca
aacttttctacagagtatgtcatccacagacaaagtcattgtcttcgtttctcgaaaagctgttg
cggatcacttatcaagtgacctaatacttggaaatatatcagtagagtctctgcatggagataga
gaacagagagatcgggagaaagcattagagaactttaaaacaggcaaagtgagaatactaattgc
aactgatctagcctctagaggacttgatgtccatgacgttacacatgtctataattttgactttc
cacggaatattgaagaatacgtacaccgaatagggcgcacgggaagagcagggaggactggtgtt
tccattacaactttgactagaaatgattggagggttgcctctgaattgattaatattctggaaag
agcaaatcagagtattccagaggagcttgtatcaatggctgagaggtttaaggcacatcaacaga
aaagggaaatggaaagaaaaatggaaagacctcaaggaaggcccaagaagtttcattaa
[Chemical Formula 28]
DDX46 DNA sequence
(SEQ ID NO: 45)
ggtcgggagtcacgccactatcgaaaacgatcggcatcccggggtcgctctggaagtcggtctag
aagtcgctcaccctcagacaaaagaagtaaacgtggagatgacagacggtctagaagtagagata
gagataggaggagagagaggtctcgtagcagggataaaagaagatctcggtcaagggacaggaag
cgtctgagacgttccagaagtagagagagagacagaagccgagagcgaagaagatctcgaagtag
agacaggagacgctcaaggagtagaagccggggccggcgatcccgatcctccagtcctggaaata
aaagcaagaaaactgagaatagatctaggtccaaagagaaaactgatggtggggaaagttctaaa
gagaagaaaaaagacaaagatgacaaggaggatgaaaaagaaaaagatgctggcaacttgaccag
aataagctggaagaagaaatgagaaagcgaaaagaaagagtagaaaaatggcgagaagagcaacg
taaaaaggctatggaaaacataggagaactgaaaaaggaaatcgaagagatgaaacaagggaaaa
agtggagtttagaggacgatgatgatgacgaagatgatcctgcagaagctgaaaaggagggaaat
gaaatggagggtgaggagttagatccattagatgcttacatggaagaagtgaaagaggaagtaaa
aaaatttaacatgagaagtgtaaaagctggtgggggaaatgaaaagaagtctgggccaacggtca
caaaagttgtcactgttgtgacaaccaaaaaagcagutgtggattctgataagaagaaaggtgag
ctgatggagaatgaccaggatgccatggagtattcttcagaggaggaagaagttgatcttcagac
agcccttacagggtatcaaacaaxacagcgaaagcttctagaaccagttgatcatggaaaaattg
agtatgagccatttaggaaaaacttctatgutgaagttccagaactagcaaaaatgtctcaagaa
gaggtaaatgtgtttcgattggaaatggagggcattacagttaaaggaaaaggttgccccaaacc
aattaaatcctgggtccagtgtggaatttccatgaagatcttaaattccctcaagaagcatggct
atgaaaagcccacgcccatccaaacccaagctattcctgctataatgtctggacgagatttgatt
ggcattgccaaaacaggaagtggaaagaccattgcttttctgttgcccatgtttagacacatcat
ggatcagaggtcattagaggaaggagaggggccaatagctgtcatcatgactccaactcgagaac
tggctttacagattactaaagagtgtaagaagttttccaagactttgggacttagagtggtctgt
gtttacggaggaacaggaatcagtgagcagattgctgagctgaaaagaggtgctgaaattattgt
ttgcacacctggtcgaatgattgacatgttagccgctaacagtggtcgggtcacaaatcttcgaa
gagtgacatatgttgttttagatgaagcagacagaatgtttgacatgggtttgaaccccaggtca
tgcgcatcgtggataatgttcgtcctgatcgacagacggttatgttttcagctactttccccaga
gctatggaggctttggctcgcaggatcctcagtaaacctattgaagtacaagttggaggcaggag
tgtggtttgctcagatgtggagcaacaagtgattgtgattgaagaagaaaagaaattcttgangt
tacttgagcttctaggccattatcaagagtcaggatctgtcattatatttgtggataagcaggaa
catgctgatggtcttcttaaggatttaatgagagcatcttatccttgcatgtctcttcatggagg
cattgatcaatatgacagagatagcatcataaatgactttaagaatgggacctgcaaacttcttg
tggctacctctgttgctgcccgaggtctagatgtgaaacatctgattcttgtagtaaattatagc
tgccccaaccattatgaggattatgtacacagagcagggcggactggaagagcaggaaacaaggg
ttatgcttatacttttatcacagaggatcaagctcgctatgctggtgacataattaaagctcttg
aattgtcagggactgcagtacctcctgatttagagaaactgtggagtgatttcaaagatcagcag
aaagctgaggggaaaataattaaaaagagtagtgggttctctggtaagggattcaagtttgatga
aacagaacaagctttggctaatgagaggaagaagttacaaaaagcagctcttggtctacaagatt
cagatgatgaggatgctgcagttgatattgatgagcaaattgaaagcatgtttaattcaaagaag
agagtaaaggatatggctgctcctggaacatcaagtgttcctgctccaactgcaggaaatgctga
gaaattagaaattgctaagagattggctcttagaatcaatgcccagaagaatttgggcatcgagt
ctcaggtagatgtgatgcagcaggccaccaatgcaattcttaggggtggcaccattctggctccc
actgtttctgcaaaaaccattgcagaacaacttgctgaaaagatcaatgccaagctcaattatgt
gccgttagagaaacaagaagaagagagacaggatggtggacagaatgaatcttttaagagatatg
aagaagaattagagatcaatgacttcccacagactgctaggtggaaagttacctctaaggaagct
ctgcagagaatcagtgaatactctgaagccgcaattacaatcagaggaacctacttccctcctgg
caaagaacccaaggaaggcgagcggaagatttacttggcaattgaaagtgccaatgaactggctg
tgcagaaagcaaaggcagaaatcaccaggctcataaaagaagagctgatccggctgcaaaattca
taccaaccaacaaataaaggaagatacaaagtcttatag
[Chemical Formula 29]
DDX47 DNA sequence
(SEQ ID NO: 46)
gcggcacccgaggaacacgattctccgaccgaagcgtcccagccgattgtggaagaggaggaaac
taaaacatttaaagacctgggtgtgacagatgtgttgtgtgaagcttgtgaccagttgggatgga
caaaacccaccaagatccagattgaagctattcctttggccttacaaggtcgtgatatcattggg
cttgcagaaactggctctggaaagacaggcgcctttgctttgcccattctaaacgcactgctgga
gaccccgcagcgtttgtttgccctagttcttaccccgactcgggagctggcctttcagatctcag
agcagtttgaagccctggggtcctctattggagtgcagagtgctgtgattgtaggtggaattgat
tcaatgtctcaatctttggcccttgcaaaaaaaccacatataataatagcaactcctggtcgact
gattgaccacttggaaaatacgaaaggtttcaacttgagagctctcaaatacttggtcatggatg
aagccgaccgaatactgaatatggattttgagacagaggttgacaagatcctcaaagtgattcct
cgagatcggaaaacattcctcttctctgccaccatgaccaagaaggttcaaaaacttcagcgagc
agctctgaagaatcctgtgaaatgtgccgtttcctctaaataccagacagttgaaaaattacagc
aatattatatttttattccctctaaattcaaggatacctacctggtttatattctaaatgaattg
gctggaaactcctttatgatattctgcagcacctgtaataatacccagagaacagctttgctact
gcgaaatcttggcttcactgccatccccctccatggacadatgagtcagagtaagcgcctaggat
cccttaataagtttaaggccaaggcccgttccattcttctagcaactgacgttgccagccgaggt
ttggacatacctcatgtagatgtggttgtcaactttgacattcctacccattccaaggattacat
ccatcgagtaggtcgaacagctagagctgggcgctccggaaaggctattacttttgtcacacagt
atgatgtggaactcttccagcgcatagaacacttaattgggaagaaactaccaggttttccaaca
caggatgatgaggttatgatgctgacagaacgcgtcgctgaagcccaaaggtttgcccgaatgga
gttaagggagcatggagaaaagaagaaacgctcgcgagaggatgctggagataatgatgacacag
agggtgctattggtgtcaggaacaaggtggctggaggaaaaatgaagaagcggaaaggccgttaa
[Chemical Formula 30]
DDX48 DNA sequence
(SEQ ID NO: 47)
gcgaccacggccacgatggcgacctcgggctcggcgcgaaagcggctgctcaaagaggaagacat
gactaaagtggaattcgagaccagcgaggaggtggatgtgacccccacgttcgacaccatgggcc
tgcgggaggacctgctgcggggcatctacgcttacggttttgaaaaaccatcagcaatccagcaa
cgagcaatcaagcagatcatcaaagggagagatgtcatcgcacagtctcagtccggcacaggaaa
aacagccaccttcagtatctcagtcctccagtgtttggatattcaggttcgtgaaactcaagctt
tgatcttggctcccacaagagagttggctgtgcagatccagaaggggctgcttgctctcggtgac
tacatgaatgtccagtgccatgcctgcattggaggcaccaatgttggcgaggacatcaggaagct
ggattacggacagcatgttgtcgcgggcactccagggcgtgtttttgatatgattcgtcgcaga
agcctaaggacacgtgctatcaaaatgttggttttggatgaagctgatgaaatgttgaataaagg
tttcaaagagcagatttacgatgtatacaggtacctgcctccagccacacaggtggttctcatca
gtgccacgctgccacacgagattctggagatgaccaacaagttcatgaccgacccaatccgcatc
ttggtgaaacgtgatgaattgactctggaaggcatcaagcaatttttcgtggcagtggagaggga
agagtggaaatttgacactctgtgtgacctctacgacacactgaccatcactcaggcggtcatct
tctgcaacaccaaaagaaaggtggactggctgacggagaaaatgagggaagccaacttcactgta
tcctcaatgcatggagacatgccccagaaagagcgggagtccatcatgaaggagttccggtcggg
cgccagccgagtgcttatttctacagatgtctgggccagggggttggatgtccctcaggtgtccc
tcatcattaactatgatctccctaataacagagaattgtacatacacagaattgggagatcaggt
cgatacggccggaagggtgtggccattaactttgtaaagaatgacgacatccgcatcctcagaga
tatcgagcagtactattccactcagattgatgagatgccgatgaacgttgctgatcttatctga
[Chemical Formula 31]
DDX49 DNA sequence
(SEQ ID NO: 48)
gcaggcttcgcggagctcgggctgtcatcgtggctcgtggaacaatgtcggcagctgggtttgaa
gcagcccacgcccgtgcagctcggctgcatccccgccatcctggagggtcgagactgcttgggct
gtgctaagacaggcagtgggaagacagcagcgtttgtccttcccatcttgcagaagctgtctgag
gatccctatggcatcttctgcctcgtcctgacacccaccagggagctggcctaccagatcgcaga
gcagttccgggtcctggggaagcctctagggctgaaagactgcatcatcgtcggtggcatggaca
tggtggcccaggcgctggagctcrctcggaaaccacacgtggtcatcgccacgccggggcgcctg
gcagatcacctgcgcagctccaacacttttagtataaagaagatccgcttcctggtgatggatga
ggcagaccggctgctggaacagggctgcactgacttcaccgtggacctggaggccatcctggcgg
ctgtgccggcccgcaggcagacactgctgttcagcgccacgctgaccgacacactccgggagctg
cagggtctggccaccaaccagcccttcttctgggaagcacaggccccggtgagcaccgtggagca
gctggaccagcgctacctgctggtgcctgagaaggtcaaggacgcctacctggtccacctgatcc
agcgcttccaggatgagcacgaggactggtccattatcatcttcaccaacacgtgcaagacctgc
cagattctgtgcatgatgctgcgcaaattcagcttccccaccgtggctctgcactccatgatgaa
gcagaaagaacgctttgccgccctagccaagttcaagtccagcatctaccggatcctgatcgcaa
cagacgtggcctcccggggcctggacatccctacggtacaggtggtcatcaaccacaacaccccc
gggctccccaagatctacatccaccgagtcggccggacggcccgtgcagggcggcagggtcaggc
catcacgctggtgacacagtacgacatccacctggtgcacgccatcgaggagcagatcaagaaga
agctggaggagttctccgtggaagaggccgaggtgctacagatcctcacacaggtcaacgtggtg
cgaagagagtgtgagatcaaactggaggcggcccactttgacgaaaagaaggagatcaacaaacg
gaagcagctgatcctggaggggaaggaccctgacctggaggccaagcgcaaggctgagctggcca
agatcaagcagaagaaccggcgcttcaaggagaaggtggaggagacgctgaagcgacagaaggct
ggcagggctggccacaaggggcgtccacccaggacaccgtctgggtcccactcaggcccagtccc
ctcccagggcctggtctga
[Chemical Formula 32]
DDX50 DNA sequence
(SEQ ID NO: 49)
cctgggaaactcctctggggggacattatggagctggaagcacccttggaggagtccgagagcca
gaagaaggagaggcaaaagagtgacagaaggaagtcaaggcaccattatgactcggatgagaaat
cagaaacaagagaaaatggtgttacagatgacctggatgctcccaaggccaaaaaatctaaaatg
aaagagaagctaaatggagacactgaagaaggatttaatagactttcagatgaattctccaaatc
tcataagtcaagaagaaaagatctaccaaatggagatatagatgaatatgaaaaaaaatcaaagc
gagtatcatctttagatacttctactcataaatcaagtgataataaactagaggagaccttaaca
cgtgaacagaaagaaggagccttctccaattttcctatttctgaagagactataaagcttctgaa
aggtcgaggggtaacatatctctttcctattcaagttaagacctttggtcctgtatatgaaggaa
aagatttaatagctcaagcacggacaggaacaggaaagacattctcttttgccatccccttaatt
gaaagactccaaagaaatcaagaaacaattaaaaaaagccgctcaccaaaggtacttgttttggc
tccaacaagggaactggcaaaccaagtagccaaagacttcaaagatataactaggaaactcagcg
tggcgtgtttttatggtggaacatcatatcaaagccaaattaatcatattcgaaatggtattgac
atcttggttggaacacctggtcgtatcaaagaccatctgcagagtggccgattggatctttctax
actgcgacatgttgtgcttgatgaagtggatcagatgttagatttaggtttcgctgaacaagttg
aagatattattcatgaatcctacaaaactgattctgaagacaatcctcagactttacttttttct
gcaacttgcccacagtgggtatacaaagttgcaaaaaaatacatgaaatccagatatgaacaggt
tgaccttgttggaaaaatgactcaaaaggctgcaactactgtggaacatttggccatccagtgtc
attggtctcagaggccagcagttattggagatgtccttcaagtctacagtgggtctgaagggagg
gctattattttctgtgagaccaagaagaatgtaactgaaatggccatgaatccacacataaxaca
gaatgcccagtgtttacatggggacattgcacagtcacaaxgagaaattacactaaaaggcttca
gagaaggtagttttaaagttttggtggcaaccaatgtggctgcccgtggtttggacattcctgaa
gttgacctggtgattcaaagttctcctcctcaggatgttgagtcctatatccatcgctctggacg
cacaggtagagctggacggacagggatttgtatatgtttttatcaaccaagagaaagaggtcaac
taagatatgtggaacaaaaagcaggaattacttttaaacgtgtaggtgttccttctacaatggat
ttagttaaatctaaaagcatggatgccatcaggtctctggcttccgtttcttacgctgctgttga
ttttttccgaccatcagctcagagactgatagaagagaaaggtgcagtggatgcattggctgcag
ctttagcccacatttctggtgcatcaagctttgaaccacgatctttgatcacctctgataagggg
tttgtgaccatgactctggaaagcctagaggaaatacaggatgtcagctgtgcttggaaagaact
taacagaaagctgagtagtaatgcagtgtctcagattaccagaatgtgcctcctgaaaggaaata
tgggtgtttgctttgatgttcctacaactgagtcagaaaggttacaggcagagtggcatgattcc
gactggatactctcagtgccagccaaattacctgaaattgaagaatattatgatggaaacacatc
ttctaattccagacagaggagtggctggtcaagtggtcgatcaggccggtcaggccggtcaggtg
gtcgatctggcggccggtcaggtagacagagtcgacaaggaagtcgctcaggaagtcgacaagat
ggtagaagacgaagtgggaatagaaatcgatcaagaagtgggggccacaaacggagttttgactg
a
[Chemical Formula 33]
DDX51 DNA sequence
(SEQ ID NO: 50)
gcgctgttctacgtcgcgcggtacccgggccccgatgcggcagctgcggggggccggagggcgcg
gaggccggggcgcacggcagggcccgcgcgctgctcgagcggctgcagagccgggcccgcgaacg
gcagcagcagcgggagcccgcgcagaccgaggcggctgcatcgaccgagccggcgaccaggaggc
gacggcggccccggcggcggcggcgggtgaacgacgcggagccggggagcccggaggcgccgcag
ggaaagcgacggaaggcggacggcgaggacgcgggcgcagaaagcaacgaggaggcgccagggga
gcccagcgcagggagcagcgaggaggcgccaggggagcccagcgcagggagcagcgaggaggcgc
cgggggagcgcagcaccagcgccagcgccgaggcggccccagatggaccggccctggaggaggcg
gccggacccctggtccccggcctggtgctgggggggttcgggaagaggaaggcgccgaaggtcca
gcctttcctgccaaggtggctggctgagcctaactgtgtcagaaggaatgtcaccgaagacctgg
ttcctatcgaggacatccctgacgtccatcctgacctgcagaagcagctggggcacacggcatct
cgtcctactttccagtccaggcagctgtgattcctgccctcctggagagcgcagcctgtgggttt
ctgggggcagaggtggctaccggcctagcgacctctgtgtttctgccccaacaggcagtgggaag
acactggccttcgtcatccctgtggtgcaggccctgctttcgagagtggtctgccacatccgtgc
cctggttgtgctgcccaccaaggagctggcccagcaggtgagcaaagttttcaacatctacacag
atgccacacctctgagagtctccctggttacgggacagaagtctctggccaaggagcaggagagc
ctcgtccagaaaacagctgatgggtaccgctgcttggctgacatcgtggtagccacccccggccg
cctggtggaccacatcgaccagaccccaggattcagcctccagcagctccgcttcctgattatcg
acgaggctgaccggatgattgacagcatgcatcagtcctggctgccgcgggtggtggcggccgcc
ttccagagcgaggaccccgcggacccctgtgccctgctccagcgaaggcaggcccaggctgtgac
agccgccagcacctgctgtccccagatgcccctgcagaagctgctcttctcagctactctgaccc
agaaccctgaaaagctgcagcagctgggcctccaccagccccggcttttctccacagggctagca
cacaggggcctggaagatacagatggggacggggattcggggaagtatgcctttcctgttgggct
cacgcaccactacgtgccctgcagcctcagctctaagccgctggtcgtcctgcacctggtcctgg
agatgggcttctcgagggttctctgcttcactaactcccgagagaactcccacaggctcttcctg
ctggtgcaagcttttgggggtgtggacgtggctgagttctcctcgcgctacgggcctggccagag
gaggatgatcctgaagcagtttgaacaggggaagatccagctgctcatcagcacggacgccaccg
cgcgaggcatcgacgtgcagggtgtggagctggtggtgaactacgacgccccccagtacctgaga
acctacgtgcaccgggttgggaggacagctcgcgctgggaaaactggacaggccttcacactgct
cctgaaagtgcaggagaggagattcctccgaatgctaactgaagctggggcacctgagttgcagc
ggcacgagctctccagcaagctgctgcagccgctggttcctcggtacgaggaggccctgtcccag
ctggaggagtctgtcaaggaagagcgcaagcagagggcggcctag
[Chemical Formula 34]
DDX52 DNA sequence
(SEQ ID NO: 51)
gacgtccacgatctctttcgccggctcggggggggccaaattcgacacgagacgcttctcggcag
acgcagctcgattccagataggaaaaaggaaatatgactttgattcttcggaggtgcttcaggga
ctggacttttttggaaacaagaagtctgtcccaggtgtgtgtggagcatcacaaacacatcagaa
gccccaaaatggagagaaaaaagaagagagcctaactgaaaggaagagggagcagagcaagaaaa
aaaggaagacgatgacttcagaaattgcttcccaagaagaaggtgctactatacagtggatgtca
tctgtagaagcaaagattgaagacaaaaaagttcagagagaaagtaaactaacttccggaaagtt
ggagaatctcagaaaagaaaagataaacttcttgcggaataaacacaaaattcacgtccaaggaa
ccgatcttcctgacccaattgctacatttcagcaacttgaccaggaatataaaatcaattctcga
ctacttcagaacattctagatgcaggtttccaaatgcctacgccaatccaaatgcaagccatcc
cagttatgctgcatggtcgggaacttctggcttctgctccaactggatctggaaaaacattagct
tttagcattcctattttaatgcagctgaaacaacccgcaaataaaggcttcagagccctgattat
atcaccaacacgagaacttgccagccagattcacagagagttaataaaaatttctgagggaacag
gat(caggatacacatgatccacaaagcagcagtggcagccaagaaatttggacctaxatcatct
aaaaagtttgatattcttgtgactactccaaatcgactaatctatttattaaagcaagatccccc
cggaatcgacctagcaagtgttgagtggcttgtagtagacgaatcagataaactgtttgaagatg
gcaaaactgggttcagagaccagctggcttccattttcctggcctgcacatcccacaaggtccga
agagctatgttcagtgcaacttttgcatatgatgttgaacagtggtgcaaactcaacctggacaa
tgtcatcagtgtgtccattggagcaaggaattctgcagtagaaactgtagaacaagagcttctct
ttgttggatctgagaccggaaaacttctggccatgagagaacttgttaaaaagggtttcaatcca
cctgttcttgtttttgttcagtccattgaaagggctaaagaactttttcatgagctcatatatga
aggtattaatgtggatgttattcatgcagagagaacacaacaacagagagataacacagtccaca
gtttcagagcaggaaaaatctgggttctgatttgtacagccttgctagcaagagggattgatttt
aaaggtgtgaacttggtgatcaactatgactttccaactagctcagtggaatatatccacaggat
aggtcgaactggaagagcagggaataagggaaaagcaattacatttttcactgaggatgataagc
cattattaagaagcgttgctaatgttatacagcaggctgggtgtcctgtaccagaatacataaaa
ggttttcagaaactactaagcaaacaaaagaaaaagatgattaagaaaccattggaaagggagag
cattagtacaactccaaaatgtttcttagaaaaagctaaggataaacagaaaaaggtcactggtc
agaacagcaagaagaaagtagctcttgaagacaaaagttaa
[Chemical Formula 35]
DDX53 DNA sequence
(SEQ ID NO: 52)
tcccactgggccccagagtggaagagggcggaggctaatccaagagaccttggggccagctggga
tgtcaggggcagcagaggcagtggctggagtggccccttcggccatcagggaccgagagcagcag
gctcccgtgaaccaccactctgctttaaaataaagaacaatatggttggtgtggtcattggttac
agtggatcaaaaataaaagatctacaacattcgacaaacactaaaatacagatcataaacgggga
atctgaagcaaaagtcagaatttttggcaatagggaaatgaaagcaaaggccaaagcggctatag
aaacacttattagaaaacaagaaagctacaactcagaatccagtgtggataatgctgcatcccaa
acccctattggaagaaatctaggcagaaatgacattgttggagaagctgagccattgtcaaattg
ggatcgcattagggcagcagtcatggagtgcgaaaagagaaaatgggcagatctaccaccagtta
agaaaaacttttacatagaatccaaagcaacaagctgcatgtctgaaatgcaggtgattaactgg
agaaaggaaaatttcaacataacgtgtgatgacttgaaaagtggtgaaaagcgtctcattccaaa
accaacttgcaggtttaaagacgcttttcagcaataccctgatcttctgaaaagcataataaggg
tagggattgtaaagccaacgccaattcagtcacaggcatggccaattattctacaaggaatagat
cttatagtagttgcacaaaccggaacagggaaaacattgtcctatctaatgcctgggtttattca
tcttgattctcaaccaatatctagagagcaaaggaatgggcctgggatgctagtccttacaccca
ctagagagttggctcttcacgtggaagctgaatgttcaaagtattcatataaaggtctcaaaagc
atttgcatatatggtggtagaaacagaaatggacaaatagaagacattagcadaggtgtagatat
cattattgcaactcctgggaggctgaatgacctacaaatgaataactctgtcaacctaagaagca
taacctacttggttatagatgaggcagataxaatgctggatatggaatttgaaccccagataagg
aagattttattagatgtgcgcccagaccgacagactgttatgacaagtgcaacttggccagatac
tgtacgtcaactagcactttcttatttgaaagatcctatgattgtttatgttggtaatctgaatc
tagtggctgtaaatacagtgaagcaaaatataattgttaccacagaaaaagaaaaacgagctctc
acccaagaattcgtagagaacatgtcacccaacgacaaagtcatcatgtttgtcagccaaaaaca
tattgctgatgacttgtcaagcgacttcaatatccaaggcatatctgcagaatcattacatggca
acagtgaacagagtgatcaagagcgagcagtagaggactttaaaagcggaaacataaagatactg
attacaactgatatagtatcccgaggtcttgatcttaatgatgtcacacatgtatataattatga
tttcccaaggaatattgacgtatatgtacacagagtagggtacattggacggacaggaaagactg
gcacatcagttaccctcatcactcagagagattcgaaaatggccggtgaattgattaaaattctg
gacagagcaaatcagagtgttccggaagatcttgtagtaatggctgagcaatacaagttaaatca
acaaaagaggcacagagaaacacgatcaagaaaacctggacaaagacgcaaggagttttattttt
taagttga
[Chemical Formula 36]
DDX54 DNA sequence
(SEQ ID NO: 53)
gcggccgacaagggcccggcggctggacctcggtcgcgagctgccatggcccagtggaggaagaa
gaaagggctccggaagcgccgaggcgcggcctcccaggcccgcggcagcgactcggaggacggcg
agtttgagatccaggcggaagatgacgcccgggcccggaagctgggacctggaagacccctgccc
accttccccacctcggaatgcacctcggatgtggagccggacacccgggagatggtgcgtgccca
gaacaagaagaagaagaagtctggaggcttccagtccatgggcctgagctacccggtgttcaaag
gcatcatgaagaaggggtacaaggtgccaacacccatccagaggaagaccatcccggtgatcttg
gatggcaaggacgtggtggccatggcccggacgggcagtggcaagacagcctgcttcctcctccc
aatgttcgagcggctcaagacccacagtgcccagaccggggcccgcgccctcatcctctcgccga
cccgagagctggccctgcagaccctgaagttcactaaggagctaggcaagttcactggcctcaag
actgccctgatcctgggggagacaggatggaagaccagtttgcagccctgcacgaaaatcccgac
ataattattgccacgcccggacggttggtgcatgtggctgtggaaatgagcctgaagctgcagag
tgtggaatacgtggtgttcgatgaagctgaccggctttttgaaatgggtttcgcagagcagctgc
aggagatcatcgcccgcctccccgggggccaccagacggtgctgttctccgccacgctgcccaaa
ctgctggtggaatttgcccgggctggcctcacggagcccgtgctcatccggcttgacgtggatac
caagctcaacgagcagctgaagacctccttcttcctcgtggggaggacaccaaggctgccgtgct
gctccacctgctgcacaacgtggtgcggccccaggaccagaccgtggtgtttgtggccacgaagc
accacgccgagtacctcactgagctgctgacgacccagcgggtgagctgcgcccacatctacagt
gccctagacccgacagcccgcaagatcaatctcgccaaattcacgcttggcaagtgctccactct
cattgtgactgacctggccgcccgaggcctggacatcccgctgctggacaatgtcatcaactaca
gcttccccgccaagggcaaactcttcctgcaccgcgtgggccgtgtggctcgggctggccgaagt
ggcacagcctactccttggtggcccctgatgaaatcccctacctgctggatctgcacctgttcct
gggccgctccctcaccctcgcccgacccctcaaggagccctcaggtgtggccggtgtggatggca
tgctgggtcgggtgccacagagtgtggtggacgaggaggacagtggtctgcagagcaccctggag
gcatcgctggagctacggggcctggcccgcgttgctgataacgcccagcagcagtatgtgcgctc
acgcccggcgccctcgcctgagtccatcaagagggccaaggagatggaccttgtggggctgggcc
tgcaccccctcttcagctcgcgttttgaggaggaggagctgcagcggctgaggctggtggacagc
ataaagaactaccgctcccggggactatctttgagatcaacgcctccagccgagacctgtgcagc
caggtgatgcgcgccaagcggcagaaggaccgcaaggccatcgcccgcttccagcagggacagca
ggggcggcaggagcagcaggagggcccagtgggcccagccccgagccgcccagcactgcaggaga
agcagcctgagaaggaggaggaggaggagggggagagagtgtggaggacattttctcagaggtcg
tgggccggaagggcagcggtcaggacccaacaggggagccaagaggcggagggaggaggcccggc
agcgggaccaggaattctacatcccctaccggcccaaggactttgacagcgagcggggcctgagc
atcagcgcggaagggggagcctttgagcagcaggcagctggcgctgtcctggacttgatggggga
tgaagcccagaacctgacgaggggccggcagcagctcaagtgggaccgtaagaagaagcggtttg
tgggacagtcaggacaggaagacaagaagaagattaagacagagagcggccgctacatcagcagc
tcctacaagcgagacctctatcagaagtggaaacagaaacagaaaattgatgatcgtgactcgga
cgaagaaggggcatctgaccggcgaggcccagagcgaagagggggaagcgagaccgtggccaagg
tgcatcccggccccacgccccaggcacccctgcaggccgagtccgcccggaactcaagaccaagc
agcagatcctgaagcagcggcgccgggcccagaagctgcacttcctgcagcgtggtggcctcaag
cagctctctgcccgcaaccgccgccgcgtccaggagctgcagcagggcgccttcggccggggtgc
ccgctccaagaagggcaagatgcggaagaggatgtga
[Chemical Formula 37]
DDXSS DNA sequence
(SEQ ID NO: 54)
gagcatgtgacagagggctcctgggagtcgctgcctgtgccgctgcacccgcaggtgctgcgcgc
gctgcgggagctgggcttcccgtacatgacgccggtgcagtccgcaaccatccctctgttcatgc
gaaacaaagatgtcgctgcagaagcggtcacaggtagtggcaaaacactcgcttttgtcatcccc
atcctggaaattcttctgagaagagaagagaagttaaaaaagagtcaggttggagccataatcat
cacccccactcgagagctggccattcaaatagacgaggtcctgtcgcatttcacgaagcacttcc
ccgagttcagccagattctttggatcggaggcaggaatcctggagaagatgttgagaggtttaag
caacaagggggaacatcattgtggccactccaggccgcttggaggacatgttccggaggaaggcc
gaaggcttggatctggccagctgtggcgatccctggatgtcctggtgttggatgaggcagacaga
cttctggacatggggtttgaggcaagcatcaacaccattctggagtttttgccaaagcagaggag
aacaggccttttctctgccactcagacgcaggaagtggagaacctggtgagagcgggcctccgga
accctgtccgggtctcagtgaaggagaagggcgtggcagccagcagtgcccagaagaccccctcc
cgcctggaaaactactacatggtatgcaaggcagatgagaaatttaatcagctggtccattttct
tcgcaatcataagcaggagaaacacctggtcttcttcagcacctgtgcctgtgtggaatactatg
ggaaggctctggaagtgctggtgaagggcgtgaagattatgtgcattcacggaaagatgaaatat
aaacgcaataagatcttcatggagttccgcaaattgcaaagtgggattttagtgtgcactgatgt
gatggcccggggaattgatattcctgaagtcaactgggttttgcagtatgaccccccagcaatgc
aagtgccttcgtgcatcgctgcggtcgcacagctcgcattggccacgggggcagcgctctggtgt
tcctcctgcccatggaagagtcatacatcaatttccttgcaattaaccaaaaatgccccctgcag
gagatgaagccccagagaaacacagcggaccttctgccaaaactcaagtccatggccctggctga
cagagctgtgtttgaaaagggcatgaaagcttttgtgtcatatgtccaagcttatgcaaagcatg
aatgcaacctgattttcagattaaaggatcttgattttgccagccttgctcgaggttttgccctg
ctgaggatgcccaagatgccagaattgagaggaaagcagtttccagattttgtgcccgtggacgt
taataccgacacgattccatttaaagataaaatcagagaaaagcagaggcagaaactcctggagc
aacaaagaagagagaaaacagaaaatgaagggagaagaaaattcataaaaaataaagcttggtca
aagcagaaggccaaaaaagaaaagaagaaaaaaatgaatgagaaaaggaaaagggaagagggttc
tgatattgaagatgaggacatggaagaacttcttaatgacacaagactcttgaaaaaacttaaga
aaggcaaaataactgaagaagaatttgagaagggcttgttgacaactggcaaaagaacaatcaag
acagtggatttagggatctcagatttggaagatgactgctga
[Chemical Formula 38]
DDX56 DNA sequence
(SEQ ID NO: 55)
gaggactctgaagcactgggcttcgaacacatgggcctcgatccccggctccttcaggctgtcac
cgatctgggctggtcgcgacctacgctgatccaggagaaggccatcccactggccctagaaggga
aggacctcctggctcgggcccgcacgggctccgggaagacggccgcttatgctattccgatgctg
cagctgttgctccataggaaggcgacaggtccggtggtagaacaggcagtgagaggccttgttct
tgttcctaccaaggagctggcacggcaagcacagtccatgattcagcagctggctacctactgtg
ctcgggatgtccgagtggccaatgtctcagctgctgaagactcagtctctcagagagctgtgctg
atggagaagccagatgtggtagtagggaccccatctcgcatattaagccacttgcagcaagacag
cctganacttcgtgactccctggagcttttggtggtggacgaagctgaccttcttttttcctttg
gctttgaagaagagctcaagagtctcctctgtcacttgccccggatttaccaggcttttctcatg
tcagctacttttaacgaggacgtacaagcactcaaggagctgatattacataacccggttaccct
taagttacaggagtcccagctgcctgggccagaccagttacagcagtttcaggtggtctgtgaga
ctgaggaagacaaattcctcctgctgtatgccctgctcaagctgtcattgattcggggcaagtct
ctgctctttgtcaacactctagaacggagttaccggctacgcctgttcttggaacagttcagcat
ccccacctgtgtgctcaatggagagcttccactgcgctccaggtgccacatcatctcacagttca
accaaggcttctacgactgtgtcatagcaactgatgctgaagtcctgggggccccagtcaagggc
aagcgtcggggccgagggcccaaaggggacaaggcctctgatccggaagcaggtgtggcccgggg
catagacttccaccatgtgtctgctgtgctcaactttgatcttcccccaacccctgaggcctaca
tccatcgagctggcaggacagcacgcgctaacaacccaggcatagtcttaacctttgtgcttccc
acggagcagttccacttaggcaagattgaggagcttctcagtggagagaacaggggccccattct
gctcccctaccagttccggatggaggagatcgagggcttccgctatcgctgcagggatgccatgc
gctcagtgactaagcaggccattcgggaggcaagattgaaggagatcaaggaagagcttctgcat
tctgagaagcttaagacatactttgaagacaaccctagggacctccagctgctgcggcatgacc
tacctttgcaccccgcagtggtgaagccccacctgggccatgttcctgactacctggttcctcct
gctctccgtggcctggtgcgccctcacaagaagcggaagaagctgtcttcctcttgtaggaaggc
caagagagcaaagtcccagaacccactgcgcagcttcaagcacaaaggaaagaaattcagaccca
cagccaagccctcctga
[Chemical Formula 39]
DDX58 DNA sequence
(SEQ ID NO: 56)
accaccgagcagcgacgcagcctgcaagccttccaggattatatccggaagaccctggaccctac
ctacatcctgagctacatggccccctggtttagggagggttattctggactttatgaagccattg
aaagttgggatttcaaaaaaattgaaaagttggaggagtatagattacttttaaaacgtttacaa
ccagaatttaaaaccagaattatcccaaccgatatcatttctgatctgtctgaatgtttaattaa
tcaggaatgtgaagaaattctacagatttgctctactaaggggatgatggcaggtgcagagaaat
tggtggaatgccttctcagatcagacaaggaaaactggcccaaaactttgaaacttgctttggag
aaagaaaggaacaagttcagtgaactgtggattgtagagaaaggtataaaagatgttgaaacaga
agatcttgaggataagatggaaacttctgacatacagattttctaccaagaagatccagaatgcc
agaatcttagtgagaattcatgtccaccttcagaagtgtctgatacaaacttgtacagcccattt
aaaccaagaaattaccaattagagcttgctttgcctgctatgaaaggaaaaaacacaataatatg
tgctcctacaggttgtggaaaaacctttgtttcactgcttatatgtgaacatcatcttaaaaaat
tcccacaaggacaaaaggggaaagttgtcttttttgcgaatcagatcccagtgtatgaacagcag
aaatctgtattctcaaaatactttgaaagacatgggtatagagttacaggcatttctggagcaac
agctgagaatgtcccagtggaacagattgttgagaacaatgacatcatcattttaactccacaga
ttcttctgaacaaccttaaaaagggaacgattccatcactatccatctttactttgatgatattt
gatgaatgccacaacactagtaaacaacacccgtacaatatgatcatgtttaattatctagatca
gaaacttggaggatcttcaggcccactgccccaggtcattgggctgactgcctcggttggtgttg
gggatgccaaaaacacagatgaagccttggattatatctgcaagctgtgtgcttctcttgatgcg
tcagtgatagcaacagtcaaacacaatctggaggaactggagcaagttgtttataagccccagaa
gtttttcaggaaagtggaatcacggattagcgacaaatttaaatacatcatagctcagctgatga
ggacacagagagtctggcaaagagaatctgcaaagacctcgaaaacttatctcaaattcaaaata
gggaatttggaacacagaaatatgaacaatggattgttacagttcagaaagcatgcatggtgttc
cagatgccagacaaagatgaagagagcaggatttgtaaagccctgtttttatacacttcacattt
gcggaaatataatgatgccctcattatcagtgagcatgcacgaatgaaagatgctctggattact
tgaaagacttcttcagcaatgtccgagcagcaggattcgatgagattgagcaagatcttactcag
agatttgaagaaaagctgcaggaactagaaagtgtttccagggatcccagcaatgagaatcctaa
acttgaagacctctgcttcatcttacaagaagagtaccacttaaacccagagacaataacaattc
tctttgtgaaaaccagagcacttgtggacgctttaaaaaattggattgaaggaaatcctaaactc
agttttctaaaacctggcatattgactggacgtggcaaaacaaatcagaacacaggaatgaccct
cccggcacagaagtgtatattggatgcattcaaagccagtggagatcacaatattctgattgcca
cctcagttgctgatgaaggcattgacattgcacagtgcaatcttgtcatcctttatgagtatgtg
ggcaatgtcatcaaaatgatccaaaccagaggcagaggaagagcaagaggtagcaagtgcttcct
tctgactagtaatgctggtgtaattgaaaaagaacaaataaacatgtacaaagaaaaaatgatga
atgactctattttacgccttcagacatgggacgaagcagtatttagggaaaagattctgcatata
cagactcatgaaaaattcatcagagatagtcaagaaaaaccaaaacctgtacctgataaggaaaa
aaaaaactgctctgcagaaagtgcaaagccttggcatgttacacagctgacgtaagagtgataga
ggaatgccattacactgtgcttggagargctttraaggaatgctttgtgagtagaccacatccca
agccaaagcagttttcaagttttgaaaaaagagcaaagatattctgtgcccgacagaactgcagc
catgactggggaatccatgtgaagtacaagacatttgagattccagttataaaaattgaaagttt
tgtggtggaggatattgcaactggagttcagacactgtactcgaagtggaaggactttcattttg
agaagataccatttgatccagcagaaatgtccaaatga
[Chemical Formula 40]
DDX59 DNA sequence
(SEQ ID NO: 57)
tttgttccaagatctctaaaaatcaagaggaatgctaatgatgatggcaaaagttgtgtggctaa
gataattaaaccagacccagaagaccttcagttggacaaaagcagagatgttcccgttgagctgt
agctacagaagcagccacaatagacaggcacatcagcgaatcatgccctttccccagcccaggtg
gccagttggcagaggttcattcagtaagtcccgagcagggtgcgaaggacagccatccttctgaa
gagcccgttaagtcattttccaaaacacagcgctgggcagaaccaggggaacccatctgtgttgt
ctgtggtcgttatggagagtatatctgtgataagacagatgaagatgtgtgtagtttggagtgta
aagcgaaacatcttctacaagttaaggaaaaggaagagaaatcaaaactcagcaatccacagaag
gctgattctgagccagagtctccactgaatgcttcctatgtctacaaagagcacccctttatttt
gaaccttcaggaagaccagattgaaaatcttaaacagcagctgggaattttagttcaagggcaag
aagtcaccaggcccattattgactttgaacattgtagtctccctgaggtcttaaatcacaacttg
aagaaatcaggctatgaggtgccaactcccattcaaatgcagatgattcctgtgggacttctggg
aagagacattctggccagtgcagatactggctcaggaaaaacagctgcttttcttcttcctctta
tcatgcgagctttattcgagagcaaaactccatctgcgctcattcttacaccaaccagagagtt
agccattcagatagagagacaagctaaagaattgatgagtggcctgccacgcatgaaaactgtgc
ttcttgtagggggcttacccttacccccacagctttatcgtctgcaacaacatgttaaggttatc
atagcaacccctgggcgacttctggatataataaagcagagctctgtagaactctgtggtgtaaa
gattgtggtagtagatgaagctgataccatgttaaagatgggttttcaacaacaagtgcttgaca
ttttggaaaacattcctaatgattgtcagaccattttggtttcagccacaattccaactagcata
gaacagctagchagccagcttctgcataatcctgtgagaattatcactggagaaaagaacctacc
ttgtgccaatgtacgtcagattattttgtgggtagaagacccagccaaaaagaaaaaattatttg
aaattttaaatgataagaaactctttaagcctccagtgttagtatttgtggactgcaaactagga
gcagatcttttgagtgaagccgttcagaaaatcacagggctgaaaagcatatctatacattcaga
gaagtcgcaaatagaaaggaaaaacatattgaagggattacttgaaggagactatgaagttgtag
tgagcacaggagtcttgggacgaggcctagacttgatcagtgtcaggctggttgtcaattttgat
atgccttcaagtatggatgagtatgtccatcagattggaagagtaggaagattaggtcaaaatgg
aacagcgattactttcatcaataataattcaaaaagactcttctgggatattgcaaaacgagtaa
agcccacaggatccattcttccccctcagttattaaattccccataccttcatgaccagaagaga
aaggaacaacagaaagataaacagacacagaatgatctggttacaggagctaatcttatggata
ttatcagaaaacatgataaaagtaattctcagaaatga

### 1-2. Method for Measuring Binding Affinity

Binding affinity can be measured by surface plasmon resonance assay. For example, an autophagy-inducing compound is immobilized on a gold thin film of a sensor chip, and a purified DEAD-box RNA helicase protein solution is flowed on the surface of the sensor chip to measure the SPR signal that changes depending on the binding state of both by BIAcore. A thermal shift assay called CETSA can be also used to measure binding affinity by detecting changes in the thermal stability of a DEAD-box RNA helicase protein caused by binding to an autophagy-inducing compound.

1-3. p62

p62 is a receptor protein that leads specific proteins and organelles to autophagy. p62 is diffusely localized in the cytoplasm and nucleus, has a half-life of about 10 hours, and is basically degraded in an autophagy-dependent manner. p62 has multiple domains, such as N-terminal Phox1 and Bem1p domains (Phox 1 and Bem1p: PB1), a zinc finger (Zinc finger: ZZ), a TRAF6-binding motif (TRAF6-binding domain: TB), an LC3 interaction region (LC3-interacting region: LIR, consisting of DDDWTHL sequence), a Keap1-interacting region (Keap1-interacting region: KIR) and a C-terminal ubiquitin-associated domain (ubiquitin-associated: UBA), and interacts with LC3, an autophagosome localization protein, at the LC3 interaction region (LIR) to lead to autophagy (see NON PATENT LITERATURE 13). The amino acid sequence of p62 and the nucleic acid sequence of the SQSTM1 gene encoding the amino acid sequence are as follows.

[Chemical Formula 41]
P62 Amino acid sequence
(SEQ ID NO: 5)
MASLTVKAYLLGKEDAAREIRRFSFCCSPEPEAEAEAAAGPGPCERLLSR
VAALFPALRPGGFQAHYRDEDGDLVAFSSDEELTMAMSYVKDDIFRIYIK
EKKECRRDHRPPCAQEAPRNMVHPNVICDGONGPVVGTRYKCSVCPDYDL
CSVCEGKGLHRGHTKLAFPSPFGHLSEGFSHSRWLRKVKHGHFGWPGWEM
GPPGNWSPRPPRAGEARPGPTAESASGPSEDPSVNFLKNVGESVAAALSP
LGIEVDIDVEHGGKRSRLTPVSPESSSTEEKSSSQPSSCCSDPSKPGGNV
EGATQSLAEQMRKIALESEGRPEEQMESDNCSGGDDDWTHLSSKEVDPST
GELQSLQMPESEGPSSLDPSQEGPTGLKEAALYPHLPPEADPRLIESLSQ
MLSMGFSDEGGWLTRLLQTKNYDIGAALDTIQYSKHPPPL
[Chemical Formula 42]
P62 DNA sequence
(SEQ ID NO: 6)
atggcgtcgctcaccgtgaaggcctaccttctgggcaaggaggacgcggc
gcgcgagattcgccgcttcagcttctgctgcagccccgagcctgaggcgg
aagccgaggctgcggcgggtccgggaccctgcgagcggctgctgagccgg
gtggccgccctgttccccgcgctgcggcctggcggcttccaggcgcacta
ccgcgatgaggacggggacttggttgccttttccagtgacgaggaattga
caatggccatgtcctacgtgaaggatgacatcttccgaatctacattaaa
gagaaaaaagagtgccggcgggaccaccgcccaccgtgtgctcaggaggc
gccccgcaacatggtgcaccccaatgtgatctgcgatggctgcaatgggc
ctgtggtaggaacccgctacaagtgcagcgtctgcccagactacgacttg
tgtagcgtctgcgagggaaagggcttgcaccgggggcacaccaagctcgc
attccccagccccttcgggcacctgtctgagggcttctcgcacagccgct
ggctccggaaggtgaaacacggacacttcgggtggccaggatgggaaatg
ggtccaccaggaaactggagcccacgtcctcctcgtgcaggggaggcccg
ccctggccccacggcagaatcagcttctggtccatcggaggatccgagtg
tgaatttcctgaagaacgttggggagagtgtggcagctgcccttagccct
ctgggcattgaagttgatatcgatgtggagcacggagggaaaagaagccg
cctgacccccgtctctccagagagttccagcacagaggagaagagcagct
cacagccaagcagctgctgctctgaccccagcaagccgggtgggaatgtt
gagggcgccacgcagtctctggcggagcagatgaggaagatcgccttgga
gtccgaggggcgccctgaggaacagatggagtcggataactgttcaggag
gagatgatgactggacccatctgtcttcaaaagaagtggacccgtctaca
ggtgaactccagtccctacagatgccagaatccgaagggccaagctctct
ggacccctcccaggagggacccacagggctgaaggaagctgccttgtacc
cacatctcccgccagaggctgacccgcggctgattgagtccctctcccag
atgctgtccatgggcttctctgatgaaggcggctggctcaccaggctcct
gcagaccaagaactatgacatcggagcggctctggacaccatccagtatt
caaagcatcccccgccgttgtag

### 1-4. Method for Detecting Interaction (Proximity) Between DEAD-Box RNA Helicase and p62

The present invention is not particularly limited in terms of a method for detecting the interaction (proximity) between DEAD-box RNA helicase and p62 and may be performed by an available method. For example, the detection can be performed using NanoLuc (registered trademark) Binary Technology Assays (NanoBIT (registered trademark) Assays) according to the following reaction scheme:

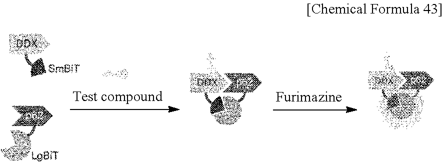

wherein, LgBiT is Large BiT and SmBiT is an abbreviation for Small BIT.

In this assay, prepared are cells expressing a modified DEAD-box RNA helicase (SmBiT-DDX or LgBiT-DDX) and p62 (LgBiT-p62 or SmBiT-p62) in which a DEAD-box RNA helicase and p62 are each fused with a partial peptide that constitutes a part of luciferase and forms a complete luciferase by both of them. When the cells are contacted with a test compound, the test compound brings the DEAD-box RNA helicase and p62 close to each other to associate the partial peptides of luciferase fused therewith, SmBiT and LgBiT, with each other, thereby restoring luciferase activity. In this condition, adding furimazine, a substrate of luciferase, results in strong luminescence, enabling detection of the proximity (interaction) between DEAD-box RNA helicase and p62.

### 1-5. Detection of Droplet Formation of DEAD-Box RNA Helicase and p62

As used herein, the “droplet” refers to a liquid-like aggregate in which a DEAD-box RNA helicase, a compound associated therewith, and p62 are concentrated and which is liquid-liquid phase-separated from the outside.

Such a “droplet” can be confirmed by labeling a DEAD-box RNA helicase and/or p62 with a fluorescent label or the like, introducing a test compound into a system in which they exist, preferably treating cells expressing at least one of these with the test compound, and observing the presence of a substance visualized by the label with a fluorescence microscope or the like. In addition, for the purpose of confirming the properties of the liquid in more detail, it is preferable to combine this method with the fluorescence recovery after photobleaching (FRAP).

2. Method for Producing Compound that Induces Degradation of Target Intracellular Molecule or Organelle by Autophagy

A compound that induces degradation of a target intracellular molecule by autophagy can be produced by linking a ligand that specifically binds to a target intracellular molecule or organelle to a compound obtained by the above-described screening method via a linker.

As used herein, the “target intracellular molecule or organelle” means an intracellular molecule or organelle targeted for degradation by autophagy. The “intracellular molecule” means a biomolecule at least a portion of which is present in a cell, and includes not only a natural biomolecule present in a cell but also a molecule artificially introduced or expressed in a cell. In addition, the “organelle” includes not only natural organelles present in a cell (for example, dysfunctional mitochondria) but also foreign viruses or microorganisms that have invaded a cell.

Examples of the “intracellular molecule” include lipids, glycolipids, proteins, glycoproteins, and the like present in a cell, and are typically proteins present in a cell, and include aggregates of proteins such as amyloid β (Aβ), tau protein, and α-synuclein. The cell type in which the “intracellular molecule” is present is not particularly limited, but a cell of a mammal (e.g., mice, rats, hamsters, rabbits, cats, dogs, cattle, sheep, monkeys, and humans) is preferred, and a human cell is most practically kept in mind. Examples of the intracellular protein include those associated with pathological conditions (particularly, those associated with human pathological conditions) as practical targets, which have been extensively reported. Such pathological condition-associated intracellular proteins can be confirmed, for example, in UniProt or Human Protein Atlas, and a few examples thereof include Aβ, tau protein, α-synuclein or aggregates thereof (involved in neurodegenerative diseases such as Alzheimer's disease and Parkinson's disease), BRD4 (involved in multiple myeloma, acute myelogenous leukemia, etc.), Ras (involved in colorectal cancer, pancreatic cancer, etc.), calcineurin, CyP, FRB, FKBP, PyL, ABI1, GID1, GA1, FKBP F36V, IAA17 TIR1, EGFR, BTK (B. Z. Stanton et al. Science; 359; eaao5902 (2018), N. Shindo et al. Bioorg. Med Chem; 47:116386 (2021) and the like, the contents of which are incorporated herein by reference).

In addition, the intracellular molecule includes, for example, an intracellular protein in which a predetermined tag is fused to an arbitrary target intracellular molecule or organelle by a tagging technology such as HaloTag (registered trademark), SNAP-tag (registered trademark), or CLIP-tag (registered trademark) to enable binding of a ligand described later. An intracellular protein fused with HaloTag (registered trademark) is one fused with a tag having an F272H mutant of Rhodococcus rhodochrous-derived dehalogenating enzyme DhaA, an intracellular protein fused with SNAP-tag (registered trademark) is one fused with a tag having a human-derived 06-alkylguanine-DNA alkyltransferase hATG mutant, and an intracellular protein fused with CLIP-tag (registered trademark) is one fused with a tag having an E30R mutant of the SNAP-tag protein. These are overviewed, for example, in Halo SNAP CLIP for Protein Labeling—Bitesize Bio, the contents of which are incorporated herein by reference.

Also, intracellular proteins tagged with a mutant FKBP, such as α-synuclein tagged with a mutant FKBP or aggregates thereof, can be targeted intracellular molecules for autophagy.

As used herein, the “ligand that specifically binds to an intracellular molecule or organelle” means a structural unit that constitutes a part of a compound that induces degradation of a target intracellular molecule or organelle by autophagy, and that has a specific binding activity to the target intracellular molecule (this includes the tagged fusion proteins described above) or the organelle. Therefore, a substance constituting the ligand is one having a specific binding activity to an intracellular molecule or organelle, and there are numerous reports on these substances. For example, these can be confirmed in BindingDB (https://www.bindingdb.org/rwd/bind/index.jsp), and only a few examples thereof include, e.g., FK506 (calcineurin), cyclosporin A (calcineurin, CyP), FKCsA (CyP, FKBP), rapamycin (FRB, FKBP), FK1012 (FKBP), abscisic acid (PyI, ABI1), gibberellin 3 (GID1, GA1), AP1903/AP20187 (FKBP F36V), Auxin (IAA17 TIR1), afatinib (EGFR), osimertinib (EGFR), dacomitinib (EGFR), neratinib (EGFR), sotorasib (EGFR), ibrutinib (BTK), acalabrutinib (BTK), zanubrutinib (BTK), tirabrutinib (BTK), adagrasib (BTK) (those in parentheses indicate binding targets).

In addition, examples of the ligand for BRD4 include:

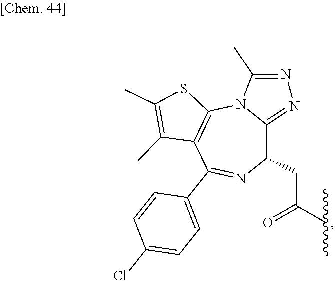 [SMILES: *C(=O)C[C@@H]1N=C(c2ccc(Cl)cc2)c2c(sc(C)c2C)-n2c(C)nnc21]

examples of the ligand for FKBP12 include:

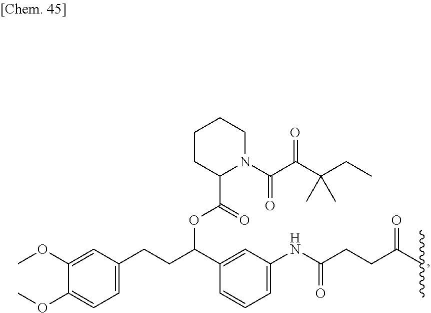 [SMILES: *C(=O)CCC(=O)Nc1cccc(C(CCc2ccc(OC)c(OC)c2)OC(=O)C2CCCCN2C(=O)C(=O)C(C)(C)CC)c1]

and examples of the ligand for MetAP2 include:

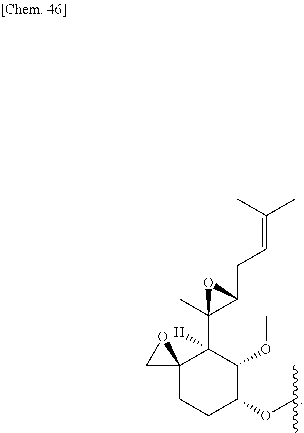 [SMILES: *O[C@@H]1CC[C@]2(CO2)[C@@H]([C@@]2(C)O[C@@H]2CC=C(C)C)[C@@H]1OC]

Examples of the ligand compound for Ras include ligands described in the following literatures; Nature, 2013, 503, 548-551; WO 2013/155223 A; Science, 2016, 351, 604-608; WO 2014/152588 A; WO 2015/054572 A; WO 2016/049524 A; Nat. Rev. Drug Discov., 2014, 13, 828-851; Chem. Soc. Rev., 2016, advance article (DOI: 10.1039/C5CS00911A). Therefore, the ligand for Ras can be composed of these compounds. Examples of the ligand for Ras include:

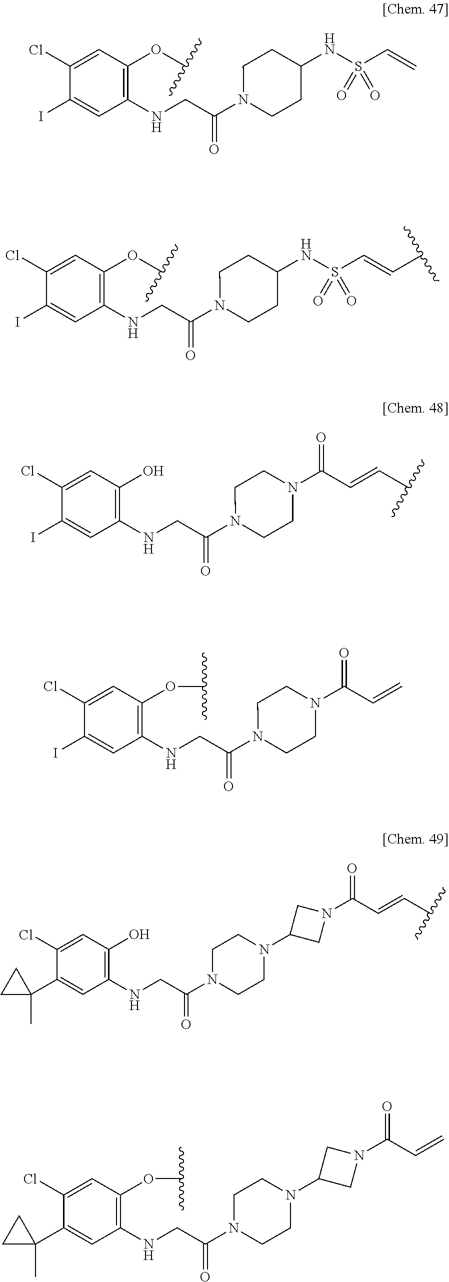 [SMILES: *Oc1cc(Cl)c(I)cc1NCC(=O)N1CCC(NS(=O)(=O)C=C)CC1 ; */C=C/S(=O)(=O)NC1CCN(C(=O)CNc2cc(I)c(Cl)cc2O*)CC1 ; */C=C/C(=O)N1CCN(C(=O)CNc2cc(I)c(Cl)cc2O)CC1 ; *Oc1cc(Cl)c(I)cc1NCC(=O)N1CCN(C(=O)C=C)CC1 ; */C=C/C(=O)N1CC(N2CCN(C(=O)CNc3cc(C4(C)CC4)c(Cl)cc3O)CC2)C1 ; *Oc1cc(Cl)c(C2(C)CC2)cc1NCC(=O)N1CCN(C2CN(C(=O)C=C)C2)CC1]

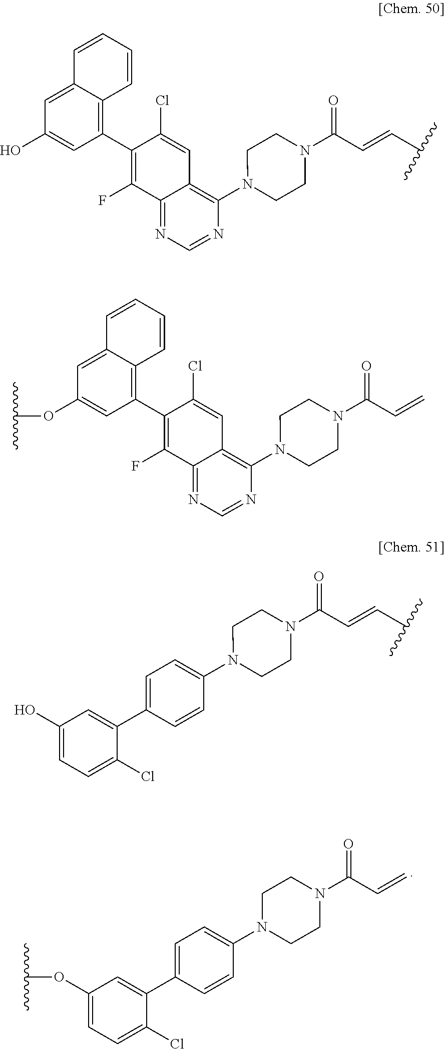 [SMILES: */C=C/C(=O)N1CCN(c2ncnc3c(F)c(-c4cc(O)cc5ccccc45)c(Cl)cc23)CC1 ; *Oc1cc(-c2c(Cl)cc3c(N4CCN(C(=O)C=C)CC4)ncnc3c2F)c2ccccc2c1 ; */C=C/C(=O)N1CCN(c2ccc(-c3cc(O)ccc3Cl)cc2)CC1 ; *Oc1ccc(Cl)c(-c2ccc(N3CCN(C(=O)C=C)CC3)cc2)c1]

Examples of the ligand for an intracellular protein containing HaloTag (registered trademark) fused therein include halogenated C₁₋₁₂ alkoxy groups (particularly, terminal carbon-halogenated C₁₋₆ alkoxy groups), and are preferably a halogenated (particularly, terminal carbon-halogenated) hexyloxy group (particularly, a chlorinated hexyloxy group).

Examples of the ligand for an intracellular protein containing SNAP-tag (registered trademark) fused therein include 06-benzylguanine.

Examples of the ligand for an intracellular protein containing CLIP-tag (registered trademark) fused therein include 06-benzylcytosine.

The ligands for the tag-fused intracellular proteins are overviewed in Halo SNAP CLIP for Protein Labeling—Bitesize Bio, the contents of which are incorporated herein by reference.

Examples of the ligand for a target intracellular molecule tagged with a mutant FKBP, such as α-synuclein tagged with a mutant FKBP or aggregates thereof, include:

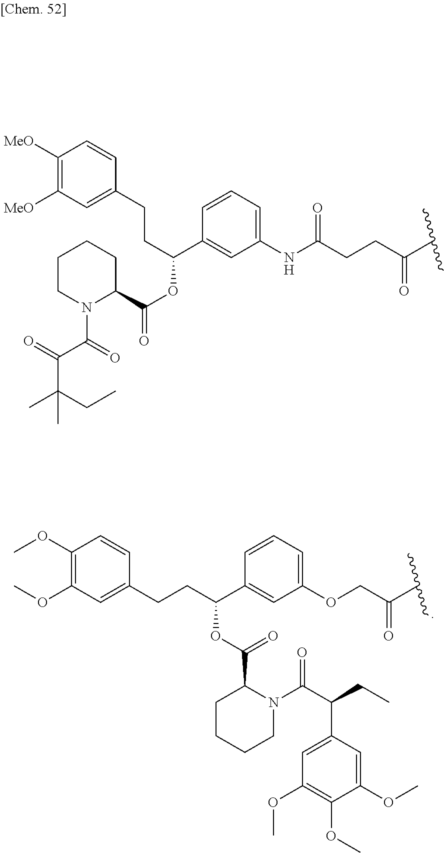

As used herein, the “linker” means a group of atoms linking the “ligand” moiety and a tag moiety that induces autophagy in a compound of the present invention. The linker structure can utilize known linker structures utilized in linking molecules having different functions. In addition, WO 2024/054876 A, JP-B-7161760, and NON PATENT LITERATURES 5, 9, and 10 describe in detail the structures of linkers which connect intracellular molecules and tag moieties that induce autophagy, and the structures described in these literatures can be used.

In addition, the linkage between the “linkers”, and the “ligands” or the tag moieties can also be performed using known reactions. For example, the reaction can be performed according to methods described in JP-B-7161760; NON PATENT LITERATURES 5, 9 and 10; The Fifth Series of Experimental Chemistry, Vol. 13 to Vol. 19 (edited by The Chemical Society of Japan); Shin Jikken Kagaku Koza (New Experimental Chemistry), Vol. 14 to Vol. 15 (edited by The Chemical Society of Japan); Reactions and Syntheses: In the Organic Chemistry Laboratory, Revised, 2nd Ed. (L. F. Tietze, Th. Eicher, Nankodo Co., Ltd.); Organic Name Reactions; The Reaction Mechanism and Essence, Revised (written by Hideo Tougo, Kodansha Ltd.); Organic Syntheses Collective Volumes I to VII (John Wiley & Sons, Inc.); Modern Organic Synthesis in the Laboratory: A Collection of Standard Experimental Procedures (written by Jie Jack Li, Oxford University Press); Comprehensive Heterocyclic Chemistry III, Vol. 1 to Vol. 14 (Elsevier Japan KK); Strategic Applications of Named Reactions in Organic Synthesis (translated by Kiyoshi Tomioka, published by Kagaku-Dojin Publishing Company, Inc.); Comprehensive Organic Transformations (VCH Publishers, Inc.) (published in 1989), “Protective Groups in Organic Synthesis, 4th Ed.” (written by Theodora W. Greene, Peter G. M. Wuts) Wiley-Interscience (published in 2007); “Protecting Groups, 3rd Ed.” (written by P. J. Kocienski) Thieme (published in 2004), or the like. The contents of these literatures are incorporated herein by reference.

Examples of the reaction to be used include a functional group protection or deprotection reaction, a reduction reaction, an oxidation reaction, an aromatic nucleophilic substitution reaction, an azidation reaction of alcohols, alkyl halides, and sulfonate esters, a reductive amination reaction, a Mitsunobu reaction, an esterification reaction, an amidation reaction, or a urea reaction, a coupling reaction, a Wohl-Ziegler reaction, a sulfon esterification reaction, a hydrolysis reaction, a bromination reaction, an N-alkylation reaction, a hydroxylation reaction via diazotization, a deamination reaction, a cyclization reaction, a carbamation reaction, an amidation by a carbon monoxide insertion reaction, an esterification, and a click reaction, and the like, and reagents and reaction conditions required for these reactions are well known to those skilled in the art to which the present invention belongs.

In the compounds obtained by the above production method, a ligand that specifically binds to a target molecule or organelle and a tag moiety that induces autophagy are linked via a linker, and the compounds have an advantage that a wide range of substrates can be targeted by replacing the ligand that binds to a target molecule or organelle depending on the target molecule or organelle.

When cells are treated with a compound obtained by the above production method in the presence of p62 in a situation in which the cells can contact with a DEAD-box RNA helicase, a target molecule or organelle is bound to a ligand to induce autophagy, and the target molecule or organelle can be degraded by autophagy. When the compound obtained by the above production method is brought into contact with a DEAD-box RNA helicase in the presence of p62 in a cell, a droplet containing the compound, a target molecule or organelle, p62 and a DEAD-box RNA helicase is formed. As a result, the phagophore extends to form autophagosomes surrounding the droplet, and the resulting autophagosomes fuse with endosomes or lysosomes to digest the contents.

Therefore, the compounds according to some embodiments of the present invention are useful as a prophylactic or therapeutic agent for a disease involving a target intracellular molecule or organelle. In particular, the compounds according to some embodiments of the present invention can be effective in the prevention or treatment of any disease involving target intracellular molecules or organelles in light of their mechanisms of action based on AUTAC. Among them, they are expected to be applied to the treatment or prevention of cancers, inflammatory diseases, autoimmune diseases, and bone and joint degenerative diseases.

### EXAMPLES

The present invention will be further described in detail by the following Examples, Test Examples, and Formulation Examples, but these do not limit the present invention, and may be changed without departing from the scope of the present invention.

Test 1. Construction of Screening Method for Compounds that Induce Interaction (Proximity) Between DDX5 and p62

Whether several compounds that degrade target intracellular molecules by autophagy or tag moieties thereof developed by the present inventors (see JP-B-7161760) induce an interaction between DDX5 and p62 was measured using the NanoBiT system. In this system, if the test compound induces an interaction between DOX5 and p62, the emission intensity increases, enabling detection of the presence of a compound with such activity.

### 1-1. Test Materials

Preparation of pLgBiT-p62 and pSmBiT-DDX5

To generate pLgBiT-p62, the pBiT1. 1-N [TK/LgBiT] Vector (Promega #N2014) was linearized by a polymerase chain reaction using oligo primers (5′-agcggtaggcatggcgtcgctcaccg-3′ and 5′-agaagatctgctagtacaacggcgggatgc-3′). p62 cDNA was synthesized from the total RNAs of HeLa cells using an oligo (dT)₁₅ primer, amplified using oligo primers (5′-agcggtaggcatggcgtcgctcaccg-3′ and 5′-agaagatctgctagtacaacggcgggatgc-3′), and inserted into a vector using an In-fusion cloning system (underlined 15 nt is In-fusion arm).

To generate pSmBiT-DDX5, the pBiT2.1-N [TK/SmBiT] Vector (Promega #N2014) was linearized by a polymerase chain reaction using oligo primers (5′-ggtggctttaccaacagtaccg-3′ and 5′-gctagcagatcttctagtcgg-3′). The DDX5 cDNA was synthesized from the total RNAs of HeLa cells using an oligo (dT)₁₅ primer, amplified using oligo primers (5′-agcggtggcatgtcgggattcgagtgaccg-3′ and 5′-agaagatctgctagcttattgggaatcctgttggcattg-3′), and inserted into a vector using an In-fusion cloning system (underlined 15 nt is In-fusion arm).

The nucleic acid sequences of pLgBiT-p62 and pSmBiT-DDX5 carried by the respective vectors are shown below.

[Chemical Formula 53]
Nucleic acid sequence of pLgBiT-p62
(SEQ ID NO: 15)
ggcctaactggccggtacctgagtctaaatgagtcttcggacctcgcggg
ggccgcttaagcggtggttagggtttgtctgacgcggggggagggggaag
gaacgaaacactctcattcggaggcggctcggggtttggtcttggtggcc
acgggcacgcagaagagcgccgcgatcctcttaagcacccccccgccctc
cgtggaggcgggggtttggtcggcgggtggtaactggcgggccgctgact
cgggcgggtcgcgcgccccagagtgtgaccttttcggtctgctcgcagac
ccccgggcggcgccgccgcggcggcgacgggctcgctgggtcctaggctc
catggggaccgtatacgtggacaggctctggagcatccgcacgactgcgg
tgatattaccggagaccttctgcgggacgagccgggtcacgcggctgacg
cggagcgtccgttgggcgacaaacaccaggacggggcacaggtacactat
cttgtcacccggaggcgcgagggactgcaggagcttcagggagtggcgca
gctgcttcatccccgtggcccgttgctcgcgtttgctggcggtgtccccg
gaagaaatatatttgcatgtctttagttctatgatgacacaaaccccgcc
cagcgtcttgtcattggcgaagtcgaacacgcagatgcagtcggggcggc
gcggtcccaggtccacttcgcatattaaggtgacgcgtgtggcctcgaac
accgagcgaccctgcagcgacccgcttaaaagcttggcaatccggtactg
ttggtaaagccaccatggtcttcacactcgaagatttcgttggggactgg
gaacagacagccgcctacaacctggaccaagtccttgaacagggaggtgt
gtccagtttgctgcagaatctcgccgtgtccgtaactccgatccaaagga
ttgtccggagcggtgaaaatgccctgaagatcgacatccatgtcatcatc
ccgtatgaaggtctgagcgccgac
[Chemical Formula 54]
Nucleic acid sequence of pSmBIT-DDX5
(SEQ ID NO: 16)
ggcctaactggccggtacctgagtctaaatgagtcttcggacctcgcggg
ggccgcttaagcggtggttagggtttgtctgacgcggggggagggggaag
gaacgaaacactctcattcggaggcggctcggggtttggtcttggtggcc
acgggcacgcagaagagcgccgcgatcctcttaagcacccccccgccctc
cgtggaggcgggggtttggtcggcgggtggtaactggcgggccgctgact
cgggcgggtcgcgcgccccagagtgtgaccttttcggtctgctcgcagac
ccccgggcggcgccgccgcggcggcgacgggctcgctgggtcctaggctc
catggggaccgtatacgtggacaggctctggagcatccgcacgactgcgg
tgatattaccggagaccttctgcgggacgagccgggtcacgcggctgacg
cggagcgtccgttgggcgacaaacaccaggacggggcacaggtacactat
cttgtcacccggaggcgcgagggactgcaggagcttcagggagtggcgca
gctgcttcatccccgtggcccgttgctcgcgtttgctggcggtgtccccg
gaagaaatatatttgcatgtctttagttctatgatgacacaaaccccgcc
cagcgtcttgtcattggcgaagtcgaacacgcagatgcagtcggggcggc
gcggtcccaggtccacttcgcatattaaggtgacgcgtgtggcctcgaac
accgagcgaccctgcagcgacccgcttaaaagcttggcaatccggtactg
ttggtaaagccaccatggtgaccggctaccggctgttcgaggagattctc
gggagttccggtggtggcgggagcggaggtggaggcatgtcgggttattc
gagtgaccgagaccgcggccgggaccgagggtttggtgcacctcgatttg
gaggaagtagggcagggcccttatctggaaagaagtttggaaaccctggg
gagaaattagttaaaaagaagtgg

### Resources and Reagents

TABLE 1
HeLa Cells: RIKEN BRC Cell Bank #RCB0007
DMEM: FUJIFILM Wako #043-30085
FBS: Thermo Fisher Scientific #10437028
Non-essential amino acids: FUJIFILM Wako #139-15651
Opti-MEM I: Thermo Fisher Scientific #11058-021
96-Well culture plate. Promega #E5650
Lipofectamine3000 transfection reagent: Thermo Fisher Scientific
#L3000008
Nano-Glo (registered trademark) Live Cell Reagent: Promega #N2014
GloMax Discover Microplate Reader: Promega #GM3000
PrimeSTAR Max DNA polymerase: TaKaRa #R045A
TRIzol reagent (for total RNA extraction): Thermo Fisher
Scientific #15596026
PrimeScript (trademark) RT reagent Kit (for cDNA synthesis):
TaKaRa #RR037A
In-Fusion HD Cloning Kit: TaKaRa #Z9649N

### Test Compounds

The following compounds were used to verify the screening method of the present invention.

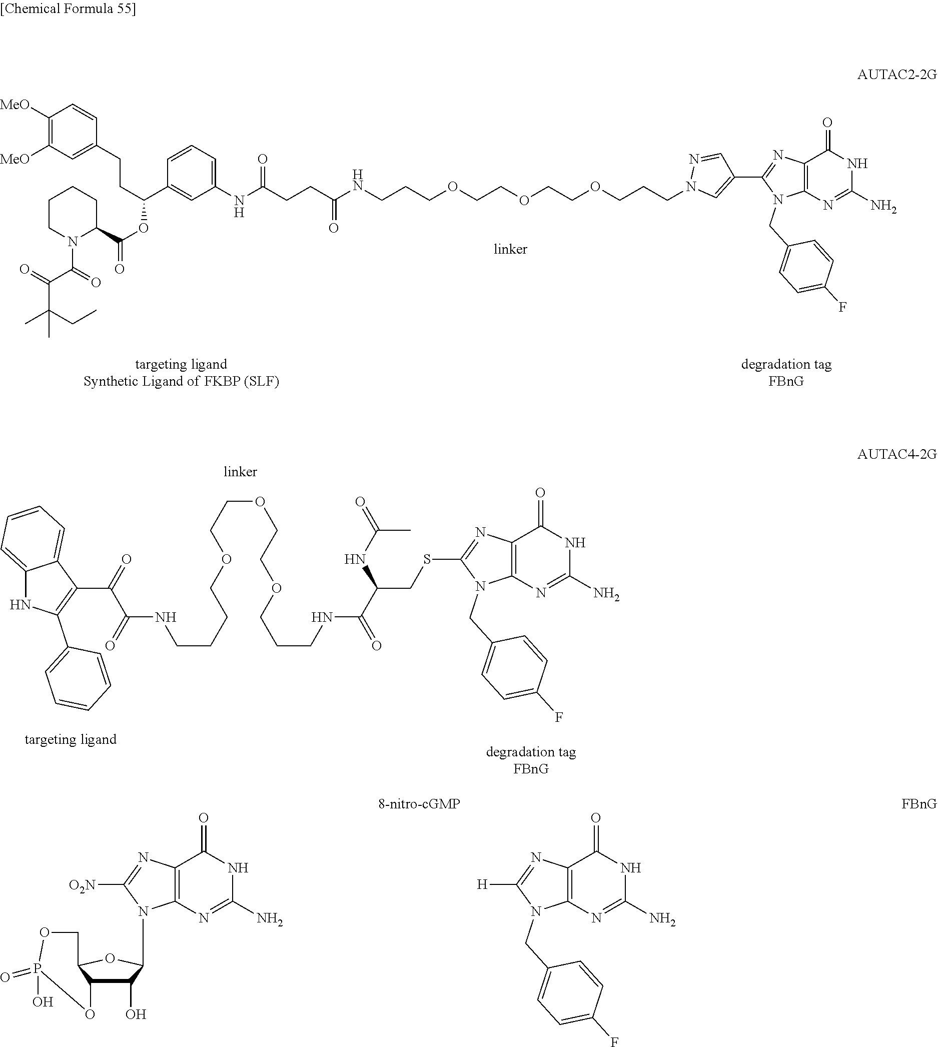

AUTAC4-2G and 8-nitro-cGMP are a compound and a tag moiety disclosed in JP-B-7161760, and AUTAC2-2G and FBnG are a compound and a tag moiety thereof newly synthesized to verify whether compounds screened by the method of the present invention actually induce autophagy (synthesis schemes of FBnG and AUTAC2-2G are as follows).

### Synthesis Scheme of FBnG

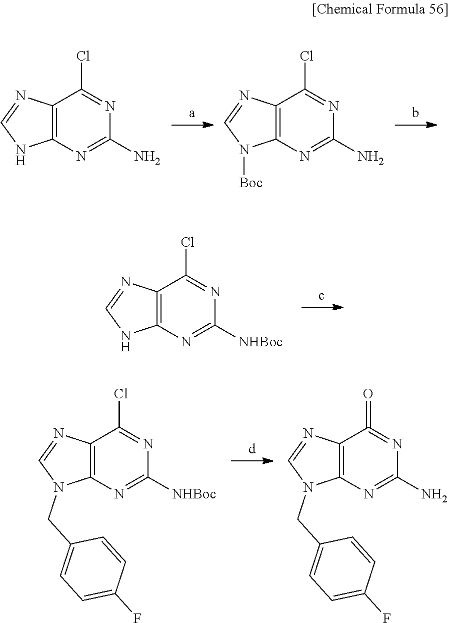

(a) Boc20, DMAP, DMSO, rt, 12 h; (b) NaH, THF, 0° C., 1 h, 62% (2 steps); (c) 4-fluorobenzyl alcohol, DEAD, PPh3, THE, rt, 3 h, 72%; (d) 80% HCO₂H, 80° C., 16 h, 83%.

### Synthesis Scheme of AUTAC2-2G

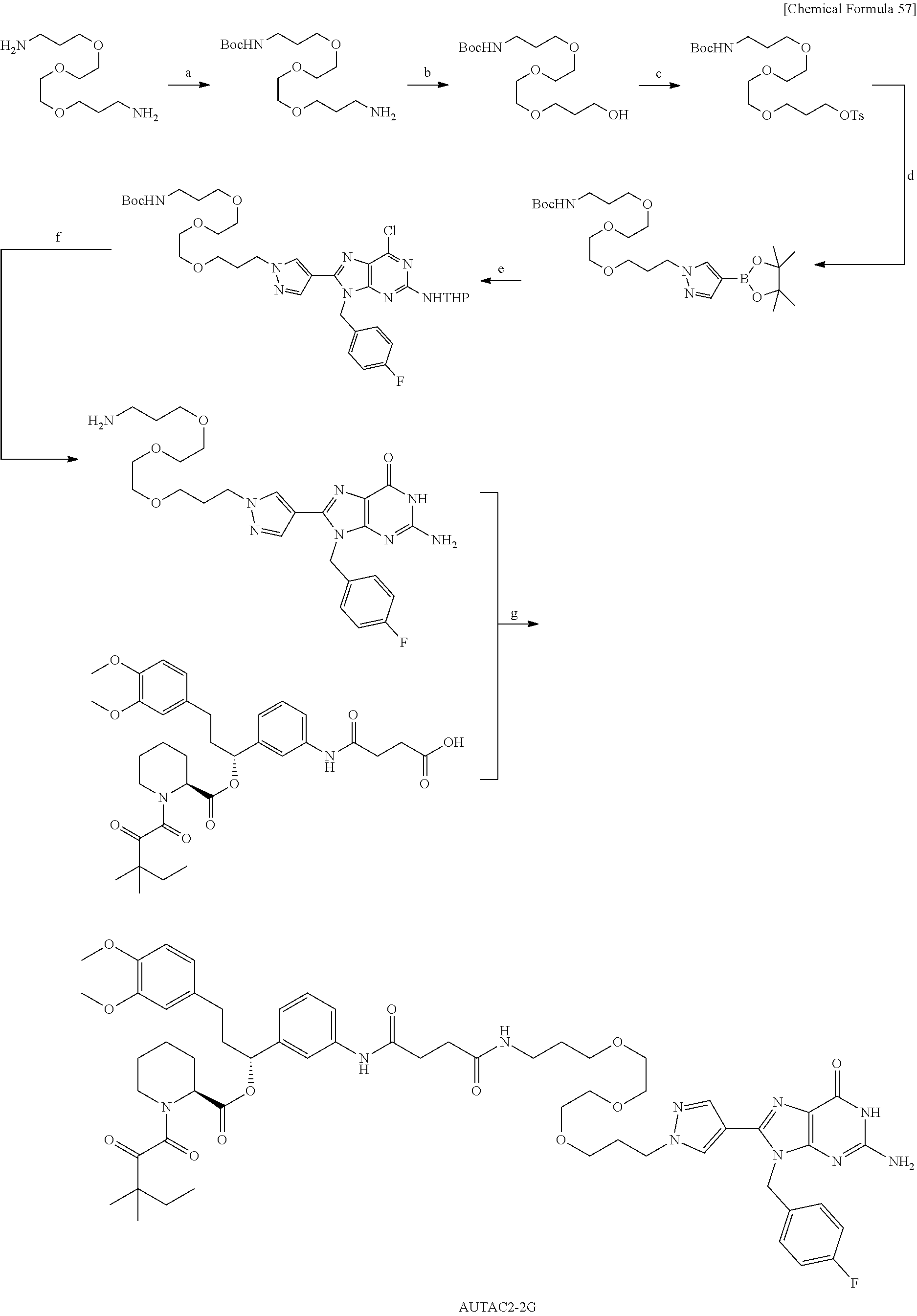

(a) Boc20, CH₂Cl₂, rt, 3 h; (b) NaNO₂, 20% CH COOH, rt, 3 h, 40%; (c) TsCl, (CH₃)₃N·HCl, NEt₃, CH₂Cl₂, rt, 2 h, 96%; (d) 4-pyrazoleboronic acid pinacol ester, K₂CO₃, DMF, 75° C., overnight, 71%; (e) 6-chloro-9-(4-fluorobenzyl)-8-iodo-N-(tetrahydro-2H-pyran-2-yl)-9H-purin-2-amine, Pd(PPh₃)₄, K₂CO₃, 66% N,N′-dimethylethyleneurea, 120° C., 2 h, 61%; (f) 80% HCOOH, 80° C., overnight, 85%; (g) PyBOP, DIPEA, rt, 18 h, 33%.

### 1-2. Test Procedure

### Example 1

HeLa cells grown in 96-well plates with growth medium were transfected with pLgBiT-p62 and pSmBiT-DDX5 using Lipofectamine3000 (registered trademark) transfection reagent (Thermo Fisher Scientific Inc.) according to the attached instruction. 24 hours after the addition of the reagent, AUTAC2-2G was added to the wells in such an amount as to achieve a concentration of 10 μM to treat the transfected cells with AUTAC2-2G for 12 hours. The growth medium was then replaced with 100 μL Opti-MEM (registered trademark) I (Thermo Fisher Scientific Inc.). 25 μL of 5×Nano-Glo Live Cell Reagent containing a substrate, furimazine, was added, and luminescence was immediately measured with GloMax (registered trademark) Discover Microplate Reader (Promega Corporation).

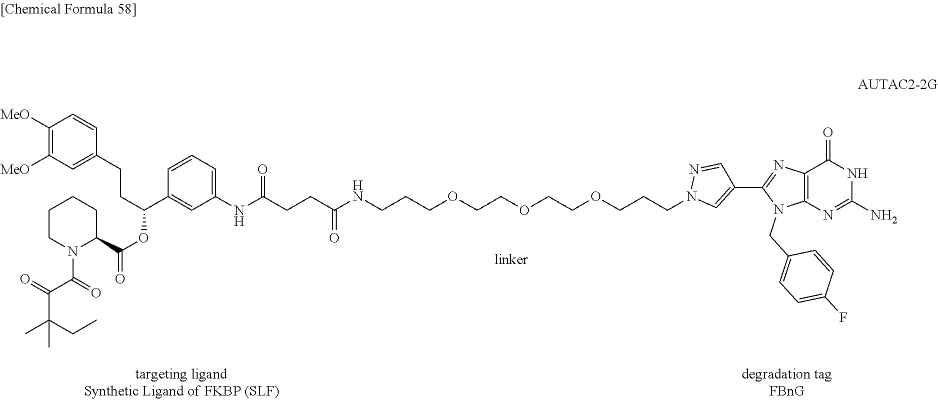 [SMILES: CCC(C)(C)C(=O)C(=O)N1CCCC[C@H]1C(=O)O[C@H](CCc1ccc(OC)c(OC)c1)c1cccc(NC(=O)CCC(=O)NCCCOCCOCCOCCCn2cc(-c3nc4c(=O)[nH]c(N)nc4n3Cc3ccc(F)cc3)cn2)c1]

### Example 2

The cells were treated in the same manner as in Example 1, except that AUTAC4-2G was added to the wells in such an amount as to achieve a concentration of 10 μM to treat the transfected cells with AUTAC4-2G for 12 hours, and luminescence was measured.

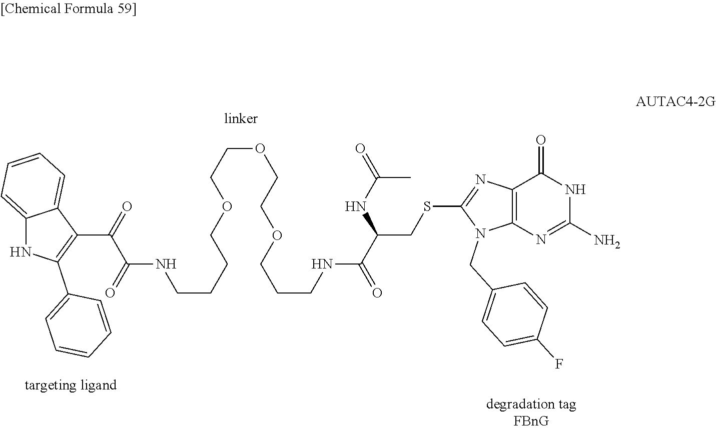 [SMILES: CC(=O)N[C@@H](CSc1nc2c(=O)[nH]c(N)nc2n1Cc1ccc(F)cc1)C(=O)NCCCOCCOCCOCCCCNC(=O)C(=O)c1c(-c2ccccc2)[nH]c2ccccc12]

### Example 3

The cells were treated in the same manner as in Example 1, except that 8-nitro-cGMP was added to the wells in such an amount as to achieve a concentration of 10 μM to treat the transfected cells with 8-nitro-cGMP for 24 hours, and luminescence was measured.

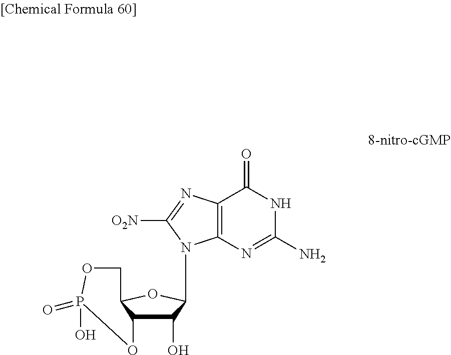

### Example 4

The cells were treated in the same manner as in Example 1, except that FBnG was added to the wells in such an amount as to achieve a concentration of 10 μM to treat the transfected cells with FBnG for 24 hours, and luminescence was measured.

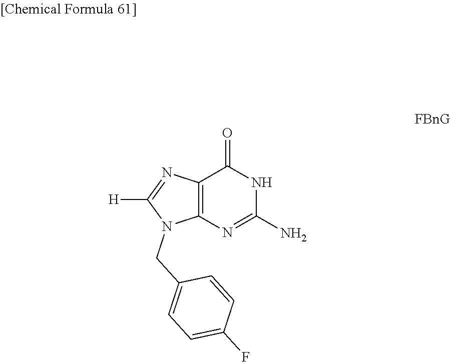 [SMILES: Nc1nc2c(ncn2Cc2ccc(F)cc2)c(=O)[nH]1]

### Comparative Example 1

The cells were treated in the same manner as in Example 1, except that DMSO (dimethyl sulfoxide) was added to the wells in such an amount as to achieve a concentration of 10 AM to treat the transfected cells with DMSO for 24 hours, and luminescence was measured.

### 1-3. Measurement Results

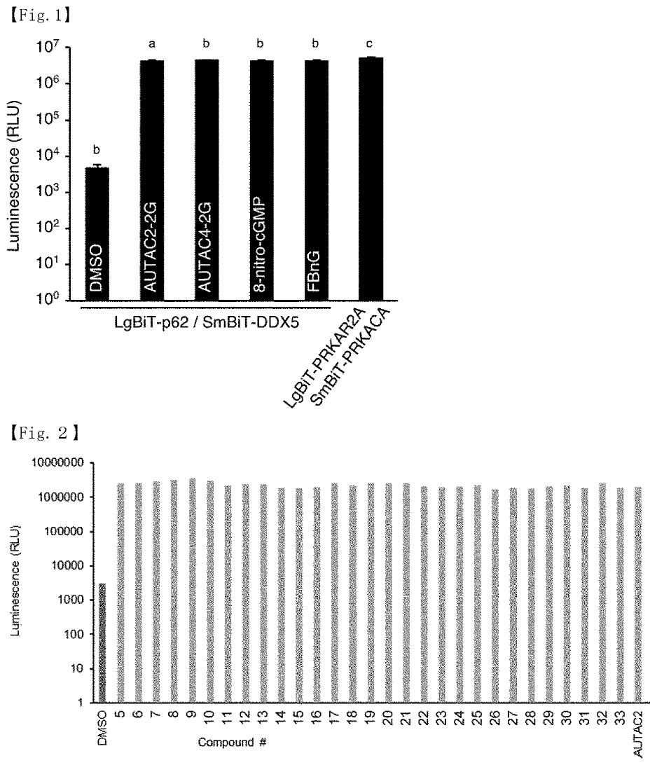

FIG. 1 shows the emission intensity 10 minutes after the start of measurement of luminescence in Examples 1 to 4. Data are shown as mean values from three independent experiments. The graph indicates 10⁷ as the upper limit, but actually has emission intensity equal to or greater than 10⁷. Error bars indicate standard deviation. Statistical analysis was performed using Turkey-Kramer multiple comparison test. P-values<0.01 were considered statistically significant.

Cells treated with AUTAC2-2G, AUTAC4-2G, 8-nitro-cGMP, and FBnG exhibited emission intensity 1000 times or more than that of cells treated with DMSO, demonstrating that p62 and DDX5 were in close proximity to each other in the presence of these compounds, and it was demonstrated that this method can screen compounds that bring p62 and DDX5 close to each other.

Test 2. Additional Validation of Screening Method for Compounds that Induce Interaction (Proximity) Between DDX5 and p62

Whether additional compounds developed by the present inventors that degrade target intracellular molecules by autophagy (J Med Chem. 2023 Sep. 14; 66 (17): see 12342-12372) induce an interaction between DDX5 and p62 was measured using the NanoBiT system.

### 2-1. Test Materials

As test materials, the same materials as in Test 1 (pLgBiT-p62 and pSmBiT-DDX5, Resources and Reagents) were used except that the following compounds were used to verify the screening method of the present invention.

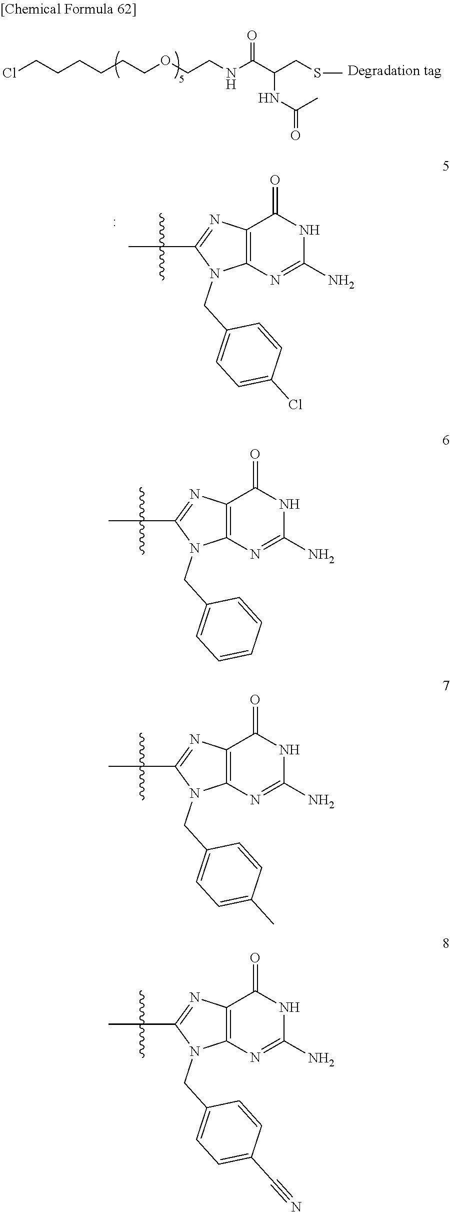

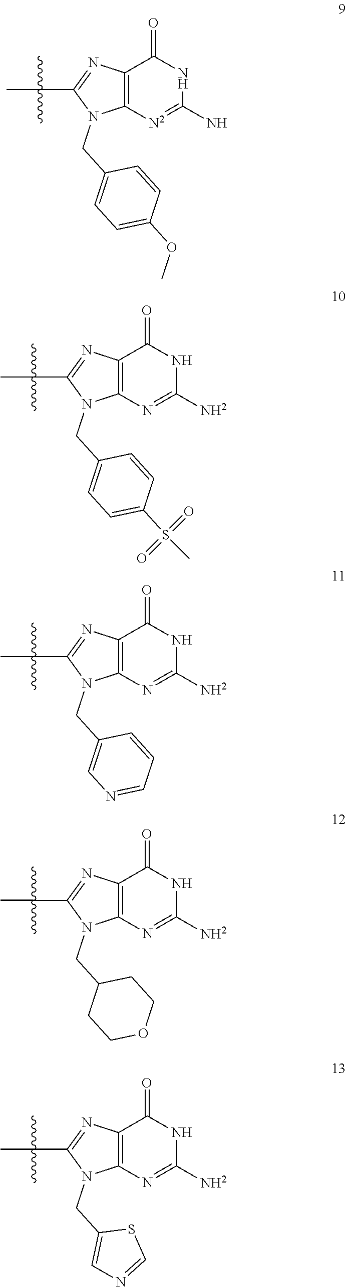

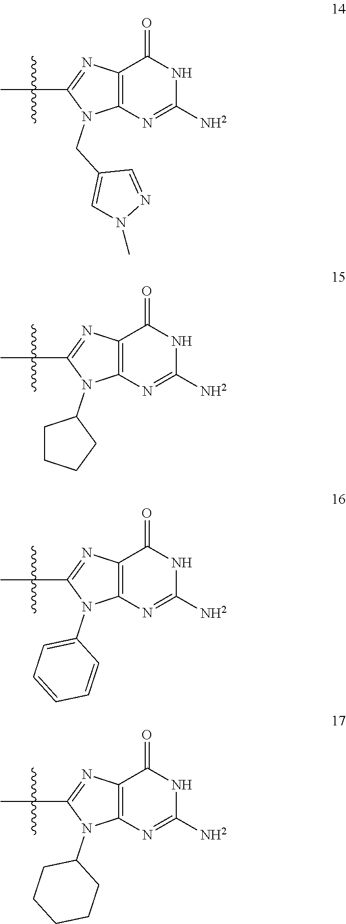

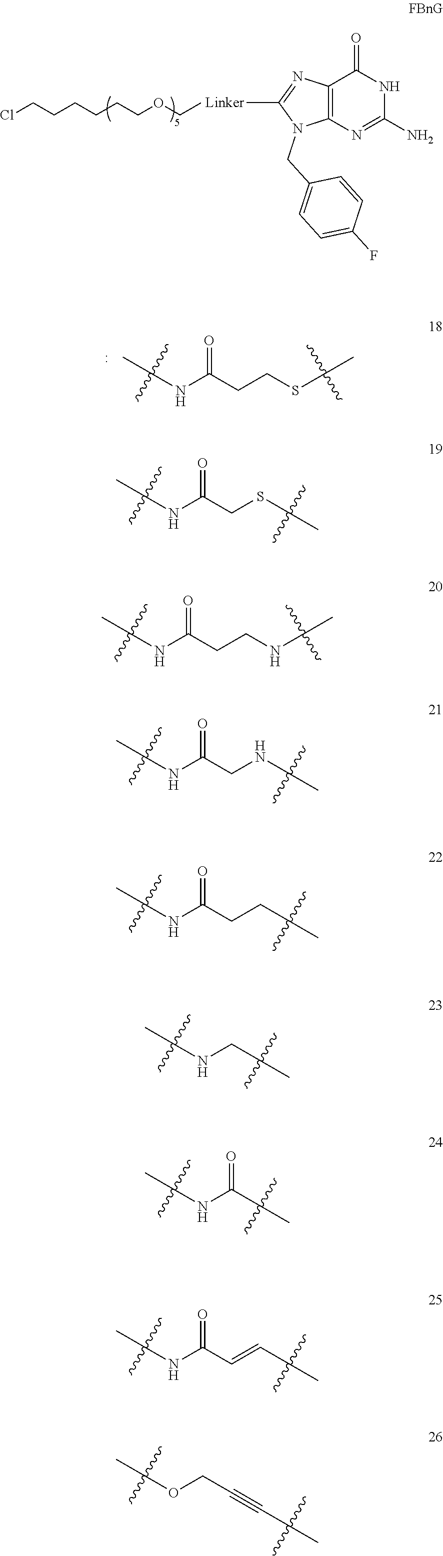

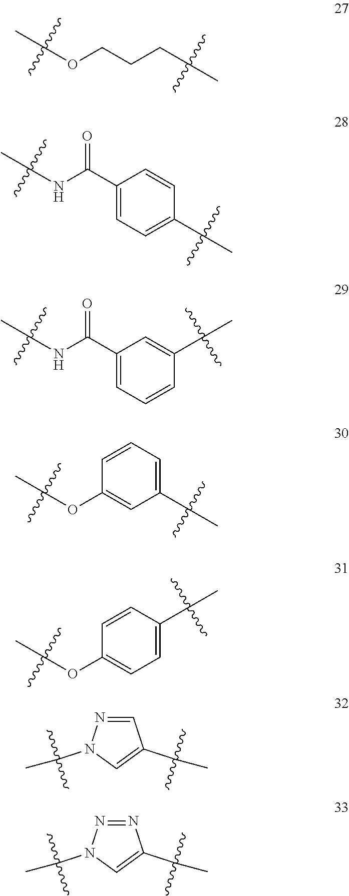

The compounds were synthesized according to the description of Second-Generation AUTACs for Targeted Autophagic Degradation, Daiki Takahashi et al., Journal of Medicinal Chemistry 66 12342-12372, and the like.

### 2-2. Test Procedure

### Examples 5 to 33

The cells were treated in the same manner as in Example 1, except that each of the compounds 5 to 33 was added to the wells in such an amount as to achieve a concentration of 10 μM to treat the transfected cells with each compound for 24 hours, and luminescence was measured.

### 2-3. Test Results

FIG. 2 shows the emission intensity 10 minutes after the start of measurement of luminescence in Examples 5 to 33, together with the emission amount 10 minutes after that in the transfected cells treated with AUTAC2-2G. Data are shown as mean values from three independent experiments. The graph indicates 10⁷ as the upper limit but actually has emission intensity equal to or greater than 10⁷. Error bars indicate standard deviation. Statistical analysis was performed using Turkey-Kramer multiple comparison test. P-values<0.01 were considered statistically significant.

Similar to cells treated with AUTAC2-2G, cells treated with the compounds 5 to 33 showed emission intensity 1000 times or more than that of cells treated with DMSO, demonstrating that p62 and DDX5 were in close proximity to each other even in the presence of these compounds. It has been confirmed by the present inventors that the compounds 5 to 33 induce degradation of a target intracellular molecule by autophagy (Daiki Takahashi et al., Journal of Medicinal Chemistry, 2023 Sep. 14; 66 (17): see 12342-12372)). Thus, it was also demonstrated that compounds that induce degradation of a target intracellular molecule by autophagy can be obtained by screening compounds that bring p62 and DDX5 close to each other.

Test 3. Construction of Screening Method for Compounds that Induce Interaction (Proximity) Between Other DEAD-Box RNA Helicase Members and p62

As a means for detecting a compound that degrades a target intracellular molecule by autophagy or a tag moiety thereof, a screening method for detecting a compound that induces an interaction between p62 and a DEAD-box RNA helicase member other than DDX5 was constructed. In this system, when a test compound induces an interaction between a DEAD-box RNA helicase member and p62, emission intensity increases, enabling detection of the presence of a compound with such activity.

### 3-1. Test Materials

Preparation of pLgBIT-DDXs (DDXs is Abbreviation of DEAD-Box RNA Helicase Member) and pSmBiT-p62

To generate pLgBIT-DDXs, the pBiT1.1-N [TK/LgBiT] Vector (Promega #N2014) was linearized by a polymerase chain reaction with oligo primers (5′-gcctccacctgctc-3′ and 5′-gctagcagatcttagagtcgg-3′). DDXs cDNA was synthesized from the total RNAs of HeLa cells using an oligo (dT)₁₅ primer, amplified using oligo primers (S′-agcggtaggcatggcgtcgctcaccg-3′ and 5′-agaagatctgctagtacaacggcgggatgc-3′), and inserted into a vector using an In-fusion cloning system (underlined 15 nt is In-fusion arm).

To generate pSmBiT-p62, the pBiT2.1-N [TK/SmBiT] Vector (Promega #N2014) was linearized by a polymerase chain reaction using oligo primers (5′-ggtggctttaccaacagtaccg-3′ and 5′-gctagcagatcttctagtcgg-3′). The p62 cDNA was synthesized from the total RNAs of HeLa cells using an oligo (dT)₁₅ primer, amplified using oligo primers (5′-agcggtggcatgtcgggattcgagtgaccg-3′ and 5′-agaagatctgctagcttattgggaatcctgttggcattg-3′), and inserted into a vector using an In-fusin cloning system (underlined 15 nt is In-fusion arm).

The nucleic acid sequences of pSmBiT-p62 and pLgBiT-DDXs carried by the respective vectors are shown below.

[Chemical Formula 63]
Nucleic acid sequence of pSmBiT-P62
(SEQ ID NO: 57)
ggcctaactggccggtacctgagtctaaatgagtcttcggacctcgcgggggccgcttaagcggtggttagggtttgtctgacgcggggggagg
ggaaggaacgaaacactctcattcggaggcggctggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctggggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtca
ttggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtgaccggctaccggctgttcgaggagattctcgggagtt
ccggtggtggcgggagcggaggtggaggcatggcgtcgctcaccgtgaaggcctaccttctgggcaaggaggacgcggcgcgcgagattcgc
cgcttcagcttctgctgcagccccgagcctgaggcggaagccgaggctgcggcgggtccgggaccctgcgagcggctgctgagccgggtggcc
gccctgttccccgcgctgcggcctggcggcttccaggcgcactaccgcgatgaggacggggacttggttgccttttccagtgacgaggaattgaca
atggccatgtcctacgtgaaggatgacatcttccgaatctacattaaagagaaaaaagagtgccggcgggaccaccgcccaccgtgtgctcaggag
gcgccccgcaacatggtgcaccccaatgtgatctgcgatggctgcaatgggcctgtggtaggaacccgctacaagtgcagcgtctgcccagacta
cgacttgtgtagcgtctgcgagggaaagggcttgcaccgggggcacaccaagctcgcattccccagccccttcgggcacctgtctgagggcttctc
gcacagccgctggctccggaaggtgaaacacggacacttcgggtggccaggatgggaaatgggtccaccaggaaactggagcccacgtcctcct
cgtgcaggggaggcccgccctggccccacggcagaatcagcttctggtccatcggaggatccgagtgtgaatttcctgaagaacgttggggagag
tgtggcagctgcccttagccctctgggcattgaagttgatatcgatgtggagcacggagggaaaagaagccgcctgacccccgtctctccagagag
ttccagcacagaggagaagagcagctcacagccaagcagctgctgctctgaccccagcaagccgggtgggaatgttgagggcgccacgcagtct
ctggcggagcagatgaggaagatcgccttggagtccgaggggcgccctgaggaacagatggagtcggataactgttcaggaggagatgatgact
ggacccatctgtcttcaaaagaagtggacccgtctacaggtgaactccagtccctacagatgccagaatccgaagggccaagctctctggacccctc
ccaggagggacccacagggctgaaggaagctgccttgtacccacatctcccgccagaggctgacccgcggctgattgagtccctctcccagatgc
tgtccatgggcttctctgatgaaggcggctggctcaccaggctcctgcagaccaagaactatgacatcggagcggctctggacaccatccagtattc
aaagcatcccccgccgttgtga
[Chemical Formula 64]
Nucleic acid sequence of pLgBiT-DDX1
(SEQ ID NO: 58)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtca
ttggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatggcggccttctc
cgagatgggtgtaatgcctgagattgcacaagctgtggaagagatggattggctcctcccaactgatatccaggctgaatctatcccattgatcttagg
aggaggtgatgtacttatggctgcagaaacaggaagtggcaaaactggtgcttttagtattccagttatccagatagtttatgaaactctgaaagacca
acaggaaggcaaaaaaggaaaaacaacaattaaaactggtgcttcagtgctgaacaaatggcagatgaacccatatgacagaggatctgcttttgcaa
ttgggtcagatggtctttgttgtcaaagcagagaagtaaaggaatggcatgggtgtagagctactaaaggattaatgaaagggaaacactactatgaa
gtatcctgtcatgaccaagggttatgcagggtcgggtggtctaccatgcaggcctcttggacctaggtactgacaagtttggatttggctttggtggaa
caggaaagaaatcccataacaaacaatttgataattatggagaggaattcactatgcatgataccattggatgttacctggatatagataagggacatg
tcaagttctccaaaaatggaaaagatcttggtctggcatttgaaataccaccacatatgaaaaaccaagccctctttcctgcctgtgttttgaagaatg
ctgaactgaaatttaacttcggtgaagaggaatttaagtttccaccaaaagatggctttgttgctctttccaaggcaccggafggttacattgtcaaat
cacagcactcaggtaatgcacaggtgacacaaacaaagtttctccccaatgctccgaaagctctcattgttgaaccttcccgggagttagctgaacaaa
ctttgaacaacatcaagcagtttaagaaatacattgataatcctaaattaagggagcttctgataattggaggtgttgcagcccgggatcagctctctg
ttttggaaaatggagtagatatagttgtaggtactccgggaagactagatgacttggtgtcaactggaaagctgaacttatctcaagttagattcctgg
tcctggatgaagctgatgggcttctttctcaaggttattctgattttataaataggatgcacaatcagattcctcaggttacctctgatggaaaaagac
ttcaggtgattgtttgctctgccactttgcattctttcgatgtaaagaaactgtccgagaagataatgcattttcctacatgggttgacttaaaaggag
aagactctgttccagatactgtacaccatgttgttgtcccagtaaatcccaaaactgacagactctgggaaaggcttggaaagagccacattagaactg
atgatgtacatgcaaaagataacacaagacctggtgctaatagtccagagatgtggtctgaagctattaaaatcctgaaaggggagtatgctgtccggg
caatcaaggaacataagatggatcaagcaattatcttctgtagaaccaaaattgactgtgataacttggagcagtactttatacaacaaggaggaggac
ctgataaaaaaggacaccagttctcatgtgtttgtcttcatggtgacagaaagcctcatgagagaaagcaaaacttggaaagatttaagaaaggagatg
taagattcttgatttgcacagatgtagctgctagaggaattgatatccacggtgttccttatgttataaatgtcactctgcccgatgaaaagcaaaact
acgtacatcgaattggcagagtaggaagagctgaaaggatgggtctggcaatttccctggtggcaacagaaaaagaaaaggtttggtaccatgtatgta
gcagccgtggaaaagggtgttataacacaagactcaaggaagatggaggctgtaccatatggtacaacgagatgcagttactatctgagatagaagaac
acctgaactgtaccatttctcaggttgagccggatataaaggtaccagtggatgaatttgatgggaaagttacctacggtcagaaaagggctgctggtg
gtggaagctataaaggccatgtggatattttggcacctactgttcaagagttggctgcccttgaaaaggaggcgcagacatctttcctgcatcttggct
accttcctaaccagctgttcagaaccttctga
[Chemical Formula 65]
Nucleic acid sequence of pLgBiT-DDX2A
(SEQ ID NO: 60)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgtctgcgagcca
ggattcccgatccagagacaatggccccgatgggatggagcccgaaggcgtcatcgagagtaactggaatgagattgttgacagctttgatgacat
gaacctctcggagtcccttctccgtggcatctacgcgtatggttttgagaagccctctgccatccagcagcgagccattctaccttgtatcaagggtta
tgatgtgattgctcaagcccaatctgggactgggaaaacggccacatttgccatatcgattctgcagcagattgaattagatctaaaagccacccaggc
cttggtcctagcacccactcgagaattggctcagcagatacagaaggtggtcatggcactaggagactacatgggcgcctcctgtcacgcctgtatc
gggggcaccaacgtgcgtgctgaggtgcagaaactgcagatggaagctccccacatcatcgtgggtacccctggccgtgtgtttgatatgcttaacc
ggagatacctgtcccccaaatacatcaagatgtttgtactggatgaagctgacgaaatgttaagccgtggattcaaggaccagatctatgacatattcc
aaaagctcaacagcaacacccaggtagttttgctgtcagccacaatgccttctgatgtgcttgaggtgaccaagaagttcatgagggaccccattcgg
attcttgtcaagaaggaagagttgaccctggagggtatccgccagttctacatcaacgtggaacgagaggagtggaagctggacacactatgtgact
tgtatgaaaccctgaccatcacccaggcagtcatcttcatcaacacccggaggaaggtggactggctcaccgagaagatgcatgctcgagatttcac
tgtatccgccatgcatggagatatggaccaaaaggaacgagacgtgattatgagggagtttcgttctggctctagcagagttttgattaccactgacct
gctggccagaggcattgatgtgcagcaggtttctttagtcatcaactatgaccttcccaccaacagggaaaactatatccacagaatcggtcgaggtg
gacggtttggccgtaaaggtgtggctattaacatggtgacagaagaagacaagaggactcttcgagacattgagaccttctacaacacctccattgag
gaaatgcccctcaatgttgctgacctcatctga
[Chemical Formula 66]
Nucleic acid sequence of pLgBiT-DDX3X
(SEQ ID NO: 61)
ggcctaactggccggtacctgagtctaaatgagtcttcggacctcgcgggggccgcttaagcggtggttagggtttgtctgacgcgggggggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggggtaactggcgggccgctgactcgggcgggcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgagtcatgtggc
agtggaaaatgcgctcgggctggaccagcagtttgctggcctagacctgaactcttcagataatcagagtggaggaagtacagccagcaaaggg
gctatattcctcctcatttaaggaaccgagaagctactaaaggtttctacgataaagacagttcagggtggagttctagcaaagataaggatgcgtata
gcagttttggatctcgtagtgattcaagagggaagtctagcttcttcagtgatcgtggaagtggatcaaggggaaggtttgatgatcgtggacggagtg
attacgatggcattggcagccgtggtgacagaagtggctttggcaaatttgaacgtggtggaaacagtcgctggtgtgacaaatcagatgaagatgat
tggtcaaaaccactcccaccaagtgaacgcttggaacaggaactcttttctggaggcaacactgggattaattttgagaaatacgatgacattccagtt
gaggcaacaggcaacaactgtcctccacatattgaaagtttcagtgatgttgagatgggagaaattatcatgggaaacattgagcttactcgttatact
cgcccaactccagtgcaaaagcatgctattcctattatcaaagagaaaagagacttgatggcttgtgcccaaacagggtctggaaaaactgcagcattt
ctgttgcccatcttgagtcagatttattcagatggtccaggcgaggctttgagggccatgaaggaaaatggaaggtatgggcgccgcaaacaatacc
caatctccttggtattagcaccaacgagagagttggcagtacagatctacgaggaagccagaaaattttcataccgatctagagttcgtccttgcgtgg
tttatggtggtgccgatattggtcagcagattcgagacttggaacgtggatgccatttgttagtagccactccaggacgtctagtggatatgatggaaa
gaggaaagattggattagacttttgcaaatacttggtgttagatgaagctgatcggatgttggatatggggtttgagcctcagattcgtagaatagtcg
aacaagatactatgcctccaaagggtgtccgccacactatgatgtttagtgctacttttcctaaggaaatacagatgctggctcgtgatttcttagatg
aatatatcttcttggctgtaggaagagttggctctacctctgaaaacatcacacagaaagtagtttgggtggaagaatcagacaaacggtcatttctgc
ttgacctcctaaatgcaacaggcaaggattcactgaccttagtgtttgtggagaccaaaaaggggcagattctctggaggatttcttataccatgaagg
atacgcatgtaccagcatccatggagaccgttctcagagggatagagaagaggcccttcaccagttccgctcaggaaaaagcccaattttagtggctac
agcagtagcagcaagaggactggacatttcaaatgtgaaacatgttatcaattttgacttgccaagtgatattgaagaatatgtacatcgtattggtcg
tacgggacgtgtaggaaaccttggcctggcaacctcattctttaacgagaggaacataaatattactaaggatttgttggatcttcttgttgaagctad
acaagaagtgccgtcttggttagaaaacatggcttatgaacaccactacaagggtagcagtcgtggacgttctaagagtagcagatttagtggagggtt
tggtgccagagactaccgacaaagtagcggtgccagcagttccagcttcagcagcagccgcgcaagcagcagccgcagtggcggaggtggccacggta
gcagcagaggatttggtggaggtggctatggaggcttttacaacagtgatggatatggaggaaattataactcccagggggttgactggtggggtaa
ctga
[Chemical Formula 67]
Nucleic acid sequence of pLgBiT-DDX4
(SEQ ID NO: 62)
ggcctaactggccggtacctgagtctaaatgagtcttcggacctcgcgggggccgcttaagcggtgcttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcatgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagccttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgggagatgaag
attgggaagcagaaatcaaccctcatatgtcttcctatgttcccatatttgagaaggataggtattctggagaaaatggagacaattttaacaggactc
agcttcatcatcagaaatggatgatggaccttctcgaagagatcatttcatgaaaagtggatttgcctctgggcggaattttggaaacagagatgctg
gagagtgtaataagcgagataatacatccacaatgggggttttggagttggaaagagttttggaaacagaggtttttcaaacagcaggtttgaagatgg
gatagctctggtttctggagagagtctagtaatgactgcgaagataatccaacacggaacagagggttttccaagagaggcggctatcgagatggaa
ataattcagaagcttcagggccatacagaagaggtggaagaggtagtttccgaggttgccgtggaggatttggtctaggaagtccaaataatgactta
gacccagacgaatgtatgcagcgcactggtggcctttttggttctagaagaccagtattaagtggcacaggtaatggtgatacttctcaaagcagaagt
ggcagtggaagtgaacgaggtggttacaaaggtttaaatgaagaagtaataacaggcfctggaaagaattcttggaagtcagaagcagaaggagg
agaaagtagtgatactcaaggaccaaaagtgacctacataccccctcctccacctgaggatgaggactccatctttgcacattatcagacaggcataa
acttcgacaaatacgacactattcttgtggaagtgtctggacatgatgcaccaccagcaattctgacttttgaagaagctaatctctgtcagacactga
aacaacattgctaaagctggttatactaagcttactcctgtgcaaaaatacagtattcctatcatacttgcaggacgagatttgatggcttgcgctc
aaacagggtctgggaagactgcggcttttctcctaccaattttggctcatatgatgcatgatggaataactgccagtcgttttaaagagttgcaggaac
cagagtgtattattgtagcaccaactcgagaattggtcaaccagatttatttggaagccagaaaattttcttttgggacttgtgtaagagctgttgtta
tatatgggggaacccagctgggacattcaattcgacaaatagtacaaggctgtaatatattatgtgctactcctggaagactgatggatatcataggca
aagaaaagattggtctcaaacagatcaaatacttagttttggatgaagctgatcgcatgttggatatgggttttggtccagaaatgaagaagttaattt
cttgcccaggaatgccatcaaaggaacagcgccaaacccttatgttcagtgcaacttttccagaggaaattcaaaggttggctgcagagtttttaaagt
caaattatctgtttgttgctgttggacaagtgggtggagcatgtagagatgttcagcagaccgttctccaagttggccagttctcaaaaagagaaaagc
tcgttgaaattctgcgaaacataggggatgaaagaactatggtctttgttgaaactaagaaaaaagcagattttattgcaacttttctttgtcaagaaa
aaatatcaactacaagtattcatggtgatcgggaacagagagagcgggagcaagctcttggagattttcgctttggaaagtgcccagttcttgttgcta
cttcagtagctgccagagggctggatattgaaaatgtgcaacatgttatcaaftttgatcttccttctaccattgatgaatatgttcatcgaattgggc
gtactggtcgttctgggaatactggcagagcaatttccttttttgatcttgaatcggataaccatttagcacagcctctagtaaaagtattgacagatg
ctcaacaggatgttcctgcatggttggaagaaattgcctttagtacatacattcctggcttcagtggtagtacaagaggaaacgtgtttgcatcagttg
ataccagaaagggcaagagcactttgaacacagctgggttttcttcttcacaagctcccaatccagtagatgatgagtcatgggatgggattaa
[Chemical Formula 68]
Nucleic acid sequence of pLgBiT-DDX5
(SEQ ID NO: 63)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtgggggagcggaggtggaggcatgtcgggttattcg
agtgaccgagaccgcggccgggaccgagggtttggtgcacctcgatttggaggaagtagggcagggcccttatctggaaagaagtttggaaaccc
tggggagaaattagttaaaaagaagtggaatcttgatgagctgcctaaatttgagaagaatttttatcaagagcaccctgatttggctaggcgcacagc
acaagaggtggaaacatacagaagaagcaaggaaattacagttagaggtcacaactgcccgaagccagttctaaatttttatgaagccaatttccctg
caaatgtcatggatgttattgcaagacagaatttcactgaacccactgctattcaagctcagggatggccagttgctctaagtggattggatatggttg
gagtggcacagactggatctgggaaaacattgtcttatttgcttcctgccattgtccacatcaatcatcagccattcctagagagaggcgatgggccta
tttgtttggtgctggcaccaactcgggaactggcccaacaggtgcagcaagtagctgctgaatattgtagagcatgtcgcttgaagtctacttgtatct
acggtggtgctcctaagggaccacaaatacgtgatttggagagaggtgtggaaatctgtattgcaacacctggaagactgattgactttttagagtgtg
gaaaaaccaatctgagaagaacaacctaccttgtccttgatgaagcagatagaatgcttgatatgggctttgaaccccaaataaggaagattgtggatc
aaataagacctgataggcaaactctaatgtggagtgcgacttggccaaaagaagtaagacagcttgctgaagatttcctgaaagactatattcatataa
acattggtgcacttgaactgagtgcaaaccacaacattcttcagattgtggatgtgtgtcatgacgtagaaaaggatgaaaaacttattcgtctaatgg
aagagatcatgagtgagaaggagaataaaaccattgtttttgtggaaaccaaaagaagatgtgatgagcttaccagaaaaatgaggagagatgggtggc
ctgccatgggtatccatggtgacaagagtcaacaagagcgtgactgggttctaaatgaattcaaacatggaaaagctcctattctgattgctacagatg
tggcctccagagggctagatgtggaagatgtgaaatttgtcatcaattatgactaccctaactcctcagaggattatattcatcgaattggaagaactg
ctcgcagtaccaaaacaggcacagcatacactttctttacacctaataacataaagcaagtgagcgaccttatctctgtgcttcgtgaagctaatcaag
caattaatcccaagttgcttcagttggtcgaagacagaggttcaggtcgttccaggggtagaggaggcatgaaggatgaccgtcgggacagatactctg
cgggcaaaaggggtggatttaatacctttagagacagggaaaattatgacagaggttactctagcctgcttaaaagagattttggggcaaaaactcaga
atggtgtttacagtgctgcaaattacaccaatgggagctttggaagtaattttgtgtctgctggtatacagaccagttttaggactggtaatccaacag
ggacttaccagaatggttatgatagcactcagcaatacggaagtaatgttccaaatatgcacaatggtatgaaccaacaggcatatgcatatcctgcta
ctgcagctgcacctatgattggttatccaatgccaacaggatattcccaataa
[Chemical Formula 69]
Nucleic acid sequence of pLgBiT-DDX6
(SEQ ID NO: 64)
ggcctaactggccggtacctgagtctaaatgagtcttcggacctcgcgggggccgcttaagcggtggttaggctttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaagccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgagcacggcca
gaacagagaaccctgttataatgggtctgtccagtcaaaatggtcagctgagaggccctgtgaaacccactggtggccctggaggagggggcaca
cagacacagcaacagatgaaccagctgaaaaacaccaacacaatcaataatggcactcagcagcaagcacagagtatgaccaccactattaaacc
tggtgatgactggaaaaagactttaaaactccctccaaaggatctaagaatcaaaacttcggatgtgacctccacaaaaggaaatgagtttgaagatta
ctgtttgaaacgggagttactgatgggaatttttgaaatgggctgggaaaagccatctcctattcaggaggagagcattcccattgctttatctggtag
ggatatcttagctagagcaaaaaatggaacaggcaagagcggtgcctacctcattcccttacttgaacggctagacctgaagaaggacaatatacaag
caatggtgattgttcccactagagaacttgctctacaggtcagtcaaatttgcatccaggtcagcaaacacatgggaggggccaaagtgatggcaac
cacaggaggaaccaatttacgagatgacataatgaggcttgatgatacagtgcacgtggtgattgctacccctcggagaatcctggatcttattaaga
aaggagtagcaaaggttgatcatgtccagatgatagtattggatgaggcagataagttgctgtcacaggattttgtgcagataatggaggatattattc
tcacgctacctaaaaacaggcagattttactatattccgctactttccctcttagtgtacagaagttcatgaattcccatttgcagaaaccctatgaga
ttaacctgatggaggaactaactctgaagggagtaacccagtactacgcatatgtaactgagcgccaaaaagtacactgcctcaacacacttttctcca
ggcttcagataaaccagtcgatcattttctgtaactcctctcagcgagttgaattgctagccaagaagatttctcaactgggttattcttgcttctata
ttcatgctaaaatgaggcaggaacatcgaaatcgtgtatttcatgatttccgaaatggcttagccgcaatcttgtttgcactgatctgtttacccgagg
tattgatatacaagctgtgaatgtggtaataaactttgatttcccaaagctggcagagacctatctccatcgtattggaagatcaggtcgctttggtca
tcttggcttagccatcaacttgatcacatatgatgatcgcttcaacctgaaaagtattgaggagcagctgggaacagaaattaaacctattccgagcaa
cattgataagagcctgtatgtggcagaataccacagcgagcctgtagaagatgagaaaccttaa
[Chemical Formula 70]
Nucleic acid sequence of pLgBiT-DDX10
(SEQ ID NO: 65)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactgggggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgggcaaaacgg
ccaactctccgggttcgggagcccgacccgacccggtgcggagcttcaatcgctggaagaaaaaacacagccataggcagaacaaaaagaagc
agttgaggaagcaactgaagaaacccgaatggcaggtcgagcgcgagagtatcagccgcctcatgcagaactatgaaaagataaatgtaaatgaa
atcacaagattttcagattttcccttgtccaaaaaaacattgaaaggtttgcaagaagctcagtaccgtttggtgactgagatacagaagcagaccatt
ggattggctttgcaaggtaaagatgtacttggagcggccaaaactggatctggcaagactctggcttttcttgttccagtgctggaagccttatatcgt
ctgcaatggacttcaacagatgggctgggggttctcataatatcacctacgagagaactggcctatcagacctttgaggttctccgaaaagtaggaaag
aatcatgacttctcagctggtctcatcattggtggaaaggatctaaaacacgaagctgagaggatcaacaacataaatatactcgtgtgcacaccaggt
cggcttcttcaacacatggatgaaacagtatcttttcatgctaccgacctccaaatgttagttcttgatgaagcagatagaatcttggatatgggcttt
gctgataccatgaatgctgttattgaaaatctccccaagaaacgtcagactttacttttctcagcaacacaaactaaatctgtaaaggaccttgcacgc
ttgagtttgaaaaaccctgagtatgtctgggttcatgaaaaagcaaaatatagcacccctgccactttggaacagaactacatagtctgtgagctgcag
caaaaaataagtgtgtgtattcctttttgagaagccatctgaagaagaagagcattgtatttuttccagttgcaaagaggtccagtatctgtaccgagt
gttttgccggctacgtcctggtgtttctatccttgcactccatggtcgacagcagcaaatgagaagaatggaagtctataatgagtttgtccgtaagag
agctgcagtactctttgctactgatattgcagccaggggtctggatttcccggccgtgaattgggttcttcagtttgattgtcctgaggatgccaacac
atatattcacagagcaggtagaactgccaggtacaaagaggatggtgaagctttgctaattttgctaccctcagaaaaagctaggtgcagcagcttctt
cagaagaaagtacctgtgaaggaaatcaaaatcaatccagaaaaacttatagatgtccagaaaaaattggaatctattttagctcaagatcaagattta
aaagaaagagctcaaaggtgtttcgtctcctatgtacgatctgtatatctgatgaaggataaagaagtatttgatgtgagcaagttacctatacctgaa
tatgccctgtctcttggtcttgctgggcaccacgcgtaagatttcttcagaaaatgcagaaacaacccaccaaagaattggtaaggagccaagccgata
aagtaattgagccaagggctccctccctcaccaatgacgaagtggaagaatttagagcctacttcaatgagaaaatgtccatccttcaaaaaggtggaa
aaagactcgaagggacagagcacagacaggataatgatactggtaatgaagaacaggaagaagaagaagacgatgaagaagaaatggaagagaaactgg
caaaagcaaaaggatctcaagccccatctcttcctaacaccagtgaggcacagaagatcaaggaagttcctacacagttcttggacagagatgaggag
gaagaagatgctgatttcttgaaggtgaagcggcataatgtgtttggattggaccttaaagacgagaaaacattacagaagaaagaaccttctaaatcc
agcatcaagaaaaaaatgaccaaagttgcagaagcaaaaaaagtaatgaagagaaattttaaagtgaataagaagataacatttactgatgaagggg
agttggttcagcagtggccacaaatgcagaaatctgccatcaaggatgctgaggaagatgatgacacaggtggtatcaacttacataaagcaaagg
aaagacttcaggaagaggacaaatttgacaaagaagaatataggaaaaaaattaaggcaaagcatcgggagaaaagactgaaagaaagggaagc
cagaagagaagccaacaagagacaagcaaaggccaaagatgaagaggaagcctttctggattggagtgatgatgatgatgatgatgatgatggatt
tgatccaagcacactcccagatccagataaatacagaagctctgaagattcagatagtgaagatatggaaaataaaataagtgataccaagaagaag
caggggatgaagaagaggagcaacagtgaagtggaagacgtgggaccaacaagtcataacagaaagaaggccaggtgggacactttagagcct
ttggataccggcctgtctttagcagaggatgaagagctggtgttacatctgctaagaagtcaaagctaa
[Chemical Formula 71]
Nucleic acid sequence of pLgBiT-DDX11
(SEQ ID NO: 66)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatggctaatgaaac
acagaaggttggtgccatccattttccttttcccttcacaccctattccatccaggaagacttcatggcagagctgtaccgggttttggaggctggcaa
gattgggatatttgagagtccaactggcactgggaagtccttaagtcttatttgtggggccctctcttggctccgtgactugaacagaagaagcgtgaa
gaagaggcacgactccttgaaactggaactggccccttacatgatgagaaagatgaatccctgtgtctgtcttcftcctgcgaaggggctgcaggcacc
ccgaggcctgctggagaaccggcctgggttactcagtttgtgcagaagaaagaagagagggacctggtggaccgactaaaggcggagcaggcc
aggaggaagcagcgagaagaacgcctgcagcagctgcagcacagggtgcagctcaagtatgcagccaagcgcctgaggcaggaagaagaag
aaagagagaatctcctccgcctcagcagggagatgctagagacaggcccggaggctgagcggctggagcagctggagtctggggaggaggag
ctggtcctcgccgaatacgagagtgatgaggagaaaaaggtggcgagcagagtggatgaggatgaggatgacctggaggaagaacacataacta
agatttattactgtagtcggacacactcccagctggcccagtttgtgcatgaggtgaagaagagcccctttggcaaggatgttcggctggtctcccttg
gctcccggcagaacctttgtgtaaatgaagacgtgaaaagcctaggttctgtgcagcttatcaacgaccgctgtgtggacatgcagagaagcaggca
cgagaagaagaaaggagctgaggaggagaagccaaagaggaggaggcaggagaagcaggcagcctgccccttctacaaccacgagcagatg
ggccttctccgggatgaggccctggcagaggtgaaggacatggagcagctgctggcccttgggaaggaggcccgggcctgtccctattacggga
gccgccttgccatccctgcagcccagctggtggtgctgccctatcagatgctgctgcatgcggccactcggcaggccgcgggcatccggctgcag
gaccaggtggtgatcatcgacgaggcgcacaacctgatcgacaccatcacgggcatgcacagcgtggaggtcagcggctcccagctctgccagg
cccattcccagctgctgcagtacgtggagcgatacgggaagcgtttgaaggccaagaacctgatgtacctgaagcagatcctgtatttgctggagaa
attcgtggctgtgctaggggggaacattaagcaaaatcccaatacacagagtctgtcacagacagggacggagctgaagaccatcaacgactttctc
ttccagagccagatcgacaacatcaacctgttcaaggtgcagcgatactgtgagaagagcatgatcagcagaaagctctttggattcactgaacggt
acggagcagtgttctcatcccgggagcagcccaaactggctgggtttcagcaattcctgcagagcctgcagcccaggacgactgaagctcttgcag
cccctgcagacgagagtcaggccagcaccctgcgaccagcttctccactgatgcacatccaaggcttcctggcagctctcactacggccaaccag
gacggcagggtcatcctgagccgccaaggcagcctcagtcagagcaccctgaagtttttgctcctgaatccagctgtgcactttgcccaagtggtga
aggaatgccgggcagtggtcattgcggggggtaccatgcagccggtgtctgacttccggcagcagctgctggcctgtgccggggtggaagctga
gcgcgtggtggagttttcctgtggtcacgtgatccctccagacaacatcctgcccctcgtcatctgcagcgggatctccaaccagccgctggaattca
cgttccagaaqagagagctgcctcagatgatggacgaggtgggtcgcattctctgtaacctgtgcggtgtggttcctggaggggggtctgtttcttcc
cctcctacgagtacctgcgccaggtccatgcccactgggagaagggtggcctgctgggccgtctggctgccaggaagaagatattccaggaacct
aagagcgcacaccaggtggagcaggtgctgctggcatattccaggtgcatccaggcctgtggccaggagagaggccaggtgacaggggccctg
ctcctctctgtggttggaggaaagatgagtgaagggatcaacttctctgacaacctaggccggtgtgtggtgatggtgggcatgcccttccccaacat
caggtctgcagagctgcaggagaagatggcctacttggatcaaaccctcagcccccggccaggcacccccagggaaggctctggggagaacct
gtgcatgaaggccgtcaaccagtccataggcagggccatcaggcaccagaaggattttgccagcgtagtgctcctggaccagcgatatgcccggc
cccctgtcctggccaagctgccggcctggatccgagcccgtgtggaggtcaaagctacctttggccccgccattgctgctgtgcagaagtttcaccg
ggagaagtcggcctcttcc
[Chemical Formula 72]
Nucleic acid sequence of pLgBiT-DDX17
(SEQ ID NO: 67)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcggggggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgagcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgfcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccgctggtgggggagcggaggtggaggcatgcccaccggctt
tgtagccccgattctctgtgttttgctcccgtctccgacgagagaggcggcgacggtggcgtctgcgacgggagacagcgcgtcggagcgagaga
gcgctgcgcctgtcgccgccccaacagcggaggcgccgccgccatcggtcgtcaccagaccggagccgcaggccctcccgagcccggccatc
cgtgccccgctcccagatctctatccttttgggaccatgcgcggaggaggctttggggaccgggaccgggatcgtgaccgtggaggatttggagca
agaggtggtggtggccttcccccgaagaaatttggtaatcctggggagcgtttgcgtaaaaaaaagtgggatttgagtgagctccccaagtttgagaa
aaatttttatgtggaacatccggaagtagcaaggctgacaccatatgaggttgatgagctacgccgadagaaggagattacagtgagggggggaga
tgtttgtcctaaacccgtgtttgccttccatcatgctaacttcccacaatatgtaatggatgtgttgatggatcagcactttacagaaccaactccaat
tcagtgccagggatttccgttggctcttagtggccgggatatggtgggcattgctcagactggctctgggaagacgttggcgtatctcctgcctgcaat
tgttcatattaccaccagccatacttggaaaggggagatggcccaatctgtctagttctggctcctaccagagagcttgcccagcaagtacagcaggtg
gccgatgactatggcaaatgttctagattgaagagtacttgtatttatggaggtgctcctaaaggtccccagattcgagacttggaaagaggtgttgag
atctgcatagccactcctggacgtctgatagatttcctggagtcaggaaagacaaatcttcgccgatgtacttaccttgtattggacgaagctgacaga
atgcttgatatggggtttgaaccccagatccgtaaaattgttgaccaaatcaggcctgataggcagacactgatgtggagtgcaacctggccaaaagaa
gtaagacagcttgcagaggatttccttcgtgattacacccagatcaacgtaggcaatctggagttgagtgccaaccacaacatcctccagatagtggat
gtctgcatggaaagtgaaaaagaccacaagttgatccaactaatggaagaaataatggctgaaaaggaaaacaaaacaataatatttgtggagacaaa
gagacgctgtgatgatctgactcgaaggatgcgcagagatggttggccagctatgtgtatccatggagacaagagtcaaccagaaagagattgggt
acttaatgagttccgttctggaaaggcacccatccttattgctacagatgtagcctcccgtgggctagatgtggaagatgtcaagtttgtgatcaacta
tgactatccaaacagctcagaggattatgtgcaccgtattggccgaacagcccgtagcaccaacaagggtaccgcctataccttcttcaccccagggaa
cctaaaacaggccagagagcttatcaaagtgctggaagaggccaatcaggctatcaatccaaaactgatgcagcttgtggaccacagaggaggcg
gcggaggcgggggtggtcgttctcgttaccggaccacttcttcagccaacaatcccaatctgatgtatcaggatgagtgtgaccgaaggcttcgagg
agtcaaggatggtggccggagagactctgcaagctatcgggatcgtagtgaaaccgatagagctggttatgctaatggcagtggctatggaagtcc
aaattctgcctttggagcacaagcaggccaatacacctatggtcaaggcacctatggggcagctgcttatggcaccagtagctatacagctcaagaat
atggtgctggcacttatggagctagtagcaccacctcaactgggagaagttcacagagctctagccagcagtttagtgggataggccggtctgggca
gcagccacagccactgatgtcacaacagtttgcacagcctccgggagctaccaatatgataggttacatggggcagactgcctaccaataccctcct
cctcctccccctcctcctccttcacgtaaatga
[Chemical Formula 73]
Nucleic acid sequence of pLgBiT-DDX18
(SEQ ID NO: 68)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggggtaactggcgggccgctgactcgggcgggcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgtcacacctgcc
gatgaaactcctgcgtaagaagatcgagaagcggaacctcaaattgcggcagcggaacctaaagtttcagggggcctcaaatctgaccctatcgga
aactcaaaatggagatgtatctgaagaaacaatgggaagtagaaaggttaaaaaatcaaaacaaaagcccatgaatgtgggcttatcagaaactcaa
aatggaggcatgtctcaagaagcagtgggaaatataaaagttacaaagtctccccagaaatccactgtattaaccaatggagaagcagcaatgcagt
cttccaattcagaatcaaaaaagaaaaagaagaaaaagagaaaaatggtgaatgatgctgagcctgatacgaaaaaagcaaaaactgaaaacaaa
gggaaatctgaagaagaaagtgccgagactactaaagaaacagaaaataatgtggagaagccagataatgagaagatgagagtgaggtgccca
gtctgcccctgggactgacaggagcttttgaggatacttcgtttgcttctctatgtaatcttgtcaatgaaaacactctgaaggcaataaaagaaatgg
gttttacaaacatgactgaaattcagcataaaagtatcagaccacttctggaaggcagggatcttctagcagctgcaaaaacaggcagtggtaaaaccc
tggcttttctcatccctgcagttgaactcattgttaagttaaggttcatgcccaggaatggaacaggagtccttattctctcacctactagagaactag
ccatgcaaacctttggtgttcttaaggagctgatgactcaccacgtgcatacctatggcttgataatgggggcagtaacagatctgctgaagcacagaa
acttggtaatgggatcaacatcattgtggccacaccaggccgtctgctggaccatatgcagaataccccaggatttatgtataaaaacctgcagtgtct
ggttattgatgaagctgatcgtatcttggatgtggggtttgaagaggaattaaagcaaattattaaacttttgccaacacgtagacagactatgctctt
ttctgccacccaaactcgaaaagttgaagacctggcaaggatttctctgaaaaaggagccattgtatgttggcgttgatgatgataaagcgaatgcaac
agtggatggtcttgaacagggatatgttgtttgtccttctgaaaagagattccttctgctctttacattccttaagaagaaccgaaagaagaagcttat
ggtcttcttttcatcttgtatgtctgtgaaataccactatgagttgctgaactacattgatttgcccgtcttggccattcatggaaagcaaaagcaaaa
taagcgtacaaccacattcttccagttctgcaatgcagattcgggaacactattgtgtacggatgtggcagcgagaggactagacattcctgaagtcga
ctggattgttcagtatgaccctccggatgaccctaaggaatatattcatcgtgtgggtagaacagccagaggcctaaatgggagagggcatgccttgct
cattttgcgcccagaagaattgggttttcttcgctacttgaaacaatccaaggttccattaagtgaatttgacttttcctggtctaaaatttctgacat
tcagtctcagcttgagaaattgattgaaaagaattactttcttcataagtcagcccaggaagcatataagtcatacatacgagcctatgattcccattc
tctgaaacagatctttaatgttaataacctaaatttgcctcaggttgctctgtcatttggtttcaaggtgcctcccttcgttgatctgaacgtcaacag
taatgaaggcaagcagaaaaagcgaggaggtggtggtggatttggctaccagaaaaccaagaaagttgagaaatccaaaatctttaaacacattagca
agaaatcatctgacagcaggcagttctctcactga
[Chemical Formula 74]
Nucleic acid sequence of pLgBiT-DDX19B
(SEQ ID NO: 69)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcggggggtcgcgcgccccagagtgtgacctttt
cggtctgctcgcagacccccgggcggcgccgcccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatggccactgactc
atgggccctggcggtggacgagcaggaagctgcggctgagtcgttgagcaacttgcatcttaaggaagagaaaatcaaaccagataccaatggtg
ctgttgtcaagaccaatgccaatgcagagaagacagatgaagaagagaaagaggacagagctgcccagtccttactcaacaagctgatcagaagc
aaccttgttgataacacaaaccaagtggaagtcctgcaacgggatccaaactcccctctgtactcggtgaagtcgtttgaagagcttcggctgaaacc
acagcttctccagggagtctatgccatgggcttcaatcgaccctccaagatacaagagaacgcattacccatgatgcttgctgaacccccacagaatc
tgattgcccagtctcagtctggcactggtaaaacagctgcctttgtcttagccatgctcagccgagtggagccatcagacagatacccccagtgtctgt
gcctctccccaacatatgagctggcgcttcaaacaggaaaagtgattgagcagatgggcaaattttacccagaactgaagcttgcctatgccgttcga
ggcaataaattggaaagaggccagaagatcagtgagcagattgtcattggcacccctgggaccgtgctggactggtgctccaagctcaagttcattg
atcccaagaaaatcaaggtgtttgttctggatgaggctgatgtcatgatagccactcagggccaccaagatcagagcatccgcatccagaggatgct
gcccaggaactgccagatgctgcttttctccgccacctttgaagactctgtgtggaagtttgcccagaaagtggtcccagacccaaatgttatcaaact
gaagcgtgaggaagagaccctggataccatcaagcagtactatgtcctgtgcagcagcagagacgagaagttccaggccttgtgtaacctctacgg
ggccatcaccattgctcaagccatgatcttctgccatactcgcaaancagctagttggctggcagcagagctctcaaaagaaggccaccaggtggct
ctgctgagtggggagatgatggtggagcagagggctgcggtgattgagcgcttccgagagggcaaagagaaggttttggtgaccaccaacgtgtg
tgcccgcggcattgatgttgaacaagtgtctgtcgtcatcaactttgatcttcccgtggacaaggacgggaatcctgacaatgagacctacctgcacc
ggatcgggcgcacgggccgctttggcaagaggggcctggcagtgaacatggtggacagcaagcacagcatgaacatcctgaacagaatccagg
agcattttaataagaagatagaaagattggacacagatgatttggacgagattgagaaaatagccaactga
[Chemical Formula 75]
Nucleic acid sequence of pLgBiT-DDX20
(SEQ ID NO: 70)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcggatcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatggggcggcat
ttgaagcctcgggagccttagcagcagtggcgactgctatgccggctgagcatgtggccgtgcaggtcccggccccagagccaacacccgggcc
tgtgaggatcctgcggaccgctcaggatctcagcagcccgcggacccgcacgggggatgtgctgttggcggagccggccgacttcgagtcactg
ctgctttcgcggccggtgctggaggggctgcgggcggccggcttcgagaggccctcgccggtgcagctcaaggccatcccgttggggcgctgcg
ggctcgatttaattgttcaagctaaatctggcaccgggaaaacctgtgtgttctccaccatagctttggactctcttgttcttgaaaacttaagtacc
cagattttgatcttggctcctacaagagaaattgctgtacagatacattctgttattacagccattggaataaaaatggaaggcttagagtgtcatgtc
tttattggagggaccccattatcacaagacaaaaccagacttaaaaagtgtcatattgctgttggatctcctggcagaattaagcaactcatagaactt
gactacttgaacccaggcagtatacgcctctttattcttgatgaagcagataagcttttagaagaaggcagcttccaggagcaaataaattggatttat
tcttccttgcctgccagtaaacagatgctggcagtatcagctacttatcccgaatttttggctaatgctttgacaaagtacatgagagatcccactttt
gtaagactgaattccagtgatccaagtctcataggtttgaagcagtattacaaagttgtcaattcataccctttggcacataaggtttttgaggaaaag
actcagcatttacaggaactgttcagcagaattccatttaatcaagctttagtcttttctaatttgcacagcagagcacaacatttggctgatatcctt
tcttctacaggctttcctgctgagtgcatttcaggcaatatgaatcagaatcagcgtcttgatgctatggctaaactgaagcactttcattgcagagtc
ctcatttccacagatttgacttctcgtgggattgatgctgagaaggtgaatctggttgtaaatctggatgtaccattggattgggagacatacatgcat
cggattgggagagctggccgttttggtacattggggctgacagtgacctactgttgccggggagaggaagaaaatatgatgatgagaattgcccagaaa
tgtaatatcaaccttctccctttaccagatcccattccttctggtctgatggaagaatgtgtggattgggatgtggaagttaaagctgctgtgcatac
atatggtatagcaagtgtacctaaccaacccttaaaaaagcaaattcagaaaatagagagaacccttcaaattcagaaagctcatggtgaccacatggc
ttcctctagaaataattctgtatctggactatcagtcaaatcaaaaaataataccaaacaaaagcttcctgtgaaaagccactcagaatgtggaatcat
agaaaaagcaacgtcaccaaaagaactgggctgtgacaggcaatccgaagagcaaatgaagaattctgttcagactcccgttgaaaactccaccaacag
tcagcaccaggtcaaagaagctttacctgtgtcactcccccagattccttgtctgtcttcctttaaaatccatcagccatacacgttgacttttgctga
attggtagaggattatgaacattatattaaagaggggttagagaaacctgtggaaatcatcaggcactacacaggccctggggatcagactgtgaatcc
tcaaaatggttttgtgagaaataaagttattgaacagagagtccctgtgttggcaagtagtagccaatctggagactctgagagtgacagtgattctta
cagctcaagaacctcttcccagagcaaaggaaataagtcatacttggaaggctcttctgataatcagctgaaagactctgaatctacgcctgtggatga
tcgtatttctttggaacaaccaccaaatggaagtgacacccccaatccagagaaatatcaagaatcacctggaatccagatgaagacaagacttaaag
agggggctagccagagagctaagcagagccggagaaacctacccaggcggtcttccttcagattgcagactgaagcccaggaagatgattggtatgact
gtcatagggaaatacgtctgagtttttctgatacctatcaggattatgaggagtactggagagcttactacagggcatggcaagaatattatgctgccg
cttctcattcatattattggaatgctcagagacatccaagttggatggcagcttatcacatgaataccatttatctacaagaaatgatgcatagtaacc
agtga
[Chemical Formula 76]
Nucleic acid sequence of pLgBiT-DDX21
(SEQ ID NO: 71)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtca
ttggcgaagtcgaacacgcagatgcagtcggggggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgaggccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgccgggaaaac
tccgtagtgacgctggtttggaatcagacaccgcaatgaaaaaaggggagacactgcgaaagcaaaccgaggagaaagagaaaaaagagaagc
caaaatctgataagactgaagagatagcagaagaggaagaaactgttttccccaaagctaaacaagttaaaaagaaagcagagccttctgaagttga
catgaattctcctaaatccaaaaaggcaaaaaagaaagaggagccatctcaaaatgacatttctcctaaaaccaaaagtttgagaaagaaaaaggag
cccattgaaaagaaagtggtttcttctaaaaccaaaaaagtgacaaaaaatgaggagccttctgaggaagaaatagatgctcctaagcccaagaaga
tgaagaaagaaaaggaaatgaatggagaaactagagagaaaagccccaaactgaagaatggatttcctcatcctgaaccggactgtaaccccagt
gaagctgccagtgaagaaagtaacagtgagatagagcaggaaatacctgtggaacaaaaagaaggcgctttctctaattttcccatatctgaagaaa
ctattaaacttctcaaaggccgaggagtgaccttcctatttcctatacaagcaaagacattccatcatgtttacagcgggaaggacttaattgcacagg
cacggacaggaactgggaagacattctcctttgccatccctttgattgagaaacttcatggggaactgcaagacaggaagagaggccgtgcccctca
ggtactggttcttgcacctacaagagagttggcaaatcaagtaagcaaagacttcagtgacatcacaaaaaagctgtcagtggcttgtttttatggtgg
aactccctatggaggtcaatttgaacgcatgaggaatgggattgatatcctggttggaacaccaggtcgtatcaaagaccacatacagaatggcaaact
agatctcaccaaacttaagcatgttgtcctggatgaagtggaccagatgttggatatgggatttgctgatcaagtggaagagattttaagtgtggcata
caagaaagattctgaagacaatccccaaacattgcttttttctgcaacttgccctcattgggtatttaatgttgccaagaaatacatgaaatctacata
tgaacaggtggacctgattggtaaaaagactcagaaaacggcaataactgtggagcatctggctattaagtgccactggactcagagggcagcagttat
tggggatgtcatccgagtatatagtggtcatcaaggacgcactatcatcttttgtgaaaccaagaaagaagcccaggagctgtcccagaattcagctat
aaagcaggatgctcagtccttgcatggagacattccacagaagcaaagggaaatcaccctgaaaggttttagaaatggtagttttggagttttggtggc
aaccaatgttgctgcacgtgggttagacatccctgaggttgatttggttatacaaagctctccaccaaaggatgtagagtcctacattcatcgatccgg
gcggacaggcagagctggaaggacgggggtgtgcatctgcttttatcagcacaaggaagaatatcagttagtacaagtggagcaaaaagcgggaat
taagttcaaacgaataggtgttccttctgcaacagaaataataaaagcttccagcaaagatgccatcaggcttttggattccgtgcctcccactgccat
tagtcacttcaaacaatcagctgagaagctgatagaggagaagggagctgtggaagctctggcagcagcactggcccatatttcaggtgccacgtcc
gtagaccagcgctccttgatcaactcaaatgtgggttttgtgaccatgatcttgcagtgctcaattgaaatgccaaatattagttatgcttggaaagaa
cttaaagagcagctgggcgaggagattgattccaaagtgaagggaatggtttttctcaaaggaaagctgggtgtttgctttgatgtacctaccgcatca
gtaacagaaatacaggagaaatggcatgattcacgacgctggcagctctctgtggccacagagcaaccagaactggaaggaccacgggaaggatatg
gaggcttcaggggacagcgggaaggcagtcgaggcttcaggggacagcgggacggaaacagaagattcagaggacagcgggaaggcagtag
aggcccgagaggacagcgatcaggaggtggcaacaaaagtaacagatcccaaaacaaaggccagaagcggagtttcagtaaagcatttggtcaa
taa
[Chemical Formula 77]
Nucleic acid sequence of pLgBiT-DDX23
(SEQ ID NO: 72)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatggcaggagagc
tggctgacaaaaaggaccgtgatgcatcaccttccaaggaggaaaggaaggatcacggactcctgacagagagcgggatagagaccgggacc
ggaagtcttccccatctaaagatagaaagcggcatcgttcaagggatagacgtcgaggaggcagccgttctcgctctcgttcccgttccaaatctgca
gaaagagaacgacggcacaaagaacgagaacgagataaggagcgggatcggaataagaaggaccgagatcgagacaaggatgggcacaga
cgggacaaggaccgtaaacgatccagcttatctcctggtcgaggaaaagactttaaatctcggaaggacagagactctaagaaggatgaagaggat
gaacatggtgataagaagcctaaggcccagccattatccctggaggagcttctggccaagaaaaaggctgaggaagaagctgaggctaagccca
agttcctctctaaagcagaacgagaggctgaagctctaaagcgacggcagcaggaggtggaagagcggcagaggatgcttgaagaagagagga
agaaaaggaaacagttccaagacttgggcaggaagatgttggaagatcctcaggaacgggaacgtcgggaacgcagggagaggatggaacgg
gagaccaatggaaatgaggatgaggaagggcggcagaagatccgggaagagaaggataagagcaaggaactgcatgccattaaggagcgtta
cctgggtggcatcaaaaagcggcgccgaacgagacatctcaatgaccggaaatttgtttttgagtgggatgcatctgaggacacatccattgactaca
accccctgtacaaagaacggcaccaggtgcagttgttagggcgaggcttcattgcaggcattgacctcaagcagcagaagcgagagcagtcacgt
ttctatggagacctaatggagaagaggcgaaccctggaagaaaaggagcaggaggaggcaagactccgcaaacttcgtaagaaggaagccaag
cagcgctgggatgatcgtcattggtctcagaaaaagttagatgagatgacggacagggactggcggatcttccgtgaggactacagcatcaccacc
aaaggtggcaagatccccaatcccatccgatcctggaaagactcttctctgcccccacacatcttggaggtcattgataagtgtggctacaaggaacc
aacacctatacagcgtcaggcaattcccattgggctacagaatcgtgacatcattggtgtggctgagactggcagtggcaagacagcagccttcctc
atccctctgctggtctggatcaccacacttcccaaaattgacaggatcgaagagtcagaccaaggcccttatgccatcatcctggctcccacccgtga
gttggctcaacagattgaggaagagaccatcaagtttgggaaaccgctaggtatccgcactgtggctgtcattggtggcatctccagagaagaccag
ggcttcaggctgcgcatgggttgtgagattgtgattgctacccctgggcgtttgattgatgtgctggagaaccgctacctggtgctgagccgctgtacc
tatgtggttctggatgaggcagataggatgattgacatgggctttgagccagatgtccagaagatcctggagcacatgcctgtcagcaaccagaagc
cagacacggatgaggctgaggaccctgagaagatgctggccaactttgagtcgggaaaacataagtaccgccaaacagtcatgttcacggccacc
atgcccccagcggtggagcgtctggccaggagctatcttcggcgacctgctgtggtgtacattggctccgcaggcaagccccatgagcgtgtggaa
cagaaggtcttcctcatgtcagagtcagaaaagaggaaaaagctgctggcaatcttggagcaaggctttgacccacccatcattatttttgtcaaccag
aagaagggctgcgacgtgttggccaaatccctggagaagatggggtacaatgcttgcacactgcacggtggaaaaggccaggagcagcgagagt
ttgcgttgtccaacctcaaggctggggccaaggatattttggtggctacagatgtggctggtcgtggtattgacatccaagatgtgtctatggttgtca
actatgatatggccaaaaatattgaagattacatccaccgcattggccgcacgggacgagcaggcaagagtggggggccatcaccttcctcacaaa
agaggactctgctgtgttctacgagctgaagcaagctatcctggaaagcccagtgtcttcctgtccccccgaactagccaaccacccagatgcccag
cataagccaggcaccatcctcaccaagaagcgccgggaagagaccatctttgcctga
[Chemical Formula 78]
Nucleic acid sequence of pLgBiT-DDX24
(SEQ ID NO: 73)
ggcctaactggccggtacctgagtctaaatgagtcttcggacctcgcgggggccgcttaagcggtgcttagggtttgtctgacgcggggggagg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactgggggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtgggggagcggaggtggaggcatgaagttgaagga
cacaaaatcaaggccaaagcagtcaagctgtggcaaatttcagacaaagggaatcaaagttgtgggaaaatggaaggaagtgaagattgacccaa
atatgtttgcagatggacagatggatgacttggtgtgctttgaggaattgacagattaccagttggtctcccctgccaagaatccctccagtctcttct
caaaggaagcacccaagagaaaggcacaagctgtttcagaagaagaggaggaggaggagggaaagtctagctcaccaaagaaaaagatcaagttg
aagaaaagtaaaaatgtagcaactgaaggaaccagtacccagaaagaatttgaagtgaaagatcctgagctggaggcccagggagatgacatggt
ttgtgatgatccggaggctggggagatgacatcagaaaacctggtccaaactgctccaaaaaagaagaaaaataaagggaaaaaagggttggagc
cttctcagagcactgctgccaaggtgcccaaaaaagcgaagacatggattcctgaagttcatgatcagaaagcagatgtgtcagcttggaaggacct
gtttgttcccaggccggttctccgagcactcagctttctaggcttctctgcacccacaccaatccaagccctgaccttggcacctgccatccgtgacaa
actggacatccttggggctgctgagacaggaagtgggaaaactcttgcctttgccatcccaatgattcatgcggtgttgcagtggcagaagaggaat
gctgcccctcctccaagtaacaccgaagcaccacctggagagaccagaactgaggccggagctgagactagatcaccaggcaaggctgaagct
gagtctgatgcattgcctgacgatactgtaattgagagtgaagcactgcccagtgatattgcagccgaggccagagccaagactggaggcactgtct
cagaccaggcgttgctctttggtgacgatgatgctggtgaagggccttcttccctgatcagggagaaacctgttcccaaacagaatgagaatgagga
ggaaaatcttgataaagagcagactggaaatctaaaacaggagttggatgacaaaagcgccacctgtaaggcatatccaaagcgtcctctgcttgga
ctggttctgactcccactcgagagctggccgtccaggtcaaacagcacattgatgctgtggccaggtttacaggaattaaaactgctattttggttggt
ggaatgtccacgcagaaacagcagaggatgctgaaccgtcgtcctgagattgtggttgctactccaggccggctgtgggaattaattaaagaaaagca
ttatcatttgaggaaccttcggcagctcaggtgcctggtagtggatgaggctgaccggatggttgagaaaggccattttgctgagctctcacagctgct
agagatgctcaatgactcccaatacaacccaaagagacaaacgcttgttttttctgccacactcaccctggtgcatcaggctcctgctcgaatccttca
taagaagcacaccaagaaaatggataaaacagccaaacttgacctccttatgcagaaaattggcatgaggggcaagcccaaggtcattgacctcaca
aggaatgaggccacggtggagacgctaacagagaccaagatccattgtgagactgatgagaaagacttctacttgtactacttcctgatgcagtatcc
aggccgcagcttagtgtttgccaacagtatctcctgcatcaaacgcctctctgggctcctcaaagtccttgatatcatgcccttgaccctgcatgcctg
tatgcaccagaagcagaggctcagaaacctggagcagtttgcccgtctggaagactgtgttctcttggcaacagatgtggcagctcggggtctggatat
tcctaaagtccagcatgtcatccattaccaggtcccacgtacctcggagatttatgtccaccgaagtggtcgaactgctcgagctaccaatgaaggcct
cagtctgatgctcattgggcctgaggatgtgatcaactttaagaagatttacaaaacgctcaagaaagatgaggatatcccactgttccccgtgcagac
aaaatacatggatgtggtcaaggagcgaatccgtttagctcgacagattgagaaatctgagtatcggaacttccaggcttgcctgcacaactcttggat
tgagcaggcagcagctgccctggagattgagctggaagaagacatgtataagggaggaaaagctgaccagcaagaagaacgtcggagacaaaa
gcagatgaaggttctgaagaaggagctgcgccacctgctgtcccagccactgtttacggagagccagaaaaccaagtatcccactcagtctggcaa
gccgcccctgcttgtgtctgccccaagtaagagcgagtctgctttgagctgtctctccaagcagaagaagaagaagacaaagaagccgaaggagc
cacagccggaacagccacagccaagtacaagtgcaaattaa
[Chemical Formula 79]
Nucleic acid sequence of pLgBiT-DDX25
(SEQ ID NO: 74)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgangcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatggcgtcgttact
gtggggaggcgacgcaggggcggcggagagcgagcggctgaacagccacttttcaaacctcagccaaccccggaagaaccctttggggtattaa
gagtactgcagtccgaaacatagatggttctattaataacatcaatgaagatgatgaagaagatgtagtggatttggcagctaattcactcttaaacaa
gttaatccatcaatccttagtagaatccagtcaccgtgtggaagtttttacagaaggatcccagctctccactttactcagtaaagacatttgaagagc
tgcggctaaaggaagagttactaaaaggaatctatgcaatgggatttaataggccatctaaaatccaagagatggctctccctatgatgctggcacatc
caccccagaacctcatagcacagagccagtctggaacaggaaagacagcggcatttgtgttggcaatgttaagcagagttaatgccttggaattgttcc
cacagtgcctctgcctagctcctacttatgaattggctctgcaaactggccgtgtggttgagcagatgggaaaattctgtgtggatgttcaagtgatgt
atgccattcgagggaatcgaattcccagaggcaccgacatcactaaacagattataattggcactcctgggactgtcctagattggtgttttaaacta
aaattgattgatttgactaagattcgtgtgtttgtcctggatgaagcagatgtgatgattgacactcaaggattctcagatcatagtattcgtattcaa
agagctctaccctccgaatgccaaatgctcctcttttcagcaacctttgaggactctgtgtggcactttgctgagcgaatcatccctgaccctaatgtt
atcaagttacgcaaagaggagctcacactgaacaacatccggcaatattacgtgctgtgtgagcacaggaaagacaaataccaagctctgtgcaacatt
tatggcagcatcaccattggtcaggccatcatcttctgccagactcgtcgaaacgctaagtggttgaccgtggagatgatacaggatggccaccaggtg
tctttgttaagcggggagctgaccgtggagcagcgagcttccatcattcagaggtttcgggatgggaaagagaaggttctcataacaactaatgtttgt
gcccgagggattgatgtgaagcaggtcacaattgttgtgaactttgatctccctgtaaaacaaggagaggagccggactatgagacctacctccaccgc
atagggcggacggggcgctttgggaaaaaaggccttgccttcaacatgattgaagtagatgagctgccctcgctcatgaaaatccaggaccactttaac
agcagtattaagcaactcaacgctgaagacatggatgaaattgaaaagattgactattga
[Chemical Formula 80]
Nucleic acid sequence of pLgBiT-DDX27
(SEQ ID NO: 75)
ggcctaactggccggtacctgagtctaaatgagtcttcggacctcgcgggggccgcttaagcggtggttagggtttgtctgacgcggggggaggg
ggaaggaacgaaacactctcattcggaggggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgcttgcggacct
cggcttaatcggaaccataggcgaggatgacgaggtgccggtggagcccgagtctgactccggggacgaggaagaggaggggcccattgtgct
gggcagacgacaaaaagctttggggaagaaccgcagtgctgatttcaaccctgatttcgttttcactgagaaggaggggacgtacgatggcagctg
ggccctggctgatgtcatgagccaactcaagaagaagagggcagccactacattagatgagaagattgagaaagttcgaaagaaaaggaaaaca
gaggataaagaagccaagtctgggaagttggaaaaggagaaagaagcaaaggaaggctctgaaccaaaggagcaggaagaccttcaagagaat
gatgaggaaggctcagaagatgaagcctcggagactgactactcatcagctgatgagaacatcctcaccaaagcagatacactcaaagtaaaggat
cggaagaagaagaagaagaaaggacaggaagcaggaggattttttgaagatgcatctcagtacgatgaaaacctctcgttccaggacatgaacctt
tcccgccctcttctgaaggccattacagccatgggcttcaagcagcccaccccgatccagaaggcgtgcatacctgtgggtctattggggaaggac
atctgtgcctgtgcagccactgggacaggtaaaactgccgcctttgccctgcctgttttggagcgtctgatttataaaccccgccaggctccagtcacc
cgcgtgctggtgctagtgcccacccgagagctgggcatccaggtgcactctgtcaccagacagctggcccagttctgcaacatcaccacctgcctg
gctgtgggggcttggatgtgaagtctcaggaagcagctcttcgggcagcgcctgacatcctcatcgccaccccaggccggctcatcgatcacctc
cacaactgcccttccttccacctgagcagcatcgaggtgctcatcctggacgaggctgacaggatgctggatgagtactttgaggagcagatgaag
gagatcatccgaatgtgttcccaccaccgccagaccatgctcttctcggccaccatgacagacgaggtgaaagatctggcttctgtctccttgaagaa
tcctgtccggatatttgtgaacagcaacacagatgtggctcccttcctgcggcaggagttcatccggatccggcctaatcgtgaaggagaccgagaa
gccatcgtggcagctttgttgacgaggaccttcactgaccatgtgatgctgttcacgcaaaccaagaagcaggcccaccgcatgcacatcctcctgg
ggctcatggggctgcaggtgggtgagctccatggcaacttgtcacagacgcagcggctggaggccctccggcgttttaaggatgaacagattgac
atcctcgtggccactgatgtggcagcccgtggacttgacattgagggggtcaaaacggtaatcaacttcacaatgcctaataccatcaaacattatgtc
caccgggggggcgaacagcacgtgctggcagggctgggcgctcagtctctctggtgggagaagatgagcggaagatgctgaaggagattgtaa
aagctgccaaggcccctgtgaaggccaggatacttccccaagatgtcatcctcaaattccgggacaagattgagaaaatggagaaagatgtgtatgc
agttctgcagctagaggggaggaaaaagagatgcagcagtcagaagcccagatcaatacagcaaagcggctcctggagaaggggaaggagg
cagtggtccaagagcccgagaggagctggttccagaccaaagaagagaggaagaaggagaaaattgccaaagctctgcaggaatttgacttggc
cttaagaggaaagaagaaaaggaagaagtttatgaaggatgccaaaaaaaagggggagatgacagcagaggaaaggtctcagtttgaaatcctca
aggcgcagatgtttgctgaacggctagcgaagaggaatcgcagagccaagcgggcccgagcaatgcccgaggaggagccagtgagaggtcct
gccaagaagcaaaagcaggggaagaaatctgtatttgatgaagaactcaccaacacaagcaagaaggccctgaaacagtatcgagctggcccttc
ctttgaagaaaggaaacagttgggcttgccccaccagagacgaggaggaaactttaaatctaaatccagatacaagaggaggaagtag
[Chemical Formula 81]
Nucleic acid sequence of pLgBiT-DDX28
(SEQ ID NO: 76)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaagctgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacaggggagttccggtggtgggggagcggaggtggaggcatggctctaacgcg
gccggtgcggctcttttccctcgtgactcggttgctcctggcgccgcgacggggcctcacggtccgcagtcccgacgaacccctgccggtggtgcg
catcccagtggctctacagcggcagttggaacagcggcagagcaggcggcggaacctcccgaggccggtgctggttcgacccggaccgctgct
ggtttcggcgcggcggccggagttgaaccagccggcgcgcctcacactgggccgttgggagcgcgcgccgctagcctctcaaggctggaagag
tcgacgcgcgcgtcgggaccacttctccatcgagcgcgcgcaacaggaggcgccagcggtgcgaaagctctcgtctaagggcagctttgctgac
ctgggcctggagccccgtgtgctgcacgcactacaggaggctgcgcctgaagtcgttcagcccacaaccgtgcagtctagcaccatcccctcacta
cttcgcggccgccatgtcgtttgcgccgcagaaaccggcagtggcaagactctcagctacctcctgccgctgcttcaacggctcttgggccagcca
agcctggactcccttcctatccccgcgccccgaggcctggtccttgttccttcccgagaattggcccaacaggtgcgggctgtggcccaacccttgg
gccgctccttgggcctgctggtgcgggacctggagggaggccacggcatgcgtaggatcaggctgcagctgtccagacagccttcagcagatgt
gcttgtggccactccaggggctctgtggaaggccctgaaaagtcgactgatcagtctggagcaactctccttcttggtgttggatgaggcagacaca
ctgctggatgaaagcttcctggaactggtggactacatcttagagaagagccacatagcagaaggcccagctgacttggaagaccccttcaatccca
aagctcagttagtgctggtaggagccacatttcccgaaggtgtaggccagttgctgaataaagtcgccagcccagatgctgtcaccaccatcaccag
ctccaagctccactgtatcatgcctcatgtgaaacagacatttctgagactgaagggagcagataaggtggccgagctggtgcacatcctcaagcat
cgtgacagagcagaaaggactggtccctcaggaactgttctggtgttctgtaatagctccagcactgtgaactggctgggatatattctggatgacca
caaaatccaacacctaaggttgcaggggcaaatgccagccttgatgagggtaggaatcttccagtccttccagaagagctcccgagacatacttctct
gcacagacatagcctctcggggcctggacagcactggtgtggagctggttgtcaattatgatttccccccaacgctgcaagattacatccacagagc
agggagagtgggccgtgtggggagcgaggtgccaggcaccgtcatcagttttgtgacccatccctgggatgtgagcctggttcagaagattgagct
ggcggctcgccgaaggagaagtcttccaggactagcatcctcggtgaaagagcctttgccccaagcaacctga
[Chemical Formula 82]
Nucleic acid sequence of pLgBiT-DDX31
(SEQ ID NO: 77)
ggcctaactggccggtacctgagtctaaatgagtcttcggacctcgcgggggccgcttaagcggtggttagggtttgtctgacgcggggggagg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtgggggagcggaggtggaggcatggcagccgccg
acggttcgctcttcgacaaccccaggacgttctccagacgtcccccagcccaggcgagtcggcaagcaaaggctacgaaaagaaaataccaagc
gtccagtgaggctcccccagcgaaacggaggaacgaaacttcatttctcccagccaagaaaactagtgttaaagaaactcagaggacttttaaggg
gaacgcacaaaaaatgttttctccaaagaagcattcggttagcacaagtgatagaaaccaggaggagagacagtgcattaagacttcatcactgttta
aaaacaaccctgacattccagaactccacagacctgtggtaaagcaggtgcaagaaaaagtgtttacttcagctgcttttcatgagctgggcctccac
ccacatttaatttccacaataaatacggtcttaaaaatgtctagtatgaccagtgttcagaagcaaagtattcctgtgttgctggaaggcagagatgct
ctcgtgagatcccagacgggctcaggtaaaactcttgcctattgcatccctgtggtccagtcccttcaagcaatggagtcaaaaatacagcgcagtgatg
gcccctatgccctggtgctcgtgccaacgagagagctagctctacaaagctttgacactgtccagaaactgcttaagcctttcacctggattgtgcctg
gagtgttaatgggaggagagaagagaaaatcagaaaaggccagactccgcaaaggaataaatatccttatctcaactcctggacgcctggtggatc
atataaaatccacaaagaacattcattttagtcggctgcggtggttggtgtttgatgaagcagacagaatcttggatttgggttttgaaaaggacatca
cagtgatacttaatgtgtaaatgctgaatgccaaaaacgacagaatgtcttgctatcagcgacactcacagaaggtgtaacgcggctagctgatatcag
tttgcatgatccagtcagtatttctgtcctggacaagagccatgaccagttgaacccaaaggacaaagcggtccaggaggtctgtcctccaccagctg
gcgacaagctggacagctttgcaataccagagagtctcaagcagcatgtgactgtggttcccagcaaactgaggcttgtctgcctagcggccttcatc
cttcagaaatgcaagtttgaggaagaccagaagatggttgtctttttctcaagttgcgagctggtggagttccactacagcctcttcctacagaccctg
ctgagcagctcaggggcgccggcatcagggcagttgccatctgcctccatgcgattaaaattcctacggctgcatggcggcatggagcaggaggaa
agaacagcagtgtttcaggaattttcacattccagaagaggcgtccttctttgcacggatgttgcagctcggggcttagatctccctcaagtcacgtgg
attgttcagtacaacgctccatcttcacctgcagaatacatccaccggattggaagaaccgcccggattggctgccatgggagcagcctgctcattttg
gctccttcggaggcagaatatgtcaactcgttggcttctcacaaaatcaacgtttctgagattaagatggaagatattttgtgtgttctgacaagagat
gattgttttaaagggaaacgatggggagcccagaaatcccatgctgttggcccccaggaaatccgagagcgagccacagtcttgcagacggtatttgaa
gattacgtgcactccagtgagaggagggtctcctgggcaaagaaagctctgcagtccttcatccaagcctacgccacctaccccagggagctgaag
cacatcttccacgtccgatccctccaccttgggcatgtggcgaagagcttcggactaagagatgcccccaggaatcttagtgccttgactagaaagaa
gaggaaagcacacgtgaaaaggcctgaccttcataagaagacccagagtaaacacagcctcgctgaaatcctacgttcggaatactcaagcggca
tggaggccgacatcgccaaggtcaaaaagcaaaacgcacctggagagcctggtggccggcccctgcagcacagtctgcagccgacaccctgctt
tggccgtgggaaaacattaaaatggagaaaaacccaaaaaggtgtacagcgggacagcaagacttcccagaaagtttaa
[Chemical Formula 83]
Nucleic acid sequence of pLgBiT-DDX39B
(SEQ ID NO: 78)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcggggggtcgcgcgccccagagtgtgacctttt
cggtctgctcgcagacccccgggcggcgcgcgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatggcagagaacg
atgtggacaatgagctcttggactatgaagatgatgaggtggagacagcagctgggggagatggggctgaggcccctgccaagaaggatgtcaa
gggctcctatgtctccatccacagctctggctttcgtgacttcctgctcaagccagagttgctccgggccattgtcgactgtggctttgagcatccgtc
agaagtccagcatgagtgcatccctcaggccattctgggaatggatgtcctgtgccaggccaagtcgggcatgggaaagacagcagtgtttgtcttgg
ccacactgcaacagctggagccagttactgggcaggtgtctgtactggtgatgtgtcacactcgggagttggcttttcagatcagcaaggaatatgag
cgcttctctaaatacatgcccaatgtcaaggttgctgttttttttggtggtctgtctatcaagaaggatgaagaggtgctgaagaagaactgcccgcat
atcgtcgtggggactccaggccgtatcctagccctggctcgaaataagagcctcaacctcaaacacattaaacactttattttggatgaatgtgataag
atgcttgaacagctcgacatgcgtcgggatgtccaggaaatttttcgcatgaccccccacgagaagcaggtcatgatgttcagtgctaccttgagcaaa
gagatccgtccagtctgccgcaagttcatgcaagatccaatggagatcttcgtggatgatgagacgaagttgacgctgcatgggttgcagcagtactac
gtgaaactgaaggacaacgagaagaaccggaagctctttgaccttctggatgtccttgagttcaaccaggtggtgatctttgtgaagtctgtgcagcg
gtgcattgccttggcccagctactagtggagcagaacttcccagccattgccatccaccgtgggatgccccaggaggagaggctttctcggtatcag
cagtttaaagattttcaacgacgaattcttgtggctaccaacctatttggccgaggcatggacatcgagcgggtgaacattgcttttaattatgacatg
cctgaggattctgacacctacctgcatcgggtggccagagcaggccggtttggcaccaagggcttggctatcacatttgtgtccgatgagaatgatgcc
aagatcctcaatgatgtgcaggatcgctttgaggtcaatattagtgagctgcctgatgagatagacatctcctcctacattgaacagacacggtag
[Chemical Formula 84]
Nucleic acid sequence of pLgBiT-DDX41
(SEQ ID NO: 79)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactgggggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctggggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcatgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccgggggggggagcggaggtggaggcatggaggagtcgg
aacccgaacggaagcgggctcgcaccgacgaggtgcctgccggaggaagccgctccgaggcggaagatgaggacgacgaggactacgtgcc
ctatgtgccgttacggcagcgccggcagctactgctccagaagctgctgcagcgaagacgcaagggagctgcggaggaagagcaacaggaca
gcggtagtgaaccccggggagatgaggacgacatcccgctaggccctcagtccaacgtcagcctcctggatcagcaccagcaccttaaagagaa
ggctgaagcgcgcaaagagtctgccaaggagaagcagctgaaggaagaagagaagatcctggagagtgttgccgagggccgagcattgatgtc
agtgaaggagatggctaagggcattacgtatgatgaccccatcaaaaccagctggactccaccccgttatgttctgagcatgtctgaagagcgacat
gagcgcgtgcggaagaaataccacatcctggtggagggagacggtatcccaccacccatcaagagcttcaaggaaatgaagtttcctgcagccat
cctgagaggcctgaagaagaaaggcattcaccacccaacacccattcagatccagggcatccccaccattctatctggccgtgacatgataggcat
cgctttcacgggttcaggcaagacactggtgttcacgttgcccgtcatcatgttctgcctggaacaagagaagaggttacccttctcaaagcgcgagg
ggccctatggactcatcatctgcccctcgcgggagctggcccggcagacccatggcatcctggagtactactgccgcctgctgcaggaggacagc
tcaccactcctgcgctgcgccctctgcattgggggcatgtccgtgaaagagcagatggagaccatccgacacggtgtacacatgatggtggccacc
ggggcgcctcatggatttgctgcagaagaagatggtcagcctagacatctgtcgctacctggccctggacgaggctgaccgcatgatcgacatg
ggcttcgagggtgacatccgtaccatcttctcctacttcaagggccagcgacagaccctgctcttcagtgccaccatgccgaagaagattcagaactt
tgctaagagtgcccttgtaaagcctgtgaccatcaatgtggggcgcgctggggctgccagcctggatgtcatccaggaggtagaatatgtgaagga
gaggccaagatggtgtacctgctcgagtgcctgcagaagacacccccgcctgtactcatctttgcagagaagaaggcagacgtggacgccatcc
acgagtacctgctgctcaagggggttgaggccgtagccatccatgggggcaaagaccaggaggaacggactaaggccatcgaggcattccggg
agggcaagaaggatgtcctagtagccacagacgttgcctccaagggcctggacttccctgccatccagcacgtcatcaattatgacatgccagagg
agattgagaactatgtacaccggattggccgcaccgggcgctcgggaaacacaggcatcgccactaccttcatcaacaaagcgtgtgatgagtcag
tgctgatggacctcaaagcgctgctgctagaagccaagcagaaggtgccgcccgtgctgcaggtgctgcattgcggggatgagtccatgctggac
attggaggagagcgcggctgtgccttctgcgggggcctgggtcatcggatcactgactgccccaaactcgaggctatgcagaccaagcaggtcag
caacatcggtcgcaaggactacctggcccacagctccatggacttctga
[Chemical Formula 85]
Nucleic acid sequence of pLgBiT-DDX42
(SEQ ID NO: 80)
ggcctaactggccggtacctgagtctaaatgagtcttcggacctcgcgggggccgcttaagcggtgattagggtttgtctgacgcggggggaggg
ggaaggaacgaaacactctcattcggaggggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgaactggaataa
aggtggtcctggcactaagcgaggatttggctttggaggttttgccatcagtgctgggaaaaaggaggaacccaaactcccacagcagtcccacag
tgcctttggggcaaccagctcttcttctggatttggaaagtcagctccaccacagcttccttctttctacaaaattggatctaagcgggccaactttga
tgaagaaaatgcctattttgaagatgaggaagaagattctagcaacgttgatttaccttacattcctgctgaaaactcaccaactcgccagcaattcca
ttccaagccagtagattctgacagcgatgatgatcccttggaggcattcatggctgaagtggaggatcaggcagctagagacatgaagaggcttgaaga
aaaggacaaggaaagaaaaaacgtaaagggtattcgagatgacattgaagaggaagatgaccaagaagcttattttcgatacatggcagaaaacccaa
ctgctggtgtggttcaggaggaagaggaagacaatctagaatatgatagtgacggaaatccaattgcacctaccaaaaaaatcattgatcctcttccc
cccattgatcattcagagattgactatccaccatttgaaaaaaacttttacaatgagcatgaagagataaccaacctcactccacagcagttaatagat
ctccggcataagctcaatcttcgggtctctggtgctgcacctcctagaccaggaagtagctttgctcattttgggtttgacgaacaacttatgcacca
gattcggaaatctgaatacacacagcccactccaatacagtgccagggtgtgcctgtggcattaagtggtagagacatgattggtattgccaaaacagg
tagtgggaaaactgcagccttcatttggcccatgttgattcatataatggaccagaaggagttggaaccaggtgatggaccaattgcagtgattgtgtg
tcctaccagggagctttgccagcagatccatgcagaatgtaagcggtttggaaaagcatataatcttcgatcagtggccgtatatggaggagggagtat
gtgggagcaggccaaggcccttcaggagggggcagagattgttgtgtgtaccccaggtcgactgatagatcatgtgaaaaagaaagctaccaatcttc
aaagagtctcttaccttgtgtttgatgaagcagatcgaatgtttgacatgggatttgagtaccaagttcgatccatagcaagtcatgttcgtcctgaca
ggcagactctcttatttagtgcaacttttcggaagaagattgaaaagttggccagagacatcctgatcgaccctattcgagtggtgcagggagatatt
ggagaggcaaatgaagatgtgacacagattgtggagattctccattctggacctagtaaatggaactggcttacccggcgtctggtagaatttacctct
tcagggagtgtcctcctcttgttactaaaaaagccaatgctgaagagctagcgaataaccttaaacaggagggtcataatcttgggctgctccatgggg
atatggatcagagtgagagaaacaaggtcatttcagactttaagaaaaaggacatcccagtcctggtggccacagatgttgcagcccgtggtctggaca
ttccttcaattaagactgtcattaactatgatgtggcacgagacattgatacccacacgcataggattggccgcacaggaagagcgggtgagaaaggt
gtggcctataccctactcactcccaaggacagcaattttgctggtgacctggtccggaacttggaaggagccaatcaacacgtttctaaggaactcct
agatctggcaatgcagaatgcctggtttcggaaatctcgattcaaaggagggaaaggaaaaaagctgaacattggtggaggaggcctaggctaca
gggagcggcctggcctgggctctgagaacatggatcgaggaaataacaatgtaatgagcaattatgaggcctacaagccttccacaggagctatgg
gagatcgactaacggcaatgaaagcagctttccagtcacagtacaagagtcactttgttgcagccagtttaagtaatcagaaggctggaagttctgct
gctggggcaagtgggggactagtgcagggagcttgaattctgttccaactaactcagcacaacagggccataacagtcctgacagccccgtcacc
agtgccgccaagggcatcccaggctttggcaatactggcaacatcagtggtgcccctgtgacctacccgtctgccggagcccaaggagtcaacaa
cacagcttcagggaataacagccgagaagggactgggggcagcaacgggaaaagagagagatatactgagaaccggggcagcagccgtcaca
gtcacggagagactggcaatcggcatagcgatagtccacgtcacggagatggtggtcgccatggagatggataccgccatccagaaagcagcag
ccgtcatactgatggccatcggcacggggagaacagacatggaggaagcgcaggccggcatggggagaaccggggtgcaaatgatggtcgga
atggggaaagcaggaaagaagcttttaatcgtgagagcaagatggagcccaagatggaacccaaagtggacagcagcaagatggacaaggtgg
acagcaagacagataagacagctgacggctttgctgtcccagagccgcctaaacgcaagaaaagtcgatgggacagttag
[Chemical Formula 86]
Nucleic acid sequence of pLgBIT-DDX43
(SEQ ID NO: 81)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggagggggggtttggtcgggggtggtaactggcgggccgctgactcggggggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttggggaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgtcccaccacg
gaggagctcccaaggcctctacgtgggtcgttgctagtcggcgaagctcgacagtgtcccgagcgccagagaggaggccggcggaggagttga
atcgaacaggtcctgagggatatagtgtcggcagaggtggtcgctggagaggcacctctaggcccccggaggccgtggccgctggtcacgagga
actgccgctgtgttttgctttgaagagccactttgttggcgcggtaatcggtcgtggtgggtcaaaaataaagaatatacaaagtacaacaaacaccac
aatccaaataatacaagaacaaccagaatcattagtcaaaatttttggcagcaaggcaatgcaaacgaaagcaaaagcagtgatagacaattttgtta
aaaagctagaagaaaattacaattcagaatgcggaattgatactgcattccaaccttctgttggaaaagatggaagcacagataacaatgtgttgcag
gagatcggccattgatagattgggatcaaattagagaggaaggtttgaaatggcaaaaaacaaagtgggcagatttaccaccaattaagaaaaacttt
tataaagagtccactgccacaagtgccatgtcaaaagtagaagcagatagttggaggaaagaaaattttaatataacgtgggatgacttgaaggatgg
ggagaaacgacctatccccaatcctacctgcacatttgatgacgcctttcaatgttatcctgaggttatggaaaacattaaaaaggcaggttttcaaaa
gccaacacctattcagtcacaggcatggcccattgtgttgcaaggaatagatcttataggagtagcccagactggaacaggaaagacattgtgttattt
aatgcctggatttattcatctggtccttcaacccagccttaaaggtcdaaggaatagacccggcatgttagttctaactcccactcgggaattagcact
tcaagtagaaggagaatgttgcaaatattcatataaagggcttcggagtgtttgtgtatatggtggtggaaatagagatgaacaaatagaagagctta
aaaaaggtgtagatatcataattgcaactcccggaagattgaatgatctgcaaatgagtaacttcgtcaatctgaagaatataacctacttggttttag
atgaagcagacaagatgttggacatgggatttgaaccccagataatgaagattttgttagatgtgcgcccagataggcagacagttatgaccagtgcta
catggcctcattcagttcatcgcctcgcacaatcttatttgaaagaaccaatgattgtctatgttggtacattggatctagttgctgtaagttcagtga
agcaaaatataattgtaaccaccgaggaagagaaatggagtcacatgcaaacttttctacagagtatgtcatccacagacaaagtcattgtcttcgttt
ctcgaaaagctgttgcggatcacttatcaagtgacctaatacttggaaatatatcagtagagtctctgcatggagatagagaacagagagatcgggaga
aagcattagagaactttaaaacaggcaaagtgagaatactaattgcaactgatctagcctctagaggacttgatgtccatgacgttacacatgtctata
attttgactttccacggaatattgaagaatacgtacaccgaatagggcgcacgggaagagcagggaggactggtgtttccattacaactttgactagaa
atgattggagggttgcctctgaattgattaatattctggaaagagcaaatcagagtattccagaggagcttgtatcaatggctgagaggtttaaggcac
atcaacagaaaagggaaatggaaagaaaaatggaaagacctcaaggaaggcccaagaagtttcattaa
[Chemical Formula 87-1]
Nucleic acid sequence of pLgBiT-DDX46
(SEQ ID NO: 82)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccaggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacaggggagttccggtggtggcgggagcggaggtggaggcatgggtcgggagt
cacgccactatcgaaaacgatcggcatcccggggtcgctctggaagtcggtctagaagtcgctcaccctcagacaaaagaagtaaacgtggagat
gacagacggtctagaagtagagatagagataggaggagagagaggtctcgtagcagggataaaagaagatctcggtcaagggacaggaagcgt
ctgagacgttccagaagtagagagagagacagaagccgagagcgaagaagatctcgaagtagagacaggagacgctcaaggagtagaagccg
ggccggcgatcccgatcctccagtcctggaaataaaagcaagaaaactgagaatagatctaggtccaaagagaaaactgatggggggaaagtt
ctaaagagaagaaaaaagacaaagatgacaaggaggatgaaaaagaaaaagatgctggcaactttgaccagaataagctggaagaagaaatgag
aaagcgaaaagaaagagtagaaaaatggcgagaagagcaacgtaaaaaggctatggaaaacataggagaactgaaaaaggaaatcgaagagat
gaaacaagggaaaaagtggagtttagaggacgatgatgatgacgaagatgatcctgcagaagctgaaaaggagggaaatgaaatggagggtga
ggagttagatccattagatgcttacatggaagaagtgaaagaggaagtaaaaaaatttaacatgagaagtgtaaaaggtggtgggggaaatgaaaa
gaagtctgggccaacggtcacaaaagttgtcactgttgtgacaaccaaaaaagcagttgtggattctgataagaagaaaggtgagctgatggagaat
gaccaggatgccatggagtattcttcagaggaggaagaagttgatcttcagacagcccttacagggtatcaaacaaaacagcgaaagcttctagaac
cagttgatcatggaaaaattgagtatgagccatttaggaaaaacttctatgttgaagttccagaactagcaaaaatgtctcaagaagaggtaaatgtgt
ttcgattggaaatggagggcattacagttaaaggaaaaggttgccccaaaccaattaaatcctgggtccagtgtggaatttccatgaagatcttaaatt
ccctcaagaagcatggctatgaaaagcccacgcccatccaaacccaagctattcctgctataatgtctggacgagatttgattggcattgccaaaacag
gaagtggaaagaccattgcttttctgttgcccatgtttagacacatcatggatcagaggtcattagaggaaggagaggggccaatagctgtcatcatga
ctccaactcgagaactggctttacagattactaaagagtgtaagaagttttccaagactttgggacttagagtggtctgtgtttacggaggaacaggaa
tcagtgagcagattgctgagctgaaaagaggtgctgaaattattgtttgcacacctggtcgaatgattgacatgttagccgctaacagtggtcgggtca
caaatcttcgaagagtgacatatgttgttttagatgaagcagacagaatgtttgacatgggttttgaaccccaggtcatgcgcatcgtggataatgttc
gtcctgatcgacagacggttatgttttcagctactttccccagagctatggaggctttggctcgcaggatcctcagtaaacctattgaagtacaagttg
gaggcaggagtgtggtttgctcagatgtggagcaacaagtgattgtgattgaagaagaaaagaaattcttgaagttacttgagcttctaggccattat
caagagtcaggatctgtcattatatttgtggataagcaggaacatgctgatggtcttcttaaggatttaatgagagcatcttatccttgcatgtctctt
catggaggcattgatcaatatgacagagatagcatcataaatgactttaagaatgggacctgcaaacttcttgtggctacctctgttgctgcccgaggt
ctagatgtgaaacatctgattcttgtagtaaattatagctgccccaaccattatgaggattatgtacacagagcagggcggactggaagagcaggaaac
aagggttatgcttatacttttatcacagaggatcaagctcgctatgctggtgacataattaaagctcttgaattgtcagggactgcagtacctcctgat
ttagagaaactgtggagtgatttcaaagatcagcagaaagctgaggggaaaataattaaaaagagtagtgggttctctggtaagggattcaagtttgat
gaaacagaacaagctttggctaatgagaggaagaagttacaaaaagcagctcttggtctacaagattcagatgatgaggatgctgcagttgatattgat
gagcaaattgaaagcatgtttaattcaaagaagagagtaaaggatatggctgctcctggaacatcaagtgttcctgctccaactgcaggaaatgctgag
aaattagaaattgctaagagattggctcttagaatcaatgcccagaagaatttgggcatcgagtctcaggtagatgtgatgcagcaggccaccaatgca
attcttaggggtggcaccattctggctcccactgtttctgcaaaaaccattgcagaacaacttgctgaaaagatcaatgccaagctcaattatgtgccg
ttagagaaacaagaagaagagagacaggatggtggacagaatgaatcttttaagagatatgaagaagaattagagatcaatgacttcccacagactgct
aggtggaaagttacctctaaggaagctctgcagagaatcagtgaatactctgaagccgcaattacaatcagaggaacctacttccctcctggcaaaga
acccaag
[Chemical Formula 87-2]
gaaggcgagcggaagatttacttggcaattgaaagtgccaatgaactggctgtgcagaaagcaaaggcagaaatcaccaggctcataaaagaaga
gctgatccggctgcaaaattcataccaaccaacaaataaaggaagatacaaagtcttatag
[Chemical Formula 88]
Nucleic acid sequence of pLgBiT-DDX47
(SEQ ID NO: 83)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcggggggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatggcggcacccg
aggaacacgattctccgaccgaagcgtcccagccgattgtggaagaggaggaaactaaaacatttaaagacctgggtgtgacagatgtgttgtgtg
aagcttgtgaccagttgggatggacaaaacccaccaagatccagattgaagctattcctttggccttacaaggtcgtgatatcattgggcttgcagaaa
ctggctctggaaagacaggcgcctttgctttgcccattctaaacgcactgctggagaccccgcagcgtttgtttgccctagttcttaccccgactcggg
agctggcctttcagatctcagagcagtttgaagccctggggtcctctattggagtgcagagtgctgtgattgtaggtggaattgattcaatgtctcaat
ctttggcccttgcaaaaaaaccacatataataatagcaactcctggtcgactgattgaccacttggaaaatacgaaaggtttcaacttgagagctctca
aatacttggtcatggatgaagccgaccgaatactgaatatggattttgagacagaggttgacaagatcctcaaagtgattcctcgagatcggaaaaca
ttcctcttctctgccaccatgaccaagaaggttcaaaaacttcagcgagcagctctgaagaatcctgtgaaatgtgccgtttcctctaaataccagaca
gttgaaaaattacagcaatattatatttttattccctctaaattcaaggatacctacctggtttatattctaaatgaattggctggaaactcctttat
gatattctgcagcacctgtaataatacccagagaacagctttgctactgcgaaatcttggcttcactgccatccccctccatggacaaatgagtcaga
gtaagcgcctaggatcccttaataagtttaaggccaaggcccgttccattcttctagcaactgacgttgccagccgaggtttggacatacctcatgtag
atgtggttgtcaactttgacattcctacccattccaaggattacatccatcgagtaggtcgaacagctagagctgggcgctccggaaaggctattactt
ttgtcacacagtatgatgtggaactcttccagcgcatagaacacttaattgggaagaaactaccaggttttccaacacaggatgatgaggttatgatgc
tgacagaacgcgtcgctgaagcccaaaggtttgcccgaatggagttaagggagcatggagaaaagaagaaacgctcgcgagaggatgctggagataatg
atgacacagagggtgctattggtgtcaggaacaaggtggctggaggaaaaatgaagaagcggaaaggccgttaa
[Chemical Formula 89]
Nucleic acid sequence of pLgBiT-DDX48
(SEQ ID NO: 84)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcggggggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatggcgaccacgg
ccacgatggcgacctcgggctcggcgcgaaagcggctgctcaaagaggaagacatgactaaagtggaattcgagaccagcgaggaggtggatg
tgacccccacgttcgacaccatgggcctgcgggaggacctgctgcggggcatctacgcttacggttttgaaaaaccatcagcaatccagcaacgag
caatcaagcagatcatcaaagggagagatgtcatcgcacagtctcagtccggcacaggaaaaacagccaccttcagtatctcagtcctccagtgttt
ggatattcagcttcgtgaaactcaagctttgatcttggctcccacaagagagttggctgtgcagatccagaaggggctgcttgctctcggtgactacat
gaatgtccagtgccatgcctgcattggaggcaccaatgttggcgaggacatcaggaagctggattacggacagcatgttgtcgcgggcactccagg
gcgtgtttttgatatgattcgtcgcagaagcctaaggacacgtgctatcaaaatgttggttttggatgaagctgatgaaatgttgaataaaggtttcaa
agagcagatttacgatgtatacaggtacctgcctccagccacacaggtggttctcatcagtgccacgctgccacacgagattctggagatgaccaacaa
gttcatgaccgacccaatccgcatcttggtgaaacgtgatgaattgactctggaaggcatcaagcaatttttcgtggcagtggagagggaagagtgg
aaatttgacactctgtgtgacctctacgacacactgaccatcactcaggcggtcatcttctgcaacaccaaaagaaaggtggactggctgacggaga
aaatgagggaagccaacttcactgtatcctcaatgcatggagacatgccccagaaagagcgggagtccatcatgaaggagttccggtcgggcgcc
agccgagtgcttatttctacagatgtctgggccagggggttggatgtccctcaggtgtccctcatcattaactatgatctccctaataacagagaatt
gtacatacacagaattgggagatcaggtcgatacggccggaagggtgtggccattaactttgtaaagaatgacgacatccgcatcctcagagatatcg
agcagtactattccactcagattgatgagatgccgatgaacgttgctgatcttatctga
[Chemical Formula 90]
Nucleic acid sequence of pLgBiT-DDX49
(SEQ ID NO: 85)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtgggggagcggaggtggaggcatggcaggcttcgc
ggagctcgggctgtcatcgtggctcgtggaacaatgtcggcagctgggtttgaagcagcccacgcccgtgcagctcggctgcatccccgccatcct
ggagggtcgagactgcttgggctgtgctaagacaggcagtgggaagacagcagcgtttgtccttcccatcttgcagaagctgtctgaggatccctat
ggcatcttctgcctcgtcctgacacccaccagggagctggcctaccagatcgcagagcagttccgggtcctggggaagcctctagggctgaaaga
ctgcatcatcgtcggtggcatggacatggtggcccaggcgctggagctctctcggaaaccacacgtggtcatcgccacgccggggcgcctggca
gatcacctgcgcagctccaacacttttagtataaagaagatccgcttcctggtgatggatgaggcagaccggctgctggaacagggctgcactgact
tcaccgtggacctggaggccatcctggcggctgtgccggcccgcaggcagacactgctgttcagcgccacgctgaccgacacactccgggagct
gcagggtctggccaccaaccagcccttcttctgggaagcacaggccccggtgagcaccgtggagcagctggaccagcgctacctgctggtgcct
gagaaggtcaaggacgcctacctggtccacctgatccagcgcttccaggatgagcacgaggactggtccattatcatcttcaccaacacgtgcaag
acctgccagattctgtgcatgatgctgcgcaaattcagcttccccaccgtggctctgcactccatgatgaagcagaaagaacgctttgccgccctagc
caagttcaagtccagcatctaccggatcctgatcgcaacagacgtggcctcccggggcctggacatccctacggtacaggtggtcatcaaccacaa
cacccccgggctccccaagatctacatccaccgagtcggccggacggcccgtgcagggggcagggtcaggccatcacgctggtgacacagta
cgacatccacctggtgcacgccatcgaggagcagatcaagaagaagctggaggagttctccgtggaagaggccgaggtgctacagatcctcaca
caggtcaacgtggtgcgaagagagtgtgagatcaaactggaggcggcccactttgacgaaaagaaggagatcaacaaacggaagcagctgatcc
tggaggggaaggaccctgacctggaggccaagcgcaaggctgagctggccaagatcaagcagaagaaccggcgcttcaaggagaaggtgga
ggagacgctgaagcgacagaaggctggcagggctggccacaaggggcgtccacccaggacaccgtctgggtcccactcaggcccagtcccct
cccagggcctggtctga
[Chemical Formula 91]
Nucleic acid sequence of pLgBiT-DDX50
(SEQ ID NO: 86)
ggcctaactggccggtacctgagtctaaatgagtcttcggacctcgcgggggccgcttaagcggtgcttagggtttgtctgacgcggggggaggg
ggaaggaacgaaacactctcattcggaggggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaagctgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgcctgggaaact
cctctggggggacattatggagctggaagcacccttggaggagtccgagagccagaagaaggagaggcaaaagagtgacagaaggaagtcaa
ggcaccattatgactcggatgagaaatcagaaacaagagaaaatggtgttacagatgacctggatgctcccaaggccaaaaaatctaaaatgaaag
agaagctaaatggagacactgaagaaggatttaatagactttcagatgaattctccaaatctcataagtcaagaagaaaagatctaccaaatggagat
atagatgaatatgaaaaaaaatcaaagcgagtatcatctttagatacttctactcataaatcaagtgataataaactagaggagaccttaacacgtgaa
cagaaagaaggagccttctccaattttcctatttctgaagagactataaagcttctgaaaggtcgaggggtaacatatctctttcctattcaagttaag
acctttggtcctgtatatgaaggaaaagatttaatagctcaagcacggacaggaacaggaaagacattctcttttgccatccccttaattgaaagactc
caaagaaatcaagaaacaattaaaaaaagccgctcaccaaaggtacttgttttggctccaacaagggaactggcaaaccaagtagccaaagacttcaaa
gatataactaggaaactcagcgtggcgtgtttttatggtggaacatcatatcaaagccaaattaatcatattcgaaatggtattgacatcttggttgga
acacctggtcgtatcaaagaccatctgcagagtggccgattggatctttctaaactgcgacatgttgtgcttgatgaagtggatcagatgttagattta
ggtttcgctgaacaagttgaagatattattcatgaatcctacaaaactgattctgaagacaatcctcagactttacttttttctgcaacttgcccacag
tgggtatacaaagttgcaaaaaaatacatgaaatccagatatgaacaggttgaccttgttggaaaaatgactcaaaaggctgcaactactgtggaacat
ttggccatccagtgtcattggtctcagaggccagcagttattggagatgtccttcaagtctacagtgggtctgaagggagggctattattttctgtgag
accaagaagaatgtaactgaaatggccatgaatccacacataaaacagaatgcccagtgtttacatggggacattgcacagtcacaaagagaaattaca
ctaaaaggcttcagagaaggtagttttaaagttttggtggcaaccaatgtggctgcccgtggtttggacattcctgaagttgacctggtgattcaaagt
tctcctcctcaggatgttgagtcctatatccatcgctctggacgcacaggtagagctggacggacagggatttgtatatgtttttatcaaccaagagaa
agaggtcaactaagatatgtggaacaaaaagcaggaattacttttaaacgtgtaggtgttccttctacaatggatttagttaaatctaaaagcatggat
gccatcaggtctctggcttccgtttcttacgctgctgttgattttttccgaccatcagctcagagactgatagaagagaaaggtgcagtggatgcattg
gctgcagctttagcccacatttctggtgcatcaagctttgaaccacgatctttgatcacctctgataaggggtttgtgaccatgactctggaaagcct
agaggaaatacaggatgtcagctgtgcttggaaagaacttaacagaaagctgagtagtaatgcagtgtctcagattaccagaatgtgcctcctgaaag
gaaatatgggtgtttgctttgatgttcctacaactgagtcagaaaggttacaggcagagtggcatgattccgactggatactctcagtgccagccaaa
ttacctgaaattgaagaatattatgatggaaacacatcttctaattccagacagaggagtggctggtcaagtggtcgatcaggccggtcaggccggtc
aggtggtcgatctggcggccggtcaggtagacagagtcgacaaggaagtcgctcaggaagtcgacaagatggtagaagacgaagtgggaatagaaatc
gatcaagaagtgggggccacaaacggagttttgactga
[Chemical Formula 92]
Nucleic acid sequence of pLgBiT-DDX51
(SEQ ID NO: 87)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatggcgctgttcta
cgtcgcgcggtacccgggccccgåtgcggcagctgcggcggggccggagggcgcggaggccgggggcacggcagggcccgcgcgctgc
tcgagcggctgcagagccgggcccgcgaacggcagcagcagcgggagcccgcgcagaccgaggcggctgcatcgaccgagccggcgacca
ggaggcgacggcggccccggcggcggcggcgggtgaacgacgcggagccggggagcccggaggcgccgcagggaaagcgacggaagg
cggacggcgaggacgcgggcgcagaaagcaacgaggaggcgccaggggagcccagcgcagggagcagcgaggaggcgccaggggagc
ccagcgcagggagcagcgaggaggcgccgggggagcgcagcaccagcgccagcgccgaggcggccccagatggaccggccctggagga
ggcggccggacccctggtccccggcctggtgctgggggggttcgggaagaggaaggcgccgaaggtccagcctttcctgccaaggtggctggc
tgagcctaactgtgtcagaaggaatgtcaccgaagacctggttcctatcgaggacatccctgacgtccatcctgacctgcagaagcagctgcgggc
acacggcatctcgtcctactttccagtccaggcagctgtgattcctgccctcctggagagcgcagcctgtgggtttctggtgggcagaggtggctacc
ggcctagcgacctctgtgtttctgccccaacaggcagtgggaagacactggccttcgtcatccctgtggtgcaggccctgctttcgagagtggtctgc
cacatccgtgccctggttgtgctgcccaccaaggagctggcccagcaggtgagcaaagttttcaacatctacacagatgccacacctctgagagtct
ccctggttacgggacagaagtctctggccaaggagcaggagagcctcgtccagaaaacagctgatgggtaccgctgcttggctgacatcgtggta
gccacccccggccgcctggtggaccacatcgaccagaccccaggattcagcctccagcagctccgcttcctgattatcgacgaggctgaccggat
gattgacagcatgcatcagtcctggctgccgggtggtggcggccgccttccagagcgaggaccccgcggacccctgtgccctgctccagcga
aggcaggcccaggctgtgacagccgccagcacctgctgtccccagatgcccctgcagaagctgctcttctcagctactctgacccagaaccctgaa
aagctgcagcagctgggcctccaccagccccggcttttctccacagggctagcacacaggggcctggaagatacagatggggacggggattcgg
ggaagtatgcctttcctgttgggctcacgcaccactacgtgccctgcagcctcagctctaagccgctggtcgtcctgcacctggtcctggagatgggc
ttctcgagggttctctgcttcactaactcccgagagaactcccacaggctcttcctgctggtgcaagcttttgggggtgtggacgtggctgagttctcc
tcgcgctacgggcctggccagaggaggatgatcctgaagcagtttgaacaggggaagatccagctgctcatcagcacggacgccaccgcgcgag
gcatcgacgtgcagggtgtggagctggtggtgaactacgacgccccccagtacctgagaacctacgtgcaccgggttgggaggacagctcgcgc
tgggaaaactggacaggccttcacactgctcctgaaagtgcaggagaggagattcctccgaatgctaactgaagctggggcacctgagttgcagcg
gcacgagctctccagcaagctgctgcagccgctggttcctcggtacgaggaggccctgtcccagctggaggagtctgtcaaggaagagcgcaag
cagagggcggcctag
[Chemical Formula 93]
Nucleic acid sequence of pLgBiT-DDX52
(SEQ ID NO: 88)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtgggggagcggaggtggaggcatggacgtccacg
atctctttcgccggctcggcgcgggggccaaattcgacacgagacgcttctcggcagacgcagctcgattccagataggaaaaaggaaatatgact
ttgattcttcggaggtgcttcagggactggacttttttggaaacaagaagtctgtcccaggtgtgtgtggagcatcacaaacacatcagaagccccaaa
atggagagaaaaaagaagagagcctaactgaaaggaagagggagcagagcaagaaaaaaaggaagacgatgacttcagaaattgcttcccaag
aagaaggtgctactatacagtggatgtcatctgtagaagcaaagattgaagacaaaaaagttcagagagaaagtaaactaacttccggaaagttgga
gaatctcagaaaagaaaagataaacttcttgcggaataaacacaaaattcacgtccaaggaaccgatcttcctgacccaattgctacatttcagcaact
tgaccaggaatataaaatcaattctcgactacttcagaacattctagatgcaggtttccaaatgcctacgccaatccaaatgcaagccatcccagttat
gctgcatggtcgggaacttctggcttctgctccaactggatctggaaaaacattagcttttagcattcctattttaatgcagctgaaacaacccgcaaa
taaaggcttcagagccctgattatatcaccaacacgagaacttgccagccagattcacagagagttaataaaaatttctgagggaacaggattcaggat
acacatgatccacaaagcagcagtggcagccaagaaatttggacctaaatcatctaaaaagtttgatattcttgtgactactccaaatcgactaatcta
tttattaaagcaagatccccccggaatcgacctagcaagtgttgagtggcttgtagtagacgaatcagataaactgtttgaagatggcaaaactgggtt
cagagaccagctggcttccattttcctggcctgcacatcccacaaggtccgaagagctatgttcagtgcaacttttgcatatgatgttgaacagtggtg
caaactcaacctggacaatgtcatcagtgtgtccattggagcaaggaattctgcagtagaaactgtagaacaagagcttctctttgttggatctgagac
cggaaaacttctggccatgagagaacttgttaaaaagggtttcaatccacctgttcttgtttttgttcagtccattgaaagggctaaagaactttttc
atgagctcatatatgaaggtattaatgtggatgttattcatgcagagagaacacaacaacagagagataacacagtccacagtttcagagcaggaaaa
atctgggttctgatttgtacagccttgctagcaagagggattgattttaaaggtgtgaacttggtgatcaactatgactttccaactagctcagtgga
atatatccacaggataggtcgaactggaagagcagggaataagggaaaagcaattacatttttcactgaggatgataagccattattaagaagcgttg
ctaatgttatacagcaggctgggtgtcctgtaccagaatacataaaaggttttcagaaactactaagcaaacaaaagaaaaagatgattaagaaacc
attggaaagggagagcattagtacaactccaaaatgtttcttagaaaaagctaaggataaacagaaaaaggtcactggtcagaacagcaagaagaaag
tagctcttgaagacaaaagttaa
[Chemical Formula 94]
Nucleic acid sequence of pLgBiT-DDX53
(SEQ ID NO: 89)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtca
ttggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgtcccactgggc
cccagagtggaagagggcggaggctaatccaagagaccttggggccagctgggatgtcaggggcagcagaggcagtggctggagtggcccctt
cggccatcagggaccgagagcagcaggctcccgtgaaccaccactctgctttaaaataaagaacaatatggttggtgtggtcattggttacagtgga
tcaaaaataaaagatctacaacattcgacaaacactaaaatacagatcataaacggggaatctgaagcaaaagtcagaatttttggcaatagggaaat
gaaagcaaaggccaaagggctatagaaacacttattagaaaacaagaaagctacaactcagaatccagtgtggataatgctgcatcccaaacccc
tattggaagaaatctaggcagaaatgacattgttggagaagctgagccattgtcaaattgggatcgcattagggcagcagtcgtggagtgcgaaaag
agaaaatgggcagatctaccaccagttaagaaaaacttttacatagaatccaaagcaacaagctgcatgtctgaaatgcaggtgattaactggagaaa
ggaaaatttcaacataacgtgtgatgacttgaaaagtggtgaaaagcgtctcattccaaaaccaacttgcaggtttaaagacgcttttcagcaataccc
tgatcttctgaaaagcataataagggtagggattgtaaagccaacgccaattcagtcacaggcatggccaattattctacaaggaatagatcttatagt
agttgcacaaaccggaacagggaaaacattgtcctatctaatgcctgggtttattcatcttgattctcaaccaatatctagagagcaaaggaatgggcc
tgggatgctagtccttacacccactagagagttggctcttcacgtggaagctgaatgttcaaagtattcatataaaggtctcaaaagcatttgcatat
atggtggtagaaacagaaatggacaaatagaagacattagcaaaggtgtagatatcattattgcaactcctgggaggctgaatgacctacaaatgaat
aactctgtcaacctaagaagcataacctacttggttatagatgaggcagataaaatgctggatatggaatttgaaccccagataaggaagattttatt
agatgtgcgcccagaccgacagactgttatgacaagtgcaacttggccagatactgtacgtcaactagcactttcttatttgaaagatcctatgattg
tttatgttggtaatctgaatctagtggctgtaaatacagtgaagcaaaatataattgttaccacagaaaaagaaaaacgagctctcacccaagaattc
gtagagaacatgtcacccaacgacaaagtcatcatgtttgtcagccaaaaacatattgctgatgacttgtcaagcgacttcaatatccaaggcatatc
tgcagaatcattacatggcaacagtgaacagagtgatcaagagcgagcagtagaggactttaaaagcggaaacataaagatactgattacaactgata
tagtatcccgaggtcttgatcttaatgatgtcacacatgtatataattatgatttcccaaggaatattgacgtatatgtacacagagtagggtacatt
ggacggacaggaaagactggcacatcagttaccctcatcactcagagagattcgaaaatggccggtgaattgattaaaattctggacagagcaaatca
gagtgttccggaagatcttgtagtaatggctgagcaatacaagttaaatcaacaaaagaggcacagagaaacacgatcaagaaaacctggacaaagac
gcaaggagttttattttttaagttga
[Chemical Formula 95]
Nucleic acid sequence of pLgBiT-DDX54
(SEQ ID NO: 90)
ggcctaactggccggtacctgagtctaaatgagtcttcggacctcgcgggggccgcttaagcggtgattagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcggggggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtca
ttggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccgatactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccgctggtggcgggagcggaggtggaggcatggcggccgaca
agggcccggcggctggacctcggtcgcgagctgccatggcccagtggaggaagaagaaagggctccggaagcgccgaggcgcggcctccca
ggcccgcggcagcgactcggaggacggcgagtttgagatccaggcggaagatgacgcccgggcccggaagctgggacctggaagacccctg
cccaccttccccacctcggaatgcacctcggatgtggagccggacacccgggagatggtgcgtgcccagaacaagaagaagaagaagtctgga
ggcttccagtccatgggcctgagctacccggtgttcaaaggcatcatgaagaaggggtacaaggtgccaacacccatccagaggaagaccatccc
ggtgatcttggatggcaaggacgtggtggccatggcccggacgggcagtggcaagacagcctgcttcctcctcccaatgttcgagcggctcaaga
cccacagtgcccagaccggggcccgcgccctcatcctctcgccgacccgagagctggccctgcagaccctgaagttcactaaggagctaggcaa
gttcactggcctcaagactgccctgatcctgggtggagacaggatggaagaccagtttgcagccctgcacgaaaatcccgacataattattgccacg
cccggacggttggtgcatgtggctgtggaaatgagcctgaagctgcagagtgtggaatacgtggtgttcgatgaagctgaccggctttttgaaatgg
gtttcgcagagcagctgcaggagatcatcgcccgcctccccgggggccaccagacggtgctgttctccgccacgctgcccaaactgctggtggaa
tttgcccgggctggcctcacggagcccgtgctcatccggcttgacgtggataccaagctcaacgagcagctgaagacctccttcttcctcgtgcggg
aggacaccaaggctgccgtgctgctccacctgctgcacaacgtggtgcggccccaggaccagaccgtggtgtttgtggccacgaagcaccacgc
cgagtacctcactgagctgctgacgacccagcgggtgagctgcgcccacatctacagtgccctagacccgacagcccgcaagatcaatctcgcca
aattcacgcttggcaagtgctccactctcattgtgactgacctggccgcccgaggcctggacatcccgctgctggacaatgtcatcaactacagcttc
cccgccaagggcaaactcttcctgcaccgcgtgggccgtgtggctcgggctggccgaagtggcacagcctactccttggtggcccctgatgaaatc
ccctacctgctggatctgcacctgttcctgggccgctccctcaccctcgcccgacccctcaaggagccctcaggtgtggccggtgtggatggcatgc
tgggtcgggtgccacagagtgtggtggacgaggaggacagtggtctgcagagcaccctggaggcatcgctggagctacggggcctggcccgcg
ttgctgataacgcccagcagcagtatgtgcgctcacgcccggcgccctcgcctgagtccatcaagagggccaaggagatggaccttgtggggctg
ggcctgcaccccctcttcagctcgcgttttgaggaggaggagctgcagcggctgaggctggtggacagcataaagaactaccgctcccgggcgac
tatctttgagatcaacgcctccagccgagacctgtgcagccaggtgatgcgcgccaagcggcagaaggaccgcaaggccatcgcccgcttccag
cagggacagcaggggcggcaggagcagcaggagggcccagtgggcccagccccgagccgcccagcactgcaggagaagcagcctgagaa
ggaggaggaggaggaggcgggagagagtgtggaggacattttctcagaggtcgtgggccggaagcggcagcggtcaggacccaacagggga
gccaagaggcggagggaggaggcccggcagcgggaccaggaattctacatcccctaccggcccaaggactttgacagcgagcggggcctgag
catcagcggggaagggggagcctttgagcagcaggcagctggcgctgtcctggacttgatgggggatgaagcccagaacctgacgaggggccg
gcagcagctcaagtgggaccgtaagaagaagcggtttgtgggacagtcaggacaggaagacaagaagaagattaagacagagagcggccgcta
catcagcagctcctacaagcgagacctctatcagaagtggaaacagaaacagaaaattgatgatcgtgactcggacgaagaaggggcatctgacc
ggcgaggcccagagcgaagaggtgggaagcgagaccgtggccaaggtgcatcccggccccacgccccaggcacccctgcaggccgagtccg
cccggaactcaagaccaagcagcagatcctgaagcagcggcgccgggcccagaagctgcacttcctgcagcgtggtggcctcaagcagctctct
gcccgcaaccgccgccgcgtccaggagctgcagcagggcgccttcggccggggtgcccgctccaagaagggcaagatgcggaagaggatgt
ga
[Chemical Formula 96]
Nucleic acid sequence of pLgBiT-DDX55
(SEQ ID NO: 91)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcggctggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtca
ttggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatggagcatgtgac
agagggctcctgggagtcgctgcctctgccgctgcacccgcaggtgctggggcgctgcgggagctgggcttcccgtacatgacgccggtgcag
tccgcaaccatccctctgttcatgcgaaacaaagatgtcgctgcagaagcggtcacaggtagtggcaaaacactcgcttttgtcatccccatcctgga
aattcttctgagaagagaagagaagttaaaaaagagtcaggttggagccataatcatcacccccactcgagagctggccattcaaatagacgaggtc
ctgtcgcatttcatgaagcacttccccgagttcagccagattctttggatcggaggcaggaatcctggagaagatgttgagaggtttaagcaacaagg
tgggaacatcattgtggccactccaggccgcttggaggacatgttccggaggaaggccgaaggcttggatctggccagctgtgtgcgatccctgga
tgtcctggtgttggatgaggcagacagacttctggacatggggtttgaggcaagcatcaacaccattctggagtttttgccaaagcagaggagaaca
ggccttttctctgccactcagacgcaggaagtggagaacctggtgagagcgggcctccggaaccctgtccgggtctcagtgaaggagaagggcgt
ggcagccagcagtgcccagaagaccccctcccgcctggaaaactactacatggtatgcaaggcagatgagaaatttaatcagctggtccattttcttc
gaatcataagcaggagaaacacctggtcttcttcagcacctgtgcctgtgtggaatactatgggaaggctctggaagtgctggtgaagggcgtgaa
gattatgtgcattcacggaaagatgaaatataaacgcaataagatcttcatggagttccgcaaattgcaaagtgggattttagtgtgcactgatgtgat
ggcccggggaattgatattcctgaagtcaactgggttttgcagtatgaccctcccagcaatgcaagtgccttcgtgcatcgctgcggtcgcacagctcg
cattggccacgggggcagcgctctggtgttcctcctgcccatggaagagtcatacatcaatttccttgcaattaaccaaaaatgccccctgcaggaga
tgaagccccagagaaacacagcggaccttctgccaaaactcaagtccatggccctggctgacagagctgtgtttgaaaagggcatgaaagcttttgt
gtcatatgtccaagcttatgcaaagcatgaatgcaacctgattttcagattaaaggatcttgattttgccagccttgctcgaggttttgccctgctgag
gatgcccaagatgccagaattgagaggaaagcagtttccagattttgtgcccgtggacgttaataccgacacgattccatttaaagataaaatcagag
aaaagcagaggcagaaactcctggagcaacaaagaagagagaaaacagaaaatgaagggagaagaaaattcataaaaaataaagcttggtcaaagc
agaaggccaaaaaagaaaagaagaaaaaaatgaatgagaaaaggaaaagggaagagggttctgatattgaagatgaggacatggaagaacttct
taatgacacaagactcttgaaaaaacttaagaaaggcaaaataactgaagaagaatttgagaagggcttgttgacaactggcaaaagaacaatcaag
acagtggatttagggatctcagatttggaagatgactgctga
[Chemical Formula 97]
Nucleic acid sequence of pLgBiT-DDX56
(SEQ ID NO: 92)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtcat
tggcgaagtcgaacacgcagatgcagtcggggggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgtgttccgagtaaccatcaacagtgggagttccggtggtggcgggagcggaggtggaggcatggaggactctga
agcactgggcttcgaacacatgggcctcgatccccggctccttcaggctgtcaccgatctgggctggtcgcgacctacgctgatccaggagaaggc
catcccactggccctagaagggaaggacctcctggctcgggcccgcacgggctccgggaagacggccgcttatgctattccgatgctgcagctgtt
gctccataggaaggcgacaggtccggtggtagaacaggcagtgagaggccttgttcttgttcctaccaaggagctggcacggcaagcacagtcca
tgattcagcagctggctacctactgtgctcgggatgtccgagtggccaatgtctcagctgctgaagactcagtctctcagagagctgtgctgatggag
aagccagatgtggtagtagggaccccatctcgcatattaagccacttgcagcaagacagcctgaaacttcgtgactccctggagcttttggtggtgga
cgaagctgaccttcttttttcctttggctttgaagaagagctcaagagtctcctctgtcacttgccccggatttaccaggcttttctcatgtcagcta
cttttaacgaggacgtacaagcactcaaggagctgatattacataacccggttacccttaagttacaggagtcccagctgcctgggccagaccagtta
cagcagtttcagctggtctgtgagactgaggaagacaaattcctcctgctgtatgccctgctcaagctgtcattgattcggggcaagtctctgctctt
tgtcaacactctagaacggagttaccggctacgcctgttcttggaacagttcagcatccccacctgtgtgctcaatggagagcttccactgcgctcca
ggtgccacatcatctcacagttcaaccaaggcttctacgactgtgtcatagcaactgatgctgaagtcctgggggccccagtcaagggcaagcgtcgg
ggccgagggcccaaaggggacaaggcctctgatccggaagcaggtgtggcccggggcatagacttccaccatgtgtctgctgtgctcaactttgatct
tcccccaacccctgaggcctacatccatcgagctggcaggacagcacgcgctaacaacccaggcatagtcttaacctttgtgcttcccacggagcagt
tccacttaggcaagattgaggagcttctcagtggagagaacaggggccccattctgctcccctaccagttccggatggaggagatcgagggcttccgct
atcgctgcagggatgccatgcgctcagtgactaagcaggccattcgggaggcaagattgaaggagatcaaggaagagcttctgcattctgagaag
cttaagacatactttgaagacaaccctagggacctccagctgctgcggcatgacctacctttgcaccccgcagtggtgaagccccacctgggccatg
ttcctgactacctggttcctcctgctctccgtggcctggtgcgccctcacaagaagcggaagaagctgtcttcctcttgtaggaaggccaagagagca
aagtcccagaacccactgcgcagcttcaagcacaaaggaaagaaattcagacccacagccaagccctcctga
[Chemical Formula 98]
Nucleic acid sequence of pLgBiT-DDX58
(SEQ ID NO: 93)
ggcctaactggccggtacctgagtctaaatgagtcttcggacctcgcgggggccgcttaagcggtggttagggtttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtca
ttggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgaccaccgagc
agcgacgcagcctgcaagccttccaggattatatccggaagaccctggaccctacctacatcctgagctacatggccccctggtttagggagggtta
ttctggactttatgaagccattgaaagttgggatttcaaaaaaattgaaaagttggaggagtatagattacttttaaaacgtttacaaccagaattta
aaaccagaattatcccaaccgatatcatttctgatctgtctgaatgtttaattaatcaggaatgtgaagaaattctacagatttgctctactaagggg
atgatggcaggtgcagagaaattggtggaatgccttctcagatcagacaaggaaaactggcccaaaactttgaaacttgctttggagaaagaaaggaa
caagttcagtgaactgtggattgtagagaaaggtataaaagatgttgaaacagaagatcttgaggataagatggaaacttctgacatacagattttct
accaagaagatccagaatgccagaatcttagtgagaattcatgtccaccttcagaagtgtctgatacaaacttgtacagcccatttaaaccaagaaat
taccaattagagcttgctttgcctgctatgaaaggaaaaaacacaataatatgtgctcctacaggttgtggaaaaacctttgtttcactgcttatatg
tgaacatcatcttaaaaaattcccacaaggacaaaaggggaaagttgtcttttttgcgaatcagatcccagtgtatgaacagcagaaatctgtattct
caaaatactttgaaagacatgggtatagagttacaggcatttctggagcaacagctgagaatgtcccagtggaacagattgttgagaacaatgacatc
atcattttaactccacagattcttgtgaacaaccttaaaaagggaacgattccatcactatccatctttactttgatgatatttgatgaatgccacaa
cactagtaaacaacacccgtacaatatgatcatgtttaattatctagatcagaaacttggaggatcttcaggcccactgccccaggtcattgggctga
ctgcctcggttggtgttggggatgccaaaaacacagatgaagccttggattatatctgcaagctgtgtgcttctcttgatgcgtcagtgatagcaaca
gtcaaacacaatctggaggaactggagcaagttgtttataagccccagaagtttttcaggaaagtggaatcacggattagcgacaaatttaaatacat
catagctcagctgatgagggacacagagagtctggcaaagagaatctgcaaagacctcgaaaacttatctcaaattcaaaatagggaatttggaacac
agaaatatgaacaatggattgttacagttcagaaagcatgcatggtgttccagatgccagacaaagatgaagagagcaggatttgtaaagccctgttt
ttatacacttcacatttgcggaaatataatgatgccctcattatcagtgagcatgcacgaatgaaagatgctctggattacttgaaagacttcttcag
caatgtccgagcagcaggattcgatgagattgagcaagatcttactcagagatttgaagaaaagctgcaggaactagaaagtgtttccagggatccca
gcaatgagaatcctaaacttgaagacctctgcttcatcttacaagaagagtaccacttaaacccagagacaataacaattctctttgtgaaaaccaga
gcacttgtggacgctttaaaaaattggattgaaggaaatcctaaactcagttttctaaaacctggcatattgactggacgtggcaaaacaaatcagaa
cacaggaatgaccctcccggcacagaagtgtatattggatgcattcaaagccagtggagatcacaatattctgattgccacctcagttgctgatgaag
gcattgacattgcacagtgcaatcttgtcatcctttatgagtatgtgggcaatgtcatcaaaatgatccaaaccagaggcagaggaagagcaagaggt
agcaagtgcttccttctgactagtaatgctggtgtaattgaaaaagaacaaataaacatgtacaaagaaaaaatgatgaatgactctattttacgcct
tcagacatgggacgaagcagtatttagggaaaagattctgcatatacagactcatgaaaaattcatcagagatagtcaagaaaaaccaaaacctgtac
ctgataaggaaaaaaaaaactgctctgcagaaagtgcaaagccttggcatgttacacagctgacgtaagagtgatagaggaatgccattacactgtgc
ttggagatgcttttaaggaatgctttgtgagtagaccacatcccaagccaaagcagttttcaagttttgaaaaaagagcaaagatattctgtgcccga
cagaactgcagccatgactggggaatccatgtgaagtacaagacatttgagattccagttataaaaattgaaagttttgtggtggaggatattgcaac
tggagttcagacactgtactcgaagtggaaggactttcattttgagaagataccatttgatccagcagaaatgtccaaatga
[Chemical Formula 99]
Nucleic acid sequence of pLgBiT-DDX59
(SEQ ID NO: 94)
ggcctaactggccggtacctgagtctaaatgagtcttcggacctcgcgggggccgcttaagcggtggttaggctttgtctgacgcggggggaggg
ggaaggaacgaaacactctcattcggaggcggctcggggtttggtcttggtggccacgggcacgcagaagagcgccgcgatcctcttaagcaccc
ccccgccctccgtggaggcgggggtttggtcggcgggtggtaactggcgggccgctgactcgggcgggtcgcgcgccccagagtgtgacctttt
cggtctgctcgcagacccccgggcggcgccgccgcggcggcgacgggctcgctgggtcctaggctccatggggaccgtatacgtggacaggct
ctggagcatccgcacgactgcggtgatattaccggagaccttctgcgggacgagccgggtcacgcggctgacgcggagcgtccgttgggcgaca
aacaccaggacggggcacaggtacactatcttgtcacccggaggcgcgagggactgcaggagcttcagggagtggcgcagctgcttcatccccg
tggcccgttgctcgcgtttgctggcggtgtccccggaagaaatatatttgcatgtctttagttctatgatgacacaaaccccgcccagcgtcttgtca
ttggcgaagtcgaacacgcagatgcagtcggggcggcgcggtcccaggtccacttcgcatattaaggtgacgcgtgtggcctcgaacaccgagcgac
cctgcagcgacccgcttaaaagcttggcaatccggtactgttggtaaagccaccatggtcttcacactcgaagatttcgttggggactgggaacagac
agccgcctacaacctggaccaagtccttgaacagggaggtgtgtccagtttgctgcagaatctcgccgtgtccgtaactccgatccaaaggattgtcc
ggagcggtgaaaatgccctgaagatcgacatccatgtcatcatcccgtatgaaggtctgagcgccgaccaaatggcccagatcgaagaggtgttta
aggtggtgtaccctgtggatgatcatcactttaaggtgatcctgccctatggcacactggtaatcgacggggttacgccgaacatgctgaactatttcg
gacggccgtatgaaggcatcgccgtgttcgacggcaaaaagatcactgtaacagggaccctgtggaacggcaacaaaattatcgacgagcgcctg
atcacccccgacggctccatgctgttccgagtaaccatcaacagtgggagttccggtggtggcgggagcggaggtggaggcatgtttgttccaaga
tctctaaaaatcaagaggaatgctaatgatgatggcaaaagttgtgtggctaagataattaaaccagacccagaagaccttcagttggacaaaagcag
agatgttcccgttgatgctgtagctacagaagcagccacaatagacaggcacatcagcgaatcatgccctttccccagcccaggtggccagttggca
gaggttcattcagtaagtcccgagcagggtgcgaaggacagccatccttctgaagagcccgttaagtcattttccaaaacacagcgctgggcagaa
ccaggggaacccatctgtgttgtctgtggtcgttatggagagtatatctgtgataagacagatgaagatgtgtgtagtttggagtgtaaagcgaaacatc
ttctacaagttaaggaaaaggaagagaaatcaaaactcagcaatccacagaaggctgattctgagccagagtctccactgaatgcttcctatgtctac
aaagagcacccctttattttgaaccttcaggaagaccagattgaaaatcttaaacagcagctgggaattttagttcaagggcaagaagtcaccaggcc
cattattgactttgaacattgtagtctccctgaggtcttaaatcacaacttgaagaaatcaggctatgaggtgccaactcccattcaaatgcagatga
ttcctgtgggacttctgggaagagacattctggccagtgcagatactggctcaggaaaaacagctgcttttcttcttcctgttatcatgcgagcttta
ttcgagagcaaaactccatctgcgctcattcttacaccaaccagagagttagccattcagatagagagacaagctaaagaattgatgagtggcctgcc
acgcatgaaaactgtgcttcttgtagggggcttacccttacccccacagctttatcgtctgcaacaacatgttaaggttatcatagcaacccctgggc
gacttctggatataataaagcagagctctgtagaactctgtggtgtaaagattgtggtagtagatgaagctgataccatgttaaagatgggttttcaa
caacaagtgcttgacattttggaaaacattcctaatgattgtcagaccattttggtttcagccacaattccaactagcatagaacagctagcaagcca
gcttctgcataatcctgtgagaattatcactggagaaaagaacctaccttgtgccaatgtacgtcagattattttgtgggtagaagacccagccaaaa
agaaaaaattatttgaaattttaaatgataagaaactctttaagcctccagtgttagtatttgtggactgcaaactaggagcagatcttttgagtgaa
gccgttcagaaaatcacagggctgaaaagcatatctatacattcagagaagtcgcaaatagaaaggaaaaacatattgaagggattacttgaaggaga
ctatgaagttgtagtgagcacaggagtcttgggacgaggcctagacttgatcagtgtcaggctggttgtcaattttgatatgccttcaagtatggatg
agtatgtccatcagattggaagagtaggaagattaggtcaaaatggaacagcgattactttcatcaataataattcaaaaagactcttctgggatatt
gcaaaacgagtaaagcccacaggatccattcttccccctcagttattaaattccccataccttcatgaccagaagagaaaggaacaacagaaagataa
acagacacagaatgatctggttacaggagctaatcttatggatattatcagaaaacatgataaaagtaattctcagaaatga

### Resources and Reagents

The same resources and reagents as in Test 1 are used.

### 3-2. Test Procedure

### Examples 34 to 70

HeLa cells grown in 96-well plates with growth medium are transfected with pLgBiT-DDXs (SEQ ID NOs: 58 to 93) and pSmBiT-p62 (SEQ ID NO: 57) using Lipofectamine3000 (registered trademark) transfection reagent (Thermo Fisher Scientific Inc.) according to the attached instruction. 24 hours after the addition of the reagent, the test compound is added to the wells in such an amount as to achieve a concentration of 10 μM to treat the transfected cells with the test compound for 12 hours. Thereafter, the growth medium is replaced with 100 μL of Opti-MEM (registered trademark) I (Thermo Fisher Scientific Inc.), 25 μL of 5× Nano-Glo Live Cell Reagent containing a substrate, furimazine, is added, and measurement of luminescence is immediately started with GloMax (registered trademark) Discover Microplate Reader (Promega Corporation) to measure the emission amount over time.

### Test 4. Validation of Autophagy Inducing Action of Screened Test Compounds

Tc demonstrate the effectiveness of the screening methods of the present invention, with Compound AUTAC2-2G, which has been newly synthesized by the inventors, cells transfected with FKBP12 was treated to examine whether FKBP12 could be degraded.

4-1. Preparation of pEGFP-FKBP12

Tc prepare pEGFP-FKBP12, pEGFP-HaloTag [Takahashi et al., Mol. Cell 76 797-810.e10 (2019)] was linearized by a polymerase chain reaction using oligo primers (5′-cgcaaatctagacttgtacagctcgtcc-3′ and 5′-ggccgcttcgagcagatg-3′). The cDNA of FKBP12 was synthesized from the total RNAs of Hela cells using an oligo (dT)₁₅ primer, amplified with oligo primers (5′-aagtctagatttgcgatgggagtgcaggtgg-3′ and 5′-ctgctcgaagcggcctcattccagttagaagc-3′), and inserted into a vector using an Infusion cloning system (underlined 15 nt is Infusion arm).

[Chemical Formula 100]
Code sequence of pEGFP-FKBP12 is as follows.
(SEQ ID NO: 21)
tcaatattggccattagccatattattcattggttatatagcataaatcaatattggctattggccattgcatacgttgtat
ctatatcataatatgtacatttatattggctcatgtccaatatgaccgccatgttggcattgattattgactagttattaat
agtaatcaattacggggtcattagttcatagcccatatatggagttccgcgttacataacttacggtaaatggcccgcctgg
ctgaccgcccaacgacccccgcccattgacgtcaataatgacgtatgttcccatagtancgccaatagggactttccattga
cgtcaatgggtggagtatttacggtaaactgcccacttggcagtacatcaagtgtatcatatgccaagtccgccccctattg
acgtcaatgacggtaaatggcccgcctggcattatgcccagtacatgaccttacgggactttcctacttggcagtacatcta
cgtattagtcatcgctattaccatggtgatgcggttttggcagtacaccaatgggcgtggatagcggtttgactcacgggga
tttccaagtctccaccccattgacgtcaatgggagtttgttttggcaccaaaatcaacgggactttccaaaatgtcgtaata
accccgccccgttgacgcaaatgggcggtaggcgtgtacggtgggaggtctatataagcagagctggtttagtgaaccgtca
gatcactagaagctttattgcggtagtttatcacagttaaattgctaacgcagtcagtgcttctgacacaacagtctcgaac
ttaagctgcagaagttggtcgtgaggcactgggcaggtaagtatcaaggttacaagacaggtttaaggagaccaatagaaac
tgggcttgtcgagacagagaagactcttgcgtttctgataggcacctattggtcttactgacatccactttgcctttctctc
cacaggtgtccactcccagttcaattacagctcttaaggc

### 4-2. Resources and Reagents

HeLa Cells: RIKEN BRC Cell Bank #RCB0007
Dulbecco's Modified Eagle Medium (DMEM): FUJIFILM Wako #043-30085
FBS: Thermo Fisher Scientific #10437028
Non-essential amino acids: FUJIFILM Wako #139-15651
Opti-MEM I: Thermo Fisher Scientific #11058-021
96-Well culture plate. Thermo Fisher Scientific #165305
Lipofectamine3000 transfection reagent: Thermo Fisher Scientific #L3000008
Live cell imaging solution: Thermo Fisher Scientific #A14291DJ
Fluorescence microscope: Keyence #BZ-X800
PrimeSTAR Max DNA polymerase: TaKaRa #R045A
TRIzol reagent (for total RNA extraction): Thermo Fisher Scientific #15596026
PrimeScript (trademark) RT reagent Kit (for cDNA synthesis): TaKaRa #RR037A
In-Fusion HD Cloning Kit: TaKaRa #Z9649N

### 4-3. Test Procedure

### Example 71

Hela cells were grown on 96-well plates and transfected with pEGFP-FKBP12 using Lipofectamine3000 (registered trademark) transfection reagent. After 24 hours, the transfected cells were incubated in fresh DMEM containing 0.01 mM AUTAC2-2G and in fresh DMEM containing DMSO as a comparative control for an additional 24 hours. The medium was then removed and replaced with 100 μL of live cell imaging solution. EGFP-FKBP12 levels were analyzed by measuring EGFP fluorescence.

### Example 72

The cells were treated and EGFP fluorescence was measured as in Example 71, except that the transfected cells were incubated in fresh DMEM with 0.1 mM AUTAC2-2G for an additional 24 hours.

### Example 73

The cells were treated and EGFP fluorescence was measured as in Example 71, except that the transfected cells were incubated in fresh DMEM with 1 mM AUTAC2-2G for an additional 24 hours.

### Example 74

The cells were treated and EGFP fluorescence was measured as in Example 71, except that the transfected cells were incubated in fresh DMEM with 10 mM AUTAC2-2G for an additional 24 hours.

### Comparative Example 2

The cells were treated and EGFP fluorescence was measured as in Example 72, except that the transfected cells were incubated in fresh DMEM with DMSO for an additional 24 hours.

### 4-4. Measurement Results

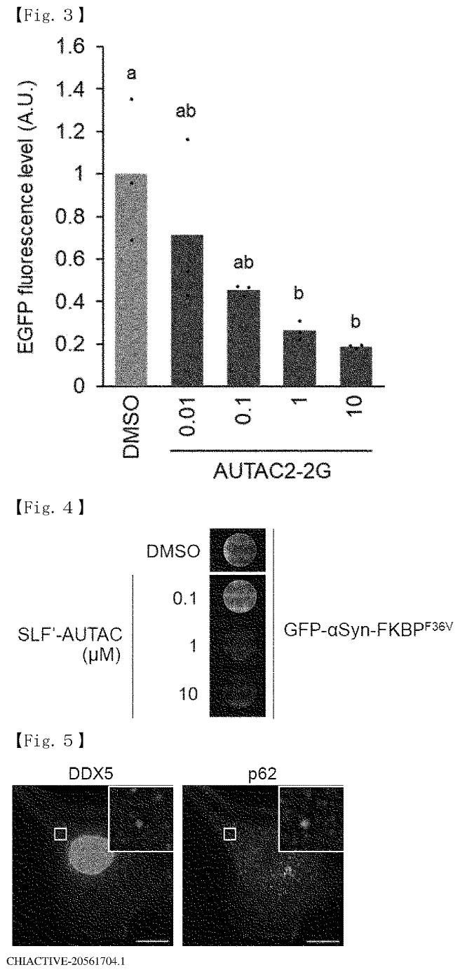

FIG. 3 shows EGFP fluorescence levels in the respective conditions. Each numerical value shows the mean EGFP fluorescence level from three independent experiments. Data with cut the same letters are statistically significant using Turkey-Kramer multiple comparison test. P values<0.01 were considered statistically significant. Data were normalized to the mean value of DMSO control.

When cells transfected with pEGFP-FKBP12 were cultured in a medium containing AUTAC2-2G, fluorescence levels reduced in an AUTAC2-2G concentration-dependent manner, and it was confirmed that AUTAC2-2G induces degradation of FKBP12 by autophagy. Therefore, a screening method of the present invention makes it possible to search for a novel compound that induces autophagy.

### Test 5. Measurement of Formation of Droplet Containing DEAD-Box RNA Helicase and p62

### Example 75

A compound SLF′-AUTAC, which was newly synthesized and confirmed to degrade synuclein by autophagy, was tested for whether to induce the formation of a droplet containing DDX5 and p62. The structure and synthesis scheme of SLF′-AUTAC and the summary of synuclein degradation test are as follows.

### Structural Formula of SLF′-AUTAC

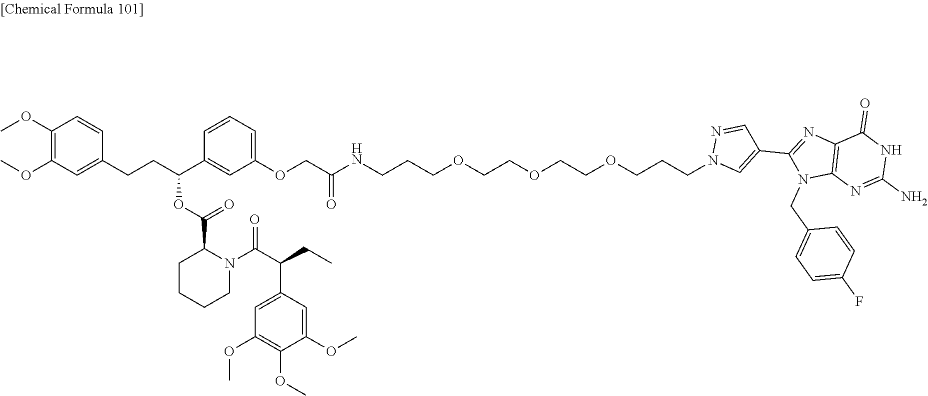 [SMILES: CC[C@H](C(=O)N1CCCC[C@H]1C(=O)O[C@H](CCc1ccc(OC)c(OC)c1)c1cccc(OCC(=O)NCCCOCCOCCOCCCn2cc(-c3nc4c(=O)[nH]c(N)nc4n3Cc3ccc(F)cc3)cn2)c1)c1cc(OC)c(OC)c(OC)c1]

### Synthesis Scheme of SLF′-AUTAC

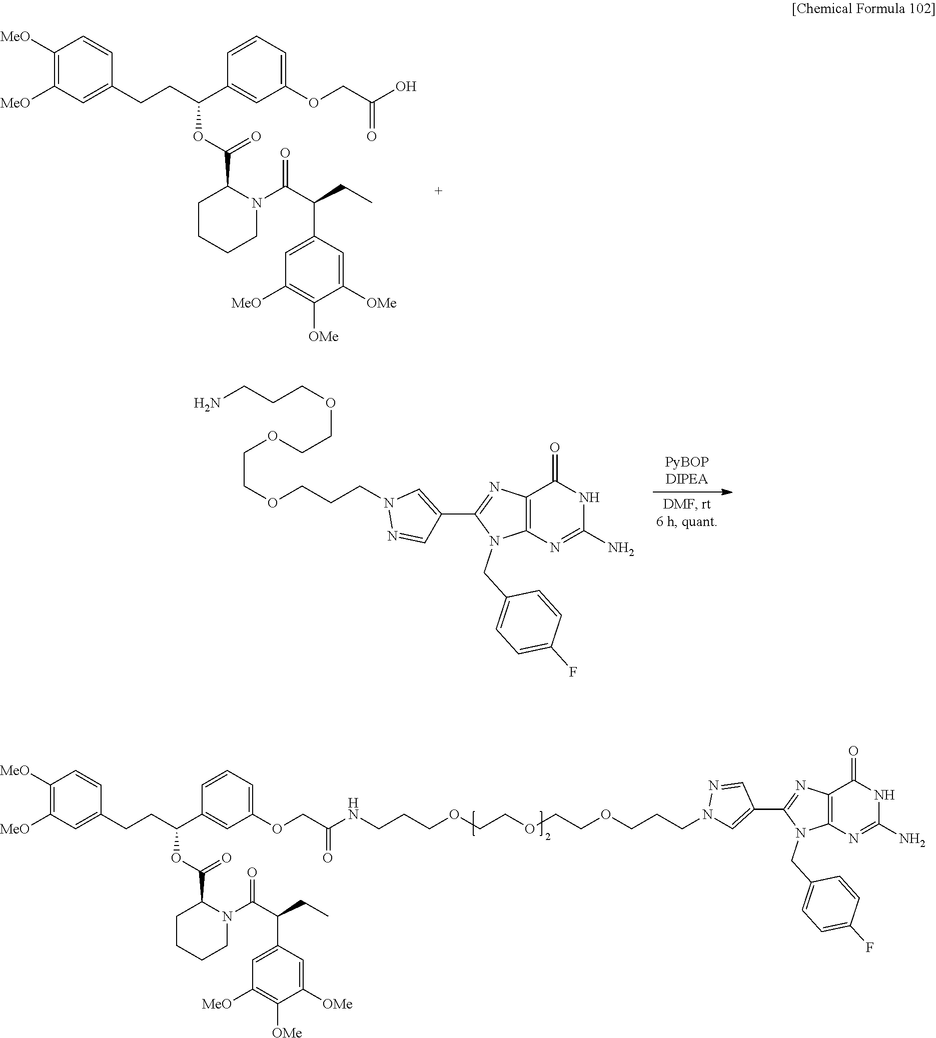

### Overview of Mutant FKBP-Tagged Synuclein Degradation Test by SLF′-AUTAC

HeLa cells transiently expressing GFP-α-synuclein tagged with a mutant FKBP (GFP-αSyn-FKBP^{F36V}) were treated with SLF′-AUTAC for 48 hours. In each of the wells of non-treated cells and treated cells, GFP fluorescence was photographed with a fluorescence microscope. Degradation of GFP-αSyn-FKBP^{F36V} was evaluated from the GFP fluorescence intensity. As shown in the fluorescence microscopic images of FIG. 4, in the cells treated with SLF′-AUTAC at 1 μM and 10 μM, the degradation of GFP-αSyn-FKBP^{F36V} was observed as compared with the untreated group (DMSO).

### Test Procedure

HeLa cells transiently expressing GFP tagged with a mutant FKBP (GFP-FKBP^{F36V}) were treated with 1 μM SLF′-AUTAC for 12 hours. The treated cells were fixed with 4% paraformaldehyde, then permeabilized with 0.1% TritonX-100/PBS, blocked with 1% BSA/PBS, immersed in PBS containing 1% rabbit-derived anti-DDX5 antibody and 1% mouse-derived anti-p62 antibody, and incubated at 4° C. overnight. Subsequently, the cells were immersed in PBS containing 1% AlexaFluor555-labeled anti-rabbit IgG and 1% AlexaFluor555-labeled anti-mouse IgG, and incubated at rccm temperature for 1 hour. The immunostained cells were observed with a fluorescence microscope.

### Test Results

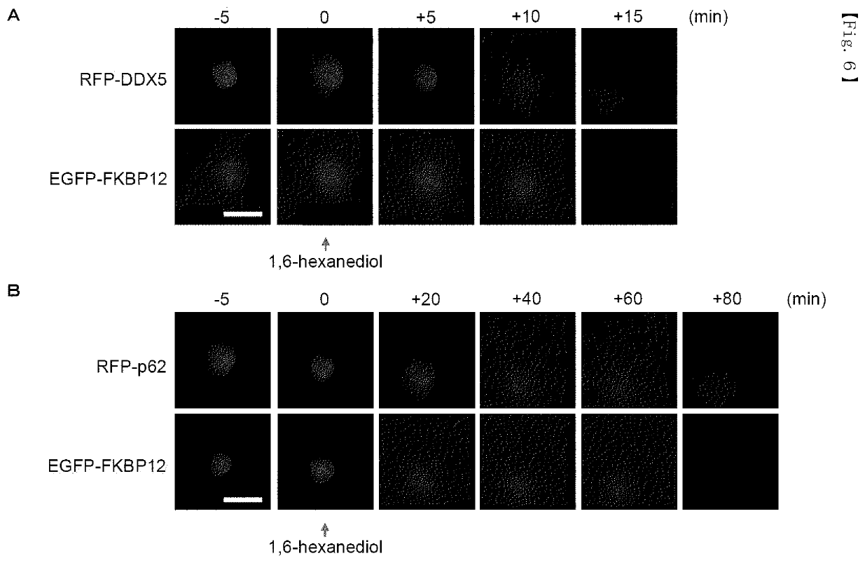

FIG. 5 shows images observed with a fluorescence microscope (scale bar: 10 μm).

By adding SLF′-AUTAC with FBnG structure to HeLa cells, it was observed that DDX5 and p62 formed droplets in the cells. In this experiment, DDX5 and p62 were observed after the cells were treated with SLF′-AUTAC and then immobilized, and DDX5 and p62 were observed at the same position in the cell, demonstrating that they were present in the same droplet.

### Example 76

### AUTAC2-2G was Used to Examine Whether it Induces the Formation of a Droplet Containing DDX5 and p62

### Test Procedure

HeLa cells transiently expressing GFP-FKBP12 and RFP-DDX5, or GFP-FKBP12 and RFP-p62 were treated with AUTAC2-2G added in such an amount as to achieve a concentration of 10 μM for 13 hours. The treated cells were subjected to live cell imaging by a fluorescence microscope, and fluorescence derived from GFP and RFP was continuously observed. On the way, 5% 1,6-hexanediol was added 5 minutes after the start of observation to observe whether a dot-like structure in the field of view disappeared.

1,6-Hexanediol is known to have an action of eliminating droplets, and this makes it possible to confirm whether the dot-like structure in the field of view of the microscope is a droplet.

### Test Results

FIG. 6 shows images observed with a fluorescence microscope (scale bar: 10 μm). FIG. 5A shows images observed with a fluorescence microscope in case Hela cells transiently expressing GFP-FKBP12 and RFP-DDX5 were treated with AUTAC2-2G, and FIG. 5B shows images observed with a fluorescence microscope in case HeLa cells transiently expressing GFP-FKBP12 and RFP-p62 were treated with AUTAC2-2G.

In each HeLa cell, d a dot-like structure containing GFP-FKBP12 and RFP-DDX5 and a dot-like structure containing GFP-FKBP12 and RFP-p62 were observed. These structures disappeared after addition of 1,6-hexanediol, and thus it was confirmed to be droplets.

1. A method for screening autophagy-inducing compounds, comprising:
(i) contacting test compound(s) one or more test compounds with a DEAD-box RNA helicase to measure a binding affinity of the test compound for the DEAD-box RNA helicase, and selecting a compound having a binding affinity greater than or equal to a predetermined value;
or
(ii) contacting one or more test compounds with a DEAD-box RNA helicase in the presence of p62, and (a) or (b):
(a) measuring an interaction between the DEAD-box RNA helicase and the p62, and selecting a compound that induces the interaction; or
(b) measuring the formation of a droplet containing the DEAD-box RNA helicase and the p62, and selecting a compound that induced the formation of the droplet.

2. (canceled)

3. The method according to claim 1, wherein the DEAD-box RNA helicase and the p62 are expressed in a cell and the cell is contacted with the one or more test compounds.

4. (canceled)

5. The method according to claim 1, wherein the DEAD-box RNA helicase is DDX5 or DDX17.

6. A method for producing a compound that induces degradation of a target intracellular molecule or organelle by autophagy, comprising:
(I) screening for autophagy-inducing compounds by:
(i) contacting one or more test compounds with a DEAD-box RNA helicase to measure a binding affinity of the test compound for the DEAD-box RNA helicase, and selecting a compound having a binding affinity greater than or equal to a predetermined value;
or
(ii) contacting one or more test compounds with a DEAD-box RNA helicase in the presence of p62, and (a) or (b):
(a) measuring an interaction between the DEAD-box RNA helicase and the p62 and selecting a compound that induced the interaction; or
(b) measuring the formation of a droplet containing the DEAD-box RNA helicase and the p62 and selecting a compound that induced the formation of the droplet; and
(II) linking a ligand that specifically binds to the target intracellular molecule or organelle to the resulting selected compound via a linker to produce the compound that induces degradation of a target intracellular molecule or organelle by autophagy.

7. The method according to claim 6, further comprising the step of:
(III) contacting the compound to which the ligand is linked that induces degradation of a target intracellular molecule or organelle by autophagy with a cell having the target intracellular molecule or organelle to confirm the degradation of the target intracellular molecule or organelle by autophagy.

8. A method for inducing degradation of a target intracellular molecule or organelle by autophagy comprising:
contacting the compound that induces degradation of a target intracellular molecule or organelle by autophagy of claim 6 with a DEAD-box RNA helicase in the presence of p62 in a cell.

9. The method according to claim 8, wherein the compound that induces degradation of a target intracellular molecule or organelle by autophagy is contacted with a DEAD-box RNA helicase in the presence of p62 in a cell to form a droplet containing the compound, the p62 and the DEAD-box RNA helicase.

10. The method according to claim 8, wherein the method prevents or treats cancer or a cell degenerative disease.

11. The method according to claim 9, wherein the method prevents or treats cancer or a cell degenerative disease.
